(19) United States
(12) Patent Application Publication
(10) Pub. No.: US 2022/0098234 A1
(43) Pub. Date: Mar. 31, 2022

(54) METHODS OF PROTEIN PURIFICATION

(71) Applicant: Ohio State Innovation Foundation, Columbus, OH (US)

(72) Inventors: Ivan SUSIN PIRES, Piracicaba, Sao Paulo (BR); Andre PALMER, Bexley, OH (US); Donald BELCHER, Columbus, OH (US)

(21) Appl. No.: 17/427,547

(22) PCT Filed: Jan. 31, 2020
(86) PCT No.: PCT/US2020/016267

§ 371 Date: Jul. 30, 2021

Related U.S. Application Data
(60) Provisional application No. 62/940,439, filed on Nov. 26, 2019.
(60) Provisional application No. 62/875,952, filed on Jul. 18, 2019.
(60) Provisional application No. 62/800,129, filed on Feb. 1, 2019.

(51) Int. Cl. C07K 1/34; C07K 1/36

(52) U.S. Cl. CPC C07K 1/34; C07K 14/775; C07K 1/36

(57) ABSTRACT

Provided herein are methods for purifying proteins from mixtures of proteins using ultrafiltration, such as tangential-flow filtration (TFF). Provided herein are methods for isolating an apoprotein from a protein solution comprising a conjugated protein, wherein the conjugated protein comprises the apoprotein and a hydrophobic ligand associated with the apoprotein.

### CROSS REFERENCE TO RELATED APPLICATIONS

This application claims the benefit of priority of U.S. Provisional Application No. 62/800,129,589, filed Feb. 1, 2019, U.S. Provisional Application No. 62/875,952, filed Jul. 18, 2019, and U.S. Provisional Application No. 62/940,439, filed Nov. 26, 2019, each of which is incorporated by reference herein in its entirety.

### STATEMENT REGARDING FEDERALLY SPONSORED RESEARCH OR DEVELOPMENT

This invention was made with Government Support under Grant Nos. R56HL123015, R01HL126945, R01EB021926, and R01HL138116 awarded by the National Institutes of Health. The Government has certain rights in the invention.

### BACKGROUND

Several methods are currently available to separate and purify molecules of biological interest, such as proteins, from mixtures thereof. Existing methods for the purification of proteins include using precipitation agents such as salts (e.g., ammonium sulfate), non-salt substances such as polyethylene glycol (PEG), organic solvents, or altering pH. Unfortunately, in many cases the protein yields are less than desired, the methods are often time consuming, employ undesirable solvents, and/or require large capital investments due to the use of specialized equipment such as large centrifuges. Also, it is often difficult to scale up a precipitation method and, even if this can be done, dissolution of the resulting precipitate can be slow and is not always complete.

An alternative method for protein purification is affinity chromatography. In affinity chromatography, proteins are separated on the basis of specific and selective binding of a desired molecule to an affinity matrix or stationary phase. Affinity matrices typically include a ligand-binding moiety immobilized on a gel support. For example, affinity chromatography can be used to purify hemoglobin and its chemically modified derivatives based on the fact that native hemoglobin binds specifically to polyanionic moieties of certain affinity matrices. In this process, unmodified hemoglobin is retained by the affinity matrix, while chemically modified hemoglobin, which cannot bind as avidly to the matrix because its polyanion binding site is covalently occupied by the modifying agent, is eluted. Furthermore, the state-of-the-art method for monoclonal antibody purification is to use protein A affinity chromatography in which the antibodies bind to protein A, while impurities are washed from the immobilized support. Affinity chromatography columns are highly specific and thus yield very pure products; however, affinity chromatography is a relatively expensive process and can also be difficult to scale up. Furthermore, the commonly used protein A affinity columns require harsh denaturing steps to unbind the antibody from protein A.

Accordingly, improved methods for purifying proteins are needed.

### SUMMARY

Provided herein are methods for isolating an apoprotein from a protein solution comprising a conjugated protein, wherein the conjugated protein comprises the apoprotein and a hydrophobic ligand associated with the apoprotein. Methods of isolating the apoprotein protein can comprise (i) contacting the conjugated protein with an aqueous solution comprising a water-miscible solvent and a pH modifier, thereby forming a protein solution having a pH of less than 6.5 or greater than 8; and (ii) filtering the protein solution by ultrafiltration against a filtration membrane having a pore size that separates the apoprotein from the hydrophobic ligand, thereby forming a retentate fraction comprising the apoprotein and a permeate fraction comprising the hydrophobic ligand. In some embodiments, methods for isolating an apoprotein can further comprise (iii) neutralizing the retentate fraction to isolate the apoprotein.

In some cases, the hydrophobic ligand can be non-covalently associated with the apoprotein. In some cases, the hydrophobic ligand can be ionically or electrostatically associated with the apoprotein. In some cases, the hydrophobic ligand can be covalently associated with the apoprotein. In some examples, the conjugated protein can comprise a lipoprotein, the apoprotein can comprise an apolipoprotein, and the hydrophobic ligand can comprise a lipid. In some examples, the conjugated protein can comprise isolated human serum proteins, the apoprotein can comprise a lipid-binding protein such as human serum albumin and the hydrophobic ligand can comprise lipids. In some examples, the conjugated protein can comprise a heme protein, the apoprotein can comprise an apo-heme protein such as apohemoglobin, and the hydrophobic ligand can comprise a heme.

The protein solution can have an acidic or basic pH, selected so as to facilitate dissociation of the hydrophobic ligand and the apoprotein. In some cases, the protein solution can have a pH of 6 or less (e.g., a pH of from 2 to 6, such as from 3 to 6). In other cases, the protein solution can have a pH greater than 8 (e.g., a pH of from greater than 8 to 11).

Generally, the filtration membrane can have a range of pore sizes effective to separate the hydrophobic ligand from the apoprotein (e.g., a pore size which allows the hydrophobic ligand to pass through the filtration membrane but retains the apoprotein). For example, the filtration membrane can be rated for retaining solutes having a molecular weight ranging from the molecular weight of the hydrophobic ligand to the molecular weight of the apoprotein. By way of example, in embodiments where the conjugated protein is hemoglobin, the filtration membrane can be rated for retaining solutes having a molecular weight ranging from the molecular weight of the heme to the molecular weight of the apohemoglobin (e.g., such as a membrane rated for retaining solutes having a molecular weight of from about 1 kDa to 100 kDa, such as from about 1 kDa to about 10 kDa). In some cases, the filtration membrane can be rated for retaining solutes having a molecular weight of from about 1 kDa to 4,000 kDa, such as from about 1 kDa to about 1,000 kDa or from about 1 kDa to about 500 kDa.

In certain embodiments, the ultrafiltration modality can comprise tangential-flow filtration (TFF).

In some examples, the ligand can comprise a prosthetic group, a cofactor, a lipid, a metabolite, or a combination thereof.

In some examples, the conjugated protein can comprise a heme protein. For example, in some embodiments, the conjugated protein can comprise hemoglobin, the apoprotein can comprise apohemoglobin, and the ligand can comprise heme. In these embodiments, the hemoglobin can be present in the protein solution (e.g., unfolded) at a concentration from 0.1 mg/mL to 10 mg/mL, such as from 0.1 mg/mL to 5 mg/mL or from 0.5 mg/mL to 3 mg/mL. In other embodiments, the conjugated protein can comprise human serum albumin (HSA). In these embodiments, the HSA can be present in the protein solution (e.g., unfolded) at a concentration from 0.5 mg/mL to 25 mg/mL, such as from 1 mg/mL to 20 mg/mL.

The water-miscible solvent can comprise a polar protic solvent. In some embodiments, the water-miscible solvent can comprise an alcohol (e.g., ethanol, methanol, or a combination thereof). In some embodiments, the aqueous solution can comprise from 10% to 90% by volume alcohol. In one example, the conjugated protein can comprise hemoglobin and the aqueous solution can comprise from 60% to 90% by volume alcohol (e.g., 60% to 90% by volume ethanol). In another example, the conjugated protein can comprise HSA and the aqueous solution can comprise from 30% to 60% by volume alcohol.

In some embodiments, filtering step (ii) can comprise buffer exchange. In certain embodiments, filtering step (ii) can comprise continuous diafiltration or dialysis.

Optionally, the retentate fraction can be spectroscopically monitored during the continuous diafiltration to monitor separation of the hydrophobic ligand from the apoprotein. Spectroscopically monitoring the retentate fraction can comprise monitoring a spectroscopic peak associated with the apoprotein and a spectroscopic peak associated with the conjugated protein. In some embodiments, filtering step (ii) can comprise performing the continuous diafiltration until a relative magnitude of the spectroscopic peak associated with the apoprotein and the spectroscopic peak associated with the conjugated protein suggest that the apoprotein and the conjugated protein are present in the retentate fraction at a molar ratio of at least 9:1.

Methods for isolating an apoprotein can further comprise (iii) neutralizing the retentate fraction to isolate the apoprotein. In some embodiments, neutralizing step (iii) can comprise continuous diafiltration with water (e.g., deionized water) or a buffer solution having a pH of from 6.8 to 7.6. In certain embodiments, the apoprotein isolated in step (iii) can comprise less than 1% residual hydrophobic ligand relative to the concentration of apoprotein isolated in step (iii), as measured by a suitable spectroscopic technique (e.g., UV Vis spectroscopy).

The apoprotein isolated in step (iii) can exhibit excellent storage stability relative to apoproteins isolated using other conventional methodologies. In some embodiments, the apoprotein isolated in step (iii) can be stable for a period of at least 7 days at 22° C. In certain embodiments, at least 75% of the apoprotein remains soluble in solution after storage at 22° C. for 7 days. In certain embodiments, at least 75% of the apoprotein remains soluble in solution after storage at 4° C. for 180 days. In certain embodiments, at least 75% of the apoprotein remains soluble in solution after storage at −80° C. for 180 days.

In certain embodiments, the apoprotein can comprise apohemoglobin. In some of these embodiments, at least 65% of the apohemoglobin can retain its activity (i.e., retain its ability to bind heme) after storage at 22° C. for 7 days. In some of these embodiments, at least 65% of the apohemoglobin can retain its activity (i.e., retain its ability to bind heme) after storage at 4° C. for 180 days. In some of these embodiments, at least 65% of the apohemoglobin can retain its activity (i.e., retain its ability to bind heme) after storage at −80° C. for 180 days.

In some embodiments, methods can further comprise lyophilizing the apoprotein isolated in step (iii).

Also described are methods of isolating a ligand from a protein solution comprising a conjugated protein. These methods can comprise (i) mildly denaturing the conjugated protein to form a protein solution; and (ii) filtering the protein solution by ultrafiltration against a filtration membrane having a pore size that separates the apoprotein from the hydrophobic ligand, thereby forming a retentate fraction comprising the apoprotein and a permeate fraction comprising the hydrophobic ligand. In some embodiments, methods for isolating an apoprotein can further comprise (iii) neutralizing the retentate fraction to isolate the apoprotein.

In some examples, mildly denaturing the conjugated protein can comprise heating the conjugated protein (e.g., to a temperature of from 40° C. to 60° C.). In some examples, mildly denaturing the conjugated protein can comprise contacting the conjugated protein with a pH modifier (e.g., with an acid and/or a base). In some examples, mildly denaturing the conjugated protein can comprise contacting the conjugated protein with a non-aqueous solvent, such as an alcohol. In some examples, mildly denaturing the conjugated protein can comprise contacting the conjugated protein with a chaotropic agent, such as guanidinium chloride, lithium perchlorate, lithium acetate, magnesium chloride, calcium chloride, sodium dodecyl sulfate, thiourea, urea, or a combination thereof.

Also provided is apohemoglobin produced by the filtration methods described herein. Apohemoglobin prepared by the filtration methods described herein can exhibit improved storage stability and purity as compared to apohemoglobin prepared by existing precipitation or liquid-liquid extraction methodologies. In some embodiments, the apohemoglobin produced by the filtration methods described herein can be storage stable for a period of at least 7 days at 22° C. In certain embodiments, at least 75% of the apohemoglobin produced by the filtration methods described herein remains soluble in solution after storage at 22° C. for 7 days. In certain embodiments, at least 75% of the apohemoglobin produced by the filtration methods described herein remains soluble in solution after storage at 4° C. for 180 days. In certain embodiments, at least 75% of the apohemoglobin produced by the filtration methods described herein remains soluble in solution after storage at −80° C. for 180 days. In certain embodiments, at least 65% of the apohemoglobin can retain its activity (i.e., retain its ability to bind heme) after storage at 22° C. for 7 days. In certain embodiments, at least 65% of the apohemoglobin can retain its activity (i.e., retain its ability to bind heme) after storage at 4° C. for 180 days. In certain embodiments, at least 65% of the apohemoglobin can retain its activity (i.e., retain its ability to bind heme) after storage at −80° C. for 180 days. In certain embodiments, apohemoglobin produced by the filtration methods described herein can be characterized by a Soret peak having a maximum absorption ranging from 411 to 417 nm (after renaturation/neutralization).

Also provided herein are methods for isolating haptoglobin from plasma or a fraction thereof. In some embodiments, methods for isolating haptoglobin from plasma or a fraction thereof can comprise (i) clarifying the plasma or fraction thereof; and (ii) filtering the plasma or a fraction thereof by ultrafiltration against a filtration membrane, thereby forming a retentate fraction comprising haptoglobin having a molecular weight of greater than about 100 kDa and a permeate fraction comprising serum proteins and other impurities having a molecular weight of less than about 100 kDa.

The plasma or fraction thereof can comprise plasma fraction IV, plasma fraction V, a fraction of precipitated plasma (from salting out, or equivalent), or a combination thereof.

Clarifying the plasma or a fraction thereof can comprise removing suspended solids from the plasma or fraction thereof. Removing suspended solids from the plasma or fraction thereof can comprise filtering the (via ultrafiltration, microfiltration, depth filtration or equivalent) plasma or a fraction thereof, contacting the plasma or a fraction thereof with a salting out agent (e.g., ammonium sulfate), an adsorbing agent (e.g., ethacridine lactate), or a combination thereof. Further clarification may be implemented through addition of a lipid-binding agent such as fumed silica (such as fumed silica sold under the tradename Aerosil 380®, or similar), clay, bentonite, terra alba, active carbon, or a combination thereof.

In some embodiments, the ultrafiltration can comprise tangential-flow filtration (TFF).

In some cases, the method can further comprise filtering the permeate fraction comprising serum proteins and other impurities by ultrafiltration against a second filtration membrane, thereby forming a second retentate fraction comprising a blend of proteins having a molecular weight below about 100 kDa and above a cutoff value and a second permeate fraction comprising serum proteins and other impurities having a molecular weight below the cutoff value, wherein the blend of proteins comprises low molecular weight haptoglobin, transferrin, hemopexin, albumin, or a combination thereof. The cutoff value can be from about 20 kDa to about 70 kDa, such as from about 25 kDa to about 50 kDa

The haptoglobin-containing fractions prepared by these methods can be used in therapeutic applications. For example, haptoglobin-containing fractions can be administered in a subject in need thereof to sequester cell-free hemoglobin. Accordingly, these fractions can be administered to a subject in need thereof, for example, to treat hemolytic anemia and other conditions characterized by or associated with hemolysis (e.g., sickle cell anemia, malaria, red blood cell transfusions, thalassemia, autoimmune disorders, bone marrow failure, infections, surgery, severe burns, acute lung injury, the administration of chemotherapeutics, radiation therapy, etc.). These proteins may also be used in wound-healing applications and to prevent bacterial proliferation/resistance.

In the methods described above, the second retentate fraction can include a blend of proteins (e.g., low molecular weight haptoglobin, transferrin, hemopexin, albumin, or a combination thereof) that can bind and detoxify cell-free hemoglobin, free iron, and free heme. Accordingly, this fraction can be administered to a subject in need thereof, for example, to treat hemolytic anemia and other conditions characterized by or associated with hemolysis (e.g., sickle cell anemia, malaria, red blood cell transfusions, thalassemia, autoimmune disorders, bone marrow failure, infections, surgery, severe burns, acute lung injury, the administration of chemotherapeutics, radiation therapy, etc.). These proteins may also be used in wound-healing applications and to prevent bacterial proliferation/resistance.

In other embodiments, methods for isolating haptoglobin from plasma or a fraction thereof can comprise (i) filtering the plasma or fraction thereof by ultrafiltration against a first filtration membrane, thereby forming a first retentate fraction comprising serum proteins having a molecular weight above a first cutoff value (e.g., selected so as to retain a minimal amount of Hp) and a first permeate fraction comprising the haptoglobin and serum proteins having a molecular weight below the first cutoff value; and (ii) filtering the first permeate fraction by ultrafiltration against a second filtration membrane, thereby forming a second retentate fraction comprising Hp2-1, Hp2-2, and serum proteins having a molecular weight below the first cutoff value and above a second cutoff value; and a second permeate fraction comprising Hp2-1, Hp2-2, and serum proteins having a molecular weight below the second cutoff value. In some cases, the method can further comprise (iii) filtering the second permeate fraction by tangential-flow filtration against a third filtration membrane, thereby forming a third retentate fraction comprising Hp2-1 and Hp2-2 having a molecular weight below the second cutoff value and above a third cutoff value; and a third permeate fraction comprising low molecular weight haptoglobin, serum proteins and other impurities having a molecular weight below the third cutoff value. In some cases, the method can further comprise (iv) filtering the third permeate fraction comprising low molecular weight haptoglobin, serum proteins and other impurities by ultrafiltration against a fourth filtration membrane, thereby forming a fourth retentate fraction comprising a blend of proteins having a molecular weight below the third cutoff value and above a fourth cutoff value and a fourth permeate fraction comprising serum proteins and other impurities having a molecular weight below the fourth cutoff value, wherein the blend of proteins comprises low molecular weight haptoglobin, transferrin, hemopexin, albumin, or a combination thereof.

The first cutoff value can be from about 650 kDa to about 1000 kDa. The second cutoff value can be from about 300 kDa to about 700 kDa. The third cutoff value can be from about 70 kDa to about 200 kDa. The fourth cutoff value can be from about 20 kDa to about 70 kDa. In certain examples, the first cutoff value can be about 750 kDa, the second cutoff value can be about 500 kDa, and the third cutoff value can be about 100 kDa. The fourth cutoff value can be about 30 kDa or about 50 kDa.

The plasma or fraction thereof can comprise plasma fraction IV, plasma fraction V, fraction of precipitated plasma (from salting out, or equivalent) or a combination thereof.

Clarifying the plasma or a fraction thereof can comprise removing suspended solids from the plasma or fraction thereof. Removing suspended solids from the plasma or fraction thereof can comprise filtering the plasma (via ultrafiltration, microfiltration, depth filtration or equivalent) or a fraction thereof, contacting the plasma or a fraction thereof with a salting out agent (e.g., ammonium sulfate), an adsorbing agent (e.g., ethacridine lactate), or a combination thereof. Further clarification may be implemented through addition of a lipid-binding agent such as fumed silica (such as fumed silica sold under the tradename Aerosil 380®, or similar), clay, bentonite, terra alba, active carbon, or a combination thereof.

In some embodiments, the ultrafiltration can comprise tangential-flow filtration.

In these methods, the fourth retentate fraction can include a blend of proteins (e.g., low molecular weight haptoglobin, transferrin, hemopexin, albumin, or a combination thereof) that can bind and detoxify free hemoglobin, free iron, and free heme. Accordingly, this fraction can be administered to a subject in need thereof, for example, to treat hemolytic anemia and other conditions characterized by or associated with hemolysis (e.g., sickle cell anemia, malaria, red blood cell transfusions, thalassemia, autoimmune disorders, bone marrow failure, infections, surgery, severe burns, the administration of chemotherapeutics, radiation therapy, etc.). These proteins may also be used in wound-healing applications and to prevent bacterial proliferation/resistance.

Also provided herein are methods for isolating a target protein from a solution comprising a plurality of proteins that exploit molecular size changes induced by protein complex formation. Such methods can comprise (i) filtering the protein solution by ultrafiltration against a first filtration membrane, thereby forming a first retentate fraction comprising impurities having a molecular weight above a first cutoff value and a first permeate fraction comprising the target protein and impurities having a molecular weight below the first cutoff value; (ii) contacting the first permeate fraction with a binding molecule that selectively associates with the target protein to form a target protein complex having a molecular weight above the first cutoff value; and (iii) filtering the first permeate fraction by ultrafiltration against a second filtration membrane, thereby forming a second retentate fraction comprising the target protein complex having a molecular weight above the first cutoff value and a second permeate fraction comprising the impurities having a molecular weight below the first cutoff value.

In some embodiments, the method can further comprise (iv) contacting the second retentate fraction with a dissociating agent, thereby inducing dissociation of the target protein complex to the target protein and the binding molecule.

In some embodiments, the method can further comprise (v) filtering the second retentate fraction to separate the target protein from the binding molecule and the dissociating agent, thereby isolating the target protein. In some of these embodiments, step (v) can comprise filtering the second retentate fraction by ultrafiltration against a third filtration membrane, thereby forming a third retentate solution comprising the target protein having a molecular weight above a second cutoff value and a second permeate fraction comprising the impurities having a molecular weight below the second cutoff value.

The binding molecule can comprise, for example, an antibody, antibody fragment, antibody mimetic, peptide, protein (e.g., protein A), oligonucleotide, DNA, RNA, aptamer, organic molecule, intein, split-intein, or combination thereof. The dissociating agent can comprise, for example, a pH modifier, a salt, a polyelectrolyte, a chaotropic agent, a non-aqueous solvent, or a combination thereof.

In some embodiments, the ultrafiltration can comprise tangential-flow filtration.

### DESCRIPTION OF DRAWINGS

FIG. 1 is a schematic illustration of processes used to produce active apoHb.

FIG. 2 is a schematic illustration of the apoHb TFF production process.

FIG. 3A shows the absorbance spectra of apoHb, DCNh and rHbCN

FIG. 3B shows DCNh-titration assay plots for apoHb produced via EtOH-TFF. The top graph corresponds to the processed data of the equilibrium 420 nm absorbance of a fixed apoHb concentration with increasing DCNh concentration (major and minor lines fits are shown to demonstrate the presence of an inflection point, which corresponds to the concentration of active apoHb on a per heme basis). The middle graph shows the absorbance values subtracted by the pure DCNh absorbance to highlight the inflection point determined by the apoHb assay. The bottom graph presents the residuals of the major and minor line fits.

FIG. 3C shows DCNh-titration assay plots for apoHb produced by acetone extraction. The top graph corresponds to the processed data of the equilibrium 420 nm absorbance of a fixed apoHb concentration with increasing DCNh concentration (major and minor lines fits are shown to demonstrate the presence of an inflection point, which corresponds to the concentration of active apoHb on a per heme basis). The middle graph shows the absorbance values subtracted by the pure DCNh absorbance to highlight the inflection point determined by the apoHb assay. The bottom graph presents the residuals of the major and minor line fits.

FIG. 4A shows the electrospray mass spectra of Hb under native conditions. Dotted lines indicate deconvoluted spectra. The superscripts (α/β)^{a} and (α/β)^{h} indicate the apo- or holo-protein, respectively.

FIG. 4B shows the electrospray mass spectra of Hb under acidic/denaturing conditions. Dotted lines indicate deconvoluted spectra. The superscripts (α/β)^{a} and (α/β)^{h} indicate the apo- or holo-protein, respectively.

FIG. 4C shows the electrospray mass spectra of apoHb under native conditions. Dotted lines indicate deconvoluted spectra. The superscripts (α/β)^{a} and (α/β)^{h} indicate the apo- or holo-protein, respectively.

FIG. 4D shows the electrospray mass spectra of apoHb under acidic/denaturing conditions. Dotted lines indicate deconvoluted spectra. The superscripts (α/β)^{a} and (α/β)^{h} indicate the apo- or holo-protein, respectively.

FIG. 5A shows the SEC profile of TFF-apoHb and Hb (the elution peak of Hb with UV-visible detection at 280 nm was normalized to a value of 1, and all other values were normalized so that the same mass of apoHb and Hb was shown on the chromatograms).

FIG. 5B compares the SEC elution volume of apoHb-TFF with molecular weight (MW) standards (conalbumin 76 kDa, human Hb 64 kDa, carbonic anhydrase 29 kDa, ribonuclease A 14 kDa, and aprotinin 6.5 kDa).

FIG. 5C shows apoHb samples used for residual heme analysis.

FIG. 5D shows a data table with results from the residual heme analysis of apoHb samples.

FIG. 5E shows a plot of residual heme content with cutoff curves representing 1% and 0.5% residual heme in solution.

FIG. 5F shows the SDS-PAGE of apoHb and Hb.

FIG. 5G shows the SEC profiles of apoHb, Hp and apoHb-Hp mixtures with excess Hp and excess apoHb.

FIG. 5H shows the SEC profiles within the elution region of interest of apoHb, Hp and apoHb-Hp mixtures with excess Hp and excess apoHb.

FIG. 6A shows the SEC-HPLC of concentrated (con) and unconcentrated (uncon) TFF-apoHb samples.

FIG. 6B shows the SEC-HPLC of concentrated (con) and unconcentrated (uncon) TFF-apoHb samples within the elution region of interest and magnified 10× for the tetrameric apoHb elution region.

FIG. 6C shows the RP-HPLC of concentrated (con) and unconcentrated (uncon) TFF-apoHb samples.

FIG. 6D shows the far UV CD of concentrated (con) and unconcentrated (uncon) TFF-apoHb samples.

FIG. 7A show the absorbance spectra of native Hb, pure heme, and hemichrome (the molar concentration on a per heme basis for each species was approximately 60 μM).

FIG. 7B show the absorbance spectra of rHb from TFF-apoHb, pure heme, and hemichrome (the molar concentration on a per heme basis for each species was approximately 60 μM).

FIG. 7C shows a schematic of the hemichrome removal process.

FIG. 7D shows representative absorbance spectra of rHb at each stage of the hemichrome removal process with curve fits from the spectral deconvolution software standardized to a total heme concentration of 25 μM.

FIG. 7E shows the final results from spectral deconvolution at each stage of the hemichrome removal process. The lines trace the fate of unwanted species (i.e. heme and hemichromes).

FIG. 8A show O₂ equilibrium curves for native Hb, and rHb from TFF-apoHb and acetone extraction methods. Native hHb, TFF rHb and acetone rHb had P₅₀s of 11.33±0.02, 11.52±0.02 and 10.50±0.02 mm Hg and n of 2.60±0.01, 2.37±0.01 and 2.14±0.01, respectively.

FIG. 8B is a plot showing the representative O₂ dissociation kinetic time courses for native Hb and rHb produced via TFF. Data was fit to a single exponential equation to yield k_{off,O2} of 35.8±0.2 s⁻¹ and 36.8±0.3 s⁻¹ for TFF rHb and native Hb, respectively.

FIG. 8C shows representative CO association kinetic time courses for native Hb and TFF rHb.

FIG. 8D is a plot of kₐₚₚ for CO association at varying CO concentrations. Data was fit to a linear function to regress k_{on,CO} of 180±7 and 175±4 nM/s for native Hb and rHb, respectively.

FIG. 9A shows the storage of apoHb in liquid form at 37° C. Samples were stored at initial apoHb concentrations of either 33.80±0.36 mg/mL active apoHb with 41.4±2.77 mg/mL total protein (con) or 1.47±0.01 mg/mL active apoHb with 1.99±0.17 mg/mL total protein (uncon).

FIG. 9B shows the storage of apoHb in liquid form at 22° C. Samples were stored at initial apoHb concentrations of either 33.80±0.36 mg/mL active apoHb with 41.4±2.77 mg/mL total protein (con) or 1.47±0.01 mg/mL active apoHb with 1.99±0.17 mg/mL total protein (uncon).

FIG. 9C shows the storage of apoHb in liquid form at 4° C. Samples were stored at initial apoHb concentrations of either 33.80±0.36 mg/mL active apoHb with 41.4±2.77 mg/mL total protein (con) or 1.47±0.01 mg/mL active apoHb with 1.99±0.17 mg/mL total protein (uncon).

FIG. 9D shows the storage of apoHb at −80° C. Samples were stored at initial apoHb concentrations of either 33.80±0.36 mg/mL active apoHb with 41.4±2.77 mg/mL total protein (con) or 1.47±0.01 mg/mL active apoHb with 1.99±0.17 mg/mL total protein (uncon).

FIG. 9E shows the storage of apoHb in lyophilized form at −80° C.

FIG. 10A shows the RP-HPLC of stored TFF-apoHb.

FIG. 10B shows the far UV CD of stored TFF-apoHb.

FIG. 10C shows the change in UV-visible spectra of TFF-apoHb at 22° C. as a function of storage time.

FIG. 10D shows the change in UV-visible spectra of stored and fresh TFF-apoHb at 22° C.

FIG. 10E shows the SEC-HPLC of TFF-apoHb stored for more than one year at 4° C. and after transferring the year-old samples to storage at 22° C.

FIG. 10F shows the SEC-HPLC within the elution region interest of TFF-apoHb stored for more than one year at 4° C. and after transferring the year-old samples to storage at 22° C. (with 10× magnification of the elution region of tetrameric apoHb).

FIG. 11A shows the full SEC-HPLC chromatogram of a single TFF-apoHb sample at different concentrations and injection volumes.

FIG. 11B shows FIG. 11A in the region of interest for TFF-apoHb tetramers and dimers.

FIG. 11C shows the decrease in tetrameric TFF-apoHb content upon addition of Hp to the sample.

FIG. 11D shows the elution volume shift to higher elution volumes for Hb and TFF-apoHb at low protein concentrations.

FIG. 12 schematically illustrates the general process for the purification of haptoglobin via tangential flow filtration of Cohn Fraction IV without the use of fumed silica (grey arrows indicate the fluid flow direction).

FIG. 13 schematically illustrates the general process for the purification of haptoglobin via tangential flow filtration of Cohn Fraction IV using fumed silica (grey arrows indicate the fluid flow direction).

FIG. 14 schematically illustrates the general process used to purify both Hp (Stages 2 and 3), and the Hb, heme and iron scavenging protein cocktail (Stage 4). HMW=high MW fraction. LMW=low MW fraction. Arrows indicate the direction of flow.

FIG. 15A shows a representative graph for the quantification of hemoglobin binding capacity (HbBC) of Hp samples based on fluorescence titration.

FIG. 15B shows a representative graph for the quantification of hemoglobin binding capacity (HbBC) of Hp samples based on the SEC-HPLC AUC method.

FIG. 16A is a plot showing the analysis of HbBC and total protein recovery at each stage of processing based on the TFF method without the use of fumed silica.

FIG. 16B shows the HPLC-SEC analysis at each stage of the Hp purification process via tangential flow filtration of Cohn Fraction IV. Dotted lines show the Hp-Hb complexes formed with the samples exposed to excess Hb. The chromatogram of samples exposed to excess Hb (dotted lines) ends prior to the elution time of free Hb (9.6 min) given that the signal from excess free Hb peak would overwhelm the signal from the Hp-Hb peak.

FIG. 17A shows SDS-PAGE under non-reducing conditions at each of the processing stages of Hp purification via TFF of Cohn Fraction IV without the use of fumed silica. Lanes were loaded with 30 μg of protein. Densitometric analysis indicated Hp eluting bands composed of >70% of Stage 2 proteins and >75% of Stage 3 proteins.

FIG. 17B shows SDS-PAGE under reducing conditions at each of the processing stages of Hp purification via TFF of Cohn Fraction IV without the use of fumed silica. Lanes were loaded with 27 μg of protein. Densitometric analysis indicated Hp eluting bands composed of >70% of Stage 2 proteins and >75% of Stage 3 proteins.

FIG. 17C shows the normalized total ion intensity from trypsin digestion mass spectrometry at each of the processing stages of Hp purification via TFF of Cohn Fraction IV without the use of fumed silica. Abbreviations: AT: α-1 antitrypsin, ACT: α-1 antichymotrypsin, Hb: hemoglobin, Tf: transferrin, ApoA1: apolipoprotein A1, Hpr: haptoglobin-related protein, ApoA2: apolipoprotein A2, ApoJ: apolipoprotein J, HSA: human serum albumin, Hp: haptoglobin, A2M: α-2 macroglobulin, Cp: ceruloplasmin, ITH4: Inter-alpha-trypsin inhibitor H4, IgHA1: immunoglobulin heavy constant alpha 1, IgKC: immunoglobulin kappa constant, IgKLC: immunoglobulin kappa light chain, PON1: paraoxonase 1, IgHG2: immunoglobulin heavy constant gamma 2, CFB: complement factor B, AGT: angiotensinogen, Hpx: hemopexin, VDB: vitamin-D binding protein.

FIG. 17D shows the normalized total ion intensity of selected protein components. Abbreviations: AT: α-1 antitrypsin, ACT: α-1 antichymotrypsin, Hb: hemoglobin, Tf: transferrin, ApoA1: apolipoprotein A1, Hpr: haptoglobin-related protein, ApoA2: apolipoprotein A2, ApoJ: apolipoprotein J, HSA: human serum albumin, Hp: haptoglobin, A2M: α-2 macroglobulin, Cp: ceruloplasmin, ITH4: Inter-alpha-trypsin inhibitor H4, IgHA1: immunoglobulin heavy constant alpha 1, IgKC: immunoglobulin kappa constant, IgKLC: immunoglobulin kappa light chain, PON1: paraoxonase 1, IgHG2: immunoglobulin heavy constant gamma 2, CFB: complement factor B, AGT: angiotensinogen, Hpx: hemopexin, VDB: vitamin-D binding protein.

FIG. 18A shows the results from the analysis of total protein and hemoglobin binding capacity recovery after addition of fumed silica and the two washing steps.

FIG. 18B shows the HPLC-SEC chromatograms of FIV suspension and of the supernatants after addition of fumed silica and two washes with PBS. Dotted lines show the sample mixed with excess Hb.

FIG. 19A shows an analysis of total protein and HbBC recovery at each stage of processing for the TFF purification method with the use of fumed silica.

FIG. 19B shows the SEC-HPLC chromatograms of the end-product of each stage of TFF purification with the use of fumed silica. The chromatogram of samples with excess Hb (dotted lines) ends prior to the elution time of free Hb (9.6 min) given that the signal from the excess free Hb peak overwhelmed the signal from the Hp-Hb peak.

FIG. 20A shows the SDS-PAGE analysis of Hp fractions in Stages 0-3 from the TFF process used to purify Hp from FIV using fumed silica under non-reducing conditions. Stage 0 and 1 were run on 4-20% polyacrylamide gels while Stage 2 and Stage 3 were run on 10-20% polyacrylamide gels, leading to the difference in elution pattern of the polymeric species. Purity via densitometric analysis of the SDS-PAGE gels was shown to be >80% pure for Stage 2 and >95% pure for Stage 3.

FIG. 20B shows the SDS-PAGE analysis of Hp fractions in Stages 0-3 from the TFF process used to purify Hp from FIV using fumed silica under reducing conditions. Stage 0 and 1 were run on 4-20% polyacrylamide gels while Stage 2 and Stage 3 were run on 10-20% polyacrylamide gels, leading to the difference in elution pattern of the polymeric species. Purity via densitometric analysis of the SDS-PAGE gels was shown to be >80% pure for Stage 2 and >95% pure for Stage 3.

FIG. 20C shows the normalized total ion intensity from trypsin digestion mass spectrometry for Stages 0-3 of the Hp purification process via TFF of Cohn Fraction IV with the use of fumed silica. Abbreviations: AT: α-1 antitrypsin, ACT: α-1 antichymotrypsin, Hb: hemoglobin, Tf: transferrin, ApoA1: apolipoprotein A1, Hpr: haptoglobin-related protein, ApoA2: apolipoprotein A2, ApoJ: apolipoprotein J; HSA: human serum albumin, Hp: haptoglobin, A2M: α-2 macroglobulin, ITH4: inter-alpha-trypsin inhibitor H4, IgHA1: immunoglobulin heavy constant alpha 1, IgKC: immunoglobulin kappa constant, Hpx: hemopexin, VDB: vitamin-D binding protein. PZP: pregnancy zone protein, HCII: heparin cofactor II, CFB: complement factor B.

FIG. 20D shows the normalized total ion intensity from trypsin digestion mass spectrometry of selected proteins for Stages 0-3 of the Hp purification process via TFF of Cohn Fraction IV with the use of fumed silica. Abbreviations: AT: α-1 antitrypsin, ACT: α-1 antichymotrypsin, Hb: hemoglobin, Tf: transferrin, ApoA1: apolipoprotein A1, Hpr: haptoglobin-related protein, ApoA2: apolipoprotein A2, ApoJ: apolipoprotein J; HSA: human serum albumin, Hp: haptoglobin, A2M: α-2 macroglobulin, ITH4: Inter-alpha-trypsin inhibitor H4, IgHA1: immunoglobulin heavy constant alpha 1, IgKC: immunoglobulin kappa constant, Hpx: hemopexin, VDB: vitamin-D binding protein. PZP: pregnancy zone protein, HCII: heparin cofactor II, CFB: complement factor B.

FIG. 21A shows the SDS-PAGE of a representative batch of the protein scavenging cocktail under non-reducing conditions.

FIG. 21B shows the SDS-PAGE of a representative batch of the protein scavenging cocktail under reducing conditions.

FIG. 21C shows trypsin digest mass spectrometry of a representative batch of the protein scavenging cocktail. Abbreviations: human serum albumin, HSA; transferrin, Tf; haptoglobin, Hp; ceruloplasmin, Cp; vitamin-D binding protein, VDB; hemopexin, Hpx; haptoglobin-related protein, Hpr; immunoglobulin gamma 1 heavy chain, IgG1HC; α-1-B glycoprotein, A1BG; immunoglobulin kappa constant, IgkC.

FIG. 22 schematically illustrates a general manufacturing strategy for the purification of a target protein via tangential flow filtration which employs protein complex formation using a target protein binding protein.

FIG. 23 schematically illustrates a general manufacturing strategy for the purification of low molecular weight (MW) haptoglobin (Hp) via tangential flow filtration which employs protein complex formation.

FIG. 24 shows the general production scheme for the purification of the Hb-Hp complex from Cohn Fraction IV paste using tangential flow filtration (grey arrows indicate the fluid flow direction).

FIG. 25 shows a general schematic for dissociation and purification of pure Hp from the purified Hb-Hp complex (grey arrows indicate the fluid flow direction).

FIG. 26 shows SDS-PAGE of purified Hb-Hp complex and mixture of Hp and Hp-Hb obtained from dissociation and separation of Hb from the purified Hb-Hp complex.

FIG. 27 shows a comparison of hypothetical and experimental HPLC-SEC elution at each purification stage of the Hb-Hp complex.

FIG. 28 shows combined HPLC-SEC chromatograms at different stages of the Hb-Hp purification process.

FIG. 29 schematically illustrates a general procedure to remove hydrophobic ligands from proteins.

FIG. 30 shows the SEC-HPLC of apoHb, PEG-apoHb and Hb.

FIG. 31 shows the difference in heme-binding capacity of apoHb compared to PEG-apoHb. The PEG-apoHb spectra was normalized to the same mass concentration of apoHb.

FIG. 32 shows Hp binding to apoHb and PEG-apoHb.

FIG. 33A shows the SEC-HPLC chromatogram of PEG-apoHb-heme mixed with Hp monitoring the absorbance at 280 nm.

FIG. 33B shows the SEC-HPLC chromatogram of PEG-apoHb-heme mixed with Hp monitoring the florescence emission at 330 nm based on the excitation at 285 nm.

FIG. 34 shows the protein retention of PEG-apoHb and apoHb incubated at 37° C.

FIG. 35A shows a representative example of the Hb binding assay used to determine the binding capacity of Hp in the protein scavenging cocktail.

FIG. 35B shows a representative example of the iron binding assay used to determine the binding capacity Tf in the protein scavenging cocktail.

FIG. 35C shows a representative example of the total heme binding assay used to determine the binding capacity of HSA and Hpx in the protein scavenging cocktail.

FIG. 35D shows a representative example of the heme-Hpx binding assay used to determine the binding capacity of Hpx in the protein scavenging cocktail. hHSA=heme-albumin.

FIG. 36 shows some of the major roles of the protein components in the protein scavenging cocktail for treatment of various states of hemolysis. Proteins in the cocktail are highlighted in the green rectangles. Figure adapted with permission from P. W. Buehler.

FIG. 37 shows a general illustration for preparation of PEG-apoHb.

### DETAILED DESCRIPTION

### Definitions

As used herein, the term “tangential-flow filtration” refers to a process in which the fluid mixture containing the components to be separated by filtration is recirculated at high velocities tangential to the plane of the filtration membrane to reduce fouling of the filter. In such filtrations a pressure differential is applied along the length of the filtration membrane to cause the fluid and filterable solutes to flow through the membrane (i.e. filter). This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is continually processed downstream.

As used herein, the term “ultrafiltration” is used for processes employing membranes rated for retaining solutes having a molecular weight between about 1 kDa and 1000 kDa.

As used herein, the term “reverse osmosis” refers to processes employing membranes capable of retaining solutes of a molecular weight less than 1 kDa such as salts and other low molecular weight solutes.

As used herein, the term “microfiltration” refers to processes employing membranes in the 0.1 to 10 micron pore size range.

As used herein, the expression “transmembrane pressure” or “TMP” refers to the pressure differential gradient that is applied along the length of a filtration membrane to cause fluid and filterable solutes to flow through the filter.

The term “hydrophobic,” as used herein, refers to a ligand which, as a separate entity, exhibits a higher solubility in a non-aqueous solution (e.g., octanol) than in water.

The term “conjugated protein,” as used herein, refers to a protein complex that includes an apoprotein and one or more associated hydrophobic ligands. The one or more hydrophobic ligands may by covalently or non-covalently associated with the apoprotein. Examples of conjugated proteins include, for example, lipoproteins, glycoproteins, phosphoproteins, hemoproteins, flavoproteins, metalloproteins, phytochromes, cytochromes, opsins, and chromoproteins.

The phrase “mild denaturing,” as used herein refers to a process which reversibly disrupts the secondary, tertiary, and/or quaternary structure of the conjugated protein, thereby facilitating separation of the hydrophobic ligand from the apoprotein. Mild denaturing can be distinguished from harsher conditions, which cleave the peptide backbone, primarily produce insoluble protein upon denaturation/renaturation, and/or disrupt protein structure to a degree such that the protein loses its biological function upon refolding.

The terms “isolating,” “purifying,” and “separating,” as used interchangeably herein, refer to increasing the degree of purity of a polypeptide or protein of interest or a target protein from a composition or sample comprising the polypeptide and one or more impurities (e.g., additional proteins or polypeptides).

The term “haptoglobin” as used herein refers to a protein that is synthesized and secreted mainly in the liver. In blood plasma, haptoglobin binds to cell-free hemoglobin released from erythrocytes with high affinity and thereby inhibits hemoglobin oxidative activity. The haptoglobin-hemoglobin complex is then removed by the reticuloendothelial system (mostly in the spleen). Haptoglobin, in its simplest form, consists of two alpha and two beta chains, connected by disulfide bridges. The chains originate from a common precursor protein, which is proteolytically cleaved during protein synthesis. Hp exists in two allelic forms in the human population, so-called Hp1 and Hp2, the latter one having arisen due to partial duplication of the Hp1 gene. Three genotypes of Hp, therefore, are found in humans: Hp1-1, Hp2-1, and Hp2-2. Hp of different genotypes have been shown to have similar effects in vivo in attenuating Hb-mediated toxicity. Furthermore, a protein with >90% sequence identity to the Hp1 gene, called haptoglobin related protein (Hpr) also has high affinity for Hb. The term “haptoglobin” thus encompasses all Hp phenotypes (Hp1-1,Hp2-2 and Hp2-1).

Methods

The methods described herein generally involve methods of isolating proteins by membrane filtration. In general, membrane filtration techniques may be divided into three basic categories based on filter pore size and filtration pressure. The first of these categories, known as microfiltration, refers to filters having relatively large pore sizes and relatively low operating pressures. The second category, ultrafiltration, refers to filters having intermediate pore sizes and intermediate operating pressures. Finally, the third category, reverse osmosis, refers to filters having extremely small pore sizes and relatively high operating pressures. Predictably, microfiltration techniques are utilized when large solutes, or species, are to be filtered. Ultrafiltration is used when intermediate species are to be processed, and reverse osmosis is utilized when extremely small species are targeted.

Conventionally, ultrafiltration employs membranes rated for retaining solutes between approximately 1 and 1000 kDa in molecular weight, reverse osmosis employs membranes capable of retaining salts and other low molecular weight solutes, and microfiltration, or microporous filtration, employs membranes in the 0.1 to 10 micrometer (micron) pore size range, typically used to retain colloids and microorganisms.

Traditionally, membrane filters have functioned by placing a porous membrane perpendicularly across the path of a fluid mixture from which a selected species is to be filtered. The fluid mixture flows through the membrane and the selected species is retained by the membrane. Such methods are generally referred to as direct-flow filtration (DFF) or dead-end filtration.

A problem generally associated with DFF is the tendency of the filter to accumulate solutes from the fluid mixture that is being filtered. Accumulation of these solutes creates a layer of retained solutes (known as filter cake) on the filtration membrane and has a tendency to block, or clog, the pores of the membrane decreasing the flow of the fluid mixture, or flux, through the filtration membrane.

The decrease in flux attributable to the accumulation of the solute layer on the filtration membrane may be partially overcome by increasing the pressure differential, or transmembrane pressure that exists across the filtration membrane. Pressure increases of this type are, however, limited in their effectiveness by the tendency of the filter to become increasingly clogged as the filtration process continues. Eventually, of course, further pressure increases become impractical and the filtration process must be halted and the clogged membrane replaced. This is especially true when fragile filtration membranes are employed as they can burst at high operating pressures.

A second problem associated with the accumulation of solutes on the filtration membrane is the tendency for the solute layer to act as a secondary filter. As a result, as the layer of solutes deposited on the filtration membrane increases, passage through the filtration membrane becomes limited to smaller and smaller solutes. The tendency for the solute layer to act as a secondary filter is especially problematic because, unlike the decreased flux attributable to the same layer, it cannot be overcome by increasing the transmembrane pressure.

One solution to the problem of membrane blockage has been the development of tangential-flow filters. Filters of this type employ a membrane which is generally similar to the membrane types employed by traditional filters. In tangential-flow filters, however, the membrane is placed tangentially to the flow of the fluid mixture to cause the fluid mixture to flow tangentially over a first side of the membrane. At the same time, a fluid media is placed in contact with a second surface of the membrane. The fluid mixture and the fluid media are maintained under pressures which differ from each other. The resulting pressure differential, or transmembrane pressure, causes fluid within the fluid mixture, and species within the fluid mixture, to traverse the membrane, leaving the fluid mixture and joining the fluid media.

In operation, the tangential-flow of the fluid mixture over the membrane functions to prevent solutes within the fluid mixture from settling on the membrane surface. This occurs due to the shear force acting on the surface of the membrane due to fluid flow. As a result, the use of Cross-Flow or Tangential-Flow Filtration (TFF) has proven to be an effective means of reducing membrane blockage for membrane filters. Not surprisingly, then, a wide variety of differing designs exist for filters of the tangential-flow type, and TFF methods have been widely described. For example, Marinaccio et al., U.S. Pat. No. 4,888,115 discloses the process (termed “cross-flow”) for use in the separation of biological liquids such as blood components for plasmapheresis. In this process, blood is passed tangentially to (i.e., across) an organic polymeric microporous filter membrane, and particulate matter is removed. In another example of current art, tangential flow filtration has been disclosed for the filtration of beer solutions (Shackleton, EP 0,208,450, published Jan. 14, 1987) specifically for the removal of particulates such as yeast cells and other suspended solids. Kothe et al., (U.S. Pat. No. 4,644,056, issued Feb. 17, 1987) disclose the use of this process in the purification of immunoglobulins from milk or colostrum, and Castino (U.S. Pat. No. 4,420,398, issued Dec. 13, 1983) describes its use in the separation of antiviral substances such as interferons from broths containing these substances as well as viral particles and the remains of cell cultures from which they are derived.

Tangential flow filtration units have also been employed in the separation of bacterial enzymes from cell debris (Quirk et al., 1984, Enzyme Microb. Technol., 6(5):201). Using this technique, Quirk et al. were able to isolate enzyme in higher yields and in less time than using the conventional technique of centrifugation. The use of tangential flow filtration for several applications in the pharmaceutical field has been reviewed by Genovesi (1983, J. Parenter. Aci. Technol., 37(3):81), including the filtration of sterile water for injection, clarification of a solvent system, and filtration of enzymes from broths and bacterial cultures.

The methods described herein can employ direct-flow filtration (DFF), cross-flow or tangential-flow filtration (TFF), or a combination thereof. In certain embodiments, the methods described herein can employ TFF.

Methods of Purifying Apoproteins

Described herein are methods of isolating an apoprotein from a protein solution comprising a conjugated protein, wherein the conjugated protein comprises the apoprotein and a hydrophobic ligand associated with the apoprotein. Methods of isolating the apoprotein protein can comprise (i) contacting the conjugated protein with an aqueous solution comprising a water-miscible solvent and a pH modifier, thereby forming a protein solution having a pH of less than 6.5 or greater than 8; and (ii) filtering the protein solution by ultrafiltration against a filtration membrane having a pore size that separates the apoprotein from the hydrophobic ligand, thereby forming a retentate fraction comprising the apoprotein and a permeate fraction comprising the hydrophobic ligand. In some embodiments, methods for isolating an apoprotein can further comprise (iii) neutralizing the retentate fraction to isolate the apoprotein.

The conjugated protein can comprise any conjugated protein. In some examples, the conjugated protein can comprise, for example, a lipoprotein, glycoprotein, phosphoprotein, hemoprotein, flavoprotein, metalloprotein, phytochrome, cytochrome, opsin, or chromoprotein.

In some examples, the ligand can comprise a prosthetic group, a cofactor, a lipid, a metabolite, or a combination thereof. In some cases, the hydrophobic ligand can be non-covalently associated with the apoprotein. In some cases, the hydrophobic ligand can be ionically or electrostatically associated with the apoprotein. In some cases, the hydrophobic ligand can be covalently associated with the apoprotein.

In some examples, the conjugated protein can comprise a lipoprotein, the apoprotein can comprise an apolipoprotein, and the hydrophobic ligand can comprise a lipid.

In some examples, the conjugated protein can comprise a heme protein, the apoprotein can comprise an apo-heme protein such as apohemoglobin, and the hydrophobic ligand can comprise a heme. In these embodiments, the hemoglobin can be present in the protein solution at a concentration from 0.1 mg/mL to 5 mg/mL, such as from 0.5 mg/mL to 3 mg/mL.

In some examples, the conjugated protein can comprise human serum albumin (HSA). In these embodiments, the HSA can be present in the protein solution at a concentration from 0.5 mg/mL to 25 mg/mL, such as from 1 mg/mL to 20 mg/mL.

The protein solution can have an acidic or basic pH, selected so as to facilitate dissociation of the hydrophobic ligand and the apoprotein.

In some cases, the protein solution can have an acidic pH. In some of these embodiments, the protein solution can have a pH of 6 or less (e.g., 5.5 or less, 5 or less, 4.5 or less, 4 or less, 3.5 or less, 3 or less, or 2.5 or less). In some embodiments, the protein solution can have a pH of 2 or more (e.g., 2.5 or more, 3 or more, 3.5 or more, 4 or more, 4.5 or more, 5 or more, or 5.5 or more).

The protein solution can have a pH ranging from any of the minimum values described above to any of the maximum values described above. For example, in some embodiments, the protein solution can have a pH of from 2 to 6, such as from 3 to 6.

In other cases, the protein solution can have a basic pH. In some of these embodiments, the protein solution can have a pH of greater than 8 (e.g., 8.5 or more, 9 or more, 9.5 or more, 10 or more, or 10.5 or more). In some embodiments, the protein solution can have a pH of 11 or less (e.g., 10.5 or less, 10 or less, 9.5 or less, 9 or less, or 8.5 or less.

The protein solution can have a pH ranging from any of the minimum values described above to any of the maximum values described above. For example, in some embodiments, the protein solution can have a pH of from greater than 8 to 11, such as from greater than 8 to 10.

Generally, the filtration membrane can have a range of pore sizes effective to separate the hydrophobic ligand from the apoprotein (e.g., a pore size which allows the hydrophobic ligand to pass through the filtration membrane but retains the apoprotein). For example, the filtration membrane can be rated for retaining solutes having a molecular weight ranging from the molecular weight of the hydrophobic ligand to the molecular weight of the apoprotein. By way of example, in embodiments where the conjugated protein is hemoglobin, the filtration membrane can be rated for retaining solutes having a molecular weight ranging from the molecular weight of the heme to the molecular weight of the apohemoglobin (e.g., such as a membrane rated for retaining solutes having a molecular weight of from about 1 kDa to 100 kDa, such as from about 1 kDa to about 10 kDa).

In connection with the methods described herein, ultrafiltration can comprise direct-flow filtration (DFF), cross-flow or tangential-flow filtration (TFF), or a combination thereof. In certain embodiments, the ultrafiltration can comprise tangential-flow filtration (TFF).

The membranes useful in the filtration steps described herein can be in the form of flat sheets, rolled-up sheets, cylinders, concentric cylinders, ducts of various cross-section and other configurations, assembled singly or in groups, and connected in series or in parallel within the filtration unit. The apparatus can be constructed so that the filtering and filtrate chambers run the length of the membrane.

Suitable membranes include those that separate the desired species from undesirable species in the mixture without substantial clogging problems and at a rate sufficient for continuous operation of the system. Examples are described, for example, in Gabler F R. Tangential flow filtration for processing cells, proteins, and other biological components. ASM News 1984; 50:299-304. They can be synthetic membranes of either the microporous type or the ultrafiltration type. A microporous membrane has pore sizes typically from 0.1 to 10 micrometers, and can be made so that it retains all particles larger than the rated size. Ultrafiltration membranes have smaller pores and are characterized by the size of the protein that will be retained. They are available in increments from 1000 to 1,000,000 Dalton nominal molecular weight limits.

Generally, the filtration membrane can comprise an ultrafiltration membrane. Ultrafiltration membranes are normally asymmetrical with a thin film or skin on the upstream surface that is responsible for their separating power. They are commonly made of regenerated cellulose, polysulfone or polyethersulfone. In some cases, the filtration membrane can be rated for retaining solutes having a molecular weight of from about 1 kDa to 4,000 kDa, such as from about 1 kDa to about 1,000 kDa or from about 1 kDa to about 500 kDa.

In some cases, each filtration step can involve filtration through a single filtration membrane. In other cases, because membrane filters are not perfect and may have holes that allow some intended retentate molecules to slip through, more than one membrane (e.g., two membranes, three membranes, four membranes, or more) having the same pore size can be utilized for a given filtration step. In these embodiments, the membranes can be placed so as to be layered parallel to each other (e.g., one on top of the other) such that filtered fluid sequentially flows through each of the more than one membrane.

Membrane filters for tangential-flow filtration are available as units of different configurations depending on the volumes of liquid to be handled, and in a variety of pore sizes. Particularly suitable for use in the methods described herein, on a relatively large scale, are those known, commercially available tangential-flow filtration units.

The filtration unit useful herein is suitably any unit now known or discovered in the future that serves as an appropriate filtration module, particularly for microfiltration and ultrafiltration. The preferred filtration unit is hollow fibers or a flat sheet device. These sandwiched filtration units can be stacked to form a composite cell. One example type of rectangular filtration plate type cell is available from Filtron Technology Corporation, Northborough, Mass., under the trade name Centrasette. Another example filtration unit is the Millipore Pellicon ultrafiltration system available from Millipore, Bedford, Mass.

The water-miscible solvent can comprise a polar protic solvent. In some embodiments, the water-miscible solvent can comprise an alcohol (e.g., ethanol, methanol, or a combination thereof).

In some embodiments, the aqueous solution can comprise at least 10% by volume (e.g., at least 15% by volume, at least 20% by volume, at least 25% by volume, at least 30% by volume, at least 35% by volume, at least 40% by volume, at least 45% by volume, at least 50% by volume, at least 55% by volume, at least 60% by volume, at least 65% by volume, at least 70% by volume, at least 75% by volume, at least 80% by volume, or at least 85% by volume) alcohol. In some embodiments, the aqueous solution can comprise 90% by volume or less (e.g., 85% by volume or less, 80% by volume or less, 75% by volume or less, 70% by volume or less, 65% by volume or less, 60% by volume or less, 55% by volume or less, 50% by volume or less, 45% by volume or less, 40% by volume or less, 35% by volume or less, 30% by volume or less, 25% by volume or less, 20% by volume or less, or 15% by volume or less) alcohol.

The aqueous solution can comprise a quantity of alcohol ranging from any of the minimum values described above to any of the maximum values described above. For example, in some embodiments, the aqueous solution can comprise from 10% to 90% by volume alcohol (e.g., 20% to 90% by volume, or 30% to 90% by volume). In one example, the conjugated protein can comprise hemoglobin and the aqueous solution can comprise from 60% to 90% by volume alcohol (e.g., 60% to 90% by volume ethanol). In another example, the conjugated protein can comprise HSA and the aqueous solution can comprise from 30% to 60% by volume alcohol.

In some embodiments, filtering step (ii) can comprise buffer exchange. In certain embodiments filtering step (ii) can comprise continuous diafiltration or dialysis.

Optionally, the retentate fraction can spectroscopically monitored during the continuous diafiltration to monitor separation of the hydrophobic ligand from the apoprotein. Spectroscopically monitoring the retentate fraction can comprise monitoring a spectroscopic peak (e.g., an absorbance peak) associated with the apoprotein and a spectroscopic peak (e.g., an absorbance peak) associated with the conjugated protein. In some embodiments, filtering step (ii) can comprise performing the continuous diafiltration until a relative magnitude of the absorbance peak associated with the apoprotein and the absorbance peak associated with the conjugated protein suggest that the apoprotein and the conjugated protein are present in the retentate fraction at a molar ratio of at least 9:1 (e.g., at least 10:1, at least 15:1, at least 20:1, at least 25:1, at least 50:1, or at least 100:1).

Methods for isolating an apoprotein can further comprise (iii) neutralizing the retentate fraction to isolate the apoprotein. In some embodiments, neutralizing step (iii) comprises continuous diafiltration with a buffer solution having a pH of from 6.8 to 7.6.

The purity of isolated apoprotein can be assessed using a variety of methods known in the art, including for example, liquid chromatography and/or spectroscopic methods (UV-Vis spectroscopy, fluorescence spectroscopy, etc.). In certain embodiments, the apoprotein isolated in step (iii) can comprise less than 1% (e.g., less than 0.75%, less than 0.5%, less than 0.25%, or less than 0.1%) residual hydrophobic ligand relative to the concentration of apoprotein isolated in step (iii), as measured by a suitable spectroscopic method (e.g., UV Vis spectroscopy).

The apoprotein isolated in step (iii) can exhibit excellent stability relative to apoproteins isolated using other conventional methodologies. In some embodiments, the apoprotein isolated in step (iii) can be stable for a period of at least 7 days (e.g., at least 14 days, at least 30 days, at least 60 days, at least 120 days, or at least 180 days) at 22° C. In certain embodiments, at least 75% (e.g., at least 80%, at least 85%, at least 90%, or at least 95%) of the apoprotein remains soluble in solution after storage at 22° C. for 7 days. In certain embodiments, at least 75% (e.g., at least 80%, at least 85%, at least 90%, or at least 95%) of the apoprotein remains soluble in solution after storage at 4° C. for 180 days. In certain embodiments, at least 75% (e.g., at least 80%, at least 85%, at least 90%, or at least 95%) of the apoprotein remains soluble in solution after storage at −80° C. for 180 days.

In certain embodiments, the apoprotein can comprise apohemoglobin. In some of these embodiments, at least 65% (e.g., at least 70%, at least 75%, at least 80%, or at least 85%) of the apohemoglobin can retain its activity (i.e., retain its ability to bind heme) after storage at 22° C. for 7 days. In some of these embodiments, at least 65% (e.g., at least 70%, at least 75%, at least 80%, or at least 85%) of the apohemoglobin can retain its activity (i.e., retain its ability to bind heme) after storage at 4° C. for 180 days. In some of these embodiments, at least 65% (e.g., at least 70%, at least 75%, at least 80%, or at least 85%) of the apohemoglobin can retain its activity (i.e., retain its ability to bind heme) after storage at −80° C. for 180 days.

In some embodiments, the apoHb can be prepared using the ultrafiltration methods described below. The apoHb prepared by various methods possess the same chemical identity (primary structure) and primarily the same quaternary conformation compared to apoHb prepared by existing precipitation or liquid-liquid extraction methodologies. The apoHb produced by the ultrafiltration methods described herein can exist in aqueous solution primarily as an αβ dimer without the use of reducing agents (2-mercaptoethanol, dithiothreitol). In contrast, previous methodologies may produce non-native tetramers (α₂β₂) that require reducing agents to form αβ dimers. Furthermore, the apoHb produced in the current methodology is stable for over a week at room temperature and even more stable at 4° C., −80° C. and in lyophilized form. Previous methodologies produced apoHb that quickly precipitated (approximately 24 hours) when stored at room temperature. In certain embodiments, the apoHb can be characterized by a residual Soret peak having a maximum absorption ranging from 411-417 nm, such as 415 nm (after renaturation/neutralization, but before complexation with Hp). Previous methodologies produced apoHb which had a residual Soret peak at 402-407 nm.

In some embodiments, methods can further comprise lyophilizing the apoprotein isolated in step (iii).

Also described are methods of isolating a ligand from a protein solution comprising a conjugated protein. These methods can comprise (i) mildly denaturing the conjugated protein to form a protein solution; and (ii) filtering the protein solution by ultrafiltration against a filtration membrane having a pore size that separates the apoprotein from the hydrophobic ligand, thereby forming a retentate fraction comprising the apoprotein and a permeate fraction comprising the hydrophobic ligand. In some embodiments, methods for isolating an apoprotein can further comprise (iii) neutralizing the retentate fraction to isolate the apoprotein.

In some examples, mildly denaturing the conjugated protein can comprise heating the conjugated protein (e.g., to a temperature of from 40° C. to 60° C.).

In some examples, mildly denaturing the conjugated protein can comprise contacting the conjugated protein with a pH modifier (e.g., with an acid and/or a base). Mildly denaturing the conjugated protein can comprise contacting the conjugated protein with an effective amount of a pH modifier to produce an acidic or basic pH, selected so as to facilitate dissociation of the hydrophobic ligand and the apoprotein.

In some cases, mildly denaturing the conjugated protein can comprise contacting the conjugated protein with an effective amount of a pH modifier to produce a pH of 6 or less (e.g., 5.5 or less, 5 or less, 4.5 or less, 4 or less, 3.5 or less, 3 or less, or 2.5 or less). In some embodiments, mildly denaturing the conjugated protein can comprise contacting the conjugated protein with an effective amount of a pH modifier to produce a pH of 2 or more (e.g., 2.5 or more, 3 or more, 3.5 or more, 4 or more, 4.5 or more, 5 or more, or 5.5 or more).

Mildly denaturing the conjugated protein can comprise contacting the conjugated protein with an effective amount of a pH modifier to produce a pH ranging from any of the minimum values described above to any of the maximum values described above. For example, in some embodiments, mildly denaturing the conjugated protein can comprise contacting the conjugated protein with an effective amount of a pH modifier to produce a pH of from 2 to 6, such as from 3 to 6.

In other cases, mildly denaturing the conjugated protein can comprise contacting the conjugated protein with an effective amount of a pH modifier to produce a pH of 8 or more (e.g., 8.5 or more, 9 or more, 9.5 or more, 10 or more, or 10.5 or more). In some embodiments, mildly denaturing the conjugated protein can comprise contacting the conjugated protein with an effective amount of a pH modifier to produce a pH of 11 or less (e.g., 10.5 or less, 10 or less, 9.5 or less, 9 or less, or 8.5 or less.

Mildly denaturing the conjugated protein can comprise contacting the conjugated protein with an effective amount of a pH modifier to produce a pH ranging from any of the minimum values described above to any of the maximum values described above. For example, in some embodiments, mildly denaturing the conjugated protein can comprise contacting the conjugated protein with an effective amount of a pH modifier to produce a pH of from 8 to 11, such as from 8 to 10.

In some examples, mildly denaturing the conjugated protein can comprise contacting the conjugated protein with a non-aqueous solvent, such as an alcohol. Examples of such non-aqueous solvents include, for example, ethanol, methanol, isopropanol, butanol, 2-propanol, phenol, or combinations thereof.

In some examples, mildly denaturing the conjugated protein can comprise contacting the conjugated protein with a chaotropic agent (e.g., a salt that can disrupt the structure of a protein by shielding charges and preventing the stabilization of salt bridges) Any salt in principle may be used. Examples of suitable chaotropic agents include, but are not limited to, guanidinium chloride, lithium perchlorate, lithium acetate, magnesium chloride, sodium dodecyl sulfate, thiourea, urea, calcium chloride, and combinations thereof.

Also provided is apohemoglobin produced by the filtration methods described herein. Apohemoglobin prepared by the filtration methods described herein (after renaturation/neutralization) can exhibit improved stability and purity as compared to apohemoglobin prepared by existing precipitation and liquid-liquid extraction methodologies.

In some embodiments, the apohemoglobin produced by the filtration methods described herein can be stable for a period of at least 7 days (e.g., at least 14 days, at least 30 days, at least 60 days, at least 120 days, or at least 180 days) at 22° C. In certain embodiments, at least 75% (e.g., at least 80%, at least 85%, at least 90%, or at least 95%) of the apohemoglobin produced by the filtration methods described herein remains soluble in solution after incubation at 22° C. for 7 days. In certain embodiments, at least 75% (e.g., at least 80%, at least 85%, at least 90%, or at least 95%) of the apohemoglobin produced by the filtration methods described herein remains soluble in solution after incubation at 4° C. for 180 days. In certain embodiments, at least 75% (e.g., at least 80%, at least 85%, at least 90%, or at least 95%) of the apohemoglobin produced by the filtration methods described herein remains soluble in solution after incubation at −80° C. for 180 days.

In some of these embodiments, at least 65% of the apohemoglobin can retain its activity (i.e., retain its ability to bind heme) after storage at 22° C. for 7 days. In some of these embodiments, at least 65% of the apohemoglobin can retain its activity (i.e., retain its ability to bind heme) after storage at 4° C. for 180 days. In some of these embodiments, at least 65% of the apohemoglobin can retain its activity (i.e., retain its ability to bind heme) after storage at −80° C. for 180 days.

In some embodiments, the apoHb can be prepared using the ultrafiltration methods described below. The apoHb prepared by various methods possess the same chemical identity (primary structure) and primarily the same quaternary conformation compared to apoHb prepared by existing precipitation or liquid-liquid extraction methodologies. The apoHb produced by the ultrafiltration methods described herein can exist in aqueous solution primarily as an αβ dimer without the use of reducing agents (2-mercaptoethanol, dithiothreitol). In contrast, previous methodologies may produce non-native tetramers (α₂β₂) that require reducing agents to form αβ dimers. Furthermore, the apoHb produced in the current methodology is stable for over a week at room temperature and even more stable at 4° C., −80° C. and in lyophilized form. Previous methodologies produced apoHb that quickly precipitated (approximately 24 hours) when stored at room temperature. In certain embodiments, the apoHb can be characterized by a residual Soret peak having a maximum absorption ranging from 411-417 nm, such as 415 nm (after renaturation/neutralization, but before complexation with Hp). Previous methodologies produced apoHb which had a residual Soret peak at 402-407 nm.

Methods for the Purification of Haptoglobin

Also provided herein are method for isolating haptoglobin from plasma or a fraction thereof. In some embodiments, methods for isolating haptoglobin from plasma or a fraction thereof can comprise (i) clarifying the plasma or fraction thereof; and (ii) filtering the plasma or a fraction thereof by ultrafiltration against a filtration membrane, thereby forming a retentate fraction comprising haptoglobin having a molecular weight of greater than about 100 kDa and a permeate fraction comprising serum proteins and other impurities having a molecular weight of less than about 100 kDa.

The plasma or fraction thereof can comprise plasma fraction IV, plasma fraction V, a fraction of precipitated plasma (from salting out, polyethylene glycol, zinc chloride, or equivalent) or a combination thereof.

Clarifying the plasma or a fraction thereof can comprise removing suspended solids from the plasma or fraction thereof. Removing suspended solids from the plasma or fraction thereof can comprise filtering (via ultrafiltration, microfiltration, depth filtration or equivalent) the plasma or a fraction thereof, contacting the plasma or a fraction thereof with a salting out agent (e.g., ammonium sulfate), an adsorbing agent (e.g., ethacridine lactate), or a combination thereof. Further clarification may be implemented through addition of a lipid-binding agent such as fumed silica (such as fumed silica sold under the tradename Aerosil 380®, or similar), clay, bentonite, terra alba, active carbon, or a combination thereof.

In some embodiments, the ultrafiltration can comprise tangential-flow filtration.

In some cases, the method can further comprise filtering the permeate fraction comprising serum proteins and other impurities by ultrafiltration against a second filtration membrane, thereby forming a second retentate fraction comprising a blend of proteins having a molecular weight below about 100 kDa and above a cutoff value and a second permeate fraction comprising serum proteins and other impurities having a molecular weight below the cutoff value, wherein the blend of proteins comprises low molecular weight haptoglobin, transferrin, hemopexin, albumin, or a combination thereof. The cutoff value can be from about 20 kDa to about 70 kDa, such as from about 25 kDa to about 50 kDa

In these methods, the second retentate fraction can include a blend of proteins (e.g., low molecular weight haptoglobin, transferrin, hemopexin, albumin, or a combination thereof) that can bind and detoxify cell-free hemoglobin, free iron, and/or free heme. Accordingly, the second retentate fraction can be administered to a subject in need thereof, for example, to treat hemolytic anemia and other conditions characterized by or associated with hemolysis (e.g., sickle cell anemia, malaria, red blood cell transfusions, thalassemia, autoimmune disorders, bone marrow failure, infections, surgery, severe burns, acute lung injury, the administration of chemotherapeutics, radiation therapy etc.). In some examples, the second retentate fraction can be administered prophylactically to a subject to prevent damage associated with anticipated hemolysis (e.g., prior to surgery, radiation therapy, acute radiation injury, etc.). In some examples, the second retentate fraction can be co-administered with a therapy that induce hemolysis (e.g., a chemotherapeutic agent, an anti-infective agent, a radiation therapy, or a combination thereof). These proteins may also be used in wound-healing applications and to prevent bacterial proliferation/resistance.

In other embodiments, methods for isolating haptoglobin from plasma or a fraction thereof can comprise (i) filtering the plasma or fraction thereof by ultrafiltration against a first filtration membrane, thereby forming a first retentate fraction comprising serum proteins having a molecular weight above a first cutoff value and a first permeate fraction comprising most of the haptoglobin and serum proteins having a molecular weight below the first cutoff value; and (ii) filtering the first permeate fraction by ultrafiltration against a second filtration membrane, thereby forming a second retentate fraction comprising small amounts of Hp2-1, Hp2-2, and serum proteins having a molecular weight below the first cutoff value and above a second cutoff value; and a second permeate fraction comprising Hp2-1, Hp2-2, and serum proteins having a molecular weight below the second cutoff value. In some cases, the method can further comprise (iii) filtering the second permeate fraction by tangential-flow filtration against a third filtration membrane, thereby forming a third retentate fraction comprising Hp2-1 and Hp2-2 having a molecular weight below the second cutoff value and above a third cutoff value; and a third permeate fraction comprising low molecular weight haptoglobin, serum proteins and other impurities having a molecular weight below the third cutoff value. In some cases, the method can further comprise (iv) filtering the third permeate fraction comprising low molecular weight haptoglobin, serum proteins and other impurities by ultrafiltration against a fourth filtration membrane, thereby forming a fourth retentate fraction comprising a blend of proteins having a molecular weight below the third cutoff value and above a fourth cutoff value and a fourth permeate fraction comprising serum proteins and other impurities having a molecular weight below the fourth cutoff value, wherein the blend of proteins comprises low molecular weight haptoglobin, transferrin, hemopexin, albumin, or a combination thereof.

The first cutoff value can be from about 650 kDa to about 1000 kDa. The second cutoff value can be from about 300 kDa to about 700 kDa. The third cutoff value can be from about 70 kDa to about 200 kDa. The fourth cutoff value can be from about 20 kDa to about 70 kDa. In certain examples, the first cutoff value can be about 750 kDa, the second cutoff value can be about 500 kDa, and the third cutoff value can be about 100 kDa. The fourth cutoff value can be about 30 kDa or about 50 kDa.

The plasma or fraction thereof can comprise plasma fraction IV, plasma fraction V, a fraction of precipitated plasma (from salting out, or equivalent) or a combination thereof.

Clarifying the plasma or a fraction thereof can comprise removing suspended solids from the plasma or fraction thereof. Removing suspended solids from the plasma or fraction thereof can comprise filtering (via ultrafiltration, microfiltration, depth filtration or equivalent) the plasma or a fraction thereof, contacting the plasma or a fraction thereof with a salting out agent (e.g., ammonium sulfate), an adsorbing agent (e.g., ethacridine lactate), or a combination thereof. Further clarification may be implemented through addition of a lipid-binding agent such as fumed silica (such as fumed silica sold under the tradename Aerosil 380®, or similar), clay, bentonite, terra alba, active carbon, or a combination thereof.

In some embodiments, the ultrafiltration can comprise tangential-flow filtration.

In these methods, the fourth retentate fraction can include a blend of proteins (e.g., low molecular weight haptoglobin, transferrin, hemopexin, albumin, or a combination thereof) that can bind and detoxify free hemoglobin, free iron, and/or free heme. Accordingly, the fourth retentate fraction can be administered to a subject in need thereof, for example, to treat hemolytic anemia and other conditions characterized by or associated with hemolysis (e.g., sickle cell anemia, malaria, red blood cell transfusions, thalassemia, autoimmune disorders, bone marrow failure, infections, surgery, severe burns, acute lung injury, the administration of chemotherapeutics, radiation therapy, etc.). In some examples, the fourth retentate fraction can be administered prophylactically to a subject to prevent damage associated with anticipated hemolysis (e.g., prior to surgery, radiation therapy, acute radiation injury, etc.). In some examples, the fourth retentate fraction can be co-administered with a therapy that induce hemolysis (e.g., a chemotherapeutic agent, an anti-infective agent, a radiation therapy, or a combination thereof). These proteins may also be used in wound-healing applications and to prevent bacterial proliferation/resistance.

Also provided are haptoglobin-containing composition prepared using the methods described herein. For example, provided herein are haptoglobin-containing compositions isolated from plasma or a plasma fraction thereof that comprise from 10% by weight to 99% by weight (e.g., from 10% to 90% by weight, from 10% to 80% by weight, from 40% to 85% by weight, or from 40% by weight to 80% by weight) haptoglobin, based on the total weight of all proteins in the haptoglobin-containing composition; from greater than 0% by weight to 30% by weight (e.g., from 1% to 30% by weight) albumin (e.g., different MW albumins, including polymeric albumin species), alpha-1 antitrypsin, or a combination thereof, based on the total weight of all proteins in the haptoglobin-containing composition; and from greater than 0% by weight to 20% by weight (e.g., from greater than 0% by weight to 8% by weight, from 2% by weight to 8% by weight, or from 2% by weight to 4% by weight) macroglobulin, based on the total weight of all proteins in the haptoglobin-containing composition. In some examples, these from greater than 0% by weight to 30% by weight (e.g., from 1% to 30% by weight) albumin (e.g., different MW albumins, including polymeric albumin species), based on the total weight of all proteins in the haptoglobin-containing composition. In some examples, these from greater than 0% by weight to 30% by weight (e.g., from 1% to 30% by weight) alpha-1 antitrypsin, based on the total weight of all proteins in the haptoglobin-containing composition. In some examples, these from greater than 0% by weight to 30% by weight (e.g., from 1% to 30% by weight) albumin (e.g., different MW albumins, including polymeric albumin species) and alpha-1 antitrypsin, based on the total weight of all proteins in the haptoglobin-containing composition. Optionally, the composition can further include from greater than 0% to 15% by weight (e.g., from 1% by weight to 4% by weight) of an apolipoprotein, based on the total weight of all proteins in the haptoglobin-containing composition. Optionally, the composition can further include from greater than 0% to 15% by weight (e.g., from 1% by weight to 4% by weight) of transferrin, based on the total weight of all proteins in the haptoglobin-containing composition. Optionally, the composition can further include from greater than 0% to 40% by weight (e.g., from 1% by weight to 4% by weight) of any proteins associated with lipoproteins (e.g., high density lipoprotein (HDL)), based on the total weight of all proteins in the haptoglobin-containing composition. Optionally, the composition can further include from greater than 0% by weight to 30% by weight alpha-1 antitrypsin, based on the total weight of all proteins in the haptoglobin-containing composition. In some embodiments, the haptoglobin has an average molecular weight of from 80 kDa to 1,000 kDa, such as from 100 kDa to 1,000 kDa. In some embodiments, the composition further comprises an additional protein chosen from transferrin, hemopexin, or a combination thereof. In some embodiments, the composition can further comprise vitamin-D binding protein, and ceruloplasmin, or a combination thereof. In some embodiments, the haptoglobin is characterized by having residual hemoglobin as characterized by UV-visible spectroscopy of the Soret peak ranging from 402-407 nm. In some of these embodiments, the residual hemoglobin can be present in an amount less than 10% by weight (e.g., less than 5% by weight, less than 3% by weight, or less than 1% by weight), based on the total weight of all proteins in the haptoglobin-containing composition. In some embodiments, the haptoglobin is characterized by having residual apohemoglobin as characterized by the heme-binding capacity of the sample determined via UV-visible spectroscopy of the Soret peak formed by the heme-binding capacity assay which ranges from 411-417 nm. In some embodiments, the composition can be substantially free of immunogenic proteins (e.g., immunoglobins).

Also provided herein are haptoglobin-containing compositions isolated from plasma or a plasma fraction thereof that comprise from 5% by weight to 99% by weight (e.g., from 50% by weight to 95% by weight, from 5% by weight to 60% by weight, from 5% by weight to 25% by weight, from 5% by weight to 40% by weight, from 5% by weight to 30% by weight, or from 5% by weight to 25% by weight) haptoglobin, based on the total weight of all proteins in the haptoglobin-containing composition; 1% by weight to 95% by weight (e.g., from 1% by weight to 50% by weight, from 1% by weight to 40% by weight, from 25% by weight to 40% by weight, from 5% by weight to 60% by weight, or from 10% by weight to 40% by weight) transferrin, based on the total weight of all proteins in the haptoglobin-containing composition. Optionally, the composition can further comprise from 1% by weight to 75% by weight (e.g., from 1% by weight to 40% by weight, from 1% by weight to 10% by weight, from 2% by weight to 30% by weight, from 5% by weight to 20% by weight, or from 5% to 40% by weight) hemopexin, based on the total weight of all proteins in the haptoglobin-containing composition. In some embodiments, the composition can further include from 1% by weight to 70% by weight (e.g., from 5% by weight to 70% by weight, from 1% to 30% by weight, from 1% by weight to 15% by weight, from 30% by weight to 60% by weight, or from 5% to 30% by weight) albumin (e.g., different MW albumins, including polymeric albumin species), based on the total weight of all proteins in the haptoglobin-containing composition. In some embodiments, the composition can further include from 1% by weight to 30% by weight (e.g., from 1% by weight to 15% by weight) alpha-1 antitrypsin, based on the total weight of all proteins in the haptoglobin-containing composition. In some embodiments, the composition can further include from greater than 0% by weight to 20% by weight (e.g., from greater than 0% by weight to 15% by weight, from greater than 0% by weight to 10% by weight, from greater than 0% by weight to 8% by weight, or from 2% by weight to 4% by weight) macroglobulin, based on the total weight of all proteins in the haptoglobin-containing composition. In some embodiments, the composition can further include from greater than 0% to 40% by weight (e.g., from greater than 0% by weight to 15% by weight, from greater than 0% by weight to 10% by weight, or from 1% by weight to 4% by weight) of any proteins associated with lipoproteins (e.g., high density lipoprotein (HDL)), based on based on the total weight of all proteins in the haptoglobin-containing composition. In some embodiments, the composition can further include from greater than 0% to 10% by weight (e.g., from 1% by weight to 4% by weight) of apolipoproteins, based on the total weight of all proteins in the haptoglobin-containing composition. In some embodiments, the composition further comprises an additional protein chosen from transferrin, hemopexin, or a combination thereof. In some embodiments, the composition further comprises vitamin-D binding protein, and ceruloplasmin, or a combination thereof. In certain embodiments, the composition can comprise both transferrin and hemopexin. These compositions can be substantially free (i.e., the composition can include less than 0.5% by weight) of immunogenic proteins, such as antibodies. In some embodiments, the haptoglobin can have an average molecular weight of from 80 kDa to 1,000 kDa, such as from 100 kDa to 1,000 kDa. In some embodiments, the haptoglobin is characterized by having residual hemoglobin as characterized by UV-visible spectroscopy of the Soret peak ranging from 402-407 nm. In some of these embodiments, the residual hemoglobin can be present in an amount less than 10% by weight (e.g., less than 5% by weight, less than 4% by weight, less than 3% by weight, or less than 1% by weight), based on the total weight of all proteins in the haptoglobin-containing composition. In some of these embodiments, the residual hemoglobin can be present in an amount less than 10% by weight (e.g., less than 5% by weight, less than 4% by weight, less than 3% by weight, or less than 1% by weight), based on the total weight of haptoglobin in the haptoglobin-containing composition. In some embodiments, the composition can be substantially free of immunogenic proteins (e.g., immunoglobins).

These compositions can be administered to a subject in need thereof, for example, to treat hemolytic anemia and other conditions characterized by or associated with hemolysis (e.g., sickle cell anemia, malaria, red blood cell transfusions, thalassemia, autoimmune disorders, bone marrow failure, infections, surgery, severe burns, acute lung injury, the administration of chemotherapeutics, radiation therapy, etc.). In some examples, the second retentate fraction can be administered prophylactically to a subject to prevent damage associated with anticipated hemolysis (e.g., prior to surgery, radiation therapy, acute radiation injury, etc.). In some examples, the second retentate fraction can be co-administered with a therapy that induce hemolysis (e.g., a chemotherapeutic agent, an anti-infective agent, a radiation therapy, or a combination thereof). These proteins may also be used in wound-healing applications and to prevent bacterial proliferation/resistance.

These compositions can also be added to compositions comprising red blood cells to stabilize the compositions during storage and to improve the safety of hemoglobin-based blood substitutes.

All of the protein concentrations above (in weight percent) are concentrations determined by SDS-PAGE analysis using the standard methodology described in Example 2.

Analogous methods employing appropriate molecular weight cut-offs can be used to isolate/concentrate a variety of alternative proteins/protein mixtures. For example, the table below illustrates how the bracketing method can be employed for various proteins and plasma fractions.

| Plasma Fraction | Proteins of Interest (MW) | Purified Concentrate (MWCO) |
|---|---|---|
| Cryoprecipitate | Factor VIII (300 kDa), Fibrinogen | Von Willebrand Factor (≥500 kDa)/ |
| | (350 kDa), Von Willebrand | Factor VIII, Von Willebrand Factor, |
| | Factor (360ₙ kDa), | Fibrinogen (200 kDa)/Factor XI (100 |
| | Prothrombin complex concentrates | kDa)/Prothrombin complex |
| | (50-80 kDa), Factor XI (160 kDa) | concentrates (50 kDa) |
| I | Immunoglobulins G (150 kDa) | Von Willebrand Factor (>500 kDa)/ |
| | (150 kDa), Factor VIII (300 kDa), | Factor VIII, Von Willebrand Factor, |
| | Von Willebrand Factor (360ₙ | Fibronectin, Fibrinogen (200 kDa)/ |
| | kDa), Fibronectin (440 kDa), | Immunoglobulins G (100 kDa) |
| | Fibrinogen (350 kDa) | |
| II | Immunoglobulin G (150 kDa) | Immunoglobulins G (100 kDa) |
| III | Prothrombin (70 kDa), | alpha-2 macroglobulin (500 kDa)/ |
| | Immunoglobulins (150 kDa), | Immunoglobulins G (100 kDa)/ |
| | alpha-2 macroglobulin (760 kDa), | Prothrombin, Plasminogen (50 kDa) |
| | Plasminogen (90 kDa) | |
| II + III | Immunoglobulins G (150 kDa), | Immunoglobulins G (100 kDa)/ |
| | Plasminogen (90 kDa) | Plasminogen (50 kDa) |
| II + III supernatant | Antithrombin III (60 kDa) | antithrombin III (50 kDa) |
| IV-1 | Protein C (60 kDa), | Impurities (750 kDa)/HDL, |
| | Ceruloplasmin (130 kDa), | ceruloplasmin (70 kDa)/albumin, |
| | Prothrombin (70 kDa), | prothrombin, antithrombin III, alpha- |
| | Antithrombin III (60 kDa), alpha- | 1 antitrypsin and protein C (50 kDa) |
| | 1 antitrypsin (50 kDa), HDL | |
| | (150-400 kDa) | |
| IV-4 | High MW Hp (90ₙ kDa), | Impurities (750 kDa)/high MW Hp |
| | transferrin (80 kDa), albumin (65 | (500 kDa)/low MW Hp (100 kDa)/ |
| | kDa), hemopexin (60 kDa) | transferrin, albumin, hemopexin, |
| | | small MW Hp (50 kDa) |
| IV-1 + IV-4 (IV) | HDL (140-400 kDa), albumin (65 | Impurities (750 kDa)/high MW Hp |
| | kDa), Hp (90n kDa), transferrin | (500 kDa)/low MW Hp (100 kDa)/ |
| | (80 kDa), Protein C (60 kDa), | HDL, transferrin, albumin, |
| | Ceruloplasmin (130 kDa), | hemopexin, small MW Hp, |
| | Prothrombin (70 kDa), | prothrombin, Protein C, antithrombin |
| | Antithrombin III (60 kDa), aplha- | III, alpha-1 antitrypsin (50 kDa) |
| | 1 antitrypsin (50 kDa), hemopexin | |
| | 60 kDa | |
| V | Small MW Hp (200-90 kDa), | Small MW Hp (70 kDa)/albumin |
| | Serum Albumin (65 kDa) | (50 kDa) |

The resulting proteins/protein mixtures can be used in the applications detailed in the table below.

| Protein in | |
|---|---|
| Concentrate | Indication/Uses |
| Prothrombin | Hemophilia B, bleeding disorders due to lack of |
| complex concentrates | PCC factors, clotting factor replacement, |
| (PCC) | massive bleeding |
| Factor VIII | Hemophilia A, bleeding disorders |
| Von Willebrand | von Willebrand disease |
| Factor | |
| Factor XI | Factor XI deficiency, hemophilia C |
| Fibrinogen | Fibrin Glue |
| Prothrombin | Fibrin Glue |
| Antithrombin III | Antithrombin deficiency, heparin substitute |
| Albumin | Plasma expander, drug carrier |
| Aplha-1 antitrypsin | aplha-1 antitrypsin deficiency |
| Plasminogen | plasmin production |
| HDL | atherosclerosis, cholesterol scavenging, |
| | apolipoprotein A-1 source, antioxidant, |
| | anti-inflammatory, vasoprotection, |
| | antithrombotic |
| Ceruloplasmin | hemopoiesis, detoxification, immunemodulation, |
| | hemorrhage during surgery, anemia, traumatic |
| | shock, hemorrhagic shock, infectious toxic |
| | shock, radiation exposure, infection |
| Haptoglobin | hemolysis |
| Hemopexin | hemolysis |
| Transferrin | iron build-up, hemolysis, infection |
| Immunoglobulin G | hypogammaglobulinemia and other primary |
| | immunodeficiencies, passive immunity to |
| | infectious diseases, secondary |
| | immunodeficiencies caused by diseases |
| | or disease therapy |

Methods for the Purification of Proteins Based on Molecular Size Changes

Also provided herein are methods for isolating a target protein from a solution comprising a plurality of proteins that exploit molecular size changes induced by protein complex formation. Such methods can comprise (i) filtering the protein solution by ultrafiltration against a first filtration membrane, thereby forming a first retentate fraction comprising impurities having a molecular weight above a first cutoff value and a first permeate fraction comprising the target protein and impurities having a molecular weight below the first cutoff value; (ii) contacting the first permeate fraction with a binding molecule that selectively associates with the target protein to form a target protein complex having a molecular weight above the first cutoff value; and (iii) filtering the first permeate fraction by ultrafiltration against a second filtration membrane (at the same or above the cut-off of the first membrane), thereby forming a second retentate fraction comprising the target protein complex having a molecular weight above the first cutoff value and a second permeate fraction comprising the impurities having a molecular weight below the first cutoff value.

In some cases, the target protein complex can be isolated (e.g., if the target protein complex itself is useful, or if the target protein complex is more stable under storage than the target protein).

In other cases, the method can further involve dissociating the target protein complex to re-form the target protein, and isolating the target protein. For example, the method can further comprise (iv) contacting the second retentate fraction with a dissociating agent, thereby inducing dissociation of the target protein complex to the target protein and the binding molecule, and (v) filtering the second retentate fraction to separate the target protein from the binding molecule and the dissociating agent, thereby isolating the target protein.

In some of these embodiments, step (v) can comprise filtering the second retentate fraction by ultrafiltration against a third filtration membrane, thereby forming a third retentate solution comprising the target protein having a molecular weight above a second cutoff value and a second permeate fraction comprising the impurities having a molecular weight below the second cutoff value.

In some embodiments, ultrafiltration may be done with staging to improve separation between retained and filtered solutes and to increase product recovery.

The binding molecule can be any suitable molecule that selectively associates with the target protein, thereby forming a target protein complex having a molecular weight greater than the target protein (e.g., at least 10 kDa greater than the target protein, at least 25 kDa greater than the target protein, at least 50 kDa greater than the target protein, at least 100 kDa greater than the target protein, or greater).

The term “selectively associates”, as used herein when referring to a binding molecule, refers to a binding reaction which is determinative for the target protein in a heterogeneous population of other similar compounds. Generally, the interaction is dependent upon the presence of a particular structure (e.g., an antigenic determinant or epitope) on the target protein. By way of example, an antibody or antibody fragment selectively associates to its particular target (e.g., an antibody specifically binds to an antigen) but it does not bind in a significant amount to other proteins present in the sample or to other proteins to which the antibody may come in contact in an organism.

In some embodiments, a binding molecule that “specifically binds” a target protein has an affinity constant (Kₐ) greater than about 10⁵ M⁻¹ (e.g., greater than about 10⁶ M⁻¹, greater than about 10⁷ M⁻¹, greater than about 10⁸ M⁻¹, greater than about 10⁹ M⁻¹, greater than about 10¹⁰ M⁻¹, greater than about 10¹¹ M⁻¹, greater than about 10¹² M⁻¹, or more) with that target protein. These values represent desired affinities for binding that may be altered by use of a proper dissociating agent so that the target complex is dissociated.

Examples of suitable classes of binding molecules include, for example, antibodies, antibody fragments, antibody mimetics, proteins (e.g., protein A), peptides, oligonucleotides, DNA, RNA, aptamers, organic molecules, inteins, split-inteins, and combinations thereof. In certain embodiments, the binding molecule comprises an antibody. The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen. The term encompasses intact and/or full length immunoglobulins of types IgA, IgG (e.g., IgG1, IgG2, IgG3, IgG4), IgE, IgD, IgM, IgY, antigen-binding fragments and/or single chains of complete immunoglobulins (e.g., single chain antibodies, Fab fragments, F(ab′)2 fragments, Fd fragments, scFv (single-chain variable), and single-domain antibody (sdAb) fragments), and other proteins that include at least one antigen-binding immunoglobulin variable region, e.g., a protein that comprises an immunoglobulin variable region, e.g., a heavy (H) chain variable region (VH) and optionally a light (L) chain variable region (VL). The light chains of an antibody may be of type kappa or lambda.

An antibody may be polyclonal or monoclonal. A polyclonal antibody contains immunoglobulin molecules that differ in sequence of their complementarity determining regions (CDRs) and, therefore, typically recognize different epitopes of an antigen. Often a polyclonal antibody is derived from multiple different B cell lines each producing an antibody with a different specificity. A polyclonal antibody may be composed largely of several subpopulations of antibodies, each of which is derived from an individual B cell line. A monoclonal antibody is composed of individual immunoglobulin molecules that comprise CDRs with the same sequence, and, therefore, recognize the same epitope (i.e., the antibody is monospecific). Often a monoclonal antibody is derived from a single B cell line or hybridoma. An antibody may be a “humanized” antibody in which for example, a variable domain of rodent origin is fused to a constant domain of human origin or in which some or all of the complementarity-determining region amino acids often along with one or more framework amino acids are “grafted” from a rodent, e.g., murine, antibody to a human antibody, thus retaining the specificity of the rodent antibody.

The dissociating agent can comprise any suitable agent or agent that stimulates dissociation of the target protein and binding molecule. Suitable dissociating agents are known in the art, and include, for example, pH modifiers (e.g., acids and/or bases), salts, polyelectrolytes, chaotropic agents, non-aqueous solvents (e.g., alcohols such as ethanol, methanol, isopropanol, butanol, 2-propanol, phenol, or combinations thereof) or combinations thereof.

In some examples, contacting the second retentate fraction with a dissociating agent can comprise contacting the second retentate fraction with an effective amount of a pH modifier to produce an acidic or basic pH, selected so as to facilitate dissociation of the target protein and the binding molecule.

In some cases, contacting the second retentate fraction with a dissociating agent can comprise contacting the second retentate fraction with an effective amount of a pH modifier to produce a pH of 6 or less (e.g., 5.5 or less, 5 or less, 4.5 or less, 4 or less, 3.5 or less, 3 or less, or 2.5 or less). In some embodiments, contacting the second retentate fraction with a dissociating agent can comprise contacting the second retentate fraction with an effective amount of a pH modifier to produce a pH of 2 or more (e.g., 2.5 or more, 3 or more, 3.5 or more, 4 or more, 4.5 or more, 5 or more, or 5.5 or more).

Contacting the second retentate fraction with a dissociating agent can comprise contacting the second retentate fraction with an effective amount of a pH modifier to produce a pH ranging from any of the minimum values described above to any of the maximum values described above. For example, in some embodiments, contacting the second retentate fraction with a dissociating agent can comprise contacting the second retentate fraction with an effective amount of a pH modifier to produce a pH of from 2 to 6, such as from 3 to 6.

In other cases, contacting the second retentate fraction with a dissociating agent can comprise contacting the second retentate fraction with an effective amount of a pH modifier to produce a pH of 8 or more (e.g., 8.5 or more, 9 or more, 9.5 or more, 10 or more, or 10.5 or more). In some embodiments, contacting the second retentate fraction with a dissociating agent can comprise contacting the second retentate fraction with an effective amount of a pH modifier to produce a pH of 11 or less (e.g., 10.5 or less, 10 or less, 9.5 or less, 9 or less, or 8.5 or less.

Contacting the second retentate fraction with a dissociating agent can comprise contacting the second retentate fraction with an effective amount of a pH modifier to produce a pH ranging from any of the minimum values described above to any of the maximum values described above. For example, in some embodiments, contacting the second retentate fraction with a dissociating agent can comprise contacting the second retentate fraction with an effective amount of a pH modifier to produce a pH of from 8 to 11, such as from 8 to 10.

In some examples, contacting the second retentate fraction with a dissociating agent can comprise contacting the second retentate fraction with a non-aqueous solvent, such as an alcohol. Examples of such non-aqueous solvents include, for example, ethanol, methanol, isopropanol, butanol, 2-propanol, phenol, or combinations thereof.

In some examples, contacting the second retentate fraction with a dissociating agent can comprise contacting the second retentate fraction with a chaotropic agent, such as guanidinium chloride, lithium perchlorate, lithium acetate, magnesium chloride, sodium dodecyl sulfate, thiourea, urea, or a combination thereof.

In other embodiments, step (iv) can comprise heating the second retentate fraction to stimulate dissociation of the target protein and binding molecule (e.g., to a temperature of from 40° C. to 60° C.).

In some embodiments, the ultrafiltration can comprise tangential-flow filtration.

By way of non-limiting illustration, examples of certain embodiments of the present disclosure are given below.

### EXAMPLES

### Example 1: Scalable Production of Apohemoglobin Via Tangential Flow Filtration

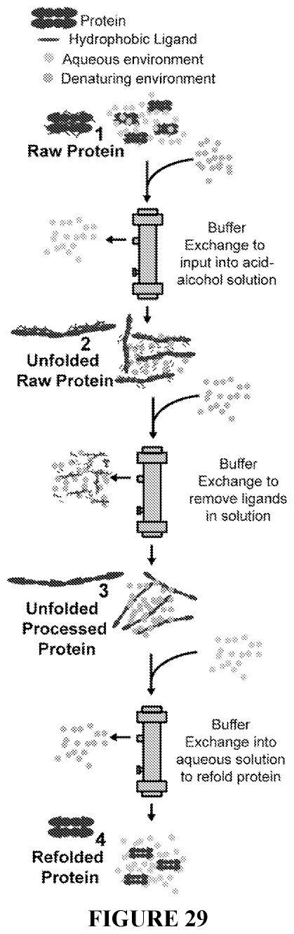

The general schematic for the procedure to remove hydrophobic ligands from proteins employing the invention presented here is shown in FIG. 29.

Apohemoglobin (apoHb) is a dimeric globular protein with two vacant heme-binding pockets that can bind heme or other hydrophobic ligands. Purification of apoHb is based on partial hemoglobin (Hb) unfolding to facilitate heme extraction into an organic solvent. However, current production methods are time consuming, difficult to scale up, and use highly flammable and toxic solvents. In this study, a novel and scalable apoHb production method was developed using an acidified ethanol solution to extract the hydrophobic heme ligand into solution and tangential flow filtration to separate heme from the resultant apoprotein. Total protein and active protein yields were >95% and ˜75%, respectively, with <1% residual heme in apoHb preparations and >99% purity from SDS-PAGE analysis. Virtually no loss of apoHb activity was detected at 4° C., −80° C., and in lyophilized form during long term storage. Structurally, size exclusion chromatography (SEC) and circular dichroism (CD) spectroscopy indicated that apoHb was dimeric with a ˜25% reduction of helical content compared to Hb. Furthermore, mass spectroscopy and reverse-phase chromatography indicated that the mass of the α and β subunits were virtually identical to the theoretical mass of these subunits in Hb and had no detectable oxidative modifications upon heme removal from Hb. SEC confirmed that apoHb bound to haptoglobin at similar ratio to that of native Hb. Finally, reconstituted Hb (rHb) was processed via a hemichrome removal method to isolate functional rHb for biophysical characterization in which the O₂ equilibrium curve, O₂ dissociation and CO association kinetics of rHb were virtually identical to native Hb. Overall, this study describes a novel and improved method to produce apoHb, as well as presents a comprehensive biochemical analysis of apoHb and rHb.

Human hemoglobin (Hb) is the major protein component contained inside human red blood cells (RBCs), and is well known for its role in oxygen (O₂) storage and transport. It is a tetrameric protein (64 kDa), which consists of two pairs of αβ dimers (32 kDa) held together by non-covalent bonds. In each of the four globin chains (2α and 2β globins), a single heme prosthetic group is tightly bound inside the hydrophobic heme-binding pocket. Upon removal of heme from Hb, the resulting protein loses some of its helical content compared to native Hb. The resulting apoprotein is referred to as apohemoglobin (apoHb). ApoHb can react with heme to form reconstituted Hb (rHb), which shows virtually no difference in biophysical properties compared to native Hb. The heme-binding ability and heme-induced structural changes of apoHb make it an interesting precursor for studies into in vivo Hb synthesis and recombinant Hb production.

ApoHb is an attractive delivery vehicle for hydrophobic drug molecules, which can bind within the vacant heme-binding pockets. Heme is highly hydrophobic and cytotoxic; however, when bound inside the heme-binding pocket of Hb, its toxicity is reduced, and aqueous solubility increases. In addition to heme, other hydrophobic molecules such as modified hemes or therapeutic drug molecules can bind to the hydrophobic heme-binding pocket of apoHb.

Another exciting property of apoHb is its' clearance through CD163+ macrophages or monocytes. Similar to Hb, apoHb binds to haptoglobin (Hp). Hp is a plasma protein mainly responsible for the clearance of cell-free Hb. The apoHb-Hp/Hb-Hp complex is then recognized and uptaken by CD163+ macrophages and monocytes. This specific mode of clearance allows for targeted drug delivery to macrophages or monocytes. Thus, the ability of apoHb to bind hydrophobic molecules and facilitate targeted delivery towards CD163+ macrophages or monocytes make it a promising hydrophobic drug delivery vehicle.

From these properties, not only can apoHb be used as a drug carrier for hydrophobic molecules (i.e. molecules insoluble in aqueous solution) and targeted drug delivery to macrophages and monocytes, but its high heme affinity could be used to scavenge heme in vivo. States of hemolysis release cell-free Hb, which can lose its heme moiety. Free heme can undergo various redox-reactions, causing oxidation of various tissues. ApoHb could scavenge free heme, and thus forming cell-free Hb that can be cleared through CD163+ macrophages or monocytes. Furthermore, the hydrophobic molecule binding properties of apoHb can be used to bind MRI contrast agent molecules such as Mn-porphyrins (similar structure to normal Hb heme but with switching the Fe metal atom to Mn). The same idea applies to binding of fluorescent molecules to the vacant hydrophobic heme-binding pocket.

Another application of apoHb is its potential use in photodynamic therapy (PDT). PDT has recently been used to effectively treat cancers and other illnesses through the production of reactive oxygen species (ROS). The ROS produced by PDT surpasses the ability of cancer cells to resist cell apoptosis, disrupts the tumor vasculature and promotes shifting the immune system against the tumor. Furthermore, this treatment could be used for cancers like triple-negative breast cancer, in which commonly targeted receptors are not expressed. However, most photosensitizers (PS) lack specificity for tumor cells, have poor solubility, and cause systemic photosensitivity, inducing phototoxic and photoallergic reactions. Many PS targeting mechanisms are expensive, complicated to develop, or leak PS. Fortunately, the high expression of CD163+ tumor associated macrophages (TAM) in cancers could be targeted through their uptake of apohemoglobin (apoHb) via CD163 mediated endocytosis. PS bound to apoHb could improve PDT treatment by not only improving its biocompatibility and effectiveness, but also by specifically targeting a form of TAM that contributes to tumor growth. Furthermore, the metals in PS provide a second pathway for ROS production and anti-tumor immune response. Thus, treatment with PS bound apoHb could enhance the immunological shift against the tumor by lowering macrophage density and stimulating TAM differentiation to an anti-cancer phenotype. This immune change can destroy secondary tumors and prevent cancer metastasis and regression. We have successfully bound aluminum-phthalocyanines (a highly potent PS molecule currently undergoing clinical trials) to apoHb, thus increasing its' solubility in aqueous solution.

The first successful method for producing active apoHb was developed by Faneli et al. in 1958. In Fanelli's acetone extraction method, Hb was added to acidified acetone at −20° C. extracting heme into solution while precipitating the apoprotein (globin). After separation of the solid protein from the liquid phase, the apoprotein was re-dissolved in deionized (DI) water followed by extensive dialysis, yielding apoHb. Another procedure for active apoHb production was later developed by Teale in 1959, and further improved upon by Yonetani. In this process, heme was removed from Hb through exposure to acidified methylethylketone (MEK), forming two immiscible liquid layers. The heme partitions into the organic layer, while the globin partitions into the aqueous layer. After liquid-liquid separation of the layers, the aqueous globin solution underwent extensive dialysis similar to the acetone extraction method to yield apoHb.

However, the aforementioned apoHb production processes have various drawbacks that complicate scale up. First, both processes use highly flammable solvents and require costly separation equipment. Additionally, in the case of MEK extraction (the solvent most likely to be used for scale up), about 40% of the water-rich phase becomes saturated with MEK. Thus, extensive treatment is required to lower the MEK concentration in the aqueous phase before the aqueous phase can be safely discarded. The high MEK concentration in the aqueous phase will also require repeated MEK extractions to significantly lower the heme content of the purified apoHb. In the case of acetone extraction, not only is there an additional safety risk associated with centrifuging a flammable solvent, but the process may also require sequential acetone exposure to sufficiently remove heme, especially due to the possibility of heme entrapment within the protein precipitate. Finally, when acetone and MEK are used as heme extraction solvents at large-scales, buffer-exchange via dialysis will require large volumes of buffer and is a slow process. In this current work, a scalable and simple process for manufacturing apoHb is described.

Tangential flow filtration (TFF) is a size exclusion filtration technique greatly used in industrial biotechnology for purification of biomolecules due to its linear scalability, economic benefits and long membrane lifetime. Additionally, TFF easily facilitates controlled buffer exchange via diafiltration, which is preferable to extensive and lengthy dialysis and can reduce the equipment footprint for production. In this example, TFF was used to produce and purify apoHb using an acidic 80% ethanol solution (v/v) as the heme extraction solvent. It is important to note that ethanol poses a much lower flammability risk compared to previously used heme extraction solvents given that its flashpoint is 20° C. compared to −18° C. and −3° C. for acetone and MEK, respectively.

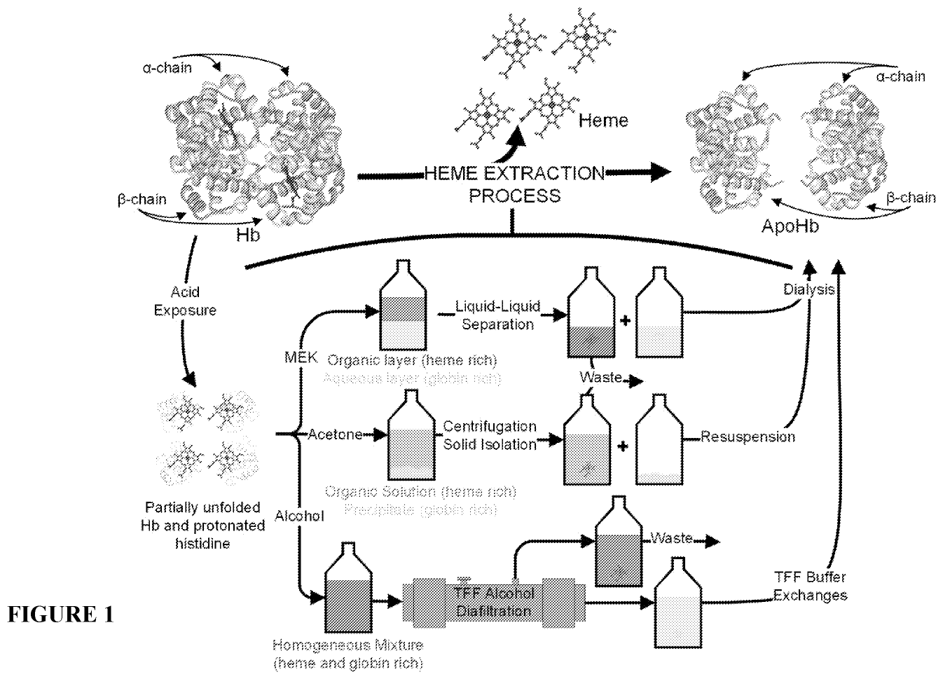

In this TFF process, the Hb precursor in aqueous solution was added to a TFF system filled with an acidic ethanol solution. Then, the mixture of acidic ethanol and Hb underwent continuous diafiltration in the TFF system with acidic ethanol as the diafiltration solution until sufficient heme was extracted from the mixture (i.e., the absorbance ratio of the Soret peak at 412 nm divided by the 280 nm protein peak was lower than 0.1). Next, DI water was used as the diafiltration solution to neutralize the acidic ethanol-Hb solution and to remove ethanol and any free heme from solution. Finally, the desired buffer for apoHb storage and analysis was used as the diafiltration solution for the last diafiltration step (a more detailed procedure for apoHb production via TFF can be found in the Methods Section). FIG. 1 summarizes the prominent methods for producing active apoHb, compared to the method presented in this example.

Analysis of apoHb produced via the TFF purification process (TFF-apoHb) was accomplished by analyzing protein yields and biochemical properties of the resultant apoHb. The stability of apoHb as a function of storage time was also examined at different concentrations and temperatures via quantification of active and total protein of stored apoHb samples. The reconstituted Hb from TFF-apoHb also had its biophysical properties analyzed and compared to native Hb via its absorbance spectrum, O₂ equilibrium curve, O₂ dissociation and carbon monoxide (CO) binding kinetics.

Materials and Methods

Materials. Na₂HPO₄ (sodium phosphate dibasic), NaH₂PO₄ (sodium phosphate monobasic), NaHCO₃(sodium bicarbonate), and hemin chloride were all procured from Sigma Aldrich (St. Louis, Mo.). KCN (potassium cyanide), HCl (hydrochloric acid), acetone, HPLC grade acetonitrile, HPLC grade trifluoroacetic acid (TFA), nylon syringe filters (rated pore size 0.22 μm), and dialysis tubing (rated pore size: 6-8 kDa) were purchased from Fisher Scientific (Pittsburgh, Pa.), while Millex-GP PES syringe filters (rated pore size: 0.2 μm) were purchased from Merck Millipore (Billerica, Mass.). Expired units of human RBCs were generously donated by the Transfusion Service in the Wexner Medical Center at The Ohio State University (Columbus, Ohio).

Hb Preparation. Human Hb for use in this study was prepared via TFF as described by Palmer et al. (Palmer, A. F., Sun, G. & Harris, D. R. Tangential flow filtration of hemoglobin. Biotechnol. Prog. 25, 189-199 (2009)). The concentration of Hb was determined spectrophotometrically based on the Winterbourn equation.

TFF-apoHb Preparation. A KrosFlo Research II TFF system (Spectrum Laboratories, Rancho Dominguez, Ca) with a single 10 kDa polysulfone (PS) hollow fiber (HF) module was used to purify apoHb from Hb. To examine the scalability of the purification process, the process was first performed on TFF filter (P/N: X11S-300-10S) with 20 cm² surface area (MicroKros, Spectrum Laboratories, Rancho Dominguez, Ca) then scaled up to TFF filter (P/N: M11S-360-01S) with 1,050 cm² surface area (MiniKros, Spectrum Laboratories, Rancho Dominguez, Calif.). For both size filters, the individual HFs were 0.5 cm in diameter and were 20 cm in length. Purified Hb was added to a 80% (v/v) EtOH/DI water mixture containing 3 mM HCl (acidic ethanol) to achieve a maximum protein concentration of 2 mg/mL. For experiments with microKros filters, 18 mg of Hb was used as the basis. However, experiments utilizing miniKros filters (larger surface area than microKros filters) consisted of three batches with 1 g Hb, three batches with 1.2 g Hb and 8 batches with 2.0 g Hb (ran with two parallel HF modules) as the basis, respectively. The Hb dispersed in the acidic ethanol solution was continuously subjected to diafiltration with 9 times its' initial volume with acidic ethanol to remove heme from solution. After heme removal, the heme-free globin was subjected to diafiltration with DI water with 5 times its volume.

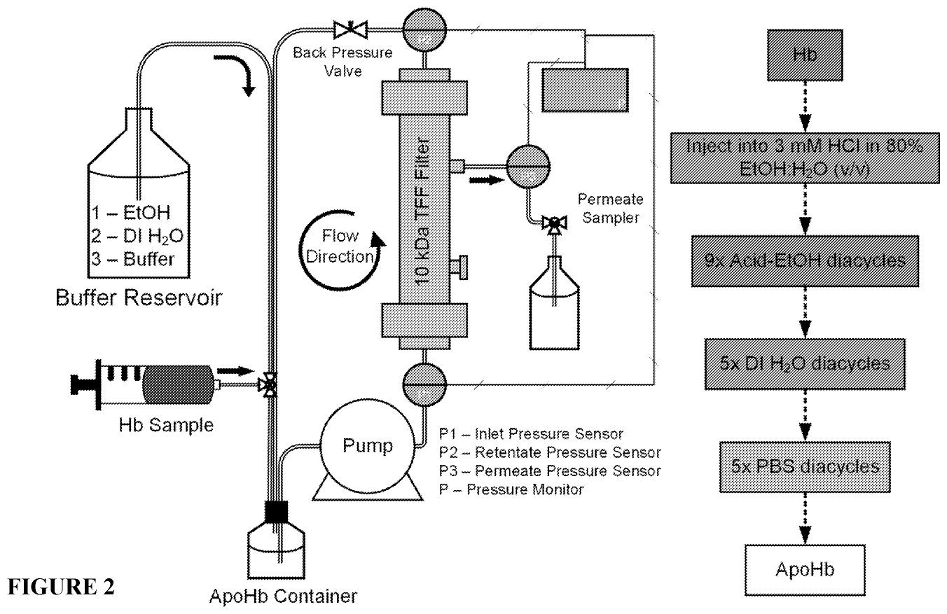

Finally, the apoHb solution was subjected to diafiltration with 5 times its initial volume using a final buffer solution consisting of either phosphate buffered saline (PBS, 10 mM phosphate, 137 mM NaCl, and 2.7 mM KCl pH 7.4) or 0.1 M phosphate buffer (PB, pH 7.0). During processing, flow rates of 25 mL/min and 1.1 L/min were used for the microKros and miniKros HF modules, respectively. The transmembrane pressure was maintained at 7±1 psi with a back-pressure valve to facilitate optimal permeate flux. For large-scale production, a final concentration step was performed in which the apoHb solution volume was reduced to 50±10 mL then further concentrated on 10 kDa PS microKros filters to a final total protein concentration of 50-115 mg/mL. The entire process was performed in a cold room maintained at 4±1° C. After each run, TFF modules were rinsed with DI water followed by sanitization with 0.5 M NaOH. The modules were stored in 0.1 M NaOH and were extensively washed with DI water prior to use. A schematic of the apoHb TFF production schematic is shown in FIG. 2.

Acetone ApoHb Preparation. The method commonly used to produce apoHb via acetone heme extraction was followed according to the protocol outlined by Fanelli et al.

Total Protein Assays. The total protein concentration of the apoHb solution was measured using a Coomassie Plus Protein assay kit (Pierce Biotechnology, Rockford, Ill.).

ApoHb Activity Assay. The activity of the heme-binding pocket of apoHb was determined via a dicyanohemin (DCNh) incorporation assay. Briefly, analysis of the equilibrium absorbance at 420 nm from apoHb and DCNh mixtures was used to determine the saturation point of apoHb heme-binding pockets with heme. The extinction coefficients of DCNh and rHbCN were 85 mM⁻¹ cm⁻¹ and 114 mM⁻¹ cm⁻¹, respectively.

ApoHb Stability. Three TFF-apoHb batches were prepared. Each batch was divided into three groups to test apoHb stability over time. These groups consisted of unconcentrated, concentrated and lyophilized apoHb. The unconcentrated group was obtained after buffer exchange of apoHb into PBS buffer at a concentration of ˜2 mg/mL. The remainder of the batch was either sent to be lyophilized or to be concentrated to ˜40 mg/mL. Immediately after production, apoHb activity and total protein was quantified via the DCNh activity assay and 280 nm absorbance, respectively. Of the concentrated and unconcentrated apoHb groups from each batch, samples were stored at either 37, 22, 4 or −80° C. for subsequent analysis. The lyophilized powder was stored in a closed container at −80° C. Additionally, the stored apoHb groups were reconstituted into rHb and had their absorbance spectra and O₂ dissociation curve measured. After measuring the initial time point (immediately after production), each storage condition was assayed at varying time intervals to measure apoHb activity over time. These time intervals were chosen to capture relevant changes in activity at each storage condition. At 37° C., apoHb was expected to quickly lose activity, so measurements were made every 12 hours. In contrast, apoHb stored at −80° C. was expected to maintain activity for longer time durations. Thus, after initial measurements on a weekly basis, the insignificant changes lead to longer intervals between measurements. Statistical analysis was performed on JMP Pro v 12.2.2 (SAS Institute, Cary, N.C.) and measured concentrations were compared to the initial time point values of each batch. A linear fit with the logarithmic value of the concentration was used to examine the effect of time with the ANOVA test. For time point differences, time was considered as discrete and the TUKEY HSD test was performed.

Mass Spectroscopy. Before analysis, Hb and apoHb samples were buffer exchanged into 100 mM ammonium acetate (Fisher Scientific; San Jose, Calif.) using Micro Bio-Spin™ 6 columns (Bio-Rad; Hercules, Calif.). Samples were tested on a Finnigan LTQ mass spectrometer (Thermo Fisher Scientific, Waltham, Mass.) and analyzed using Xcalibur 2.2 software (Thermo Fisher Scientific, Waltham, Mass.). Samples from the same stock apoHb and Hb were then denatured in 1% acetic acid acetate (Fisher Scientific; San Jose, Calif.) and retested. The mass spectrometer parameters were: spray voltage: 1.5 kV; flow rate: 5 μL/min; capillary temperature: 200° C.; 3 microscans; and 100 ms injection time. The data was deconvoluted using mMass 5.5.0, (Copyright 2018 by Martin Strohalm).

Residual Heme Analysis. The residual heme in apoHb preparations was quantified via size exclusion chromatography (SEC). ApoHb samples prepared via TFF were separated on an analytical BioSep-SEC-53000 (600×7.5 mm) column (Phenomenex, Torrance, Calif.) attached to a Waters 2535 quaternary gradient module, Waters 2998 photodiode array multi-wavelength detector, and controlled using Empower Pro software (Waters Corp., Milford, Mass.). The mobile phase consisted of 50 mM potassium phosphate, pH 7.4. Since pigment-free proteins such as apoHb absorb at 280 nm and heme bound proteins such as Hb have a sharp Soret peak at 400-450 nm, the absorption wavelength was set at λ=280 nm to detect protein (although heme and heme-bound proteins also absorb at 280 nm), and λ=405 and 413 nm to detect protein containing heme. The number of heme molecules retained in apoHb preparations produced via TFF were determined by comparing the Soret spectra of four apoHb samples with the Soret spectra of a Hb sample of known concentration. The number of heme molecules in the apoHb preparation was then compared to the total protein of the sample (on a molar basis) to obtain the percentage of residual heme in each apoHb preparation.

Quaternary Structure. To estimate the quaternary structure of TFF-apoHb, apoHb and protein standards (conalbumin, 76 kDa; hHb, 64 kDa; carbonic anhydrase, 29 kDa; ribonuclease A, 14 kDa; and aprotinin, 6.5 kDa) were analyzed on a SEC column. The known molecular weight (MW) of the standards and their elution volumes were used to determine the coefficients (A, B) of a base 10 exponential function (MW=10^{A*(elution volume)+B)} via non-linear regression. The estimated function parameters were used to estimate the MW of TFF-apoHb based on its elution volume. Samples were separated on an analytical Acclaim SEC-1000 (4.6×300 mm) column (Thermo Fisher Scientific, Waltham, Mass.) attached to a Dionex UltiMate 3000 system (Thermo Fisher Scientific, Waltham, Mass.). The mobile phase consisted of 50 mM potassium phosphate, pH 7.4. The flow rate and UV-visible spectral detection was controlled on Chromeleon 7 software with detection set to λ=280 nm to detect protein elution at a flow rate 0.35 mL/min.

Haptoglobin Binding. To analyze haptoglobin (Hp) binding to TFF-apoHb, increasing concentrations of apoHb were mixed with haptoglobin (Hp) and the resultant mixture separated on a SEC column for analysis. Large molecular weight Hp (mixture of Hp2-2 and Hp2-1) was mixed with apoHb with a molecular weight of ˜32 kDa (dimeric apoHb) and separated on an analytical Acclaim SEC-1000 (4.6×300 mm) column (Thermo Fisher Scientific, Waltham, Mass.) attached to a Dionex UltiMate 3000 system (Thermo Fisher Scientific, Waltham, Mass.). The mobile phase consisted of 50 mM potassium phosphate, pH 7.4. The flow rate and UV-visible spectral detection was controlled on Chromeleon 7 software with detection set to Δ=280 nm to detect protein elution at a flow rate 0.35 mL/min. The percent change of the area under the curve between pure apoHb and a mixture of excess apoHb and Hp was used to determine the percent of apoHb that was bound to Hp. This percentage was compared to the mass of pure apoHb loaded to determine the Hp binding capacity of apoHb. The same procedure was repeated with Hb for comparison.

Reverse Phase Chromatography. Reverse phase (RP) chromatography was performed with a BioBasic-18 column (Thermo Scientific, Waltham, Mass.) on a Thermo Scientific Dionex Ultimate UHPLC system. The flow rate of the mobile phase was set to 0.75 mL/min. The column was equilibrated with 35% acetonitrile and 65% TFA (0.5 wt %, pH 2.6) for 10 minutes. The gradient was then shifted to 43% acetonitrile over a 1 minute interval. The protein was eluted with an increasing linear gradient of 43 to 47% acetonitrile for 30 minutes. The column was then held at 47% acetonitrile for 20 minutes.

Circular Dichroism. The far UV circular dichroism (CD) spectra of TFF-apoHb was measured on a JASCO J-815 CD spectrometer. Various TFF-apoHb samples and Hb were diluted in DI water to approximately 10 μM. The ellipticity of the samples was measured from 190-260 nm using a 0.1 mm path-length quartz cuvette. The change in alpha helical content of the apoglobin was determined via the ratio of the alpha-helix peak at 222 nm between TFF-apoHb to hHb.

Hb Reconstitution and Preparation. To regenerate the 02-binding capacity of Hb (i.e. reconstituted Hb, rHb), samples of apoHb were reconstituted with hematin to yield met-rHb and then reduced to yield rHb. First, hematin was added in excess to apoHb to yield met-rHb. The reaction was left overnight at 4±0.5° C. to go to completion. Met-rHb was centrifuged and passed through a 0.22 μm filter before any experiments were conducted. Reduction of met-rHb to yield deoxy-rHb was achieved by adding sodium dithionite at 1.5 mg/mL to met-rHb. The solution was then subjected to diafiltration on a 10 kDa TFF module to remove excess dithionite and any excess heme in solution using a modified HEMOX buffer (135 mM NaCl, 30 mM TES {N-[Tris (hydroxymethyl) methyl]-2-aminoethanesulfonic acid}, 5 mM KCl, pH 7.40±0.02 at 37° C.). During diafiltration, the system was open to the atmosphere to facilitate the conversion of rHb to oxygenated rHb.

Hemichrome Removal and rHb purification. rHb presented an altered absorbance spectra compared to pure Hb corresponding to the presence of hemichromes and/or heme bound to denatured globins. Two methods were developed to remove these unwanted species from solution. For analysis of small dilute samples in a spectrophotometer, passing the solution through a 0.22 μm nylon syringe filter removed the hemichromes by binding them to the hydrophobic filter membrane. However, this method was limited to processing small volumes of material, since the filter membrane would become saturated with these globin-heme species. When larger volumes of rHb were needed for analysis (i.e. more than 1 mg), an oxy-rHb sample was placed under a CO atmosphere to convert rHb into CO-rHb, a highly stable form of Hb. Then, the CO-rHb solution with the unwanted heme-globin complexes was heated to 65±1° C., and left under a CO atmosphere during about 100 minutes to precipitate hemichrome/heme. The hemichrome/heme precipitate was removed and the resulting CO-rHb solution was converted into oxy-rHb by placing it under a pure O₂ stream for 2 hours.

rHb Analysis. Various liganded forms of rHb were analyzed via UV-Vis spectroscopy and compared against native Hb. The oxy-rHb and oxyHb equilibrium binding curves were measured using a Hemox analyzer (TCS Scientific Corp., New Hope, Pa.) at 37° C. The spectra of rHb was measured after one day reaction with excess heme (stage 1), after reduction and diafiltration with the modified HEMOX buffer (stage 2), after placing the rHb mixture under a CO atmosphere (stage 3), after heating the CO-rHb mixture and removing precipitate (stage 4), and after re-oxygenating the rHb sample (stage 5). Spectral deconvolution software was developed in the Python programming language (Python Software Foundation Beaverton, Oreg.) using the non-linear least squares function curve_fit of the SciPy package to determine the fraction of various liganded forms of rHb that contribute to the final spectra of the rHb mixture (i.e. containing metHb, hemichrome, oxyHb, HbCO and heme).

Stopped Flow Kinetics. CO binding to deoxyrHb, and O₂ release from oxyrHb were measured using an Applied Photophysics SF-17 microvolume stopped-flow spectrophotometer (Applied Photophysics Ltd., Surrey, United Kingdom). Rapid kinetic measurements were performed using protocols previously described by Rameez and Palmer (Rameez, S. et al. Encapsulation of hemoglobin inside liposomes surface conjugated with poly(ethylene glycol) attenuates their reactions with gaseous ligands and regulates nitric oxide dependent vasodilation. Biotechnol. Prog. 28, 636-645 (2012)). Unmodified human Hb was used as a control. PBS (0.1 M, pH 7.4) was used as the reaction buffer for all kinetic measurements.

Results and Discussion

History of ApoHb Production Methods. The first published report of isolating globin (i.e., mixture of apoHb in its active and inactive forms) from Hb was from 1892 by Bertin-Sans and de Moitessier. In their method, oxygenated blood was coagulated with ether and mixed with a boiling solution of 10% tartaric acid in ethanol and further processed to yield apoHb. In 1898, Schulz made the first analysis of a purified globin solution produced via a mixture of ether and alcohol. Using this method, heme was extracted into the organic ether-ethanol phase leaving the globin in the aqueous phase. Later studies continued to use this protocol with slight modifications to produce globin until 1926. At that time, Hill and Holden noted that globin solutions were not soluble at the isoelectric point of Hb and the few available analyses of rHb were unclear on its biochemical properties. These issues were attributed to extensive globin denaturation (i.e., protein unfolding) from the use of harsh organic solvents at elevated temperatures, which lowered the yield of active apoglobin. Thus, Hill and Holden developed a very rigorous low temperature procedure that avoided the use of alcohol by using kieselguhr in ether to absorb heme. Using this method, Hill and Holden theorized it would not require protein unfolding, since it appeared that kieselguhr would remove heme under non-denaturing conditions. Yet, the theory of producing more active apoglobin due to the reduced protein unfolding step was shown to be highly improbable. Furthermore, Hsien Wu later noted that the major advancement in Hill and Holden's method was the low acidity of the solution and the low temperature of the process, and not because protein unfolding was minimized or abolished. It was also shown that performing Schulz's procedure under Hill and Holden's experimental conditions provided the same results as Hill and Holden. Finally, later studies by Ansos and Mirsky demonstrated the reversibility of protein unfolding, substantiating the idea that protein unfolding was not necessarily harsh for use in heme extraction. This idea of reversible protein unfolding led to the development of the acidic acetone heme extraction method, commonly used to this day. The acid-acetone procedure produces active soluble apoglobin even after unfolding the protein (to the point of precipitation) in acidic acetone.

Upon establishment of the previously mentioned acid-acetone or acid-methyl ethyl ketone (MEK) heme extraction procedures, these methods became the standard for producing apoHb in the literature. However, no modifications or improvements on these methods were made since their conception in the 1950s. These procedures require the use of highly flammable solvents which, when combined with the requirement of centrifugation or liquid-liquid extraction equipment, possess large safety risks for large-scale production. Additionally, since these processes use highly toxic solvents (acetone or MEK), it reduces possible biomedical applications of apoHb due to the presence of residual solvent in the preparation.

More recently developed apoglobin production methods employ acidified alcohols with subsequent separation facilitated by heme agglomeration, precipitation or adsorption on activated charcoal. Yet, these alcohol-derived apoglobins were made for applications in the food industry or for heme production, and did not provide an analysis of the retention of native apoHb activity or extent of heme removal. In this current study, the use of acidic ethanol-water heme extraction combined with TFF allows for the scalable and safe production of apoHb. A key advantage of this process is the absence of strong denaturants such as acetone or other ketones in the process. There is no method in the literature that describes purification of active apoHb from Hb in which both the heme and globin remain in the same phase. Additionally, previous apoHb production methods require extensive dialysis, which can be replaced by the quicker buffer exchange process facilitated by TFF run in diafiltration mode.

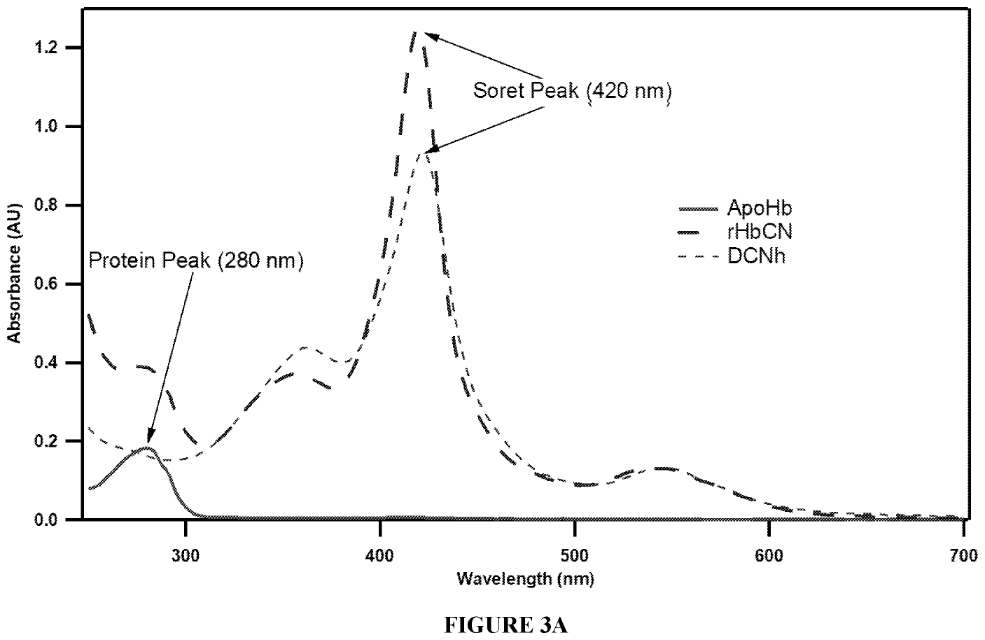

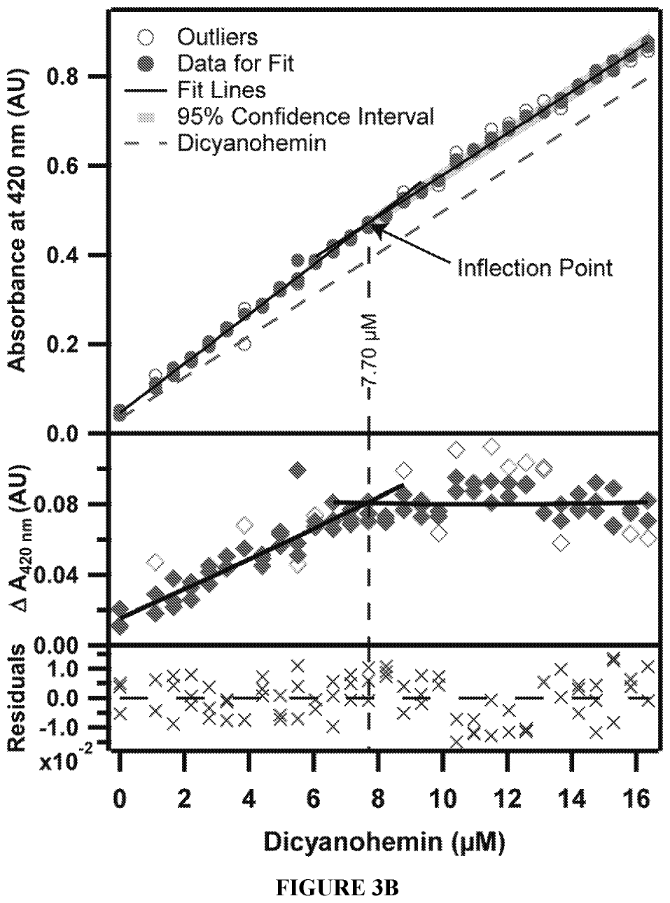

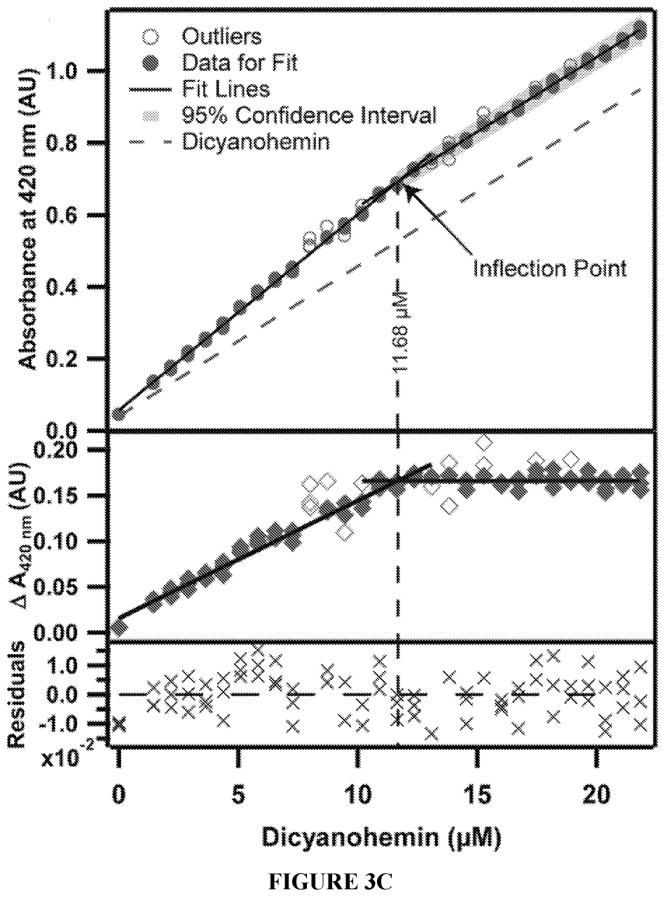

TFF Production of ApoHb. Earlier apoHb studies showed that the majority of purified apoHb product consisted of denatured apoHb with a small fraction of active apoHb in solution. Therefore, for applications where it is important to know the activity of the apoHb (i.e., defined as having a functional heme-binding pocket), apoHb quantification should be performed via an activity assay, and not total protein assays such as UV-absorbance analysis. Previous research quantified apoHb yield via analysis of the soluble apoglobin's absorbance peak at 280 nm (or via total protein assays). However, it has previously been demonstrated that quantification of soluble apoglobin does not accurately indicate the activity of the apoHb preparation, since a mixture of active and inactive apoglobins coexist in solution. As expected for a pigment-free protein, the UV-vis absorbance spectrum of apoHb consists of a single peak at 280 nm, which is shown in FIG. 3A. The presence of residual heme, which originates from porphyrin not removed during the purification process, yielded a slight Soret peak absorbance at ˜412 nm indicating the presence of hemichromes (a type of heme-bound species). A novel process to separate hemichromes from reconstituted Hb (rHb) is described later in this example. However, in previous apoHb purification studies, the residual Soret peak was observed at ˜404 nm, indicating that the bound heme yielded metHb and not hemichrome. The difference in the type of Hb formed from residual heme could have been caused by the dissociation conditions necessary for heme removal from Hb in the acidic ethanol solution of the TFF process compared to the acidic MEK and acetone used in previous apoHb purification schemes. For the latter, the residual heme originated from unextracted porphyrin, which shielded the heme from the extraction solvent. During TFF purification, the porphyrin ligand is likely maintained in dynamic equilibrium between free heme and the heme-protein complex. Since the apoprotein exists unfolded in acidic ethanol, the heme molecules in equilibrium were likely nonspecifically bound to the protein. Thus, when the apoprotein was refolded during the diafiltration process, these nonspecifically bound heme molecules yielded hemichromes. These hemichromes only constitute less than 1% of TFF-apoHb.

Upon heme addition to apoHb, the absorbance of the solution at 280 nm and in the Soret band increases due to the presence of the heme pigment and from the covalent bond of the proximal histidine (His-F8) in apoHb with the heme iron. The final absorbance spectra of reconstituted Hb (rHb) should also be the same as native Hb with the characteristic intense Soret peak and Q-bands (discussed in rHb Analysis section). Since apoHb lacks this intense Soret band, heme extraction from Hb was determined through analysis of the UV-visible spectra of the apoHb solution. Successful heme extraction was determined when less than 1% residual heme could be detected (i.e., when the absorbance ratio between the protein peak at 280 nm to the Soret peak at ˜412 nm is less than 0.1).

The increase in Soret peak absorbance compared to pure heme when the porphyrin is incorporated into apoHb is due to the formation of a covalent bond between His-F8 in apoHb with the iron atom in heme. This difference in Soret peak absorbance is shown in FIG. 3A, and is used in the DCNh-incorporation assay to quantify the number of His-F8-Fe bonds formed (i.e., active heme-binding pockets). In the DCNh-incorporation assay, DCNh is titrated against a constant apoHb concentration until all the heme-binding pockets have been saturated with DCNh and excess DCNh is present in solution. During the initial titration, all the added heme reacts with apoHb to form rHbCN which produces a straight line (major line) in FIGS. 3B and 3C (top graphs). Upon saturation of the available heme-binding pockets, free heme becomes present in solution without the formation of additional covalent bonds between the His-F8 residue of apoHb and the iron atom in DCNh. Therefore, the increase in absorbance at later titration points are due to the absorbance of pure DCNh in solution. Since rHbCN has a higher Soret absorbance than pure DCNh, a minor line with lower slope compared to the major line is observed in FIGS. 3B and 3C (top graphs). The inflection point between the major and minor lines indicates the concentration of heme necessary to saturate the heme-binding pockets of apoHb. This heme-binding assay was used to quantify the concentration of active apoHb on a per heme basis throughout this study.

As shown in FIG. 3B and FIG. 3C, by monitoring the Soret peak at 420 nm, the titration assay can be performed using DCNh (i.e., heme species used in assay) to determine the concentration of active heme-binding pockets. Since the number of active heme-binding sites is dependent on the initial concentration of apoHb in the sample to be tested, this explains the difference in the results between the TFF and acetone produced apoHb samples. FIG. 3B exemplifies the assay when 7.70 μM of active heme-binding sites were present in an apoHb solution produced via TFF. On the other hand, FIG. 3C exemplifies the same assay on an apoHb solution produced from the acetone extraction procedure with 11.86 μM of active heme-binding sites. Subtracting the pure DCNh absorbance from the absorbance of the titration mixture, allows for the detection of a more noticeable slope change as can be seen in the middle graphs in FIGS. 3B and 3C. From these graphs, it is important to note that apoHb produced via TFF (FIG. 3B) and apoHb produced via acetone extraction (FIG. 3C) had the same characteristic slope change indicating the expected heme-binding activity of apoHb. Both the residual plots showed no discernable trend, indicating good fits to the major and minor lines (bottom graphs of FIGS. 3B and 3C).

After each batch, the DCNh-incorporation assay was used to quantify the moles of active apoHb on a per heme basis and the 280 nm absorbance (6=12.7 mM⁻¹ cm⁻¹) was used to quantify moles of total protein. To determine protein yield, the moles of active apoHb and total protein were compared to the initial moles of heme in the Hb precursor. The yield of the TFF apoHb production method was compared to the acetone method using two size TFF filters (miniKros (surface area of 1,000 cm²) and microKros (surface area of 20 cm²)) to demonstrate the scalability of the TFF process. The results from this analysis are shown in Table 1.

TABLE 1
Summary of results from various apoHb production methods and
the effect of concentration on the activity of apoHb samples.

| | Overall Protein | Overall Active | |
|---|---|---|---|
| Production Method | Yield | ApoHb Yield | N |
| Acetone | 90.5% ± 17.6% | 70.3% ± 10.7%^{a} | 4 |
| MicroKros TFF | 96.6% ± 5.5% | 83.1% ± 5.4% | 6 |
| MiniKros TFF | 98.4% ± 11.0% | 73.4% ± 5.3%^{a} | 14 |

| Active Protein Loss | Total Protein Loss |
|---|---|
| 7.7% ± 1.9%^{b} | 21.3% ± 8.1%^{b} |

| State | % Active Protein |
|---|---|
| Unconcentrated | 62.3% ± 4.8%^{c} |
| Concentrated | 69.5% ± 4.0%^{c} |

^{a}p < 0.05 compared with microKros TFF
^{b,c}p < 0.05 between pairs with same letter

Studies have previously reported total apoHb yields from acetone or MEK extraction to be ˜90%. However, these studies quantified total protein (which includes both active and inactive apoHb) through methods such as protein absorbance at 280 nm. Thus, when comparing total protein yields, apoHb production via TFF (total protein yield of about 95%) had similar values to the commonly used heme extraction methods. Additionally, the total protein yield from acetone extraction agreed with previous reports. As expected, there was more total protein in solution compared to active apoHb in solution indicating that some of the resultant protein in solution lost its activity. It was observed that some protein was adsorbed on the filter membrane, which explains the loss in total protein. Yet, total protein analysis also showed that most of the protein was retained during production and that there was no significant difference between the studied production methods. Thus, since virtually no protein is lost in the TFF process, applications in which only heme-free globin is desired can still benefit from the TFF purification methodology. From the active protein analysis, acetone showed 70.3% yield compared to 83.1% and 73.4% for the microKros and miniKros TFF filters, respectively. Additionally, the yield from the small-scale miniKros filter was significantly different than both other setups. These results demonstrate that TFF production had similar or improved active apoHb yields compared to the acetone extraction method.

TFF-apoHb production with microKros filters had a significantly higher active apoHb yield than both the acetone and miniKros TFF methods (p<0.05). When scaling a TFF system, factors such as shear rate, pressure drop, filter type affect TFF efficiency. Therefore, operational parameters were kept constant when possible between the miniKros and microKros TFF systems in this example. However, the inlet pressure for the miniKros system prevented it from reaching the required flow rate to obtain the same shear rate as the microKros system. Thus, shear rates of 5,900 s⁻¹ and 4,300 s⁻¹, were achieved for the miniKros and microKros systems, respectively. The difference in shear rate could explain the lower permeate flow rate of the miniKros system, since lower shear rates may facilitate protein build up on the membrane. Since the permeate flow rate was not scaled, the diafiltration period was longer on the miniKros system, increasing the time that the protein remained unfolded in the acidified organic solvent. This longer exposure time could explain the lower active protein yield of the miniKros system compared to the microKros system and is a key variable which must be controlled to improve the yield of active apoHb.

During the concentration phase of TFF processing, protein precipitation was observed. Over time, HF membrane fouling decreased permeate flowrate up to 70%, making further concentration non-viable. To explore the effect of this concentration step on active protein yield and activity of the apoHb preparation, apoHb preparations were tested for total protein and active protein before and after concentration. Protein lost during concentration was compared to the initial mass of Hb used for apoHb production. As seen in Table 1, the loss of total protein from the sample was greater than active protein. Since more total protein was lost, the fraction of active protein in solution increased. The higher loss of inactive protein can be explained by the greater instability of inactive apoHb in solution versus active apoHb, facilitating precipitation at higher concentrations of apoHb. Stabilizing agents or alternative buffers may alter or improve these effects and should be considered in future method optimization.

The limit of 2 mg/mL of initial Hb precursor in the acidic ethanol solution was chosen to ensure full heme extraction from the sample. The dissociation of heme from unfolded globin seems to follow the equilibrium between the globin-heme complex and free heme+free globin in solution. Thus, when too high of an initial Hb concentration is loaded into the TFF circuit, the high globin concentration may limit the heme from dissociating from the globin-heme complex. Thus, in trials with a high initial Hb concentration little to no heme would permeate out of the TFF cartridge (undetectable on the absorbance spectrum). This was despite observing a significant amount of heme in the acidic ethanol solution within the TFF flow circuit (analyzed via the ratio of the Soret peak at 412 nm to the 280 nm protein peak). Additionally, the requirement of 9 diacycles for full heme extraction was evidenced by this equilibrium. If no heme retention occurred, the number of diacycles should have been closer to that of a simple buffer exchange (5 to 6 diacycles). Furthermore, when Hb solutions with concentrations of >50 mg/mL were used as the initial basis for the process, the protein rapidly denatured into a red precipitate when it made contact with the 80% acidic ethanol solution. Thus, to minimize this effect, Hb solutions with concentrations of ˜25 mg/mL were used when adding the holoprotein to the acidic ethanol solution.

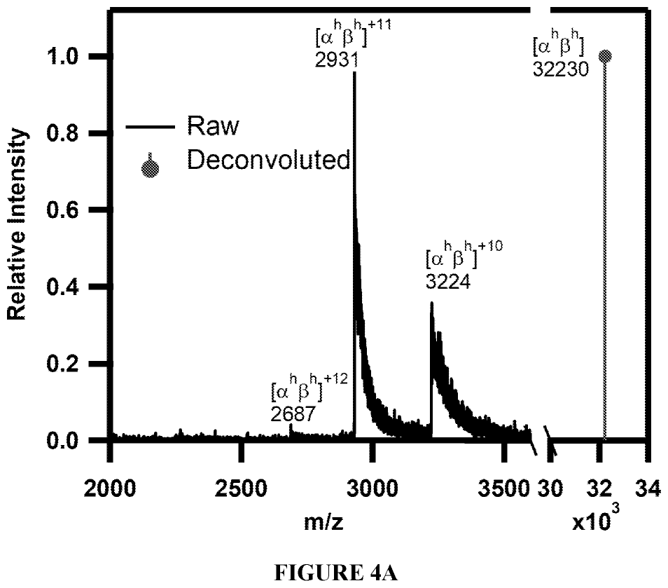

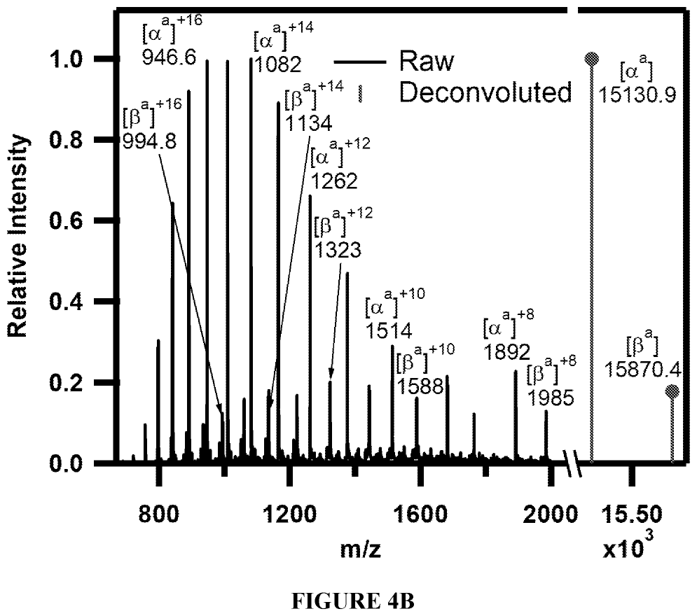

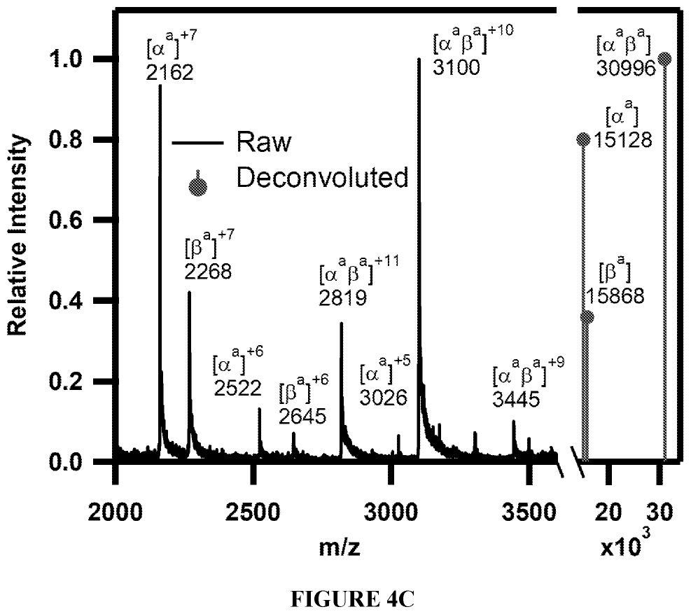

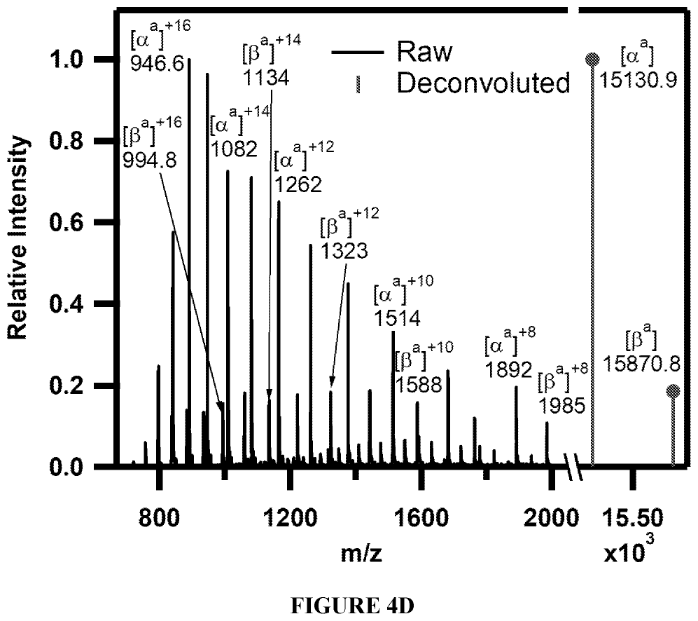

Biophysical Properties of ApoHb produced via TFF. The TFF-apoHb was analyzed via electrospray ionization mass spectroscopy (ESI-MS) to determine if processing caused any amino acid residue modifications or protein damage. FIG. 4 presents the results of these experiments. ESI-MS analysis demonstrated that native Hb was detected only as holodimers (FIG. 4A). Although a predominantly tetrameric holoprotein was expected, the MS equipment did not have the ability to detect the mass of the tetrameric species, thus only αβ dimers were observed. The detection of αβ dimers can be explained by the dimer-tetramer equilibrium of Hb in solution. Additionally, upon protein denaturing under acidic conditions (FIG. 4B), Hb dissociated into its apoglobin monomers (individual α and β chains) and heme was released into solution. Since free heme was not detected (removed during dialysis before MS analysis), the only spectra with heme (indicated by the superscript h) was Hb analyzed under native conditions. All other spectra only detected apoproteins (indicated by the superscript a) When analyzing apoHb with MS under native conditions, the lack of heme in apoHb allowed for detection of apo αβ dimers and apo α/β monomers (FIG. 4C), possibly indicating monomer-dimer equilibrium in solution. Yet, like Hb, under acidic conditions (FIG. 4D), the higher order species dissociated, making the mass spectra of apoHb and Hb nearly identical (same peaks of apo α/β globin chains when comparing FIG. 4B and FIG. 4D). Thus, during Hb MS analysis, apo α/β monomers were only detected under denaturing conditions, while for apoHb MS analysis, apo α/β monomers were detected under both conditions. Although the intensity of α and β globin chains were expected to be similar given their 1:1 molar ratio in Hb and apoHb, it has been shown that conformational differences between the α and β globins allow for more efficient competition of the α chains for charges. This difference causes a higher intensity of α chains in the spectra compared to β chains.

Under native conditions, the observed mass of holo-Hb αβ dimers (FIG. 4A) and apoHb αβ dimers (FIG. 4C) were determined to be 32,230 Da and 30,996 Da, respectively. These results were close to the theoretical mass of 32,226 and 30,994 Da for the holo- and apo-αβ dimer, respectively (difference of two 616 Da heme groups between the holo- and apo-αβ dimers). During Hb analysis under denaturing conditions (FIG. 4B), the apo α globin (theoretical mass of 15126.4 Da) detected had an observed mass of 15130.9 Da while the apo β globin (theoretical mass of 15867.4 Da) had an observed mass of 15870.4 Da. During apoHb analysis, apo α globin was detected under both native (FIG. 4C) and denaturing conditions (FIG. 4D) with observed mass of 15128 Da and 15130.0 Da, respectively. Also during apoHb mass spectral analysis, apo β globin had observed masses of 15868 Da and 15870.8 Da under native (FIG. 4C) and denaturing conditions (FIG. 4D), respectively. The results from ESI-MS analysis demonstrated that apoHb globin chains produced via TFF maintained their structural integrity and were not chemically modified.

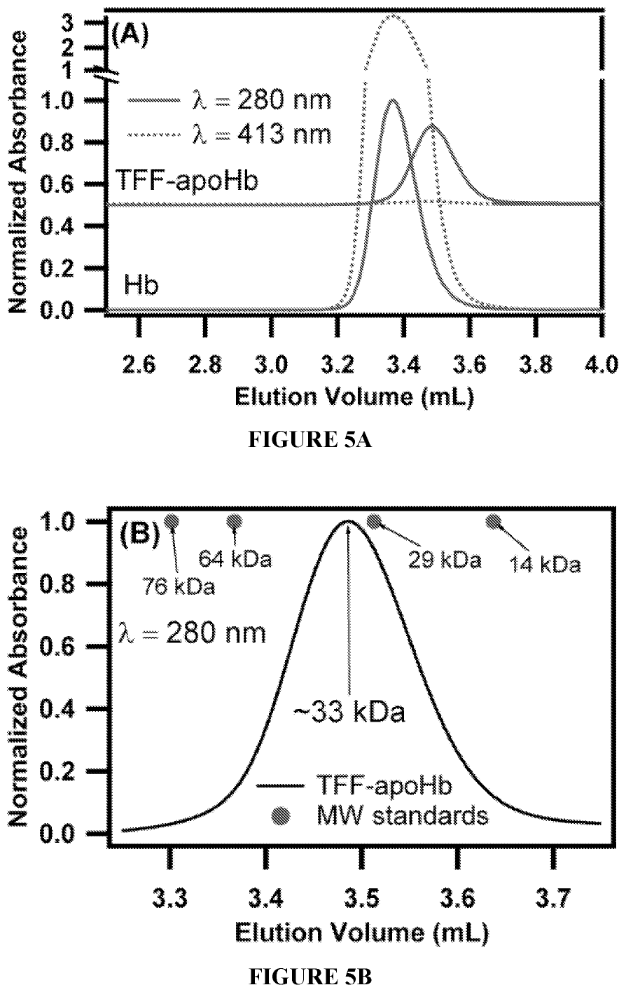

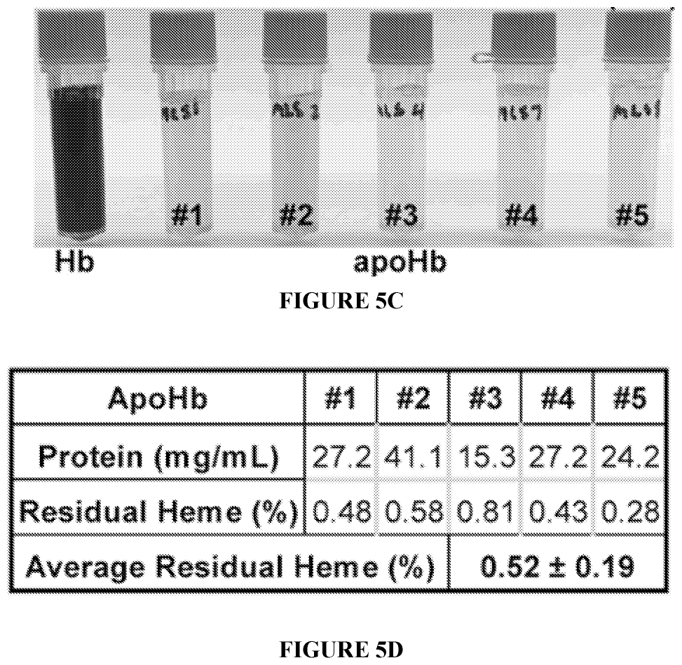

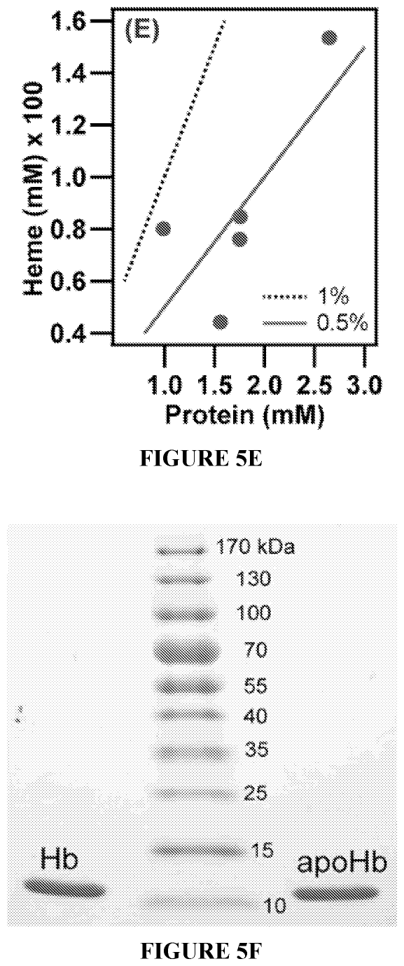

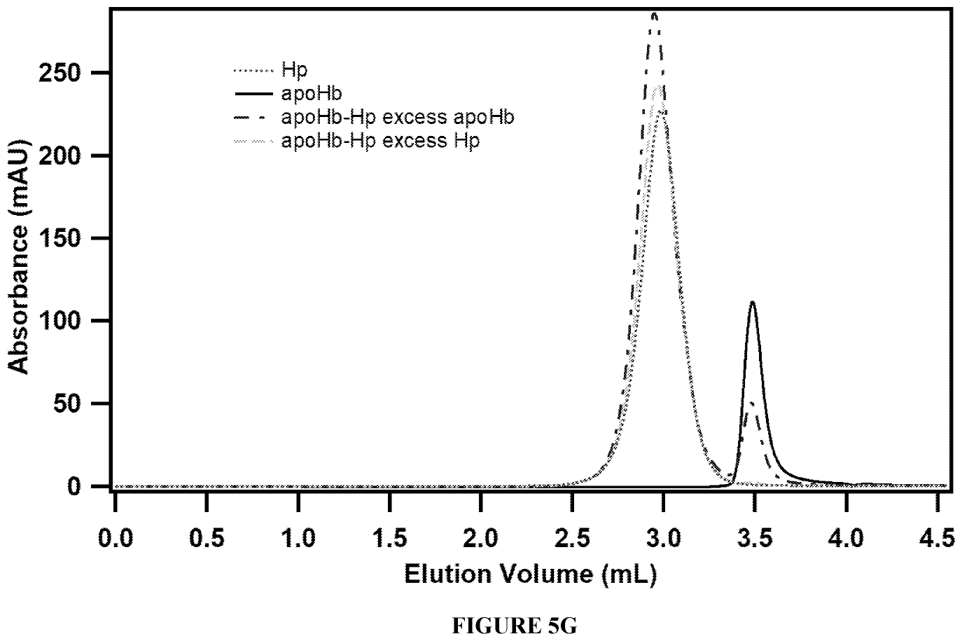

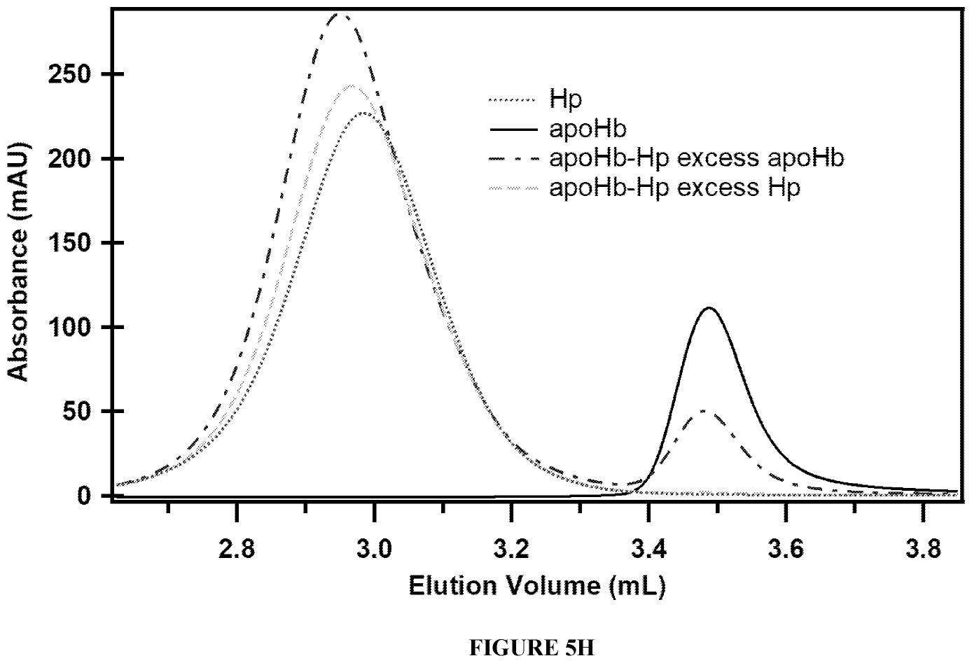

Further analysis of the quaternary structure of apoHb was performed using SEC. The HPLC-SEC profile of TFF-apoHb and human Hb is shown in FIG. 5A. Comparing the chromatogram of TFF-apoHb with that of human Hb, it was observed that there was a major reduction in the absorbance of the Soret peak between the two species. Furthermore, the absorbance at 280 nm was also reduced between the two species. Both of these observations were expected given the high absorbance of heme in the Soret region and at 280 nm. The protein standards with MWs of 76, 64, 29, 14 and 6.5 kDa eluted at 3.30, 3.37, 3.51, 3.64, and 3.82 mL, respectively. Based on these elution times, the MW of apoHb which eluted at 3.5 mL was determined to be about ˜33 kDa. This MW indicated that the apoprotein was primarily an αβ dimer in solution. FIG. 5B shows the elution of TFF-apoHb and the peaks of the MW standards. From these chromatograms, it was also noted that there are no tetrameric species in freshly prepared TFF-apoHb samples. These tetrameric species have been theorized to originate from irreversible disulfide bond formation between apoHb dimers in the apoHb denaturing pathway. Yet, there exists conflicting evidence on whether these disulfide bonds are formed after heme removal, as it has been shown that apoHb precipitates maintain functional thiol groups, while Hb precipitates forms disulfide-bonded tetramers.

SEC was also performed on Hb and apoHb samples to analyze heme content (FIG. 5C). Using the Soret peak of Hb as a reference, the Soret peak of each apoHb batch was used to estimate the amount of residual heme present in the apoprotein preparation after production. FIG. 5D and FIG. 5E shows results from this analysis confirming that less than 1% of residual heme was present in apoHb preparations produced via TFF (compared to the total protein in the sample). As shown in FIG. 5E, the final protein concentration of the sample did not influence the amount of heme remaining in the apoHb preparation, demonstrating that the protein can be concentrated maintaining its desired characteristics. Additionally, the samples normally had ˜0.5% or less residual heme confirming the effectiveness of the TFF heme extraction method in removing heme from the apoHb preparation.

A promising and important characteristic of apoHb is its clearance from the blood stream via CD163+ macrophage and monocyte mediated endocytosis. This in vivo clearance pathway for apoHb is the same for cell-free Hb. Both holo- and apo-Hb first bind serum Hp to form the (Hb/apoHb)-Hp complex, then the (Hb/apoHb)-Hp complex is captured by CD163+ macrophages and monocytes. To analyze if TFF-apoHb was capable of binding Hp, a fixed Hp concentration was mixed with increasing concentrations of apoHb and allowed to react to completion. The components of these mixtures were then separated via SEC-HPLC. As shown in FIGS. 5G and 5H, apoHb eluted at 3.5 mL, while the large MW Hp mixture eluted earlier at 3 mL. The earlier elution volume of Hp was expected, since the Hp used was a mixture of higher MW proteins (>200 kDa). When analyzing the elution profiles, the lack of an elution peak at 3.5 mL for the apoHb-Hp mixtures with excess Hp demonstrated that Hp and apoHb formed a single apoHb-Hp complex at sub-stoichiometric apoHb concentrations. Furthermore, a slightly lower elution volume was observed for the apoHb-Hp complex as indicated by a slight left shift in the elution curve. From the excess apoHb trial, the change of the area under the curve compared to the pure apoHb solution indicated the amount of apoHb that was bound to Hp. From this data, active apoHb had almost the same mass binding ratio to Hp as native Hb (<5% difference), and agreed with previous apoHb:Hp binding studies that demonstrated binding of one αβ Hb dimer per Hp αβ dimer. Therefore, this analysis demonstrates that apoHb produced via TFF retains the ability to bind to Hp.

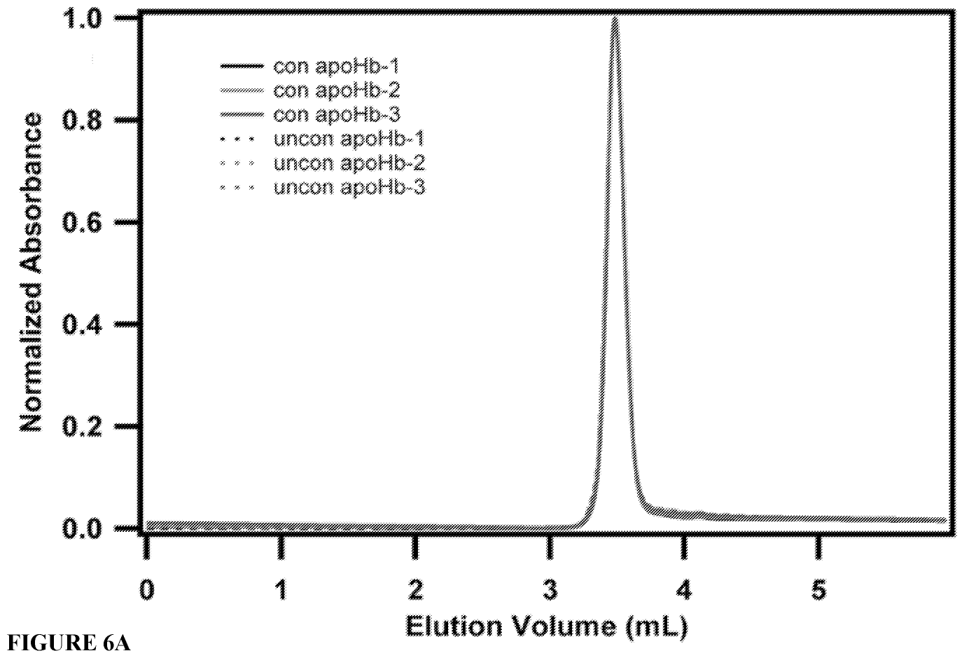

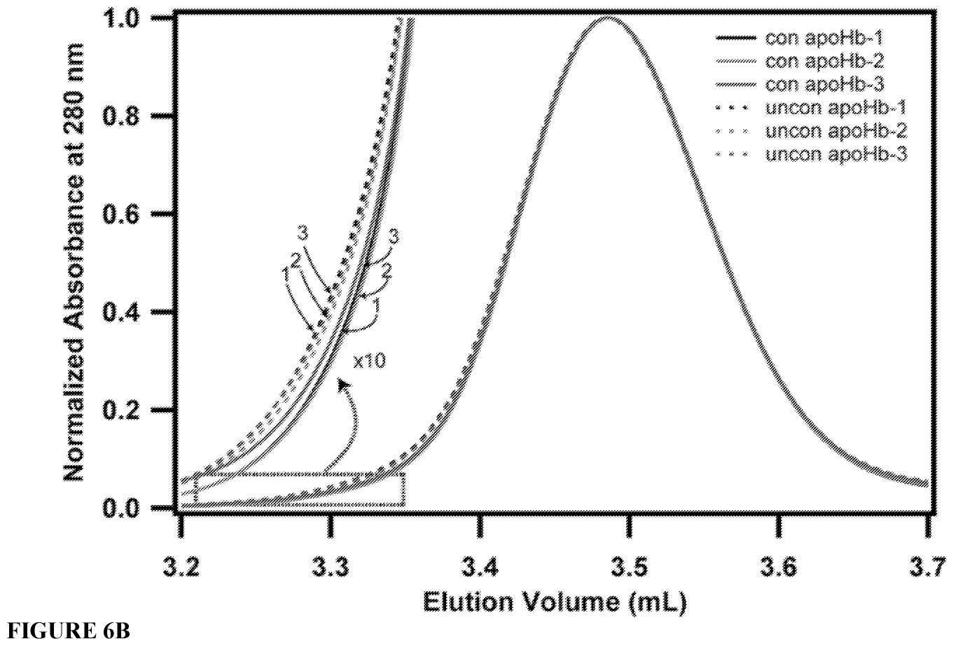

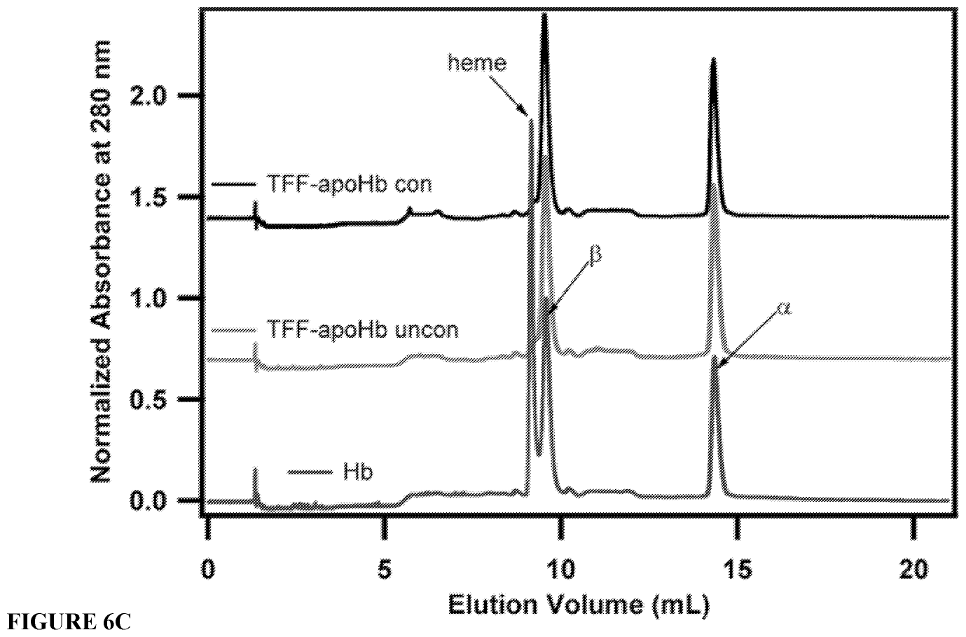

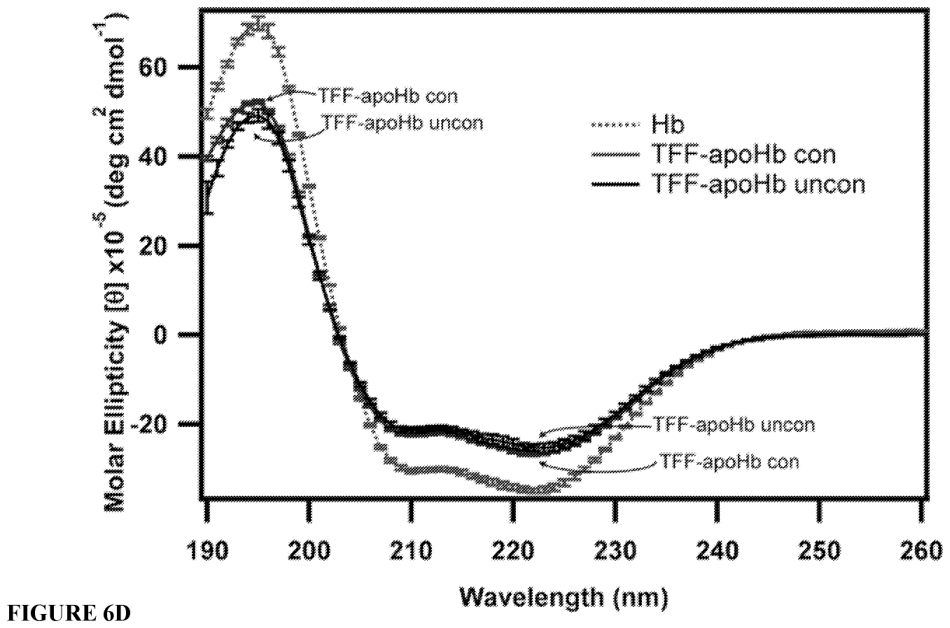

To ensure that no oxidative modifications or disulfide-bonded intermediates were present in our apoHb preparations, three different batches of TFF-apoHb were analyzed via reverse-phase HPLC (RP-HPLC). The samples were first evaluated via SEC-HPLC and, as shown in FIGS. 6A and 6B, the fresh TFF-apoHb preparations had very little variability and did not contain any noticeable amounts of tetrameric or oligomeric species. Interestingly, there may be a slight increase in the presence of apoHb tetramers in the unconcentrated (uncon) samples due to their left shifted elution peak as observed on SEC-HPLC (FIG. 6B). These higher MW species may be linked to the lower relative apoHb activity and lower alpha-helical content of the unconcentrated samples.

RP-HPLC analysis is shown in FIG. 6C. The Hb sample separated into its respective heme, α-chain and β-chain components, while TFF-apoHb samples displayed only α-chain and β-chain components. Furthermore, there was no noticeable difference in the RP-HPLC chromatograms when comparing the unconcentrated or concentrated TFF-apoHb samples with native hHb. Thus, the TFF process did not cause any oxidative modifications to the apoprotein as has been reported by some processes in the literature.

The secondary structure of TFF-apoHb was determined via CD of the far UV region (190-260 nm). This analysis is shown in FIG. 6D. The results indicated that, as previously shown in the literature, apoHb had a ˜25% reduction in alpha-helical content compared to native Hb (given by the ratio of the 222 nm peak, which corresponds to alpha-helices). Furthermore, there seemed to be a slight increase in alpha helical content upon sample concentration. This, similar to the increase in relative activity, may have been due to the removal of inactive apoHb from the sample during concentration.

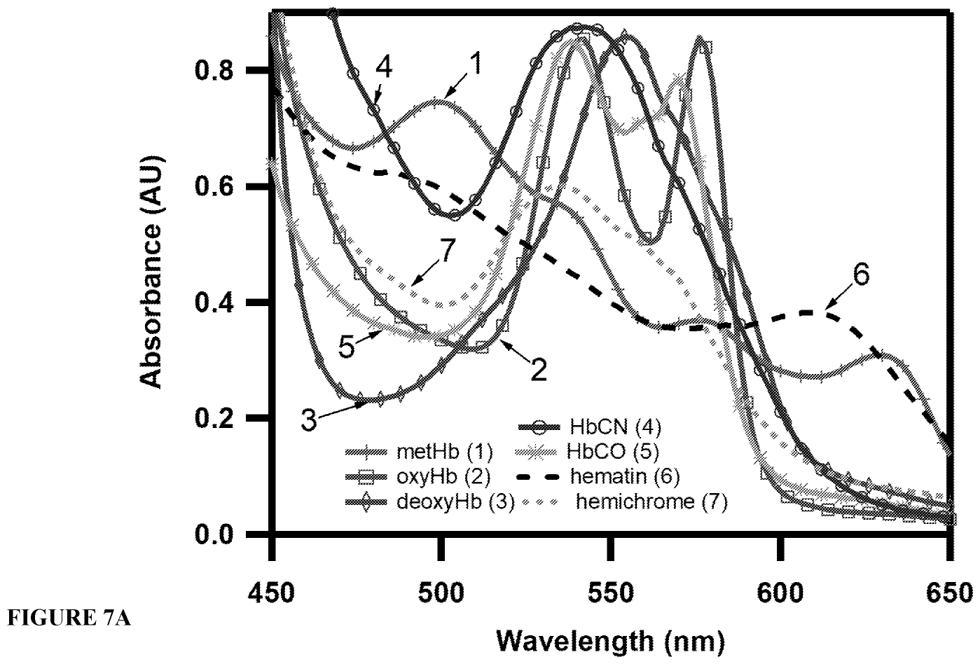

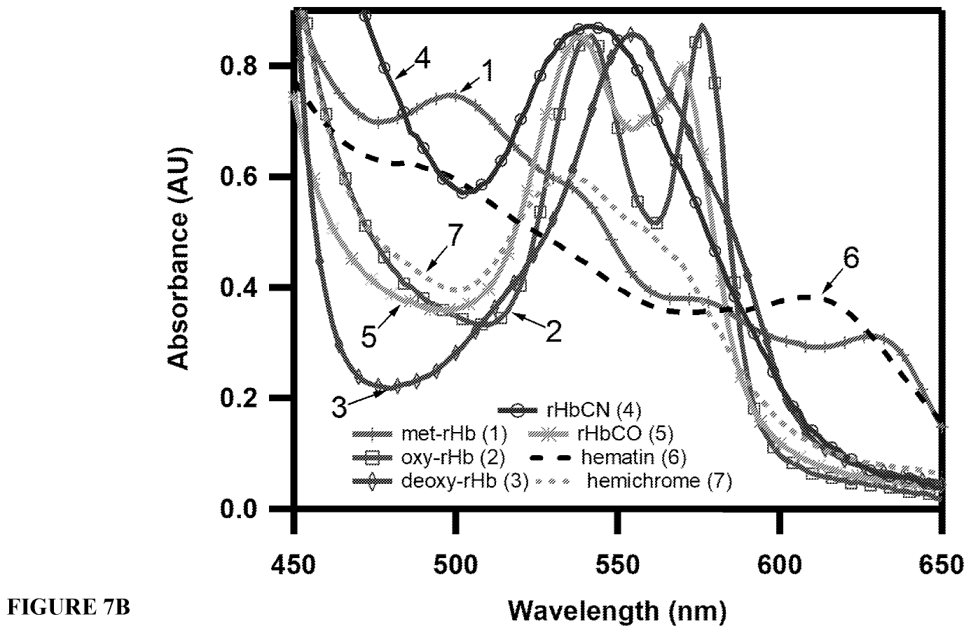

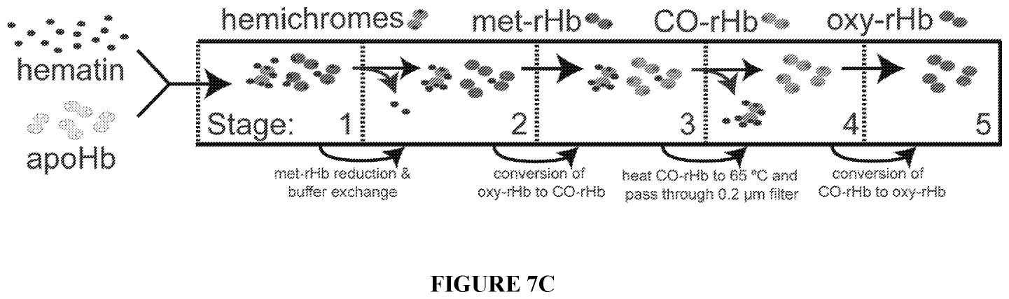

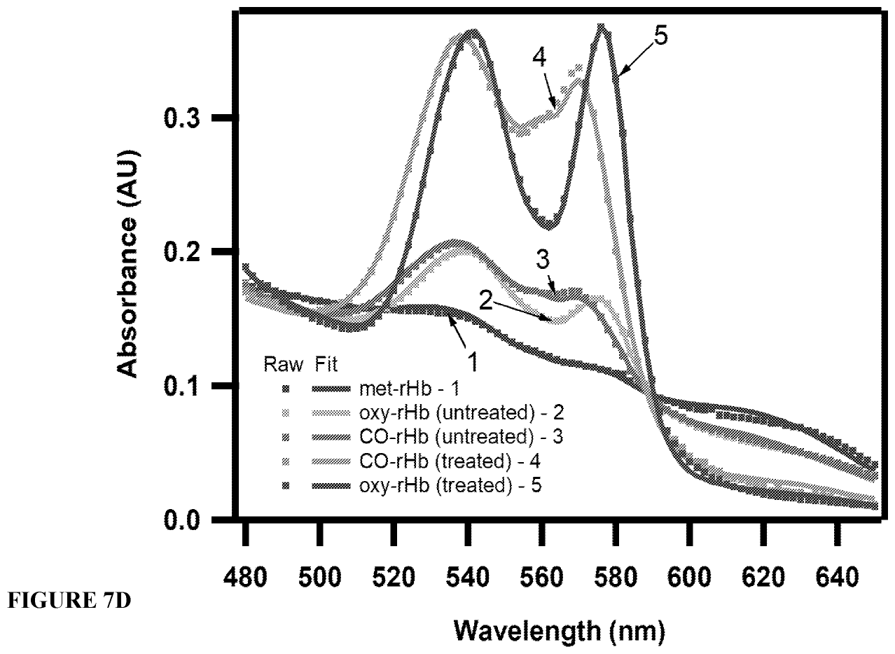

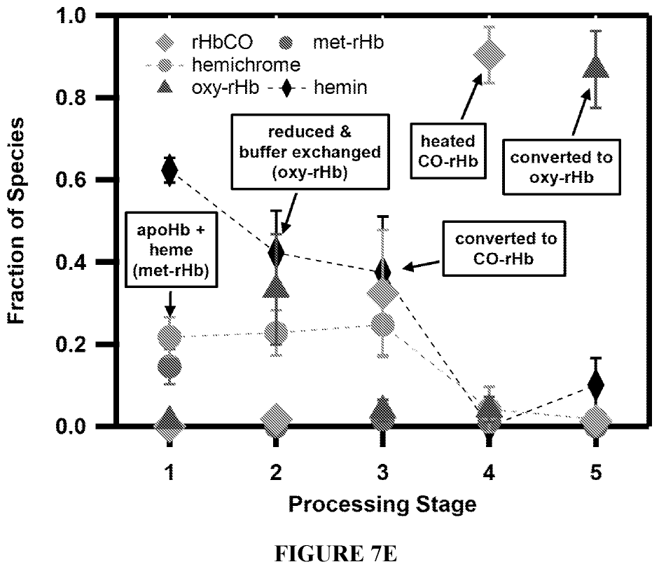

To test the activity of TFF-apoHb, the protein was reconstituted into rHb and the biophysical properties of rHb were analyzed and compared to native Hb. First, TFF-apoHb was reacted with heme and the spectral absorbance of various liganded forms of rHb were compared to native Hb. It was evident that the spectra of rHb in various liganded states shown in FIG. 7B closely resembled that of native Hb shown in FIG. 7A. This indicated that TFF-apoHb can be reconstituted to yield native-like Hb spectra. However, hemichromes or other heme-globin complexes are observed when reconstituting apoHb. Such complexes may convolute spectral analysis of rHb. As shown in FIG. 7D, the spectra of rHb before processing had a distinct offset from pure rHb. When comparing non-processed samples (before heating), it was noted that the offset was due to the presence of hemichromes and unbound heme (pure species spectra shown in FIGS. 7A and 7B), confirming the presence of hemichromes. Yet, the literature contains no method to separate or remove these denatured species. To perform accurate spectral analysis of rHb, we developed a simple method to remove these unstable species, which is shown in FIG. 7C.

Starting from a mixture of excess heme and apoHb, which reacts to form met-rHb, hemichromes, and excess heme (stage 1), the sample is processed to obtain oxy-rHb. Yet, hemichromes and excess non-specifically bound heme could be present in the sample along with oxy-rHb (stage 2). Thus, the mixture from stage 2 was placed under a CO atmosphere to transform oxy-rHb into the more heat stable CO-rHb species (stage 3). HbCO is more resistant to thermal denaturation and precipitation at elevated temperatures (65° C.) compared to other liganded forms of Hb, whereas heme-globin complexes and hemichromes are highly unstable and precipitate even at low (4° C.) temperatures. Thus, when a mixture of CO-rHb and heme-globin complexes is heated, the CO-rHb remains in solution while the unstable species precipitate out of solution (stage 4). After heating and separation of hemichromes, the CO-rHb can be reverted into oxy-rHb under a pure O₂ atmosphere and white light illumination (stage 5).

To analyze this unique hemichrome removal method and provide better information on the species present in solution, a spectral deconvolution program was developed and implemented to determine the fraction of rHb species present in solution during the various processing stages in the reconstitution process. As shown in FIG. 7E, hemichromes were present in the sample at significant levels until the sample was heated. It was also observed that, although heme would be expected to be filtered through the HF modules, much of the excess heme added to the apoHb sample was not removed during the modified HEMOX buffer-exchange step after reduction (only a slight decrease in heme content was seen between stage 1 and 2). Since the unbound heme was not removed via filtration, it indicated that some of the heme was nonspecifically bound to the protein in solution. However, it was noted that the nonspecifically bound heme was mostly attached to the hemichromes, since no heme was detected in the CO-rHb spectrum after hemichrome removal upon heat treatment (this event is also represented in FIG. 7C). The higher affinity of heme to nonspecifically bind to hemichromes would be expected since these semi-denatured species should have more exposed hydrophobic regions or altered structural motifs that lead to higher heme-binding affinity. The higher heme binding capacity of hemichromes is also supported by reports in the literature that denatured globin can bind up to thirty heme molecules. This ratio is much higher than the number of available heme-binding pockets present in tetrameric Hb (4 heme binding sites). Although some heme was detected in stage 5, this can be explained by the low signal to noise ratio of the heme species, especially since no heme was detected in the previous CO-rHb spectra of stage 4.

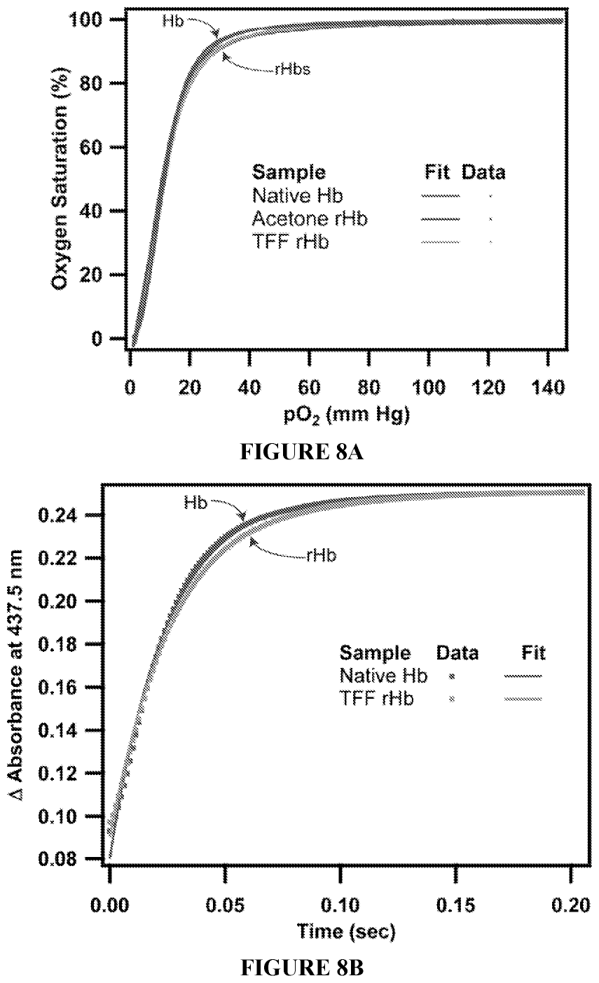

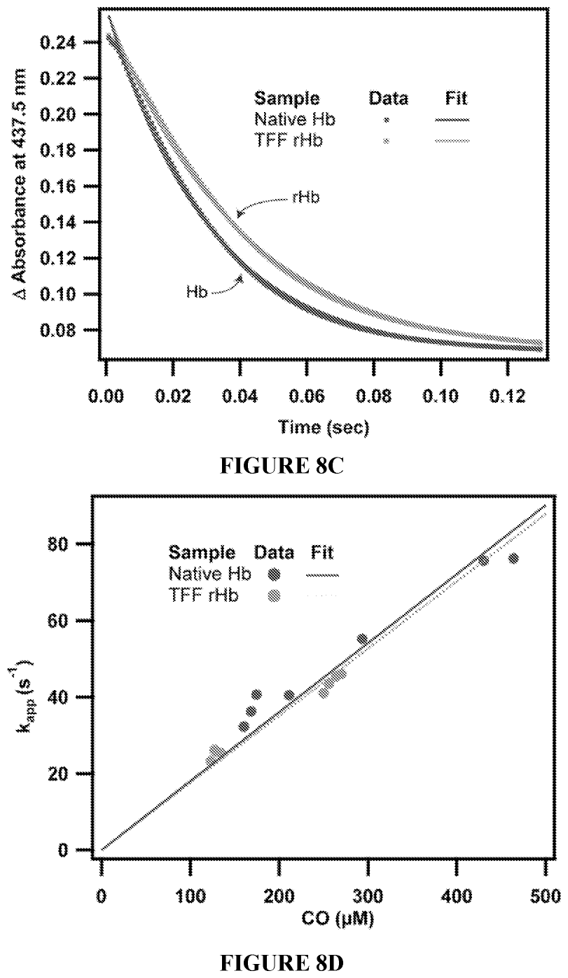

To further analyze the biophysical properties of rHb derived from TFF-apoHb heme extraction, rHb was fully reconstituted back to oxy-rHb and its O₂ equilibrium binding curve measured using a HEMOX Analyzer. From the O₂ equilibrium curve, the P₅₀ (partial pressure of O₂ required to saturate half of the heme binding sites with 02) and cooperativity coefficient (n) can be regressed. The O₂ dissociation (k_{off,O2}) and CO binding (k_{on,CO}) kinetics were also measured using stopped-flow UV-visible spectroscopy. FIG. 8 shows representative data sets for native Hb and rHb. FIG. 8A shows representative O₂ equilibrium curves for Hb and rHb. FIGS. 8B and 8C show representative O₂ dissociation and CO association kinetic time courses for Hb and rHb, respectively. Finally, FIG. 8D shows the apparent rate constant for CO association to deoxyHb at varying CO concentrations. This data was fit to a linear equation to regress k_{on,CO}.

Previous studies have shown that rHb produced from acetone extraction has the same biophysical properties compared to native Hb. This was confirmed from the quantitative results listed in Table 2. The P₅₀ of rHb (11.36±0.87 and 11.20±0.43 mmHg for concentrated and unconcentrated rHb, respectively) was similar to native Hb (11.69±0.88 mmHg), with no statistical difference (p<0.05). The cooperativity of rHb from TFF-apoHb (2.14±0.17 and 2.27±0.10 for concentrated and unconcentrated rHb, respectively) was statistically different (p<0.05) compared to native Hb (2.73±0.11). Yet, similar to native Hb, TFF derived rHb had cooperativity greater than 2, which is indicative of cooperative O₂ binding. Additionally, previous studies have shown that Hb which has been oxidized then reduced possess lower cooperativity than native Hb. This effect could have been exacerbated by the formation of reactive free radicals upon use of dithionite for reduction. It has also been shown that, upon reconstitution, the heme can enter the heme pocket in an altered orientation, which also reduces rHb cooperativity. Thus, the lower cooperativity of rHb compared to native Hb can be due to incorrect heme insertion and the necessary reduction step to form oxy-rHb. Also shown in Table 2 are the rate constants for O₂ dissociation from HbO₂ and CO association to deoxyHb. There was no statistical significant difference between the CO association rate constants for native Hb (k_{on,CO}=180±7 nM/s) and TFF rHb (k_{on,CO}=175±4 nM/s). However, the difference in O₂ dissociation between native Hb (k_{off,O2} s⁻¹=37.68±1.27) and TFF rHb (k_{off,O2} s⁻¹=33.59±1.10) was significant (p<0.05). Yet, similar to the cooperativity coefficient, the lower rate constants could be due to the processing required to obtain rHb. Overall, compared to previously developed apoHb production methods, rHb derived from the TFF production method maintained native Hb characteristics.

TABLE 2
Biophysical properties of native Hb and TFF produced rHb:
O₂ affinity (P₅₀); cooperativity coefficient (n);
CO association to deoxyHb (k_{on,CO}); and O₂
dissociation from oxyHb (k_{off,O2}).

| | O₂ Equilibrium | | | Ligand Binding Kinetics | | |
|---|---|---|---|---|---|---|
| | P₅₀ | | | k_{on,CO} | k_{off,O2} | |
| Sample | (mm Hg) | N | N | (nM/s) | (s⁻¹) | N |
| hHb | 11.69 ± 0.88 | 2.73 ± 0.11 | 10 | 180 ± 7 | 37.68 ± 1.27 | 4 |
| TFF rHb | 11.20 ± 0.43 | 2.27 ± 0.10^{a} | 4 | — | — | — |
| TFF rHb | 11.36 ± 0.87 | 2.14 ± 0.17^{a} | 10 | 175 ± 4 | 33.59 ± 1.10^{a} | 4 |
| Concentrated | | | | | | |

^{a}p < 0.05 compared with hHb

To analyze the stability of TFF-apoHb during storage, the amount of active and total apoHb was assessed under different storage conditions (37, 22, 4, −80° C. or lyophilized) and at two protein concentrations (concentrated [33.80±0.36 mg/mL active apoHb with 41.40±2.77 mg/mL total protein] or unconcentrated [1.47±0.01 mg/mL active apoHb with 1.99±0.17 mg/mL total protein]). The results from this analysis are shown in FIG. 8.

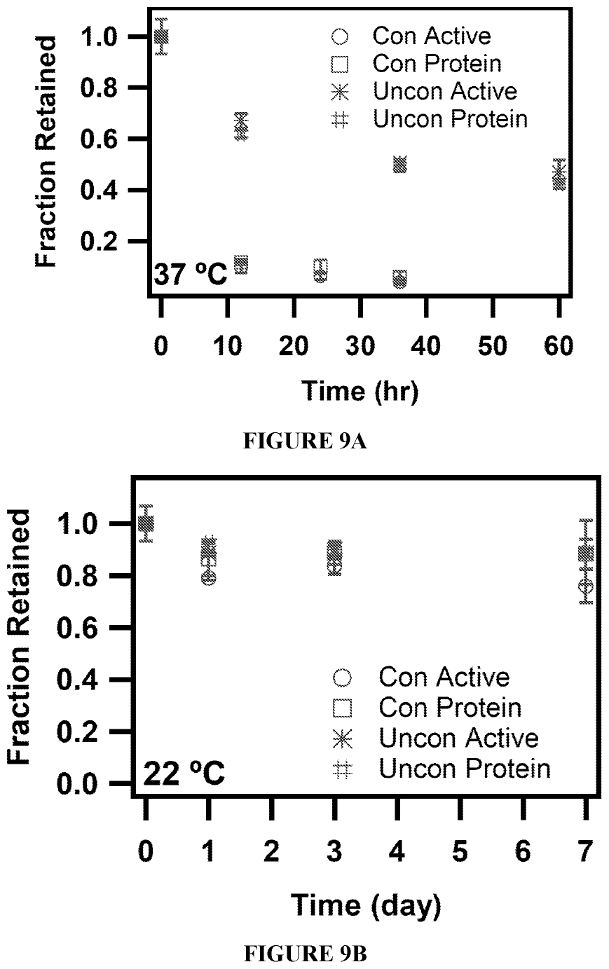

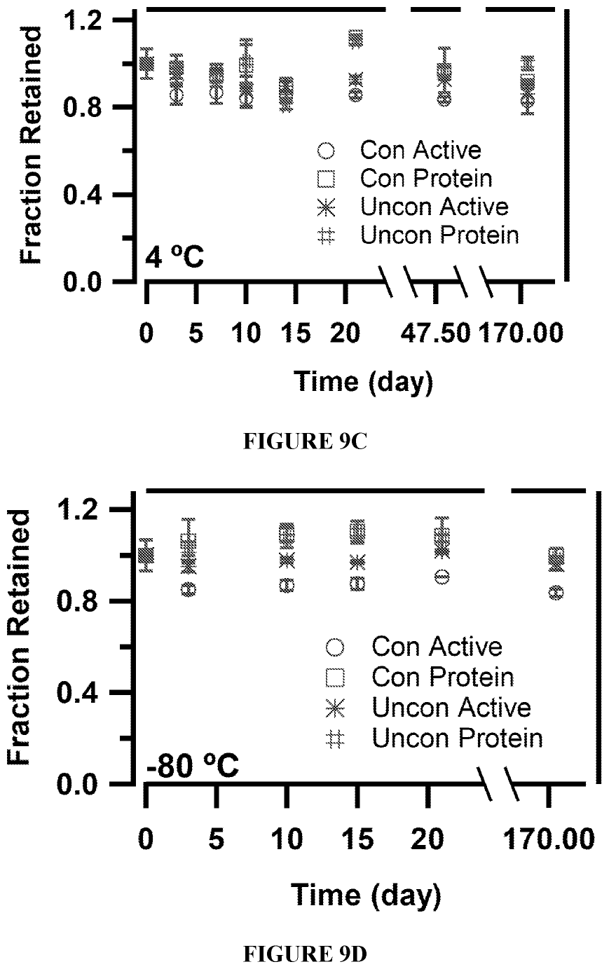

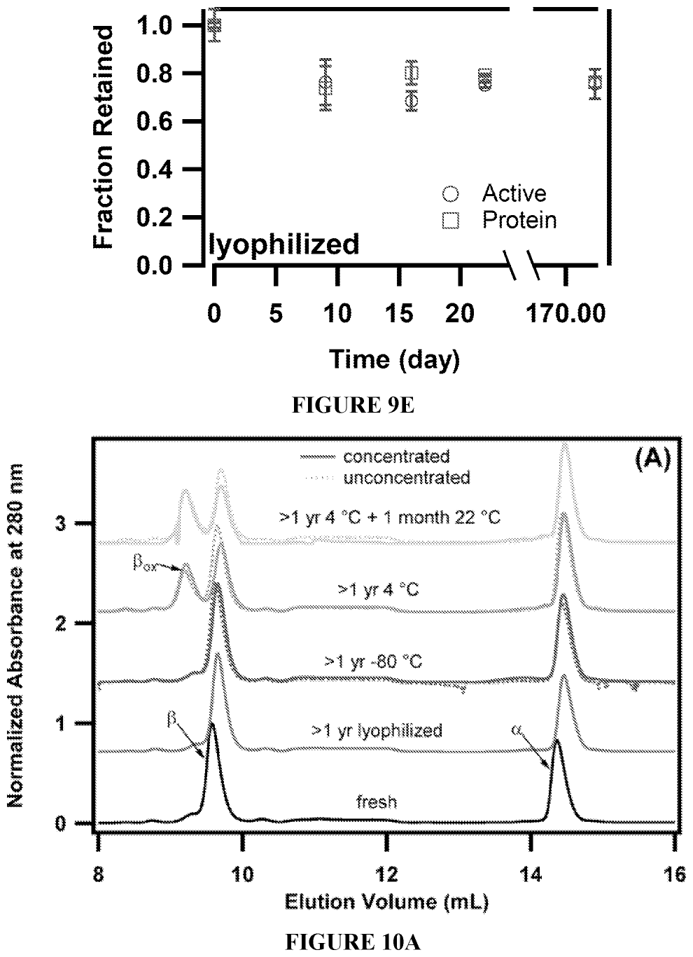

At physiological core body temperature (37° C.) (FIG. 9A), precipitation was visually apparent and the effect of time was statically significant at both apoHb concentrations. Under these conditions, apoHb quickly lost total protein and activity in solution during storage, especially for the concentrated samples. Interestingly, there was no statistically significant difference between the last two time points of the concentrated (24 and 36 h) and unconcentrated (36 and 60 h) samples, indicating some stability after the initial drops. Even though apoHb was not very stable at 37° C., this result does not deter possible biomedical applications of apoHb. When bound to Hp (expected to occur in vivo), the apoHb-Hp complex greatly increases the heat stability of the protein. At room temperature (22° C.) (FIG. 9B), the effect of time was statistically significant on activity, but no significant differences were seen for subsequent time points compared to day three. Additionally, the effect of time on total protein was insignificant. This contradicts the results of a previous study, which observed the rapid loss of apoHb after only a few minutes of storage at room temperature.

While stored at 4° C. (FIG. 9C), the effect of time was significant on the activity of unconcentrated samples, a significant difference was compared to day 0 was only seen on day 10. Additionally, no significant differences were seen after day 10. When stored at −80° C. (FIG. 9D), there was no statistically significant effect of time on apoHb activity or total protein at both concentrations. The higher stability of apoHb at −80° C. was expected, since the frozen sample lowered the likelihood of aggregate formation in solution. Lyophilized apoHb (FIG. 9E) also did not show a significant effect of time on apoHb activity or total protein. There was an initial decrease post-lyophilization, but activity was maintained afterwards (no statistically significant differences were observed). This was an expected result, since the freeze-drying process can damage the protein.

Under all concentration dependent conditions, the concentrated samples had statistically significant lower active apoHb retention compared to unconcentrated samples, indicating that higher storage concentrations led to higher active apoHb loss. This observation can be explained by the higher probability of protein aggregation at higher protein concentrations. On the other hand, total protein retention was not statistically significant between concentrated and unconcentrated samples, except for the 37° C. storage condition. These observations indicated that the concentration of stored samples only significantly influenced apoHb activity. Furthermore, lyophilized apoHb and storage at 37° C. appear to have coinciding total protein and active protein retention, indicating that the protein lost from heating or from the freeze-drying process was not selective for whether the protein was active or not. To further test the biophysical properties of the apoHb samples stored at 4° C., samples were fully reconstituted into rHb after one month of storage and the P₅₀ and cooperativity coefficient did not show any significant difference compared to apoHb samples reconstituted to rHb right after TFF production.

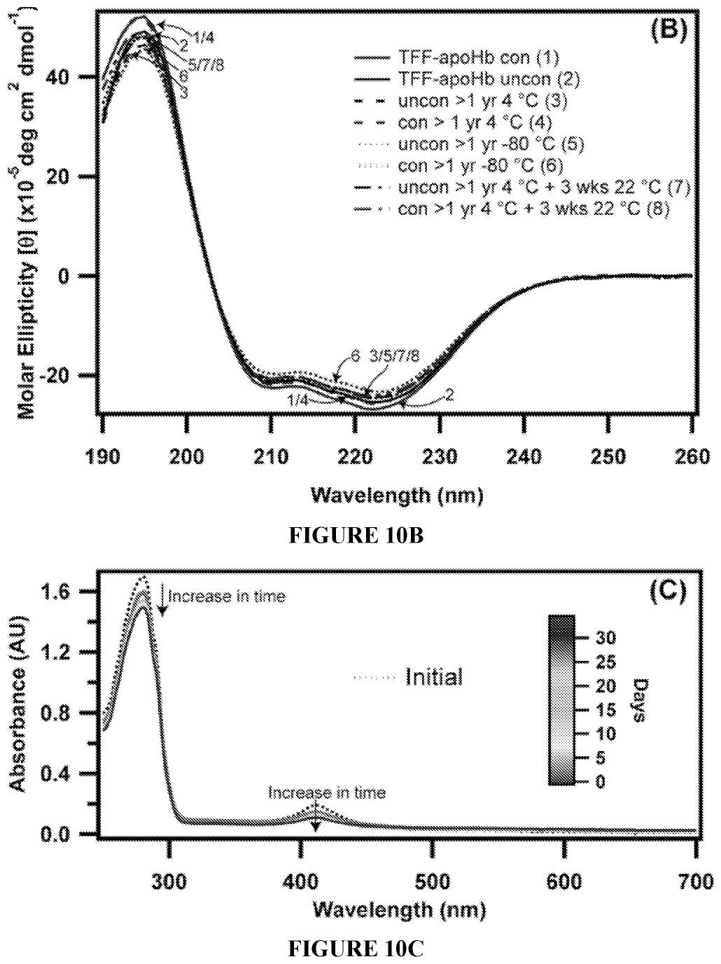

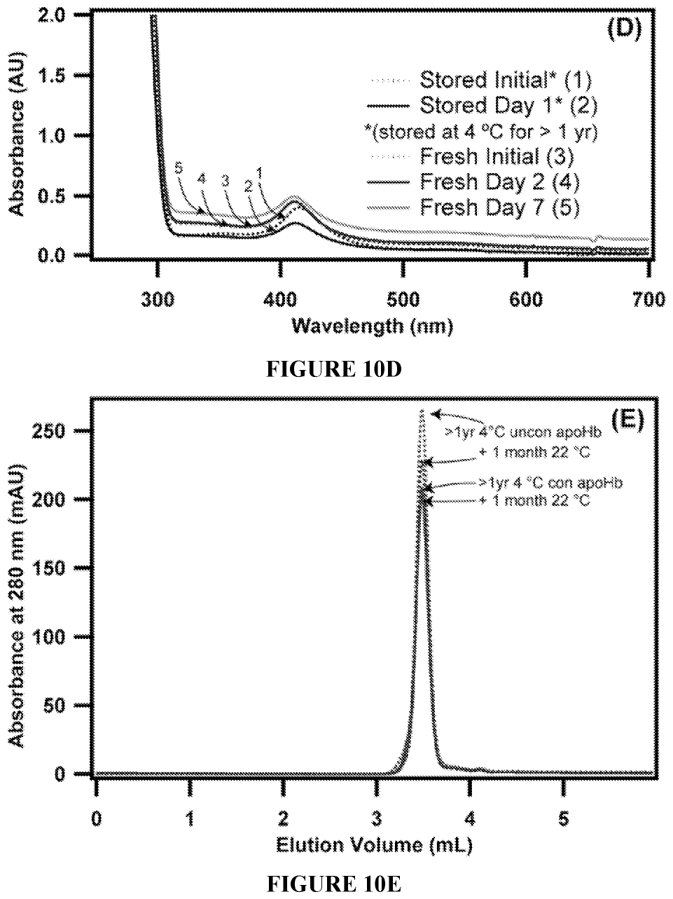

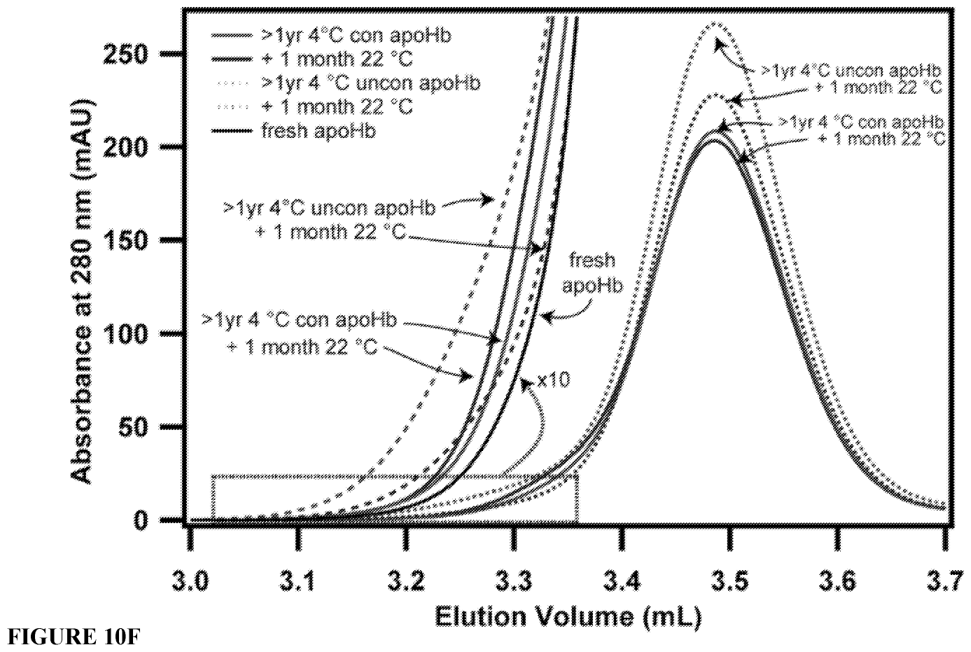

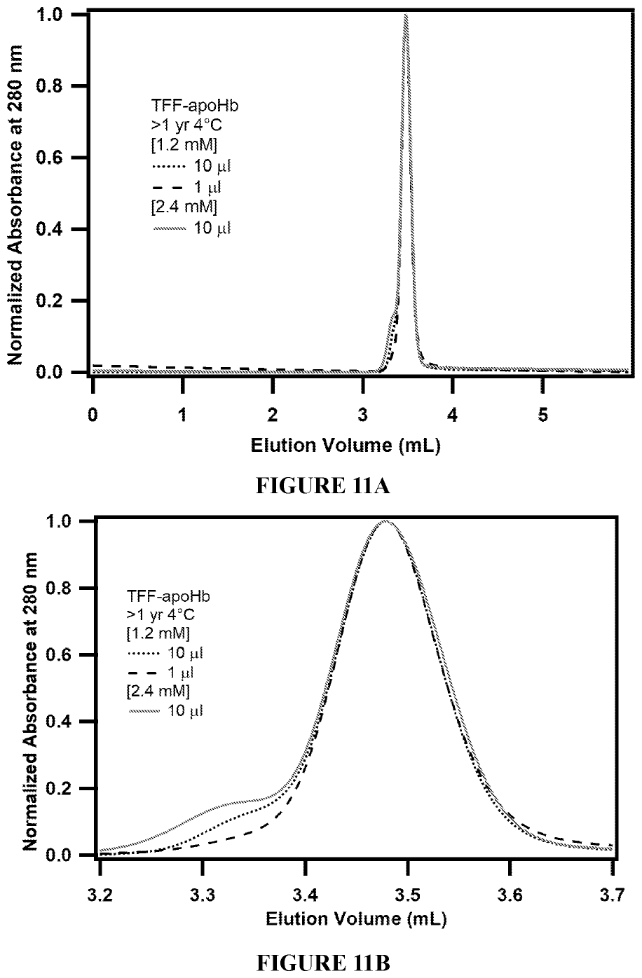

The stability of TFF-apoHb under different storage conditions was also assessed by RP-HPLC, CD and SEC-HPLC. This analysis is shown in FIG. 10. As shown previously, RP-HPLC did not show the presence of disulfide-bonded species nor oxidized forms of apoHb for freshly prepared samples. Furthermore, as shown in FIG. 10A, no oxidative modifications were observed for samples stored at −80° C. or lyophilized for over a year. Yet, samples stored at 4° C. or at 22° C. show an additional β-chain peak (βₒₓ), which is attributed to the oxidation of the methionine residue of the β-chain. This oxidative modification was attributed to oxidation of methionine and not to irreversible disulfide bond formation, since oxidation of methionine residues into methionine sulfoxide has been shown to lead to left-shifts on RP-HPLC (due to a decrease in protein hydrophobicity from oxidation of the methionine residue). If irreversible intermolecular disulfide bonds were created, it would be expected that elution of the oxidized species would have been right-shifted due to its higher MW similar to that observed for di-α crosslinked Hb.

To further investigate the effects of prolonged storage on TFF-apoHb, the CD spectra of stored samples was measured to analyze any loss of alpha helical content. All samples were diluted in DI water to approximately 10 μM to remove any interference from salt. The far UV CD spectra was measured for the diluted samples, and these results are shown in FIG. 10B. CD spectral analysis of stored samples showed that there may be slight reduction in the alpha helical content upon prolonged storage. Yet, there was no correlation of the alpha helical content to heme-binding activity or to the amount of β-chain oxidation. Overall, the apoglobin in solution did not show any relevant changes in secondary structure upon prolonged storage of TFF-apoHb.

Given that the literature on apoHb indicates that the apoprotein is unstable at room temperature and that TFF-apoHb was shown to be relatively stable at 22° C., a TFF-apoHb sample that had been stored at 4° C. for over a year was left at 22° C. in a sealed cuvette to monitor protein loss via precipitation. The results from this experiment is shown in FIGS. 10C and 10D for an unconcentrated and concentrated sample, respectively. The protein peak at 280 nm of the concentrated sample was above the upper detection limit of the spectrophotometer, so not much information could be obtained especially given the scattering due to the presence of precipitates in solution. Yet, the protein peak was detectable for the unconcentrated sample. Based on the 280 nm peak, there was an initial quick drop in protein concentration followed by a gradual decrease as a function of storage time. This was the same observation from FIG. 9 with ˜80-90% retention of total protein after one-week storage at 22° C. Since β-chain oxidation did not induce higher rates of apoprotein precipitation, the thermal stability of TFF-apoHb was not altered. Only a gradual increase in the absorbance at 700 nm was observed, which indicated that precipitates formed in solution. The samples stored at 4° C. and 22° C. were also analyzed using SEC-HPLC. Even though there was the presence of Pox chains, their elution did not result in the formation of large quantities of tetrameric species (shown on SEC-HPLC of FIG. 10E). The decrease in the area under the curve (AUC) of the samples was expected given that there was a decrease in the total protein concentration in solution as a function of storage time. Furthermore, there was a slight increase in the relative amount of tetrameric species of the concentrated sample upon storage at 22° C. Interestingly, the amount of tetramers in the unconcentrated sample reduced after it was removed from 4° C. and placed at 22° C. This may be due to the lower thermal stability of the inactive species leading to their precipitation when heated to 22° C. It is noteworthy that TFF-apoHb shows a much higher stability at 22° C. than apoHb produced via other methodologies in the literature, which are highly unstable at 22° C. leading to large precipitate formation at temperatures above 10° C. Furthermore, for both the concentrated and unconcentrated samples, a higher fraction of the heme-bound TFF-apoHb species was lost compared to the loss of heme-free TFF-apoHb. This observation was noticed due to the higher retention of the protein peak compared to the retention of the residual Soret peak as a function of storage time. The residual heme-bound species could have either precipitated or the heme could have bound to the quartz cuvette.

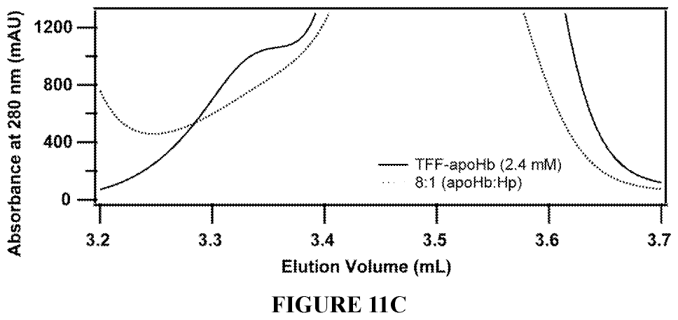

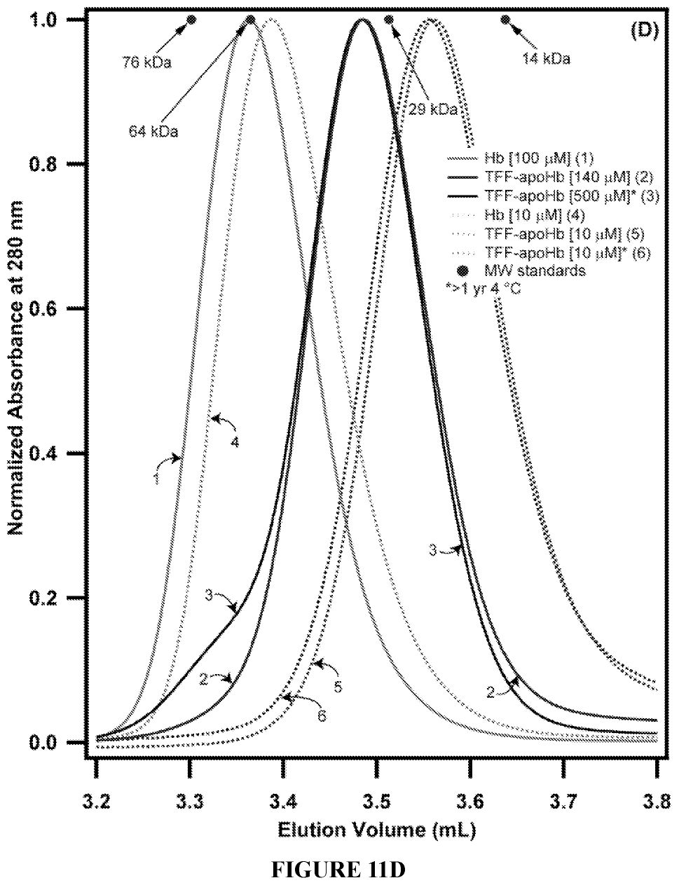

An analysis of the potential tetramer-dimer equilibrium of the apoprotein was performed by testing samples that contained detectable tetramers on the SEC-HPLC. These results are shown in FIG. 11. As shown in FIG. 11A, there was a minimal amount of tetramers even for a sample stored at 4° C. for more than a year. Furthermore, the relative amount of tetramers shown in the elution chromatogram was dependent on sample loading onto the column (FIG. 11B). Although molecules in fast equilibrium in SEC-HPLC elute at an intermediate elution volume of the two species, at slow equilibriums, the formation of two peaks is expected. Furthermore, addition of Hp to a tetramer containing sample led to a decrease in the elution peak of the tetrameric species (FIG. 11C). This decrease may indicate Hp binding to the tetrameric species similar to Hp binding to polymeric Hb. Yet, given the concentration dependence of the tetramers, it would be expected that Hp binding to apoHb dimers shifted the equilibrium towards lower tetramer concentrations. Thus, it was apparent that the relative amount of tetramers in the solution was dependent on protein concentration, which may indicate a tetramer-dimer equilibrium. Interestingly, at sufficiently low protein concentrations (10 μM), both Hb and TFF-apoHb shifted their SEC-HPLC elution to larger volumes, indicating a decrease in apparent MW. This shift is shown in FIG. 11D. For Hb, the shift indicated the tetramer-dimer equilibrium of the hologlobin at low concentrations. In the case of the TFF-apoHb, the elution indicated that at low concentrations, TFF-apoHb existed in a dimer-monomer equilibrium. The same phenomena occurred on a TFF-apoHb sample with some tetrameric apoHb. Although these equilibrium states were observed, the tetramer-dimer equilibrium was not consistent between samples given that the same injection volume at the same concentration of apoHb from two samples showed different amounts of tetrameric species. This may be related to its much lower equilibration rate. Furthermore, from the experiments we performed, there was no detectable correlation between tetramers, βₒₓ or the sample's relative activity. Therefore, there is still a need to investigate these tetrameric species, their equilibrium in solution and potential correlation to apoHb activity and βₒₓ.

Overall, given that there have been various reports in the literature on oligomerization of apoHb prepared via either acid-acetone or MEK methodologies, the formation of these non-native species may be linked to the preparation of apoHb. Not only was there minimal amounts of tetrameric species in our TFF-apoHb preparations, but there were no higher orders species detected under any of the tested storage conditions. Previous SEC-HPLC of MEK-apoHb showed a large percentage of tetrameric species and other higher order aggregates, which required the use of a reducing agent (such as dithiothreitol) during preparation to form dimeric apoHb. Furthermore, these tetrameric species were found to be dissociated at low concentrations, indicating that these species were not formed via irreversible disulfide bonds. Finally, even dimeric apoHb may also dissociate into monomers at low concentrations.

Conclusions

The possible biomedical applications of apoHb are very promising. Yet, its wide-scale use and analysis is restricted by current production methods, which are not easily scalable. New techniques to produce active apoHb have not been presented for decades even though new bioprocessing techniques have been developed. Existing apoHb production protocols require extensive dialysis and the use of highly flammable solvents. The newly proposed acidic alcohol TFF apoHb production method provides an easy and scalable method for producing active apoHb with more than 95% total protein and 75% active protein yields. Through the use of a 80% (v/v) ethanol:water solution for heme extraction, the flammability risks and toxicity issues with residual solvent are drastically lowered compared to previous apoHb production methods. Yet, the most valuable benefit of this new method is the use of an easily and cost-effective scalable process such as TFF for protein purification.

TFF-apoHb has the same characteristics as apoHb produced via previously published methodologies in the literature. These characteristics include: heme-binding activity, Hp binding activity, exists primarily as a dimeric species in aqueous solution, rHb O₂ equilibria and ligand binding kinetics. Yet, unlike apoHb produced previously in the literature, stability studies showed that TFF-apoHb can be stored at 4° C., −80° C. and in lyophilized form without appreciable changes in activity with higher stability at room temperature compared to previous apoHb storage studies. Additionally, ESI-MS analysis of TFF-apoHb demonstrated that it retained its structure without any chemical modifications. RP-HPLC demonstrated that no oxidative modifications were present in freshly prepared TFF-apoHb nor for samples lyophilized or stored at −80° C. Quaternary structure analysis via SEC-HPLC showed that TFF-apoHb αβ dimers did not form appreciable amounts of tetramers over prolonged storage at 4° C. Furthermore, new insight into apoHb oxidation and degradation was provided based on the oxidation of the β-chain of apoHb when stored at 4° C. or 22° C. for prolonged periods of time. Evidence for both tetramer-dimer and dimer-monomer equilibrium of apoHb was also presented. Finally, an improved hemichrome removal procedure was developed that could generate rHb absorbance spectra indistinguishable from native Hb. Also, rHb generated from TFF-apoHb demonstrated that the reconstituted protein maintains native Hb-like 02 dissociation and CO association kinetics.

Taken together, this example presents an improved method for producing apoHb with a comprehensive analysis of the relevant biophysical properties apoHb and rHb.

### Example 2: Tangential Flow Filtration of Haptoglobin from Plasma or Plasma Fractions

The example describes a scalable process to enable purification of haptoglobin (Hp) from serum proteins in plasma or plasma fractions through the use of tangential flow filtration (TFF). The TFF process brackets Hp in a molecular weight range with low levels of common serum proteins, yielding a product that can include polymeric forms of Hp.

Haptoglobin (Hp) is an α-2 glycoprotein mainly responsible for scavenging cell-free hemoglobin (Hb). Although found in most bodily fluids of mammals, it is present in plasma at concentrations normally ranging from 0.5-3 mg/mL. After binding to cell-free Hb, the Hb-Hp complex is scavenged by CD163+ macrophages and monocytes to clear the organism of toxic cell-free Hb. Cell-free Hb toxicity is attributed to a variety of factors, including Hb extravasation into tissue space which elicits oxidative tissue injury, nitric oxide (NO) and peroxide scavenging, and free heme release. These factors can lead to acute and chronic vascular disease, inflammation, thrombosis, and renal damage. When bound to Hp, the large size of the Hb-Hp complex prevents Hb extravasation into the tissue space, lowering nitric oxide scavenging and vasoconstriction. Furthermore, Hp binding to Hb prevents heme release from Hb, and lowers Hb oxidative damage and inflammation. For these reason, Hp is used as a therapeutic in Japan and is being researched for treatment of various states of hemolysis. Recent studies have also shown various roles Hp plays as a chaperone and in regulation of redox states. In clinical settings, high levels of Hp are indicative of acute inflammation as its expression is upregulated in response to inflammatory cytokines, and low Hp levels are indicative of hemolysis due to receptor-mediated uptake of Hp-Hb complexes.

Hp is a polymorphic protein composed of αβ dimers in which the β polypeptide chain is coded by the same gene, while the α chain can be coded by either the Hp1 or Hp2 codominant alleles. These give rise to three main Hp phenotypes: Hp1-1, Hp2-1 and Hp2-2. The α and β chains are bound through disulfide bonds with the β, α-1 and α-2 chains having a molecular weight (MW) of 36, 9 and 18 kDa, respectively. The α-1 chain has a second cysteine residue after binding to the β chain that allows it to bind to another α chain of an αβ dimer. Thus, Hp1 homozygotes produce tetrameric Hp1-1 (two β and two α-1) species with a MW of about 89 kDa. On the other hand, the α-2 chain has two free cysteine residues when bound to the β chain allowing it to bind to two αβ dimers. This extra cysteine residue leads to the formation of Hp polymers in heterozygote or Hp2 homozygote individuals. Heterozygotes produce Hp2-1, a linear Hp polymer with an average MW of about 200 kDa, which can go up to about 500 kDa. Finally, Hp2 homozygotes produce Hp2-2 which is a cyclic polymer form of Hp with average MW of about 400 kDa ranging from 200 to 900 kDa. All types of Hp bind Hb via a practically irreversible reaction, with a K_{d} ranging from 10⁻¹² to 10⁻¹⁵M. The bond occurs between the β chain of Hp to the β globin of Hb at a stoichiometry of 1:1 for each dimer. Since the MW of Hp differs between phenotypes, the mass stoichiometry is not consistent, with about a 1.3:1 mass binding ratio for Hp1-1:Hb and about a 1.6:1 mass binding ratio for Hp2-2:Hb. Furthermore, Hp 2-2 has been shown to have a higher affinity for the CD163+ receptor, but lower clearance rate through CD163+ uptake. However, the different Hp phenotypes reduce Hb toxicity to the same extent in vivo. Furthermore, Hp2-2 has not been found to have differences in the rate of heme loss, Hb oxidation, and Hb dimer association kinetics in vitro compared to Hp1-1 Although there are no significant differences with its role in Hb scavenging, differences in Hp phenotype have been associated with different rates of cardiovascular disease and cancer as well as different roles with some forms of disease.

In addition to the three main Hp phenotypes, another related Hb scavenging species is haptoglobin related protein (Hpr). Hpr is composed of smaller α and β chains than Hp1-1 and is predominantly found as single αβ dimers, but has been shown to form polymers. With >90% sequence identity to the Hp1 gene, Hpr binds to Hb with high affinity. Unlike Hp, the α chains do not covalently bind to other α chains through disulfide bonds to create αβ polymers, but are thought to connect via non-covalent interactions. The physiological role of Hpr differs from normal Hp as Hpr does not bind to the CD163 receptor and does not have increased expression during states of hemolysis. Instead, Hpr forms a complex with high-density lipoproteins called trypanosome lytic factor 1 (TLF1) and TLF2, which can have a large range of molecular sizes. These complexes have lytic activity against the African cattle parasite Trypanosoma brucei brucei, which use the trypanosoma receptor to obtain iron from the heme of Hb through binding of Hp-Hb complexes. Since Hpr can still bind to Hb in TFL1 and to the trypanosoma receptor, the Hpr-Hb in TFL1 acts as a “troj an horse” against trypanosoma parasites providing humans an innate defense against this disease.

The normal circulatory half-life of Hp is 1.5-2 days in humans, but the half-life of the Hp-Hb complex reduces to ˜20 min, with a maximum clearance rate of 0.13 mg/mL of plasma per hour. Due to the higher rate of clearance of the Hp-Hb complex, even though Hp production is upregulated during states of hemolysis, the concentration of Hp in plasma is inversely related to the concentration of cell-free Hb in plasma. In addition to its upregulation due to the presence of cell-free Hb in the circulation, Hp synthesis is heavily stimulated during acute phase reactions (inflammation, infection, trauma, and malignancy).

Hp can be used as a therapeutic during conditions that cause states of hemolysis (e.g., chronic anemia, transfusion, etc.). In these states, rupture of red blood cells (RBCs) releases cell-free Hb that can scavenge NO, leading to vasoconstriction as well as formation of free radicals and reactive oxygen species that can lead to oxidative damage of surrounding tissues. Hp upregulation during bacterial infection has been related to iron deprivation of pathogens. For this reason, Hp may be used to treat septic shock. Additionally, Hp or the Hp/serum protein mixtures presented here, may be used in RBC storage solutions to extend the ex vivo shelf-life of these cells by attenuating the side-effects associated with lysed RBCs. These solutions could also be co-administered with RBCs or hemoglobin-based oxygen carriers (HBOCs) to prevent and treat the side-effects associated with cell-free Hb.

Hp has been clinically approved in Japan since 1985. Reports of its use show positive effects against burn injuries, trauma from massive transfusions and as a prophylactic during surgical interventions such as cardiac bypass surgery with extracorporeal circulation. Hp treatment during severe burns has been shown to prevent acute renal failure and reduce kidney damage from surgery.

The biomedical applications of Hp are very promising. Its clinical use in Japan has shown positive effects against burn injuries, trauma from massive transfusions and as a prophylactic during surgical interventions such as cardiac bypass surgery with extracorporeal circulation. Hp treatment during severe burns has been shown to prevent acute renal failure, and reduce kidney damage from surgery. In general, Hp can be used to detoxify cell-free Hb that is present in the systemic circulation via various pathophysiological conditions or RBC transfusions that result in hemolysis. Hp treatment has also been shown to prevent damage from stored RBCs, potentially prolonging its shelf-life.

Yet, its wide-scale use is restricted by current production methods, which are not easily scalable and expensive. Furthermore, treatments with Hp require large quantities of material per dose. Existing Hp production protocols consist of using either Hb-affinity chromatography, hydrophobic interaction chromatography, anion-exchange chromatography, or recombinant Hp expression. Chromatography has low yields and is limited by the protein binding capacity of the column. Furthermore, chromatography requires the use of harsh denaturants to dissociate Hp from the bound chromatography matrix. Finally, recombinant Hp is expensive and cumbersome to manufacture.

This example describes a Hp production method via TFF that provides an easy, scalable and economically efficient method for producing Hp from plasma or plasma fractions. The plasma fraction chosen for this example was human Cohn Fraction IV obtained via the modified Cohn process of Kistler and Nitschmann (See, for example, Kistler, P. & Nitschmann, H. Large Scale Production of Human Plasma Fractions. Vox Sang 7, 414-424 (1962)). This plasma fraction is known to contain large MW Hp (Hp2-2 and Hp2-1) from pooled plasma. Low MW Hp (small Hp2-1 polymers and Hp1-1) are primarily found in Cohn Fraction V.

Materials and Methods

Materials. Sodium phosphate dibasic, sodium phosphate monobasic, sodium chloride, and fumed silica (S5130) were purchased from Sigma Aldrich (St. Louis, Mo.). 0.2 μm Millex-GP PES syringe filters were purchased from Merck Millipore (Billerica, Mass.). A KrosFlo® Research II tangential flow filtration (TFF) system and hollow fiber (HF) filter modules were obtained from Spectrum Laboratories (Rancho Dominguez, Calif.). Human Fraction IV Paste was purchased from Seraplex, Inc (Pasadena, Calif.).

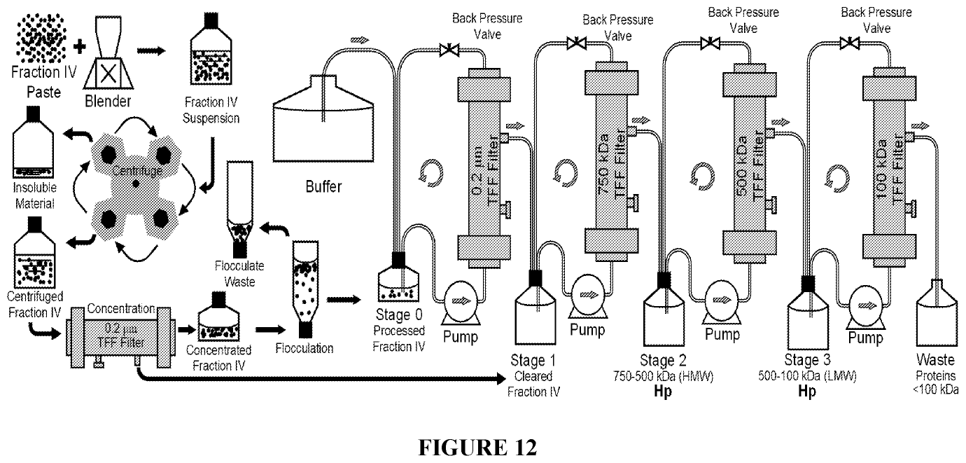

Hp Purification via TFF Without Fumed Silica. 500 g of human Fraction IV (FIV) paste from the modified Cohn process of Kistler and Nitschmann was suspended in 5 L of PBS, and homogenized in a blender. The resulting mixture was stirred overnight at 4° C. The 5 L solution was then centrifuged at 3700 g for 45 minutes to remove undissolved lipids. The supernatant was concentrated using a 0.2 μm hollow fiber (HF) filter to 2 L. The 2 L retentate was left to rest for 36 hrs to flocculate low density particles, while the filtrate was kept at 4° C. for further processing. After flocculation of the retentate, low density particles in solution were separated. The higher density fraction (Stage 0) was then concentrated to 800 mL on a 0.2 μm HF filter and subjected to 10 diafiltrations with PBS. The 0.2 μm filtrate (Stage 1) was concentrated to 150 mL and subjected to 100 diafiltrations on a 750 kDa HF filter using a mixture of the 0.2 μm permeate and PBS. The permeate of the 750 kDa (Stage 2) was then concentrated to 150 mL subjected to 40 diafiltrations using PBS on a 500 kDa HF filter. Finally, the permeate of the 500 kDa HF filter (Stage 3) was concentrated to 150 mL and subjected to 100 diafiltrations using PBS on a 100 kDa filter. A diagram of the purification process is shown in FIG. 12 and the characteristics of the filters used are shown in Table 3. The ˜150 mL solutions of both the 750-500 kDa (high molecular weight, HMW) and 500-100 kDa (low molecular weight, LMW) brackets were then concentrated on 100 kDa HF filters to ˜5-10 mL for the HMW and ˜30 mL for the LMW brackets, respectively.

TABLE 3
Hollow fiber filters (Waltham, MA) used for the purification
of Hp from Cohn Fraction IV using tangential flow filtration.

| | | | | Surface Area | |
|---|---|---|---|---|---|
| Type | Pore Size | | Membrane | (cm²) | P/N |
| miniKros | 0.2 | μm | PES | 470 | S02-P20U-10-N |
| miniKros | 750 | kDa | mPES | 790 | S02-E750-05-N |
| miniKros | 500 | kDa | PS | 1000 | S02-S500-05-N |
| miniKros | 100 | kDa | mPES | 1000 | S02-E100-05-N |
| midiKros | 100 | kDa | mPES | 115 | D02-E100-05-N |

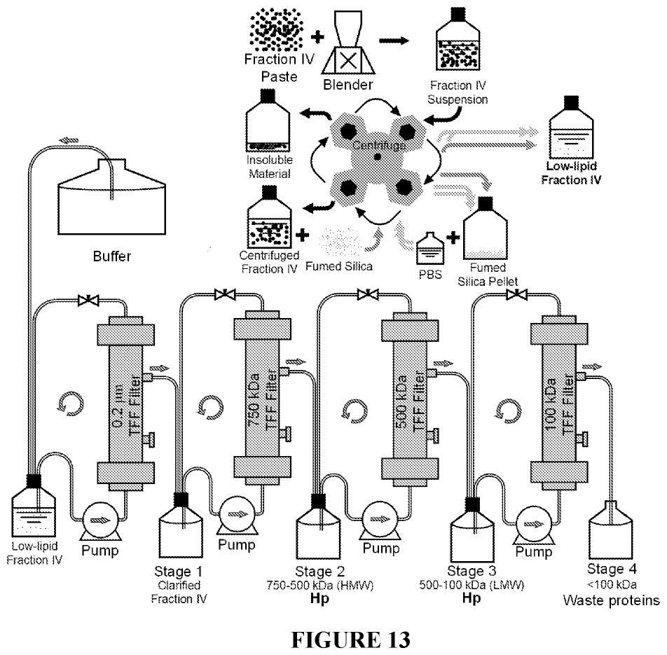

Hp Purification via TFF with Fumed Silica. 500 g of human Fraction IV (FIV) paste from the modified Cohn process of Kistler and Nitschmann was suspended in 5 L of PBS, and homogenized in a blender. The resulting mixture was stirred overnight at 4° C. The ˜5 L solution was then centrifuged at 3700 g for 45 minutes to remove undissolved lipids. Fumed silica (Sigma Aldrich P #55130; St. Louis, Mo.) was then added to the sample at 20 mg/mL concentration and left stirring overnight at 4° C. The solution was then centrifuged to remove the silica agglomerates. Furthermore, the silica pellet was washed twice with PBS to maximize protein recovery. The fumed silica supernatant solution was then concentrated to 800 mL on a 0.2 μm HF filter and filtered for 15 diafiltrations. The 0.2 μm filtrate was concentrated to 150 mL and subjected to 100 diafiltrations using PBS on a 750 kDa HF filter. The permeate was then subjected to 40 diafiltrations using PBS on a 500 kDa HF filter. Finally, the permeate of the 500 kDa HF filter was subjected to 100 diafiltrations using PBS on a 100 kDa filter. A diagram of the purification process is shown in FIG. 13 and the characteristics of the filters used are shown in Table 3. The ˜150 mL solutions of both the 750-500 kDa (high molecular weight, HMW) and 500-100 kDa (low molecular weight, LMW) brackets were then concentrated on 100 kDa HF filters to ˜5-10 mL for the HMW and ˜40 mL for the LMW brackets, respectively.

Size Exclusion Chromatography: Samples were separated via size exclusion chromatography (SEC) using an Acclaim SEC-1000 (4.6×300 mm) column (Thermo Fisher Scientific, Waltham, Mass.) attached to a Dionex UltiMate 3000 system (Thermo Fisher Scientific, Waltham, Mass.). The mobile phase consisted of 50 mM potassium phosphate, pH 7.4 at a flow rate of 0.35 mL/min controlled by Chromeleon 7 software. Wavelength absorbance detection was set to λ=280 nm to detect protein, and λ=413 nm to detect Hb. To estimate the average MW of the Hp products, protein standards (conalbumin, 76 kDa; hHb, 64 kDa; carbonic anhydrase, 29 kDa; ribonuclease A, 14 kDa; and aprotinin, 6.5 kDa) were analyzed on the SEC column. The known molecular weight (MW) of the standards and their elution time were used to determine the coefficients (A, B) of a base 10 exponential function (MW=10^{A*(elution time)+B)} via non-linear regression. The estimated function parameters were used to estimate the average MW of Hp products based on their elution time.

Hb Concentration. The concentration of Hb in the samples was measured spectrophotometrically via the Winterbourn equations.

Residual Hb in Hp Preparations. The residual Hb was quantified via the molar extinction coefficient of methemoglobin at its Soret Peak maxima of 404 nm (c404=167 mM⁻¹ cm⁻¹).

Residual Apohemoglobin in Hp Preparations. The concentration of residual apohemoglobin (apoHb) was estimated based on a modified version of the abridged dicyanohemin incorporation assay. Briefly, Hp samples were mixed with excess methemealbumin (heme bound to human serum albumin [HSA]) and the mixture left to react for 15 hours at room temperature. The change in absorbance of the reacted mixture compared to the initial sample components was used to estimate the amount of heme exchanged from methemealbumin to apoHb. Given the estimated extinction coefficients for the hemichrome-like apoHb species formed (ε₄₁₂ₙₘ≈120 mM⁻¹ cm⁻¹) and methemealbumin (ε₄₁₂ₙₘ≈70 mM⁻¹ cm⁻¹), the extinction coefficient for the change in absorbance spectra was determined to be 55 mM⁻¹ cm⁻¹ at 412 nm.

ELISA. To quantify the concentration of residual protein components in the Hp samples, ELISA kits specific for Hp, transferrin (Tf), human serum albumin (HSA), and hemopexin (Hpx) were used (R&D Systems Catalog #DHAPGO for Hp, and Eagle BioSciences HTF31-K01 for Tf, HUA39-K01 for HSA, and HPX39-K01 for Hpx).

Gel Electrophoresis: The purity of Hp fractions was analyzed via SDS-PAGE using an Invitrogen Mini Gel Tank (Thermo Fisher Scientific, Waltham, Mass.). Wide-range Tris-Glycine gels consisting of pre-cast 4-20% or 10-20% polyacrylamide were used with samples prepared according to the manufacturer's guidelines. Gels were loaded with 20 μL of sample corresponding to approximately 30 μg of protein per lane and tested under reducing (via addition of 0.1 M DTT) and non-reducing conditions. Gels were stained for one and a half hours with Coomassie® Briliant Blue R-250 staining solution, then de-stained overnight. Gels were imaged on a table-top scanner at 300 dpi. To estimate the percent composition of each band, reduced and non-reduced gels were slightly overloaded (˜60 μg of protein) and densitometric analysis was performed on the scanned images using ImageJ. The Hp composition was determined based on the sum of protein bands corresponding to Hp or Hpr a and β chains from the reduced gels subtracted by the composition of the Hb-eluting band on the non-reduced gels (due to the known coelution of Hb α and β chains with the α-1 chain of Hp).

Trypsin Digest Mass Spectrometry. Samples were reduced with 5 mM DTT, incubated at 65° C. for 30 min. Iodoacetamide was then added to a final concentration of 15 mM and the sample was incubated in the dark, at room temperature for 30 minutes. Sequencing grade trypsin was added at a 1:50 ratio and the sample was digested overnight at 37° C. The following day, the samples were acidified with trifluoro acetic acid. The sample was clarified at 13,000 rpm for 5 min in a microcentrifuge, dried in a vacufuge and resuspended in 20 μL of 50 mM acetic acid. Peptide concentration was determined by NanoDrop (i.e. absorbance at 280 nm). Protein identification was performed using nano-liquid chromatography-nanospray tandem mass spectrometry (LC/MS/MS) on a Thermo Scientific Fusion Orbitrap mass spectrometer equipped with an EASY-Spray™ Sources operated in positive ion mode. Samples were separated on an easy spray nano column (Pepmap™ RSLC, C18 2μ 100 A, 75 μm×250 mm Thermo Scientific) using a 2D RSLC HPLC system from Thermo Scientific. Each sample was injected into the μ-Precolumn Cartridge (Thermo Scientific,) and desalted with 0.1% formic acid in water for 5 minutes. The injector port was then switched to inject and the peptides were eluted off of the trap onto the column. Mobile phase A was 0.1% formic acid in water and acetonitrile (with 0.1% formic acid) was used as mobile phase B. Flow rate was set at 300 nL/min. Typically, mobile phase B was increased from 2% to 20% in 105 min and then increased from 20-32% in 20 min and again from 32%-95% in 1 min and then kept at 95% for another 4 min before being brought back quickly to 2% in 1 min. The column was equilibrated at 2% of mobile phase B (or 98% A) for 15 min before the next sample injection. MS/MS data was acquired with a spray voltage of 1.7 kV and a capillary temperature of 275° C. The scan sequence of the mass spectrometer was as follows: the analysis was programmed for a full scan recorded between m/z 375 ˜1575 and a MS/MS scan to generate product ion spectra to determine amino acid sequence in consecutive scans starting from the most abundant peaks in the spectrum in the next 3 seconds. To achieve high mass accuracy MS determination, the full scan was performed at FT mode and the resolution was set at 120,000. The AGC Target ion number for FT full scan was set at 4×10⁵ ions, and maximum ion injection time was set at 50 ms. MS/MS was performed using ion trap mode to ensure the highest signal intensity of MS/MS spectra using CID (for 2+ to 7+ charges). The AGC target ion number for ion trap scan was set at 1×10⁴ ions, and maximum ion injection time was set at 30 ms. The CID fragmentation energy was set to 35%. Dynamic exclusion was enabled with a repeat count of 1 within 60 s and a low mass width and high mass width of 10 ppm. Data sets were analyzed as the Total Ion Intensity for each protein (normalized based on the total ion current) using Scaffold 4 (Proteome Software, Inc).

Total Protein Assay. Total protein of the samples was determined via the Bradford Assay.

Hb Binding Capacity of Hp (Fluorescence). The Hb binding capacity (HbBC) of samples was determined based on the fluorescence quenching method described in the literature. Briefly, a Hp sample is mixed with increasing amounts of Hb and the fluorescence emission at 330 nm (with excitation at 285 nm) is measured. Binding of Hb to Hp quenches the fluorescence of Hp, leading to an observable titration curve. This assay was repeated on products of Stage 2 and 3 for three different batches and compared to the SEC method to quantify HbBC.

Hb Binding Capacity of Hp (SEC): The difference in molecular weight (MW) between the Hp-Hb protein complex and pure Hb was used to assess the Hb binding capacity of Hp. Briefly, samples containing Hp were mixed with excess Hb then separated via SEC. The difference in the area under the curve between the pure Hb solution, and the mixture of Hb and Hp was used to assess the HbBC of Hp.

Heme Binding Activity: The activity of the heme-binding pocket of the protein scavenger cocktail was determined via the dicyanohemin (DCNh) incorporation assay. Briefly, increasing amounts of DCNh was added to a constant concentration of sample and the inflection point of the equilibrium absorbance at the Soret maxima was used to determine the molar quantity of heme required to saturate the heme-binding sites of the sample. The mass concentration of the heme-binding proteins was estimated based on an approximate molecular weight of 65 kDa for human serum albumin and hemopexin.

Effect of Fumed Silica: To assess the total loss of protein from the use of fumed silica, three 50 mL samples of suspended Fraction IV at 100 mg/mL were characterized after removal of the unsuspended FIV (mostly lipids), after addition of 20 mg/mL fumed silica with overnight stirring (fumed silica supernatant), and after each of the two washes (washing consisted of replacing the volume of supernatant removed with fresh PBS, and mixing the fumed silica pellet). The volume reduction due to fumed silica addition was approximated to be 15% as described by the manufacturer. Characterization consisted of quantification of HbBC and total protein, and via separation via SEC-HPLC. The percent of HbBC and total protein retained at each stage after fumed silica addition was calculated based on the ratio of the concentration of HbBC and total protein compared to the FIV supernatant prior to silica addition.

Results and Discussion

Hemoglobin Binding Capacity of Hp. Throughout this study, Hp activity was quantified based on the HbBC of the sample. This parameter indicates the mass of Hb that a given volume of the Hp sample can bind. This approach for Hp quantification is also used in Japan where the HbBC is equivalent to the international units (IU) of the therapeutic compositions of Hp. Given that different Hp phenotypes have different mass binding ratios to Hb and that the main role of Hp is as a Hb binding protein, the HbBC provides a more reliable and practical measurement of Hp activity in a Hp sample. This measurement of Hp activity is of critical importance in assessing the efficacy of various Hp production methods, especially those that rely on harsh denaturing conditions to purify Hp in which some of the Hp may be denatured.

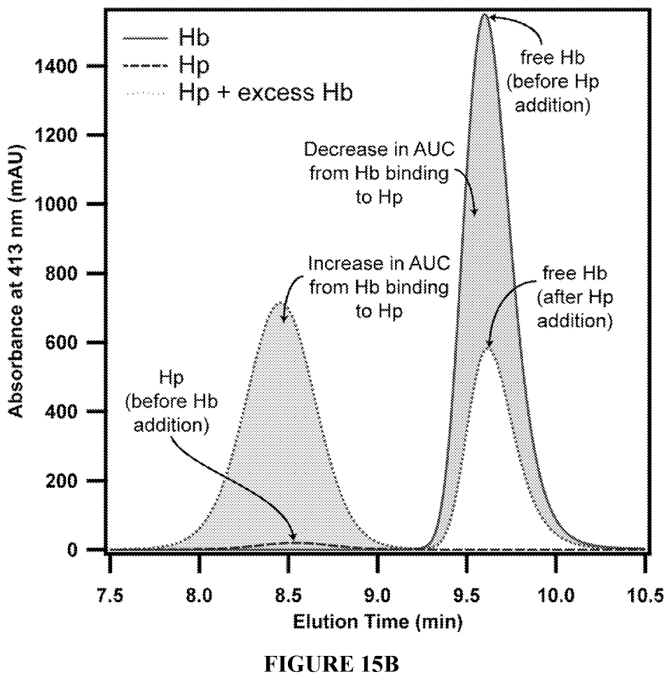

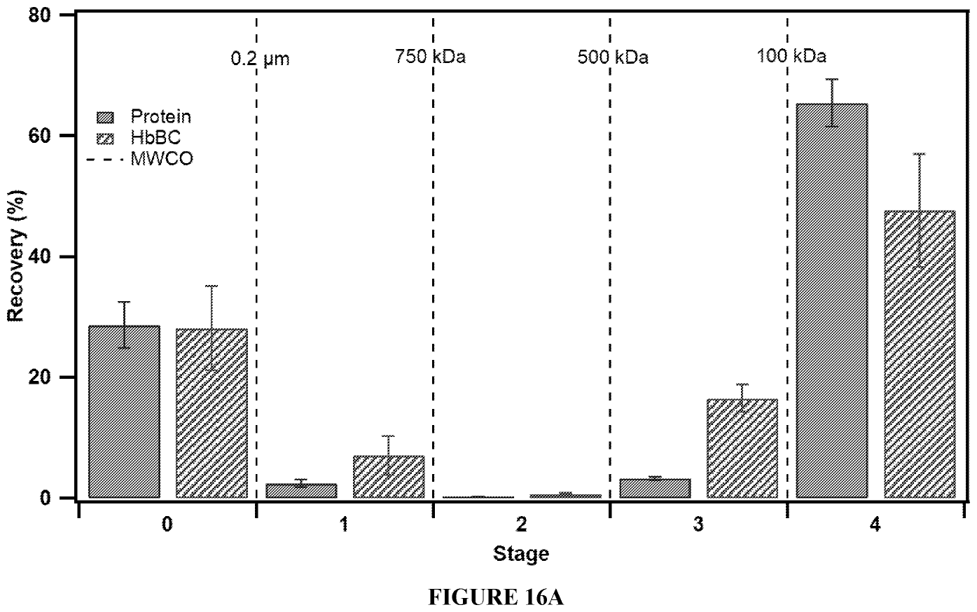

There are various methods to quantify the HbBC of Hp samples. These include spectrophotometric titrations with Hb, immunodiffusion, gel electrophoresis, spectrophotometric differences between deoxygenated Hb and the deoxygenated Hb-Hp complex, differences in peroxidase activity of Hb compared to the Hb-Hp complex, and fluorescence titration of Hp with Hb. Unfortunately, spectrophotometric assays can be dependent on the Hp phenotype and convoluted by other species. Additionally, ELISA can also be used, but differences in polymer sizes can also lead to inaccurate readings. One of the initial methods was based on the fluorescence quenching of Hp tryptophan residues upon Hb binding. In this method, a stock Hp sample is titrated with increasing Hb concentrations and the fluorescence intensity is measured after each addition. The saturation point of Hb binding sites in Hp is determined by the change in slope of the titration curve. FIG. 15A shows an example of a Hp fluorescence titration curve against Hb. Yet, this procedure can be time consuming, requires many data points, and can have large variations from the fitted lines. To streamline and improve HbBC measurements of Hp samples, a new and quick method to determine HbBC was developed. In this method, the difference in size between Hb compared to Hb bound to Hp (i.e. Hb-Hp complex) was used to determine the quantity of Hb bound in Hb-Hp complexes. This was done by monitoring the absorbance at 413 nm during SEC-HPLC of the sample and calculating the area under the curve (AUC) of a known concentration of free Hb, and then repeating this measurement after mixing the Hb with a Hp sample. The decrease in the AUC of the peak corresponding to free Hb after mixing with Hp was directly proportional to the HbBC of the sample, and could be precisely calculated based on the known concentration of Hb. Examples of HPLC-SEC chromatograms of Hp samples using this procedure are shown in FIG. 15B. The HbBC could also be calculated based on the increase in AUC of the peak corresponding to the Hb-Hp complex and is not restricted to only monitoring the 413 nm wavelength (protein peak at 280 nm and others may also be used).

Comparing the fluorescence titration and HPLC-SEC AUC Hp quantification methods for six different Hp containing samples, there was less than 5% variation of the results which, at the concentrations tested, led to less than 1 mg/mL of HbBC variation. Furthermore, the HPLC-SEC method has less than 1% variation in precision. Thus, the 5% variation seen may have been due to the intrinsic variation of the fluorescence titration method. Furthermore, these variations were similar or better than the reported values for previous HbBC quantification methods. The difference in peroxidase activity, had larger variations (7.6% using different standards and 2.6% using same standard curve). Spectrophotometric differences between free Hb and Hb-Hp had more than 10% error. Furthermore, spectrophotometric titration with Hb had about 2% variation within the same sample and ranged from 2-11% when the same sample was tested on a different day. A similar method which employed SEC-HPLC to determine HbBC has been employed previously, in which the AUC of the Hb-Hp complex was divided by the total AUC of the chromatogram. Although relying on the same concept (use of HPLC-SEC to separate Hb from the Hp-Hb complex), the previously used method requires that the Hp does not have any Hb bound to it. Furthermore, slight modifications in the absorbance of Hb-bound species may occur. For example, the change in absorbance from the binding of free cyanomethemoglobin to Hp has been used to quantify HbBC. Using the method presented here in which only the AUC of the Hb peak is used in the analysis, this method removes potential errors from analysis of the AUC from the Hp-Hb complex peak.

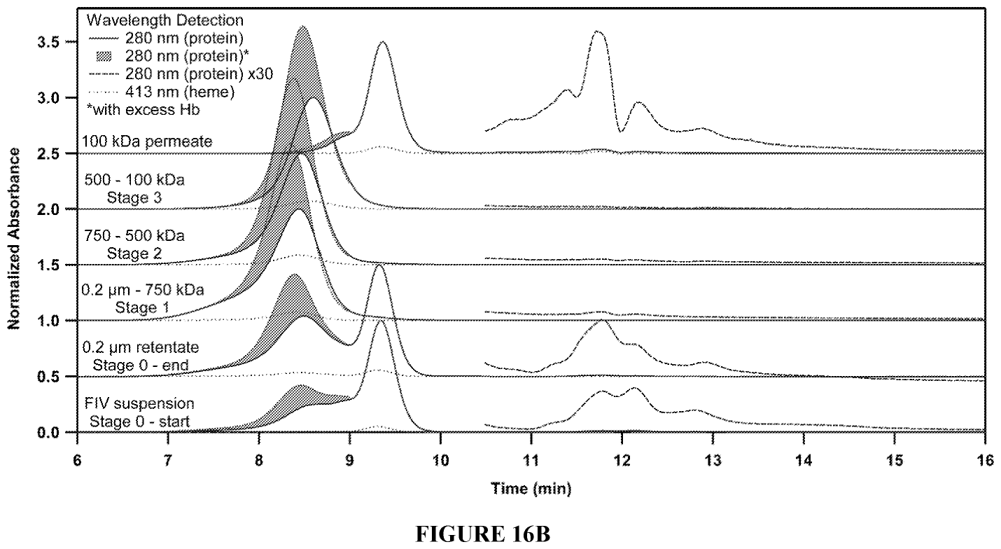

TFF Production of Hp Without Fumed Silica. Production of Hp mixtures via the tangential flow filtration (TFF) procedure described in the Methods Section yielded 5 different stages in which different molecular weight (MW) proteins could be isolated. These 5 stages were the proteins retained on the 0.2 μm HF filter (Stage 0, 0.2 μm retentate), the bracket of proteins between the 0.2 μm and 750 kDa HF filters (Stage 1, 0.2 μm-750 kDa), the bracket between the 750 kDa and the 500 kDa (Stage 2, 750-500 kDa), the bracket between the 500 and 100 kDa HF filters (Stage 3, 500-100 kDa), and lastly, the permeate of the 100 kDa HF filter (Stage 4, <100 kDa). The protein and Hb binding capacity (HbBC) yield at each stage based on the average of four batches without the use of fumed silica is shown in Table 4. The recovery data for total protein and Hb binding capacity (HbBC) at each stage of processing is also shown in FIG. 16A.

TABLE 4
Summary of total protein concentration and HbBC, and analysis of total protein and
HbBC yield of batches without the use of fumed silica.

| | Total | | Total | HbBC | Protein | HbBC |
|---|---|---|---|---|---|---|
| | Protein | HbBC | Protein | per mg | Yield | Yield |
| STAGE | (mg/mL) | (mg/mL) | (g) | (%) | (%) | (%) |
| 0′ | 22.0 ± 1.4 | 1.4 ± 0.1 | 115 ± 9 | 6.59 ± 0.97 | 100 | 100 |
| FIV | | | | | | |
| suspension | | | | | | |
| 0 | 31.6 ± 5.2 | 2.0 ± 0.4 | 33.1 ± 5.1 | 6.35 ± 1.1 | 28.69 ± 3.8 | 28.11 ± 6.98 |
| 0.2 μm | | | | | | |
| retentate | | | | | | |
| 1 | 20.7 ± 2.8 | 3.6 ± 1.8 | 2.77 ± 0.68 | 18.4 ± 6.2 | 2.42 ± 0.65 | 7.05 ± 3.18 |
| 0.2 μm-750 | | | | | | |
| kDa | | | | | | |
| 2 | 44.2 ± 7.2 | 12.2 ± 3.9 | 0.191 ± 0.019 | 27.0 ± 5.1 | 0.17 ± 0.03 | 0.71 ± 0.15 |
| 750-500 | | | | | | |
| kDa | | | | | | |
| 3 | 100.1 ± 4.4 | 32.8 ± 1.8 | 3.77 ± 0.20 | 32.8 ± 2.3 | 3.28 ± 0.26 | 16.5 ± 2.3 |
| 500-100 | | | | | | |
| kDa | | | | | | |
| 4 | <1 | — | 75.5 ± 8.1 | 4.9 ± 1.7 | 65.4 ± 3.9 | 47.6 ± 9.4 |
| 100 kDa | | | | | | |
| permeate* | | | | | | |

*values were not measured and include all losses during processing; estimates were based on the initial total protein and HbBC compared to what was recovered in the other stages.

From the results of Table 4 and FIG. 16A, it was apparent that FIV was not primarily composed of soluble proteins. Starting with a ˜100 mg/mL suspension of FIV paste, the solution was only composed of ˜22 mg/mL of soluble total protein. Thus, only approximately 20% of the mass of FIV was composed of soluble proteins. Some of the insoluble material was removed from the centrifugation steps in which approximately 150-200 g of insoluble material was removed. The remaining mass of material was likely retained on the 0.2 μm filter.

Given that most serum proteins have MW smaller than 100 kDa, most of the suspended protein permeated through the 100 kDa filter, leading to 65% of the soluble protein being lost in Stage 4. Furthermore, since Hp can be present over a wide MW range and that FIV contains mostly polymeric Hp, Stage 3 retained a high HbBC fraction of FIV, indicating that Hp was present in this >100 kDa MW bracket. Yet, given that only a small fraction of the total protein of FIV was retained in Stage 3, this stage had the highest HbBC per milligram of total protein. This high HbBC to total protein ratio indicated a high Hp purity in Stage 3. Although at similar HbBC to total protein ratio, only a small quantity (˜4 mL at ˜50 mg/mL) of Stage 2 was purified, with most of the product begin retained in Stage 3 (˜38 mL at 100 mg/mL). Low retention in Stage 2 suggested that the Hp polymers primarily permeated through the 500 kDa filter. Yet, due to the separation on a 500 kDa filter, Stage 2 contained mostly high MW (HMW) Hp polymers, while Stage 3 contained mostly lower MW (LMW) Hp which may allow for testing of the effect of Hp polymer size on the therapeutic index in various disease states. Based on these MW ranges, Hp polymers in Stages 2 and 3 mainly consisted of a mixture of Hp2-2 and Hp2-1, since any Hp1-1 present in FIV (expected to be primarily present in Cohn Fraction V) was too small to be retained in Stage 3.

Unfortunately for the purification of Hp, not only did most proteins permeate through the 100 kDa system, but almost 50% of the initial HbBC also permeated through the system, indicating that these Hp species in FIV were also permeable through the 100 kDa filter (small Hp polymers). Therefore, decreasing the numbers of diafiltrations at the last stage or using a smaller final MW filter could improve retention of these species with a potential drawback of reducing product purity. Furthermore, a large fraction of the initial HbBC (28%) and total protein (29%) was retained on the 0.2 μm filter, indicating that this filter was likely fouled during processing. Thus, starting with a different form of processed human plasma or removing some of the initial fouling particulates may benefit the initial filtration step. Possible solutions could be to introduce a larger filter pore size prior to the 0.2 μm filter or precipitating the protein suspension using ammonium sulfate or other salting-out agents. One promising fraction to use as the starting material could be Cohn Fraction IV-4, which removes many of the lipoproteins from the starting material. Furthermore, as shown later in this study, addition of fumed silica greatly enhanced filtration through the 0.2 μm stage. Stage 1 (0.2 μm ˜750 kDa) also showed potential as a product, but the sample may have to undergo further PBS diafiltration. Furthermore, via this method, Stage 1 contained large molecular weight proteins that did not seem to bind any Hb (see HPLC-SEC).

HPLC-SEC was performed on each of the processing stages and the results are shown in FIG. 16B (the dotted curve indicates the hemoglobin (Hb) binding capacity at each stage).

In FIG. 16B, it was evident that the LMW species were removed at Stages 2 (750-500 kDa) and 3 (500-100 kDa), but present in the 100 kDa permeate. Furthermore, both the 750-500 and 500-100 kDa brackets showed an almost uniform peak with Stage 2 at ˜8.5 min and Stage 3 at ˜8.6 min. These elution times yielded an estimated MW of 340±20 kDa for Stage 3 and 430±40 kDa for Stage 2. Yet, the small tail-end on the chromatograms of Stage 2 and Stage 3 indicated that the actual average MW was higher than the values obtained from the peaks. Moreover, the HPLC-SEC chromatogram for the raw FIV suspension showed how most of the proteins were smaller than 100 kDa (eluting at times larger than ˜9.3 min). Furthermore, based on the permeate of the 100 kDa, it was noticeable that the filter cut-off allowed passage of some >100 kDa species. Thus, these observations explain the 65% total protein recovery obtained in Stage 4.

From these curves, it was also possible to note that some of the high-MW protein species (elution times of ˜7.5 min or earlier) in Stages 0, 1, 2 and 3 did not show Hb-binding properties. This was noted by the lack of increase in absorbance at 280 nm of these high-MW species when excess Hb was added to the sample. When Hb binds to Hp forming Hb-Hp complexes, the absorbance at the Hp/Hp-Hb elution time increases due to the absorbance of Hb at 280 nm. Thus, if no increase was observed, it indicated that most of these high-MW species did not bind to Hb and were likely impurities that were not removed during the TFF processing. Interestingly, Stage 1 (0.2 μm ˜750 kDa) may also have therapeutic potential, but the sample had large quantities of large non-Hb binding proteins and may have to undergo further PBS diafiltrations, increasing overall processing time.

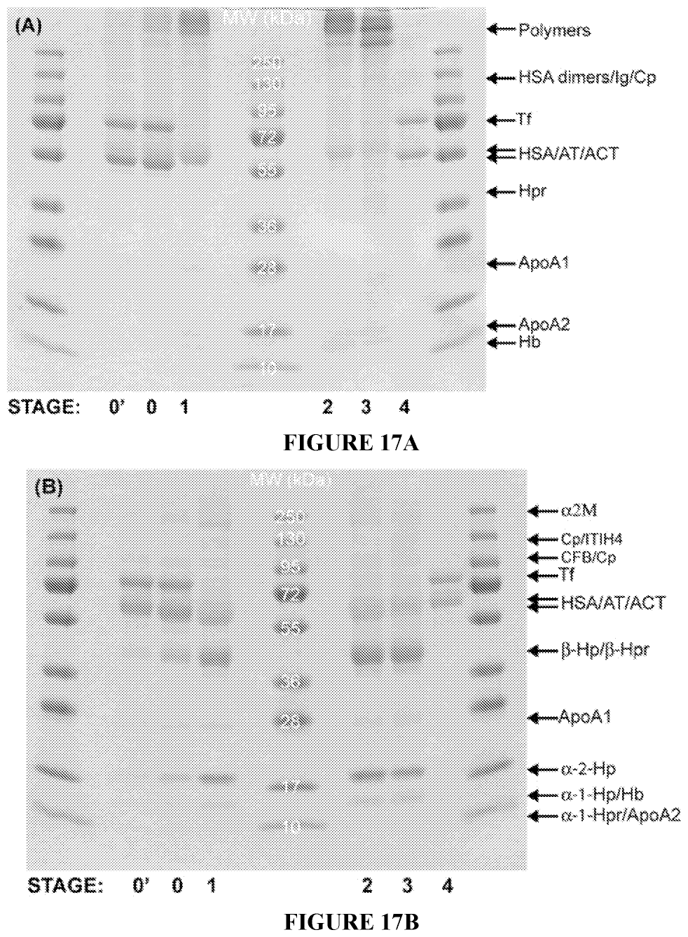

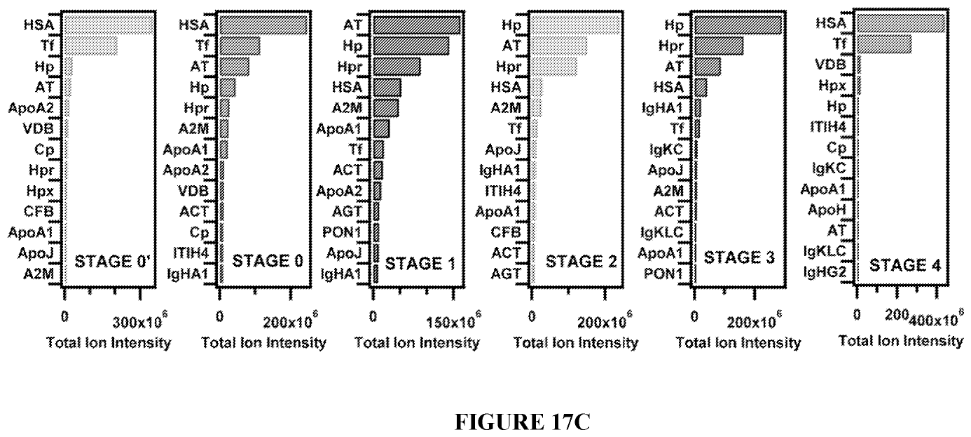

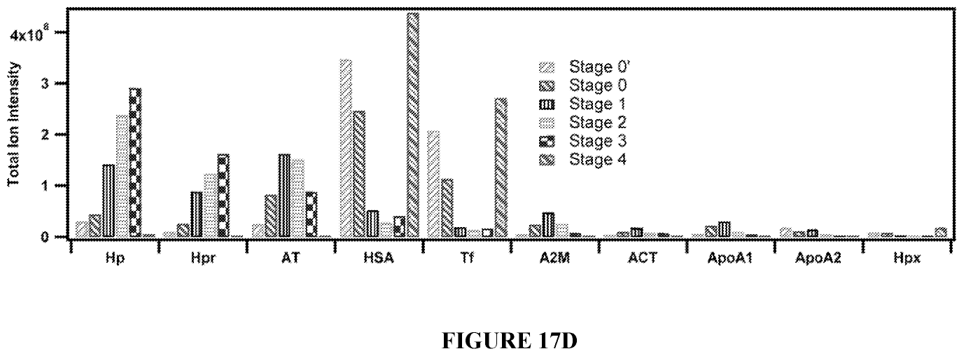

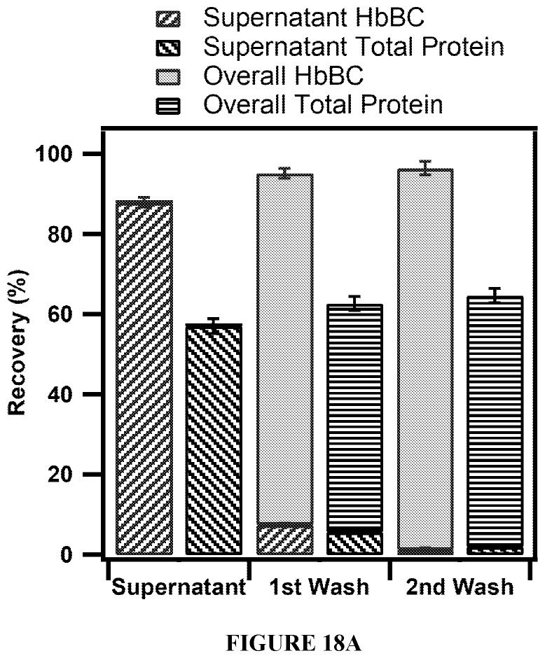

To analyze the protein species in each of the processing stages, SDS-PAGE was performed under both reducing and non-reducing conditions. Furthermore, protein identity was confirmed via trypsin digestion and mass spectroscopy (MS) of the samples. The gels from one representative batch and the percent composition based on the label-free quantitative MS analysis is shown in FIG. 17.

From the results of FIG. 17, it was noted that the most prominent protein bands of the SDS-PAGE could be attributed to the high-abundance proteins identified via MS. Furthermore, the proteins identified via MS were similar to previous reports of FIV-4 MS and the expected components of FIV. Compared to FIV-4, FIV contains proteins of FIV-1, which have large amounts of lipids, explaining the detection of the main components of HDL (apoA1 and apoA2), explaining their detection in MS and SDS-PAGE. In each sample, MS detected over 100 proteins. Yet, most of these impurities were at very low levels and were grouped into a single “Other” category shown in FIG. 17C. To ease analysis of how the proteins were distributed in each stage, the proteins which had more than >1% composition for at least three stages were shown in FIG. 17D.

Based on the increase in relative intensity of the polymeric species in Stages 1-3 of the non-reduced SDS-PAGEs, it was noted that the TFF process was capable of purifying high-MW species present in the FIV suspension (Stage 0). Although these species likely consisted of mostly Hp polymers, other large MW protein species and/or polymerized proteins may also have contributed to the “Polymers” band of FIG. 17A. Furthermore, MS analysis also showed an increase in composition of Hp and Hpr in Stages 1-3. These increases were consistent with the increase in relative HbBC shown in Table 4. Moreover, under non-reducing conditions, a band at ˜60 kDa was present at all stages which was attributed to mainly AT and α-1 antichymotrypsin (ACT) (determined via highest abundance of ˜60 kDa proteins). Transferrin (Tf) was also detected on the SDS-PAGE and in MS of Stages 0-3. Although AT, ACT and Tf are less than 100 kDa and, therefore, would be expected to have permeated through the TFF system; AT and ACT can undergo polymerization when partially denatured and various proteins including AT and Tf can associate with lipoproteins. Given that the fractionation process for production of FIV uses ethanol and acid, this process could also have contributed to partially unfolding and/or oxidizing proteins, leading to their polymerization. Thus, based on size-exclusion purification alone, it would not possible to remove these impurities from the samples. These species were then detected on the non-reducing SDS-PAGE due to dissociation of polymers and/or lipids in the presence of SDS.

Hemoglobin (Hb) and apolipoproteins were also present under the non-reducing SDS-PAGE shown in FIG. 17A. The unfolding of Hp due to the SDS potentially lead to unbinding of Hb to Hp, causing free Hb to appear on the gel. Furthermore, apolipoprotein A1 is a common contaminant in Hp preparations due to Hp binding and could also have been purified with lipoproteins. Apolipoprotein A1 is the major component (70%) of high-density lipoproteins (HDL), which, due to their size, were likely the lipoproteins purified with the high HbBC stages (3 and 4). In addition to apoA1, HDL is composed of ˜15-20% apolipoprotein A2 (apoA2). The presence of HDL corroborated the retention of haptoglobin-related protein (Hpr) which associates with HDL.

On the reduced SDS-PAGE, Hp dissociated into its α and β components. As expected, the majority of the α chains were α-2 which allow for the polymerization of Hp. Furthermore, a higher intensity of the band at ˜60 kDa was detected, likely due to reduction of disulfide bonded albumin polymers and full dissociation of proteins associated with lipoproteins. The presence of these polymeric albumin species would explain the detection of 4-5% HSA via MS on Stages 2 and 3 (FIG. 17C). A high-MW band on the non-reduced SDS-PAGE (˜200 kDa) was found which was attributed to the monomers of α-2 macroglobulin. Other small bands were also detected that were identified via proteins with similar MW of the MS data. Furthermore, various faint bands were found in the 90-130 kDa range, which were attributed to (in order of decreasing MW) inter-alpha-trypsin inhibitor H4 (ITH4), ceruloplasmin (CP), complement factor B (CPB), and plasma protease Cl inhibitor (PC1I). Finally, the other proteins shown under reducing conditions consisted of the same proteins identified on the non-reducing conditions (apoA2 was reduced into its monomeric chains). Interestingly, only a low intensity α-1 Hpr band was seen in Stage 3 which indicated that the MS ion intensity for Hpr may have been incorrectly assigned due to the high (>90%) sequence identity of Hpr to Hp1-1.

Protein purity was also estimated via densitometric analysis of the bands of Stage 2 (HMW) and 3 (LMW). The results are shown in Table 5.

TABLE 5
Purity of Hp products as determined by SDS-PAGE densitometry analysis
of samples purified via TFF without the use of fumed silica.

| | | HMW | | LMW | |
|---|---|---|---|---|---|
| | Species | Stage 2 | +/− | Stage 3 | +/− |
| NON- | Polymers | >74.3% | 3.8% | >84.7% | 3.4% |
| REDUCED | HSA/AT/ACT | <18.3% | 3.2% | <8.4% | 3.3% |
| | Hb/apoHb | <2.9% | 0.3% | <2.0% | 0.8% |
| | Tf | <1.2% | 0.1% | <1.8% | 0.5% |
| | ApoA1 | <1.3% | 0.6% | <1.2% | 0.2% |
| | Hpr | <0.7% | 0.8% | <1.0% | 0.5% |
| | ApoA2 | <1.2% | 0.1% | <0.5% | 0.1% |
| | ApoJ | <0.0% | 0.0% | <0.3% | 0.5% |
| | Other | <0.0% | 0.0% | <0.2% | 0.3% |
| REDUCED | β-Hp/β-Hpr/IgHA1 | >36.8% | 4.7% | >44.9% | 4.4% |
| | α-2Hp/IgKC | >23.9% | 0.2% | >20.7% | 0.5% |
| | AT/HSA/ACT | <19.6% | 5.1% | >11.1% | 5.2% |
| | α-1Hp/Hb/IgKLC/apoHb | <7.1% | 1.4% | <9.9% | 1.6% |
| | ApoA1 | <3.8% | 1.1% | <5.0% | 0.9% |
| | α-2M | <3.3% | 1.4% | <1.5% | 0.6% |
| | α-1Hpr/ApoA2 | <1.0% | 0.2% | <1.2% | 0.1% |
| | ITIH4/Cp | <0.7% | 0.1% | <2.0% | 1.0% |
| | Cp/CFB | <2.5% | 0.5% | <1.6% | 0.9% |
| | Tf | <1.0% | 0.5% | <0.9% | 0.1% |
| | Other (~65-70 kDa) | <0.4% | 0.2% | <1.3% | 0.4% |
| | Other (~15 kDa) | — | — | <0.5% | 0.8% |
| | Hb* | 1.0% | 0.1% | 1.0% | 0.1% |
| | ApoHb** | 2.9% | 1.1% | 3.1% | 0.3% |

| | | | | |
|---|---|---|---|---|
| Total Hp** | 68.0% | 6.6% | 75.3% | 5.0% |

*Amount of Hb was determined via the Soret peak of the sample assuming the presence of methemoglobin (ε_{405 nm} = 167 mM⁻¹cm⁻¹)⁷⁸
**ApoHb content estimated via the exchange of heme from heme-albumin to sample (Δε_{412 nm}≈55 mM⁻¹cm⁻¹ for generated heme-species).
*** Total Hp from the sum of Hp containing bands of the reduced SDS-PAGE subtracted from Hb of the non-reduced SDS-PAGE
Abbreviations: AT: α-1 antitrypsin, ACT: α-1 antichymotrypsin, ATIII: Antithrombin III, Hb: hemoglobin, Tf: transferrin, ApoA1: apolipoprotein A1, Hpr: haptoglobin-related protein, ApoA2: apolipoprotein A2, CLU: clusterin; HSA: human serum albumin, Hp: haptoglobin, α-2M: α-2 macroglobulin, CP: ceruloplasmin, ITH4: Inter-alpha-trypsin inhibitor H4, IgHA1: immunoglobulin heavy constant alpha 1, IgKC: immunoglobulin kappa constant, IgKLC: immunoglobulin kappa light chain, CFB: complement factor B, apoHb: apohemoglobin.

From the densitometry assessment, Stage 2 and 3 were composed of ˜70 and ˜75% Hp, respectively. The major impurity were proteins in the 55-65 kDa range with ˜20 and ˜10% in Stages 2 and 3, respectively. Based on MS, these impurities were primarily composed of human serum albumin (HSA) and α-1 antitrypsin (AT). Due to their similar molecular weights, the proteins were not separately quantified via densitometry. But MS indicated that Stage 2 and 3 had 19% and 11% of AT with 4% and 5% of HSA, respectively. Unfortunately, many impurities were present in the Hp samples, which may have contributed to an inaccurate estimate of Hp purity. Most of these impurities likely originated from proteins associated with lipoproteins and/or proteins that can undergo polymerization. Interestingly, these impurities showed little deviations from batch-to-batch, demonstrating that the purification process was reproducible.

Although convoluted from contaminant proteins, the Hp purity from SDS-PAGE was similar to the expected Hp content based on the average HbBC per mg of total protein of Stage 2 and 3. Using the theoretical the mass ratio of Hp2-2 to Hb, the expected purity of the samples would be approximately 50% and 60% for Stages 2 and 3, respectively. Yet, the Bradford assay may have led to overestimation of total protein due to high concentration of glycoproteins which can also react with the dye used in the assay (Hp can have ˜20% of its total mass attributed to conjugated carbohydrates). Moreover, high MW Hp polymers isolated from serum have been shown to have even higher mass binding ratios than theoretical (>2:1 Hb:Hp), potentially due to tertiary structure steric hindrance. At a mass binding ratio of 2, Stage 3 would have ˜70% Hp, similar to what was obtained from densitometric analysis.

From UV-visible spectrometry, residual Hb contributed ˜1% of the protein mass for the LMW and HMW stages. Yet, based on the SDS-PAGE densitometry, Hb chains consisted of ˜3% of the total mass, indicating that some of the Hp maybe bound to apohemoglobin (apoHb). Thus, the residual apoHb content was assessed by adding excess heme-albumin and monitoring the increase in absorbance at the Soret maxima as described in the Methods section. Given that Hp has been shown to not bind appreciably to heme, the change in absorbance was due to heme exchange from HSA to apoHb. Heme binding indicated that some of the residual Hb may have had its heme extracted during the acidic-ethanol fractioning of plasma to obtain Fraction IV. This heme-binding property of the purified Hp sample could be beneficial during hemolytic states in which free heme is also present.

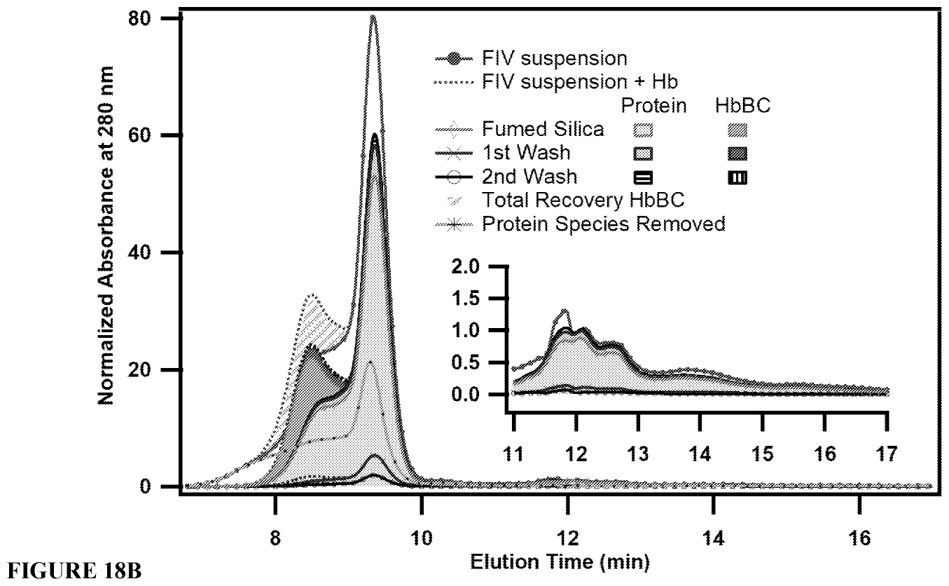

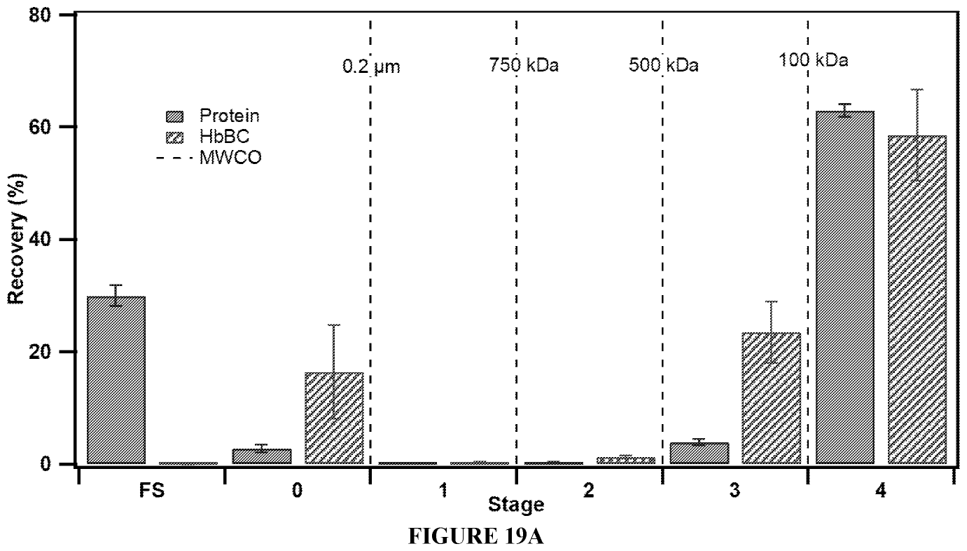

TFF Purification of Hp with Fumed Silica. From the results already shown, it was apparent that lipoproteins were likely the carriers for a large proportion of the impurities in the sample. Fumed silica is commonly used for de-lipidation of serum samples as it is capable of adsorbing lipids. Thus, the effect of using fumed silica on FIV was assessed, and the results are shown in FIG. 18.

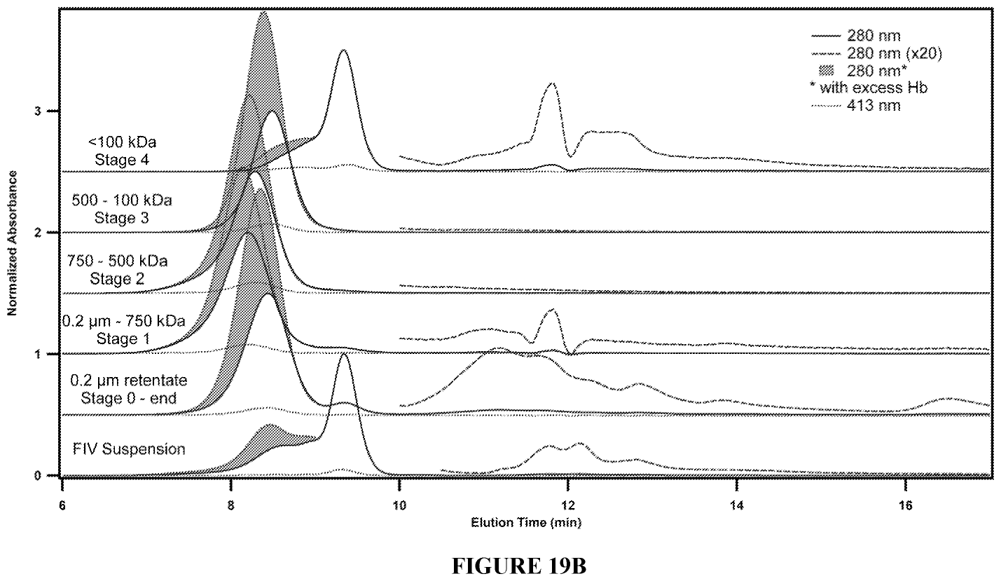

The analysis of percent recovery (FIG. 18A) showed that almost all the HbBC was recovered, but about 30% of the total protein was removed after fumed silica treatment. Thus, prior to TFF purification, the sample was already deprived of impurities. The small loss in HbBC also agreed with the low Hpr content seen on the SDS-PAGE of FIG. 17B given that these Hp species would likely be associated with HDL. The HPLC-SEC chromatogram of these impurities was estimated based on the difference of the FIV suspension and the total sum of chromatograms of the recovered supernatants (FIG. 18B). From the HPLC-SEC chromatograms, it was observed that various MW protein species were removed including the ones eluting at ˜7.5 min. The species eluting at 7.5 min species were running outside of the exclusion limit of the SEC column and, therefore, were likely >1 MDa species, indicating LDL particles were present in the FIV suspension. Furthermore, as shown in FIG. 16B, and confirmed by the high recovery in FIG. 18, these species were mainly impurities that did not bind to Hp and contributed to the left tail-end of the HPLC-SEC chromatograms of the purified Hp products (Stages 2 and 3). Furthermore, protein was also removed in the MW region of Stages 2 and 3 (˜8.5 min), indicating that these fractions could be isolated with higher Hp purity since no appreciable loss of Hp occurred. Thus, to assess the performance of the TFF system using fumed silica prior to filtration, the process described in the Methods section was tested on three batches. The results of these batches are summarized in Table 6. Total protein and HbBC recovery at each stage are also shown in FIG. 19A.

TABLE 6
Summary of products from each stage of the purification of Hp via TFF
using fumed silica.

| | Total | | Total | HbBC | Protein | HbBC |
|---|---|---|---|---|---|---|
| | Protein | HbBC | Protein | per mg | Yield | Yield |
| STAGE | (mg/mL) | (mg/mL) | (g) | (%) | (%) | (%) |
| 0′ | 24.1 ± 3.0 | 1.4 ± 0.2 | 123 ± 12.6 | 5.89 ± 0.84 | 100 | 100 |
| FIV | | | | | | |
| suspension | | | | | | |
| 0 | 5.2 ± 2.8 | 3.0 ± 2.7 | 2.48 ± 0.87 | 51.6 ± 32.6 | 1.96 ± 0.51 | 16.66 ± 8.39 |
| 0.2 μm | | | | | | |
| retentate | | | | | | |
| 1 | 9.3 ± 13.9 | 3.2 ± 4.4 | 0.0701 ± 0.055 | 39.0 ± 22.8 | 0.06 ± 0.05 | 0.27 ± 0.20 |
| 0.2 μm-750 | | | | | | |
| kDa | | | | | | |
| 2 | 53.2 ± 15.1 | 18.6 ± 3.6 | 0.259 ± 0.083 | 35.7 ± 4.5 | 0.2 ± 0.05 | 1.25 ± 0.33 |
| 75-500 | | | | | | |
| kDa | | | | | | |
| 3 | 103.7 ± 6.2 | 51.7 ± 0.6 | 3.48 ± 0.88 | 49.9 ± 2.5 | 2.77 ± 0.44 | 23.87 ± 6.02 |
| 500-100 | | | | | | |
| kDa | | | | | | |
| 4 | — | — | — | 9.65 ± 3.27 | 65.0 ± 0.93 | 57.94 ± 8.97 |
| 100 kDa | | | | | | |
| permeate* | | | | | | |
| Fumed | — | — | — | — | 30 | 0 |
| Silica** | | | | | | |

**values were not measured and include all losses during processing; estimates were based on the initial total protein and HbBC compared to what was recovered in the other stages.
**loss based on data shown in FIG. 18.

From the results of Table 6 and FIG. 19A, it was evident that fumed silica greatly improved the filtration of proteins through the 0.2 um HF filter, since practically none of the protein was retained at the end of Stage 0 or Stage 1. Moreover, use of fumed silica before TFF lead to at least a one-fold increase in the permeate flow rate of all stages during processing. In doing so, even though more centrifugation was required prior to processing, fumed silica greatly decreased the overall processing time. Finally, not only did fumed silica treatment improve processing, but the two main Hp products (Stage 2 and Stage 3) also had significant improvements. Both Stage 2 and Stage 3 showed significantly higher HbBC per total protein (p<0.05) compared to samples processed without fumed silica indicating that the fumed silica treated samples had a higher proportion of Hp. With fumed silica, although Stage 0 showed a similar Hp composition to that of Stage 3, Stage 0 contained suspended insoluble species that were not easily removable via centrifugation. Thus, this sample was not considered as a main Hp product in this study. Yet, new purification techniques may be developed so that this insoluble matter is removed from the solution. To further characterize each of the retained stages, HPLC-SEC was performed, and the chromatograms are shown in FIG. 19B.

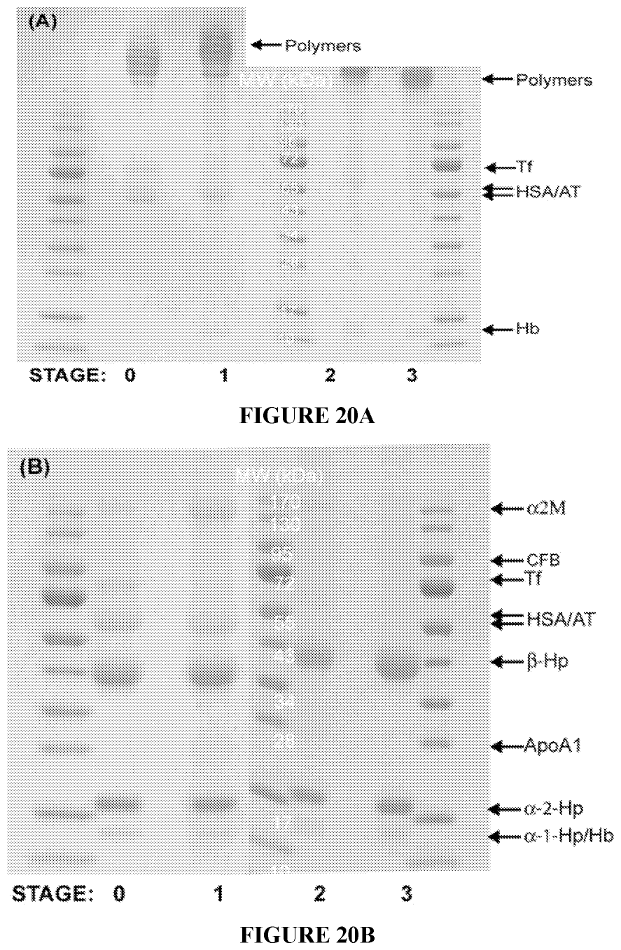

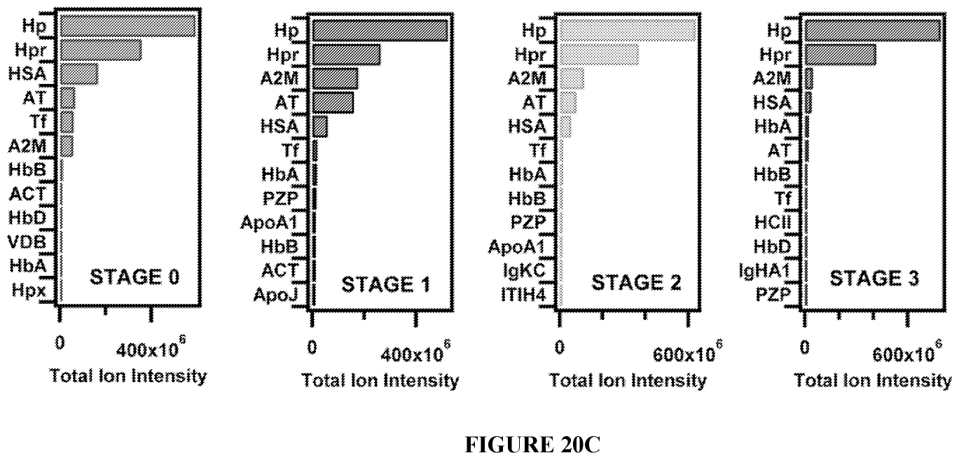

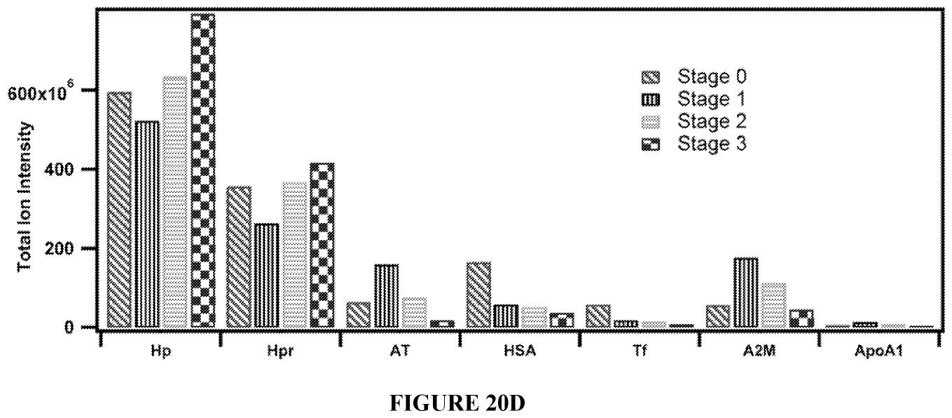

The HPLC-SEC chromatograms followed the same trends for Stages 2 to 4 as the batches that did not use fumed silica. More importantly, Stage 2 and Stage 3 had a reduced left tail-end of the non-Hb-binding proteins but the estimated molecular weight of Stages 2 and 3 increased compared to without the use of fumed silica. Stage 2 and Stage 3 had an average MW of 520±30 kDa and 390±20 kDa, respectively. Thus, even though there was removal of the large ˜7.5 min eluting species, the flux of large MW proteins through the filters improved, yielding larger average sized proteins in each stage. The increase in protein permeation through the filters is also shown by the lack of a peak at ˜9.2 min on Stage 0, indicating high passage of these ˜100 kDa species. Yet, the presence of low MW protein species in Stage 0 indicated that the filter was still fouling. The samples of the main Hp stages (Stage 2 and 3) were also analyzed via SDS-PAGE and trypsin digest MS, the result is shown in FIG. 20.

From the SDS-PAGE, it was apparent that these fractions were primarily composed of Hp. Little of the impurities from the purification method without fumed silica were detected. From a low protein loaded gel (shown in FIG. 20), the purity based on densitometry was >95% for Stage 3. This level of purity is similar or higher than of that previously reported for the commercialized polymeric Hp product produced in Japan. To better identify and estimate protein purity, densitometry analysis of slightly overloaded SDS-PAGE gels was performed and the results are shown in Table 7.

TABLE 7
Purity of Hp products as determined by SDS-PAGE densitometry analysis
of samples purified via TFF with the use of fumed silica.

| | | HMW | | LMW | |
|---|---|---|---|---|---|
| | Species | Stage 2 | +/− | Stage 3 | +/− |
| NON- | Polymers | >88.0% | 3.6% | >94.3% | 1.7% |
| REDUCED | Hb | <3.2% | 1.3% | <3.3% | 1.2% |
| | AT/DBP/ACT | <5.7% | 1.2% | <1.0% | 0.6% |
| | Tf | <0.2% | 0.2% | <0.8% | 0.2% |
| | ApoA1 | <1.5% | 0.5% | <0.2% | 0.2% |
| | ApoA2 | <0.7% | 0.3% | <0.2% | 0.2% |
| | Hpr | <0.3% | 0.3% | <0.1% | 0.1% |
| | apoL1 | <0.2% | 0.2% | <0.0% | 0.0% |
| | Other | <0.1% | 0.2% | <0.0% | 0.0% |
| REDUCED | β-Hp/β-Hpr | >47.4% | 2.7% | >54.2% | 3.4% |
| | α-2Hp | >30.6% | 1.2% | >31.7% | 1.4% |
| | α-1Hp/Hb | <4.0% | 1.5% | <7.2% | 0.9% |
| | HSA/AT | <7.7% | 2.7% | <3.4% | 1.4% |
| | a2M | <6.5% | 0.3% | <2.0% | 1.1% |
| | ApoA1 | <1.9% | 1.2% | <1.5% | 1.3% |
| | CFB | <1.1% | 0.5% | — | — |
| | Tf | <0.9% | 0.1% | — | — |
| | Hb* | 2.0% | 0.4% | 1.0% | 0.1% |
| | ApoHb** | 2.0% | 1.0% | 3.0% | 0.3% |

| | | | | |
|---|---|---|---|---|
| Total Hp*** | 78.6% | 2.9% | 89.9% | 2% |

*Amount of Hb was determined via the Soret peak of the sample assuming the presence of methemoglobin (ε_{405 nm} = 167 mM⁻¹cm⁻¹)⁷⁸
**ApoHb content estimated via the exchange of heme from heme-albumin to sample (ε_{412 nm}≈120 mM⁻¹cm⁻¹ for generated heme-species).
***Total Hp from the sum of Hp containing bands of the reduced SDS-PAGE subtracted from Hb of the non-reduced SDS-PAGE
Abbreviations: AT: α-1 antitrypsin, ACT: α-1 antichymotrypsin, Hb: hemoglobin, Tf: transferrin, ApoA1: apolipoprotein A1, Hpr: haptoglobin-related protein, ApoA2: apolipoprotein A2, ApoL1: apolipoprotein L1, HSA: human serum albumin, Hp: haptoglobin. α-2M: α-2 macroglobulin, CP: ceruloplasmin. CFB: complement factor B, apoHb: apohemoglobin . . .

From the purity assessment in Table 7, the HMW fraction was composed of ˜80% pure Hp and the LMW fraction was composed of ˜90% pure Hp. Thus, in agreement with the higher HbBC per mg, it was confirmed that the purity of the two main Hp products had greatly improved with the use of fumed silica. Yet, the purity of Hp is likely higher due to the slight overloading required to detect these impurities on the SDS-PAGE. Furthermore, similar to the process without the use of fumed silica, the method yielded consistent product compositions as demonstrated by the small deviations from densitometric analysis.

Interestingly, based on the average HbBC to total protein ratio and the Hb mass binding ratio of Hp2-2, the expected purity of the LMW Hp fraction (Stage 2) would be ˜85%. Unlike the process without the use of fumed silica, this value was in close agreement with the SDS-PAGE densitometry, indicating that very few protein contaminants were present in the samples. Furthermore, as explained before, the mass binding ratio between Hp2-2 and Hb may be larger than the theoretical value, leading to a closer Hp purity of >95% in agreement with densitometric analysis of SDS-PAGE.

Moreover, the higher purity from the Hp product purified with the use of fumed silica indicated that either the polymerized forms of the non-Hp proteins have a high affinity for the silica (potentially due to higher hydrophobicity from partial protein unfolding) or that most of these proteins were associated with the lipoproteins. Practically, no change in the α-2 macroglobulin fraction was observed indicating that this multimeric (720 kDa) protein species did not have a high affinity for the silica particles.

ELISA was performed on Stages 2 and 3 of the fumed silica treated batches to determine the mass percentage composition of Hp, HSA, and Tf to compare to the values determined by densitometric analysis of the SDS-PAGE. Unfortunately, the Hp ELISA was unable to accurately quantify the large MW Hp polymers in Stages 2 and Stage 3. The ELISA kit resulted in a Hp mass composition of 21±0.2% and 34±0.2% for Stages 2 and 3, respectively. As discussed previously, Hp ELISAs may not provide proper quantification of large MW Hp species. These large MW species may have sterically inhibited binding of Hp to immobilized Hp antibodies. The HSA content was determined to be 0.8±0.01% and 0.8±0.2% for Stages 2 and 3, respectively. This result agreed with MS data that demonstrated relatively similar HSA content for Stages 2 and 3. Thus, the difference in composition for the HSA/AT band on the reduced SDS-PAGE was mainly attributed to the higher AT composition in Stage 2 as seen by the high AT total ion intensity in the MS. The Tf composition was determined to be 0.5±0.09% and 0.3±0.09% for Stages 2 and 3, respectively. These results were similar to the values determined by SDS-PAGE, which showed <1% of Tf for both Stage 2 and Stage 3. Finally, ELISA kits were used to confirm that the hemichrome species formed from heme exchange from heme-HSA to the Hp sample was due to residual apoHb and not residual Hpx. Although Hpx was not detected on MS or apparent on the SDS-PAGE, both denatured apoHb and Hpx form similar hemichrome spectra (bis-histidine bonded heme). Hence ELISA results showed that the Hpx content was 0.1±0.02% and 0.1±0.04% of the total protein for Stages 2 and 3, respectively. Given that the calculated apoHb mass content was ˜3%, practically all heme exchange was due to residual apoHb in Stages 2 and 3 with a minor contribution from Hpx. Furthermore, since Hpx has a larger MW (on a heme basis) than apoHb, if Hpx was the heme-binding species, a mass content of >10% Hpx would have been determined

Although the purified samples were not composed of only Hp, the extra proteins in the BMW and LMW fractions yield a protein cocktail potentially useful for treatments of hemolysis. For example, the α-2 macroglobulin (α2M) protein is a broad specificity protease inhibitor. Furthermore, α2M helps maintain a balanced clotting system by both inhibiting the coagulating protein thrombin and inhibiting the anti-coagulating Protein C system. These characteristics may improve the effectiveness of the Hp product for applications in which the patient has an abnormal balance of the clotting proteins. Furthermore, α2M can help maintain hemodynamic equilibrium after scalding/burning by inhibiting prostaglandin E2 (vasodilator) and restricting loss of plasma volume. Moreover, the anti-inflammatory, anti-fibrosis and anti-oxidant functions of α2M have been linked to its role as a radioprotective agent. These characteristics can improve treatment of hemolytic states due to burn injury or radiation injury. Finally, α2M along with AT have been shown to mediate the binding of Tf to its surface receptor. In doing so, α2M can help with the removal of excess iron potentially released during prolonged states of hemolysis.

For the Hp samples purified without fumed silica, the presence of HSA, Tf and AT may improve the therapeutic effect of the protein mixture compared to pure Hp. HSA is a multifunctional protein, with major roles in the regulation of acid-base balance, oncotic pressure, binding/transport of endogenous and exogenous molecules and drugs (binds to heme upon depletion of hemopexin, potentially improving hemolysis treatment with the Hp protein cocktail), protection against exogenous toxins, maintenance of microvascular integrity and capillary permeability, antioxidant and anticoagulant activity. AT and transferrin have also been shown to bind to heme, which is a highly oxidative by-product of Hb degradation during states of hemolysis. AT, named for its ability to inhibit trypsin, can also inhibit other proteases, which is also known as: α1-antiprotease inhibitor. Due to its anti-proteolytic function, AT has general anti-inflammatory properties. In addition, AT plays a large role in vivo by preventing lung damage via inhibition of neutrophil elastase. Co-treatment of Hp with AT may constitute an improved treatment of pulmonary hypertension as both Hb (and its by-products) and neutrophil elastase have been shown to have deleterious effects. Finally, Tf is an antioxidant protein responsible for iron binding and transport⁹⁶. Thus, iron build-up due to excessive hemolysis could be neutralized by Tf.

As stated previously, apolipoprotein A-1 is the main component of high-density lipoproteins (HDL) and is found in the TLF1 complex (contains Hpr that binds to Hb). HDL therapy has been shown to treat atherosclerosis, improving blood flow and the HDL (expected to be the form in the protein cocktail on the process without fumed silica) has had greater clinical efficacy than pure apolipoprotein A-1 (likely due to its short half-life). In addition to its' well known role in lipid transport, HDL/apolipoprotein A-1 has various pleiotropic effects such as antioxidant, anti-inflammatory, antithrombotic (anticoagulant and increased NO bioavailability) and vasoprotective activities. Furthermore, HDL has been shown to negate the effects of lipopolysaccharides, reducing its pro-inflammatory responses. Finally, apolipoprotein A1 has also been shown to have antimicrobial activity.

To reduce the costs associated with the use of buffers, future studies may aim to optimize the number of diafiltrations at each stage for effective protein transmission and protein purity. Furthermore, by retaining the proteins in Stage 4 using a low MW TFF filter (<50 kDa), the permeated buffer may be used for future processing as it would contain little to no proteins. Bacterial contamination of the buffer is minimized given the filtration through the 0.2 μM and 750 kDa HF filters at the start of the next batch. The fraction retained in Stage 4 may also have therapeutic uses as it provides a promising mixture or proteins. Moreover, the protein mixture in Stage 4 could be used as the starting material for conventional chromatography purification to yield low MW Hp. Finally, the initial protein loading of FIV can also be increased. Dissolving 1 kg of FIV into 5 L of PBS before processing can yield approximately double the Hp yield with no discernable difference in purity compared to that of the 500 g batches.

Although the starting material for FIV consisted of pooled human plasma, which may have safety risks associated with infectious agents, the combination of Cohn acid-ethanol fractionation and TFF processing inherently reduces these risks. TFF clarification with the 0.2 and 750 kDa HF filters remove most pathogenic bacteria. In the case of viruses, the Cohn acid-ethanol fractionation process provides an approximate 4 log₁₀ reduction value (LRV) for various viruses. Furthermore nanofiltration using TFF can add an additional 5 (LRV). Finally, the Hp sample may undergo a final virus reduction step if desired such as solvent/detergent or pasteurization to reach the desired level of pathogen reduction.

One drawback for scalability of the current process is the use of centrifuges for removal of undissolved lipids and/or fumed silica. Cloth or depth filtration may substitute for the centrifugation step used to remove the unsuspended lipids. Furthermore, continuous centrifugation may be employed to separate the fumed silica, as it requires low relative centrifugal force to form a pellet. Finally, given that in both centrifugation steps, the goal was to remove lipids, these steps could be combined into a single centrifugation step to decrease processing time.

Conclusion

Overall, starting from 500 g of Cohn Fraction IV paste and without the use of fumed silica, 1.2 g of HbBC at ˜75% purity was obtained in solutions with ˜100 mg/mL total protein and 33 mg/mL HbBC. With the use of fumed silica, the yield increased to 1.7 g of HbBC at >95% purity in solutions with ˜100 mg/mL total protein and ˜52 mg/mL HbBC. Future studies may aim at improving processing time by optimizing the number of diafiltrations and buffer usage. Taken together, this study presents a novel and improved method for producing large quantities of large MW Hp (mixture of Hp2-2 and Hp2-1) at >95% purity

### Example 3. Selective Protein Purification Via Tangential Flow Filtration—Exploiting Molecular Size Changes Induced by Protein Complex Formation to Facilitate Separation

This example describes a process to purify a target protein (TP) from a mixture of proteins by exploiting molecular size changes that arise from the formation of a protein complex consisting of the TP and the TP binding molecule (TPBM). Briefly, the method employs tangential flow filtration (TFF) with a defined molecular weight cut off (MWCO) membrane to first permeate the TP+other protein impurities (filtrate) that are below the MWCO of the membrane, as well as set the maximum size/molecular weight of the protein species in the filtrate. A TPBM (could be another protein, antibody, aptamer or some other molecule) is then added to the filtrate to selectively create a protein complex with the TP in the protein mixture that is above the MWCO of the original membrane. With only the complexed TP above the MWCO of the original membrane, it can be selectively separated from the other low MW protein components of the filtrate using the original MWCO membrane. TFF can then be applied to buffer exchange the complexed protein under dissociative conditions and separate the TP from the TPBM using a MWCO membrane that is between the MW of the TP and TPBM.

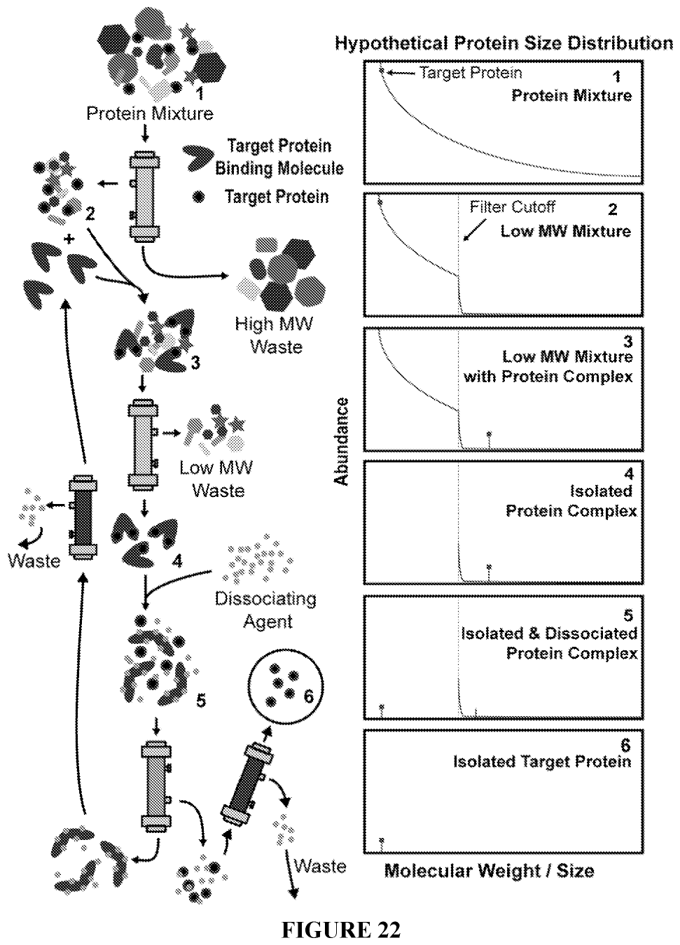

This theoretical strategy is schematically illustrated in FIG. 22. Referring to FIG. 22, starting with a mixture of proteins/particulates (1) (e.g., a cell lysate, human plasma, etc.), the mixture is filtered through a membrane with an appropriate MWCO that permeates the TP along with low MW impurities (2). Then a TPBM (e.g., an antibody or equivalent, etc.) specific to the TP is introduced into the filtrate, forming a TP-TPBM protein complex that is larger than the MWCO of the membrane (3). This solution with the newly formed TP-TPBM protein complex is then refiltered through the same MWCO membrane leading to the retention of the isolated TP-TPBM protein complex of interest (4). The isolated TP-TPBM protein complex can then be dissociated to yield free TP and TPBM via appropriate buffer exchange under conditions that would facilitate their separation (5). With the individual; species (TP and TPBM) dissociated in solution, the TP can be separated from the TP-TPBM protein complex using a MWCO membrane that is between the MW of the TP and TPBM (6). Note: both the TPBM in the retentate and TP in the filtrate can be buffer exchanged via TFF into appropriate buffers to remove the dissociating agent and concentrate the TP and TPBM.

General Strategies

Example Strategy 1—Purification of a 20 kDa TP using IgG antibody specific to TP. In a mixture of cell lysate (may need prior clarification through 0.2 micron filter and/or 50 nm filter), filter all material through a 70 kDa MWCO membrane. Add immunoglobulin G (IgG, commonly used antibody type) antibody specific to the TP into the filtrate. This will create an antibody-TP protein complex with MW>70 kDa (˜190 kDa, assuming two antigen binding-sites per antibody). The ˜190 kDa protein complex with the 20 kDa TG is now in a mixture with other proteins <70 kDa. Thus, this solution can be re-filtered through the 70 kDa MWCO membrane to retain the TP-antibody protein complex. The isolated TP-antibody complex can then be buffer exchanged into appropriate conditions to dissociate the TP-antibody complex (i.e. altered pH, salt concentration, or other appropriate denaturing condition). With the TP dissociated in solution from the antibody, refiltering the solution through the 70 kDa MWCO membrane will lead to the 20 kDa TP in the filtrate, and the 150 kDa antibody will be in the retentate. With the TP and antibody isolated, these species can be buffer exchanged into appropriate buffers on 10 kDa and 70 kDa membranes, respectively to yield purified TP and antibody. It is important to note that polyclonal antibodies may form aggregates at equimolar concentrations, thus excess target protein or excess antibody can be used for purification with these antibody species. On the other hand, monoclonal antibodies would not have the same issue as they only form specific complexes.

Example Strategy 2—Purification of a 200 kDa TP using IgG (non-reduceable protein, IgM or equivalent large TPBM could be used for its purification as in Example Strategy 1) antibody specific to TP. In a mixture of cell lysate (may need prior clarification through 0.2 micron filter and/or 50 nm filter), filter all material through a 300 kDa MWCO membrane. Add IgG antibody specific to TP of interest into the filtrate. This will create a TP-antibody protein complex with MW>300 kDa (˜350-670 kDa). The 200 kDa TP is now in an >300 kDa TP-antibody protein complex in a mixture with other proteins <300 kDa. Re-filter the protein solution through a 300 kDa MWCO membrane. The TP-antibody complex will be retained on the 300 kDa MWCO membrane. If the TP does not dissociate from the TP-antibody complex under reducing conditions, reduction of IgG will allow for its separation from the TP on a 100 kDa filter. The 200 kDa TP will be retained on the 100 kDa MWCO membrane, while the reduced components of IgG will enter the permeate. Both the TP and IgG components can be diafiltered into appropriate buffers. It is important to note that polyclonal antibodies may form aggregates at equimolar concentrations, thus excess target protein or excess antibody can be used for purification with these antibody species. On the other hand, monoclonal antibodies would not have the same issue as they only form specific complexes.

Example Strategy 3—Tagged recombinant proteins. Recombinant proteins can be synthesized with tags that facilitate their purification outlined in the Example Strategies above, removing the requirement for affinity columns. Instead of binding to affinity beads in columns, tags would bind to molecules that have larger MW than the MWCO of the employed membrane. For example, in a mixture of cell lysate with the strep-tagged 10 kDa recombinant protein (TrP), filter all material through a 30 kDa MWCO membrane. Add streptavidin to create a TrP-streptavidin complex with MW>60 kDa. The ˜60 kDa TrP-streptavidin complex is now in a mixture with other proteins <30 kDa. Thus, this solution can be re-filtered through the 30 kDa MWCO membrane to retain the TrP-streptavidin complex. The isolated protein complex can be buffer exchange into appropriate conditions to dissociate the protein complex. With the TrP dissociated in solution from the streptavidin, refiltering through the 30 kDa MWCO membrane will lead to the 10 kDa TrP in the filtrate, and the 50 kDa streptavidin will be in the retentate. With the TrP and streptavidin isolated, these species can be buffer exchanged into appropriate buffers on 1 kDa and 30 kDa membranes, respectively to yield purified TrP and streptavidin. This strategy employs currently known tagged recombinant protein technology, but improvement of the technologies for the proposed TFF purification could greatly ease its use. For example, polymerizing or developing large maltose/streptavidin/glutathione-bound molecules could improve applicability of this size exclusion method with currently used tagging systems (e.g., maltose-binding protein, strep-tag, glutathione-S-transferase, split-intein, etc.).

Purification of Haptoglobin (Hp) from Human Plasma or a Plasma-Derived Fraction Via Protein Complex Formation.

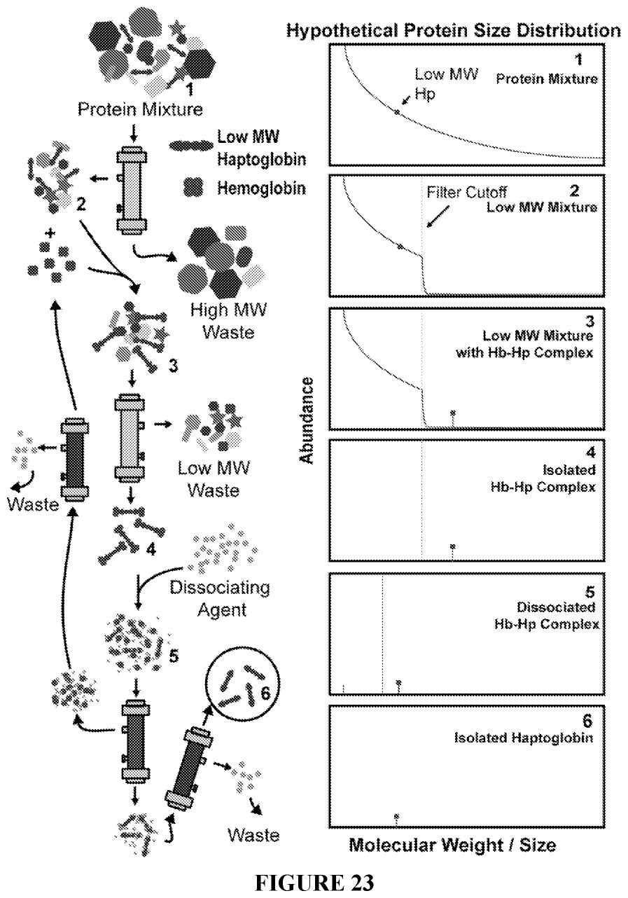

The theoretical manufacturing scheme for the presented technology is shown in FIG. 23.

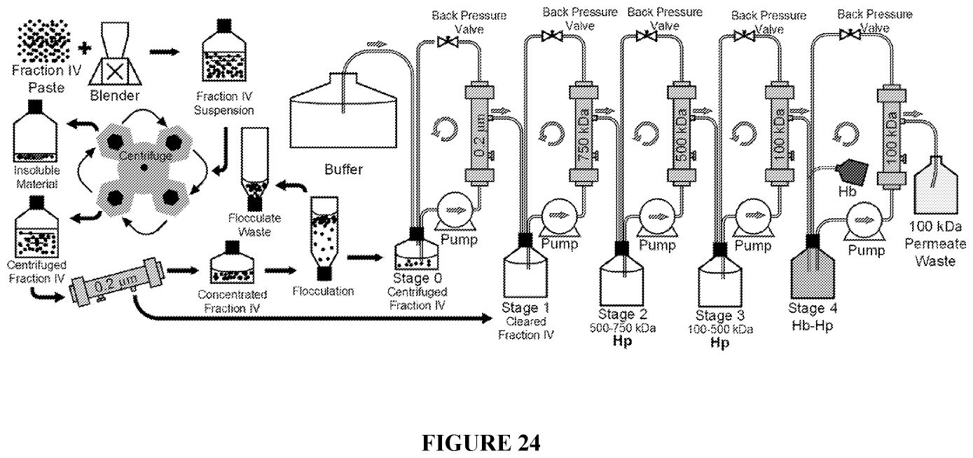

500 g of human Fraction IV (FIV) paste from the modified Cohn process of Kistler and Nitschmann was suspended in 5 L of PBS, and homogenized in a blender. The resulting mixture was stirred overnight at 4° C. The ˜5 L solution was centrifuged for 40 min at 3700 g to remove insoluble particulates (mostly lipoproteins). Then, the supernatant was concentrated using a 0.2 μm hollow fiber filter to 2 L The retentate was left to rest for 36 hrs to flocculate low density particles, while the filtrate was kept at 4° C. for further processing. After flocculation of the retentate, low density particles in solution were separated. The higher density fraction was then concentrated ˜1 L on a 0.2 μm hollow fiber filter and subjected to 10 diafiltrations with PBS. The 0.2 μm filtrate was concentrated to 150 mL and subjected to 100 diafiltrations on a 750 kDa hollow fiber filter using a mixture of the 0.2 μm permeate and PBS. The permeate was then subjected to 40 diafiltrations using PBS on a 500 kDa hollow fiber filter. Finally, the permeate of the 500 kDa hollow fiber filter was subjected to 100 diafiltrations using PBS on a 100 kDa hollow fiber filter (intermediate stages comprising of 750 and 500 kDa hollow fiber filtration were employed prior to the 100 kDa hollow fiber filter to avoid filter fouling). Hemoglobin (Hb) was then continuously added to the permeate from the 100 kDa hollow fiber filter to form the Hb-Hp complex, while maintaining the solution with excess Hb to bind all Hp in the permeate. The filtrate/Hb mixture was then subjected to diafiltration (100 or 200×) on a 100 kDa hollow fiber filter using fresh PBS to remove excess Hb and low molecular weight (MW) proteins. The resulting Hb-Hp complex was then centrifuged for 30 min at 3000 g to remove any insoluble particulates. The 100 diafiltration trial yielded 200 mL of 2 mg/mL Hb-Hp complex, while the 200 diafiltration trial yielded 200 mL of 0.8 mg/mL Hb-Hp complex. The diagram for the purification process is shown in FIG. 24. The solutions were then concentrated on a 100 kDa filter. The final products consisted of 5 mL solutions at 43.4 mg/mL and 17.2 mg/mL of Hb-Hp. The discrepancy in final product yields was attributed to differences in the number of diafiltrations performed with the Hb-Hp complex solution (100 or 200×).

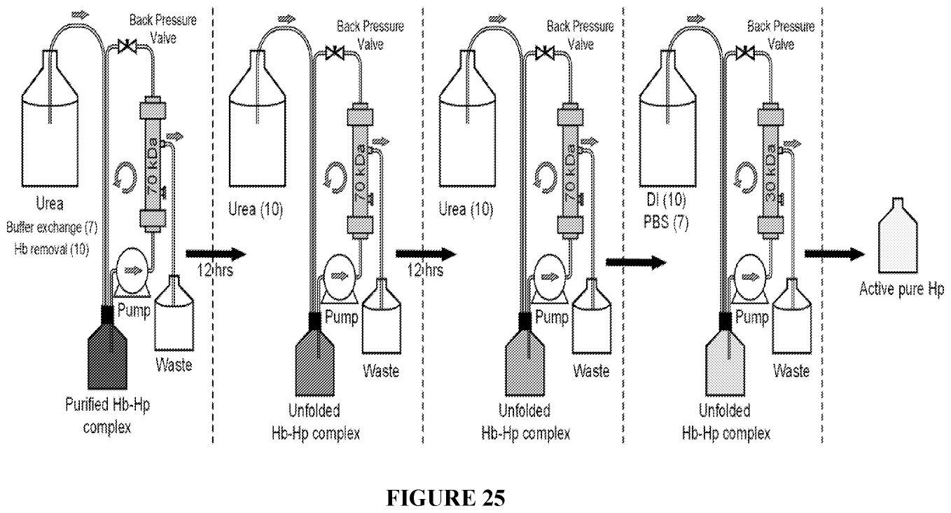

To facilitate dissociation of Hb from the purified Hb-Hp complex, 7 mL of Hb-Hp at 2 mg/mL was buffer exchanged (7 diacycles) into a 5 M Urea solution at a pH 10 using a 70 kDa hollow fiber filter. The resulting unfolded protein mixture was then subjected to 10× diafiltration using the urea solution with a rest period of 12 hr in between processing to yield a total of 30 diacycles. The solution was then diafiltered for 10 diacycles into DI water followed by 7 diacycles into PBS using a 30 kDa hollow fiber filter. The schematic of this process is shown in FIG. 25 and the characteristic of all the filters used is shown in Table 8. The final product was finally concentrated to 3.2 mL, yielding a solution with 9.2 mg/mL of protein (Bradford assay) and 1.8 mg/mL of Hb (spectrophotometric analysis).

TABLE 8
Hollow fiber filters (Spectrum Laboratories, Rancho Dominguez,
CA) used for the dissociation and purification of pure Hp
from purified Hb-Hp using tangential flow filtration.

| | | | | Surface Area | |
|---|---|---|---|---|---|
| Type | Pore Size | | Membrane | (cm²) | P/N |
| miniKros | 0.2 | μm | PES | 470 | S02-P20U-10-N |
| miniKros | 750 | kDa | mPES | 790 | S02-E750-05-N |
| miniKros | 500 | kDa | PS | 1000 | S02-S500-05-N |
| miniKros | 100 | kDa | mPES | 1000 | S02-E100-05-N |
| microKros | 70 | kDa | mPES | 20 | C02-E070-05-N |
| microKros | 30 | kDa | mPES | 20 | C02-E030-05-N |

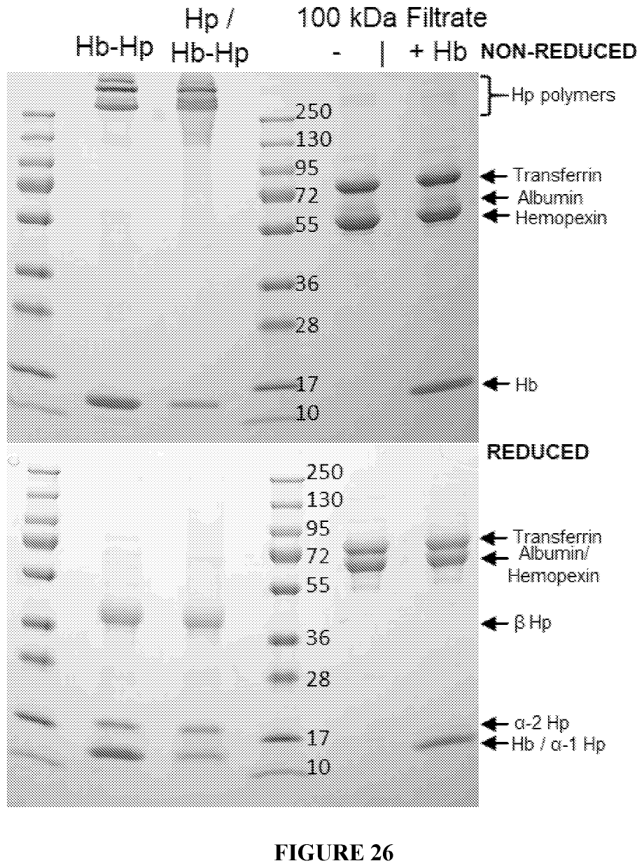

The SDS-PAGE of the purified Hb-Hp complex and recovered pure Hp from one batch is shown in FIG. 26.

From the SDS-PAGE, practically no impurities could be detected (>98% pure) for the purified Hp-Hb. Furthermore, from both SDS-PAGE band intensity analysis and the spectrophotometrically determined amount of Hb bound to the purified Hp species, the Hp to Hb mass binding ratio was calculated to be 1.6:1. This is the same mass binding ratio assuming one Hp2-2 dimer is bound to one Hb αβ dimer. Urea treatment was not successful in removing all of the bound Hb. Using the 1.6:1 mass binding ratio, SDS-PAGE analysis indicated that about 20% of the Hp was still bound to Hb. In comparison, using total protein and spectrophotometric analysis, the product consisted of 25% active Hp, 29% Hb-Hp complex and 52% inactive Hp (denatured). Furthermore, compared to the starting Hb-Hp complex, 52% of Hp was lost during diafiltration, 12% was active, 13% remained bound to Hb and 23 was denatured. These results could be improved through optimization of the protein unfolding conditions in urea to avoid protein denaturing and via selection of a lower MWCO hollow fiber filter for the diafiltrations to avoid loss of protein.

From the Hb binding fluorescence assay, the final solution had a Hb binding capacity of 0.5 mg/mL, which indicated that only 10% of the initial Hb binding capacity was recovered. This large loss of Hp was attributed to loss of protein and protein denaturing during urea diafiltration as well as retained Hb in the product.

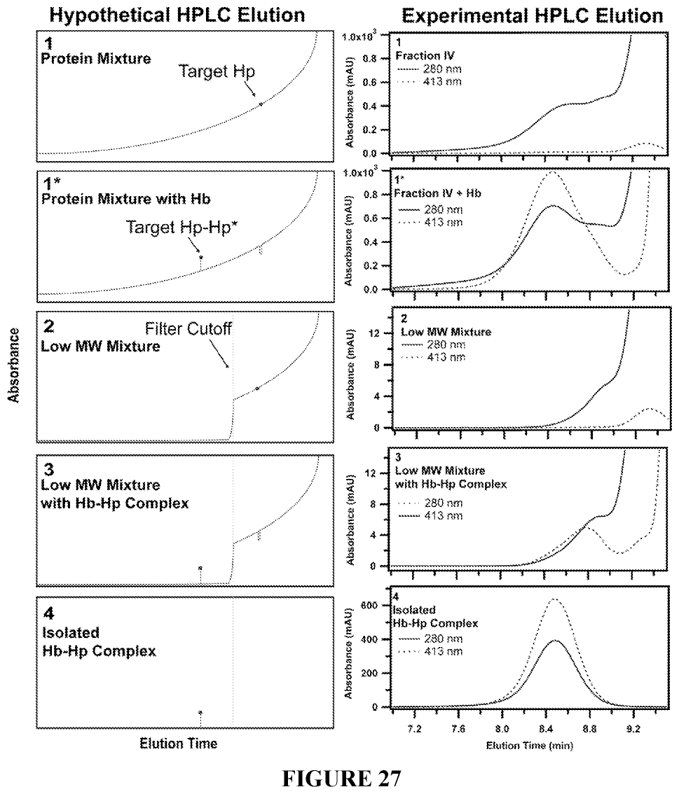

During the purification of the Hp-Hb complex, samples were taken at different stages of the process. These samples were analyzed on an HPLC-SEC column and the results were compared to the theoretically predicted separation based on the schematic in FIG. 22. The comparison of predicted versus experimental results is shown in FIG. 27.

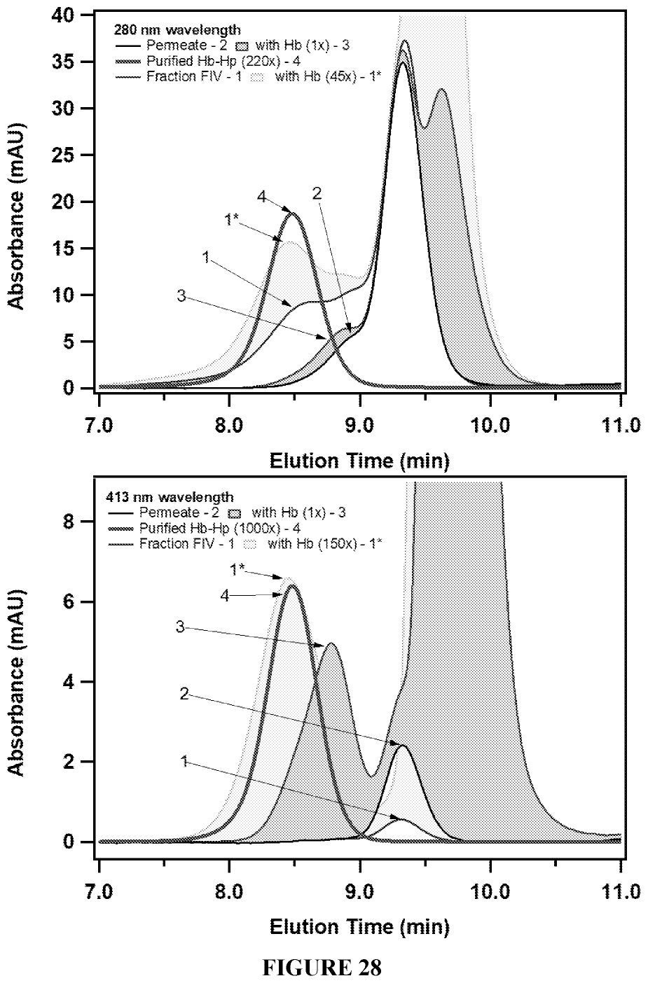

From these results, the addition of Hb to Fraction IV increased the amount of large molecular weight species (compare 1 to 1*). These species matched our purified product (4). Furthermore, the permeate analysis (2) showed that the unbound Hp did not easily permeate the 100 kDa hollow fiber filter. This can be seen due to a lower relative abundance of the Hb-Hp complex when Hb was added comparing Fraction IV to the permeate (1* to 3). Another observation was that, by comparing 2 to 4, it was noticeable that the Hb-Hp complex was capable of permeating through the 100 kDa hollow fiber filter. This observation agreed with the different Hb-Hp yields obtained via 100× or 200× diafiltrations. Combining the chromatograms into one figure better represented the statements regarding retention of unbound target Hp and permeation of the target Hb-Hp complex. The combined elution chromatograms at 280 and 413 nm wavelength detection is shown in FIG. 28.

### Example 4. Pegylation of Apohemoglobin (apoHb) to Increase Stability and Circulatory Half-Life In Vivo

Human hemoglobin (Hb) exists primarily as a tetrameric (64 kDa) protein contained inside human red blood cells (RBCs). A prosthetic heme group is buried inside each globin chain of Hb, facilitating its ability to storage and transport various gaseous ligands such as oxygen, carbon monoxide and nitric oxide. During states of hemolysis (rupture of RBCs), large quantities of toxic cell-free hemoglobin (Hb) are released into the blood stream. Cell-free Hb toxicity is attributed to a variety of factors, including Hb extravasation into tissue space which elicits oxidative tissue injury, nitric oxide (NO) and peroxide scavenging, and free heme release. These factors can lead to acute and chronic vascular disease, inflammation, thrombosis, and renal damage. Furthermore, free heme release from cell-free Hb is very toxic due to the highly reactive nature of this hydrophobic group. Free heme is a highly oxidative species that attaches to serum proteins, cell membranes and/or lipids leading to their oxidation. Free heme can also catalyze covalent cross-linking and aggregate formation of proteins, as well as its degradation into small peptides.

Fortunately, the body relies on the plasma scavenging proteins haptoglobin (Hp) and hemopexin (Hpx) to mitigate the toxic effects of cell-free Hb and free heme released during hemolysis. Upon RBC lysis, plasma Hp quickly binds to Hb preventing its extravasation and its oxidative damage. The Hp-Hb complex is then captured by CD163+ macrophages and monocytes, where the enzyme heme oxygenase-1 catalyzes the breakdown of the heme from Hb into biliverdin, carbon monoxide and iron. In a similar manner, upon heme release from cell-free Hb, plasma Hpx quickly captures heme. Heme binding by Hpx prevents its damaging oxidative reactions and allows for heme to be safely removed from the circulation.

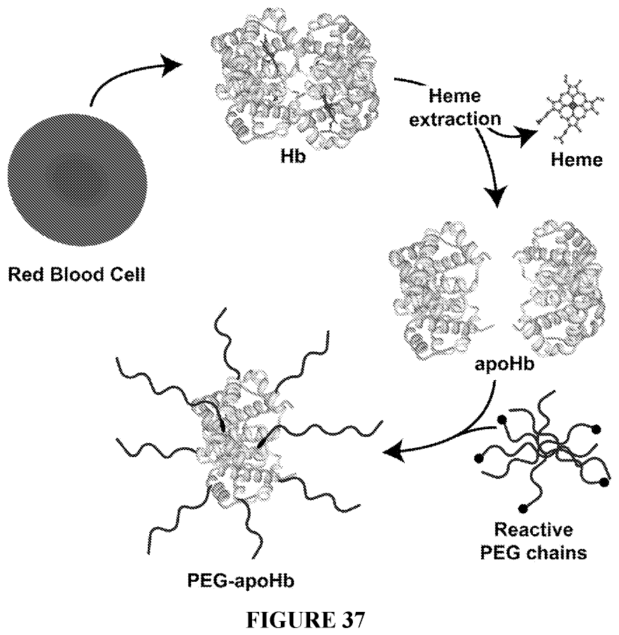

The prosthetic heme groups of Hb may be synthetically removed, yielding an apoprotein referred to as apohemoglobin (apoHb). ApoHb has been used in biomedical applications for targeted drug delivery. Unfortunately, apoHb is highly unstable at physiological temperature and quickly precipitates from solution. Furthermore, heme scavenging with unmodified apoHb would yield cell free Hb, which as already mentioned is toxic. Thus, in the present study, poly-ethylene glycol (PEG) was covalently attached to apoHb to increase the stability of the apoprotein. The resultant PEGylated apoHb molecule (PEG-apoHb) should exhibit improved in vitro and in vivo stability compared to unmodified apoHb. The general idea for PEGylation of apoHb is depicted in FIG. 37.

Materials and Methods

Sodium phosphate dibasic, sodium phosphate monobasic, and hemin chloride were purchased from Sigma Aldrich (St. Louis, Mo.). Potassium cyanide, hydrochloric acid, acetone, sodium chloride, ammonium acetate, potassium chloride, N-[Tris (hydroxymethyl) methyl]-2-aminoethanesulfonic acid (TES), 0.22 μm nylon syringe filters, and dialysis tubing (pore size: 6-8 kDa) were purchased from Fisher Scientific (Pittsburgh, Pa.). 0.2 μm Millex-GP PES syringe filters were purchased from Merck Millipore (Billerica, Mass.). Ethanol was purchased from Decon Labs (King of Prussia, Pa.). A KrosFlo® Research II tangential flow filtration (TFF) system and hollow fiber (HF) filter modules were obtained from Spectrum Laboratories (Rancho Dominguez, Calif.). Expired units of human red blood cells (RBCs) were generously donated by the Transfusion Service in the Wexner Medical Center at The Ohio State University (Columbus, Ohio).

Hb Preparation. Human Hb was purified via TFF. The Hb concentration was determined spectrophotometrically via the Winterbourn equations.

ApoHb Production. Apohemoglobin was produced from Hb via TFF heme extraction in acidic-ethanol as previously described.

Hp Purification. Hp was purified via TFF of human Cohn Fraction IV as previously described.

Total Protein Assay. The total protein concentration of the apoHb solution was measured using the molar extinction coefficient of apoHb at 280 nm (12.7 mM⁻¹ cm⁻¹).

PEGylation of ApoHb. ApoHb was dethawed from −80° C. at 4° C. over the course of 5 hours. ApoHb was then diluted to ˜10 mg/mL in phosphate buffered saline (PBS) and filtered through a 0.2 μm syringe filter prior to the PEGylation reaction. The sample was incubated with 10 times molar excess (on α globin basis of apoHb) of fresh 2-iminothiolane and 20 times molar excess of maleimide-PEG-5000 for 16 hours at 4° C. The excess maleimide-PEG-5000 and 2-iminothiolane was removed using a 30 kDa mPES hollow fiber membrane.

Stability of PEG-ApoHb. Protein stability was measured by aliquoting 3 0.5 mL samples of either apoHb or PEG-apoHb and placing them in an incubator at 37° C. Samples were taken from each aliquot using sterile pipette tips and diluted to 0.8 mL. Samples were then centrifuged in a table-top centrifuge for 2 minutes. The supernatant was then measured in a UV-vis spectrophotometer to determine the total protein in solution.

ApoHb/PEG-ApoHb Activity Assay. The activity of the vacant heme-binding pocket of apoHb or PEG-apoHb was determined via the dicyanohemin (DCNh) incorporation assay. The extinction coefficients of DCNh and rHbCN used were 85 mM⁻¹ cm⁻¹ and 114 mM⁻¹ cm⁻¹, respectively.

Hp Binding to PEG-ApoHb and Heme-Bound PEG-ApoHb. The binding capacity of Hp to Hb and apoHb was determined using SEC-HPLC as described previously. Briefly, the change in the area under the curve of free Hb/apoHb when Hp is added to the sample is used to quantify the amount of Hb/apoHb bound to Hp. To analyze the binding of Hp to PEG-apoHb, 2× excess PEG-apoHb was mixed with Hp (based on the Hb/apoHb binding capacity of Hp). The mixture was then separated on an SEC-HPLC column. The change in the chromatogram when monitoring the 280 nm absorbance was then used to determine the formation of Hp-PEG-apoHb complexes. The same process was repeated using a PEG-apoHb sample incubated with 5× excess heme-albumin to saturate the heme-binding pockets of PEG-apoHb. Furthermore, the fluorescence quenching of Hp (monitored via 285 nm excitation and 330 nm emission) due to heme-PEG-apoHb binding was used to determine the amount of bound heme-PEG-apoHb to Hp.

Size Exclusion Chromatography. Samples were separated on an analytical Acclaim SEC-1000 (4.6×300 mm) column (Thermo Fisher Scientific, Waltham, Mass.) attached to a Dionex UltiMate 3000 system (Thermo Fisher Scientific, Waltham, Mass.). The mobile phase consisted of 50 mM potassium phosphate, pH 7.4. The flow rate and UV-visible spectral detection was controlled on Chromeleon 7 software with detection set to λ=280 nm to detect protein elution at a flow rate 0.35 mL/min.

Results

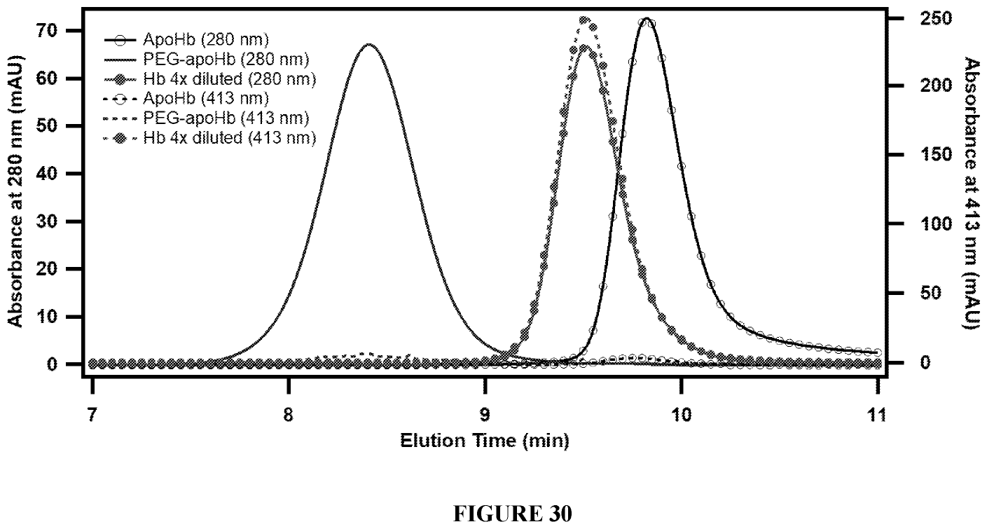

PEGylation of apoHb lead to more than 95% yield of PEG-apoHb. Based on the SEC-HPLC of FIG. 30, it was shown that the size of PEG-apoHb was effectively increased to an apparent MW of approximately 400 kDa.

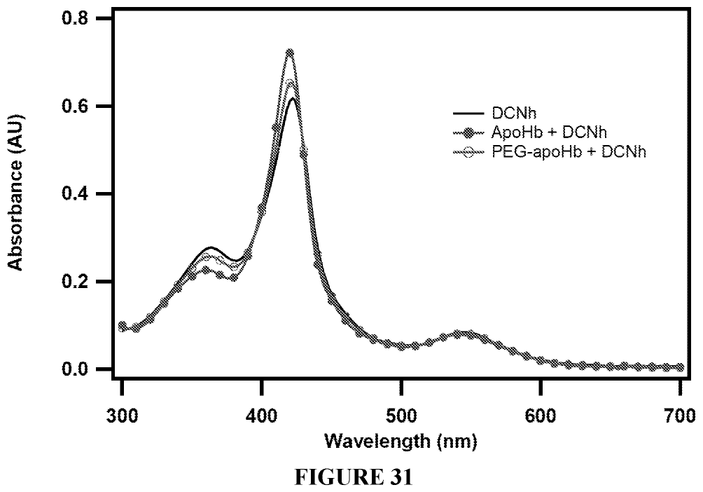

The initial heme-binding capacity of apoHb decreased from 74% to 28% when PEGylated, indicating a loss of heme-binding capacity of PEG-apoHb compared to apoHb. This decrease in heme-binding capacity is shown in FIG. 31.

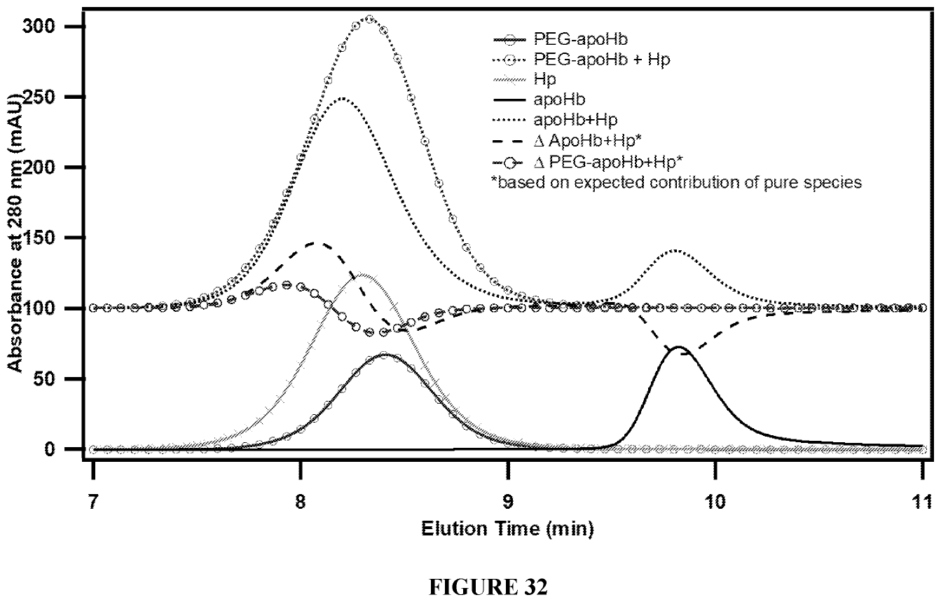

PEG-apoHb in addition to retaining its ability to bind heme, also retained its ability to bind Hp. This is shown in FIG. 32.

From FIG. 32, it was shown that, similar to apoHb, a new high-MW complex formed when PEG-apoHb and Hp were mixed together. Yet, given the smaller change in area, PEG-apoHb showed lower binding to Hp versus apoHb.

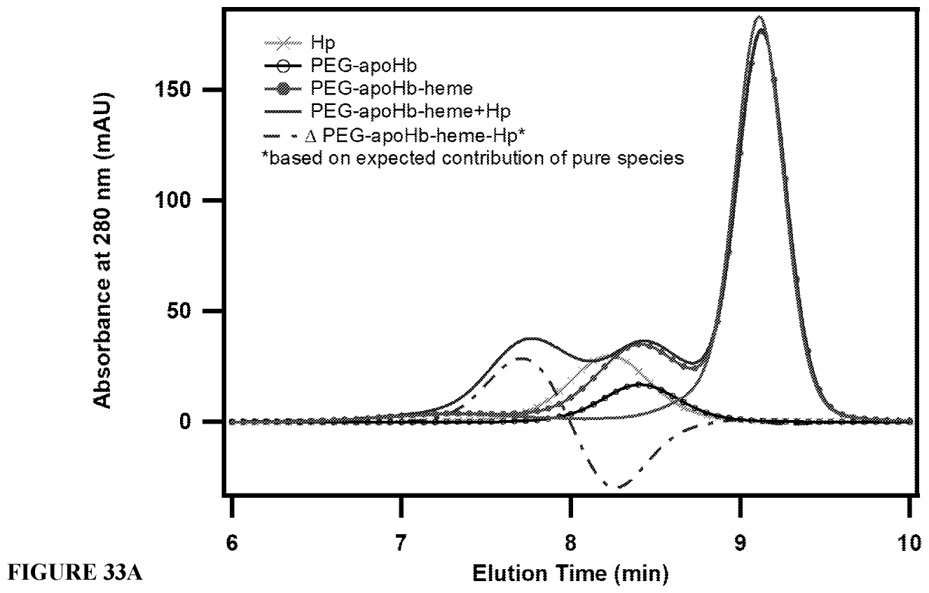

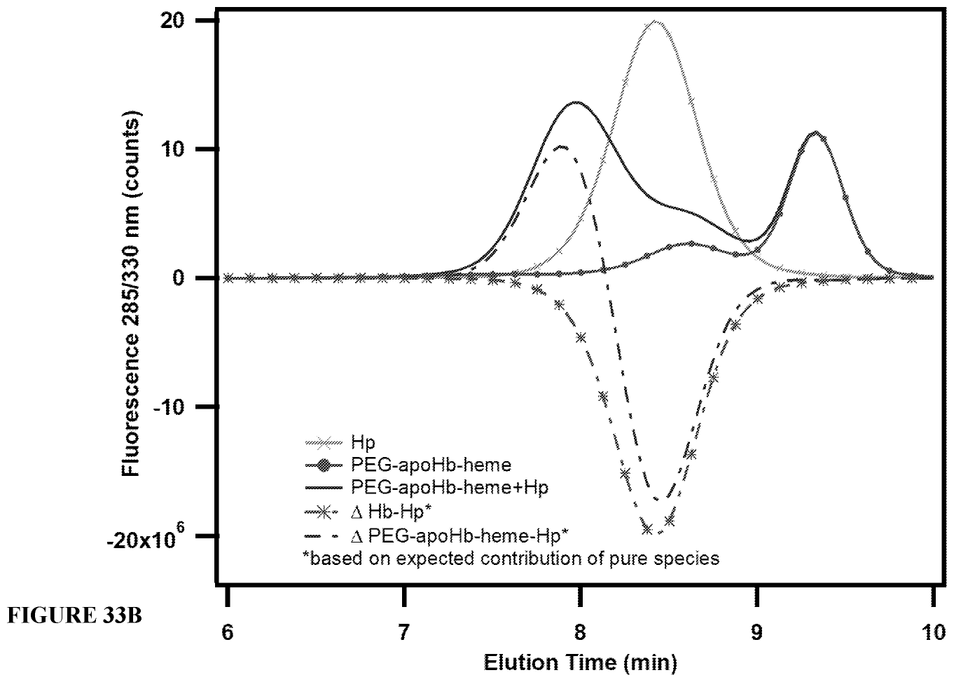

To determine the difference in Hp binding when PEG-apoHb was bound to heme, PEG-apoHb was incubated with 5× excess heme albumin and then mixed with Hp. The SEC-HPLC chromatogram of this mixture is shown in FIG. 33A.

From the HPLC-SEC chromatograms shown in FIG. 33A, it was shown that PEG-apoHb-heme bound to Hp. Furthermore, based on the fluorescence quenching of Hp, it was estimated that 2× PEG-apoHb bound to only 50% of the Hb binding sites of Hp (FIG. 33B). This indicated that higher quantities of PEG-apoHb were required to saturate the binding sites of Hp. Maintenance of Hp binding may allow for PEG-apoHb-Hp to be cleared via CD163+ macrophages and monocytes.

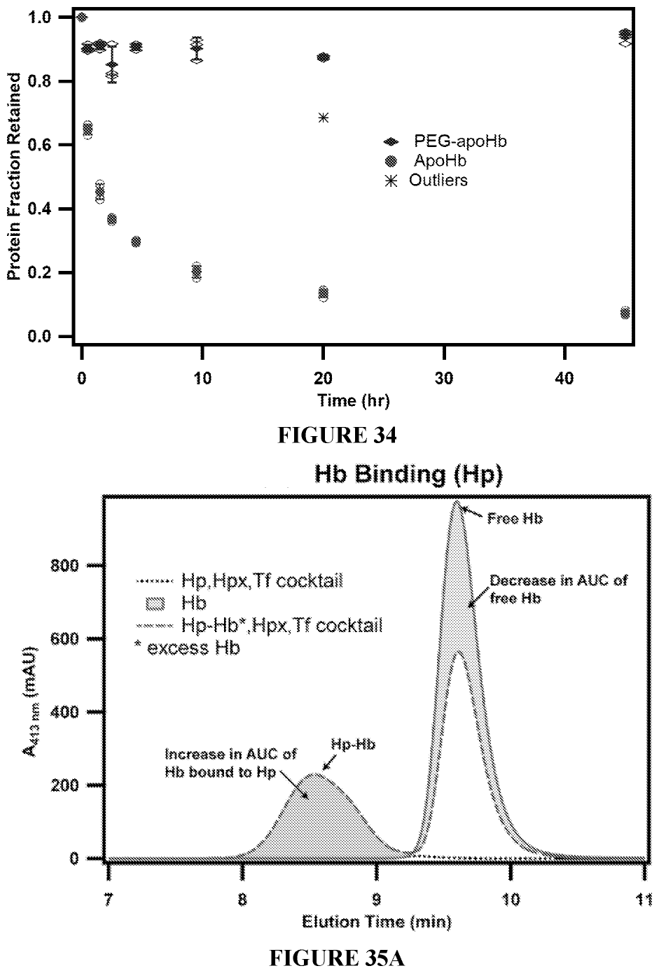

The stability of PEG-apoHb at 37° C. was assessed and the results are shown in FIG. 34.

From FIG. 34 it was evident that PEG-apoHb had much greater stability than apoHb when incubated at 37° C., greatly improving potential therapeutic applications of apoHb.

### Example 5. Scavenger Protein Cocktail for Treating States of Hemolysis

In vivo hemolysis of red blood cells (RBCs) can lead to the accumulation of toxic cell-free hemoglobin (Hb), free heme and free iron. These reactive molecules are normally scavenged by the plasma proteins haptoglobin (Hp), hemopexin (Hpx) and transferrin (Tf). However these scavenging proteins can become depleted or saturated during hemolytic conditions. Unfortunately, purification of these scavenger proteins at the scale required for treatment presents a significant biomanufacturing challenge. Moreover, even though hemolysis is a multifaceted disease state, most research strategies have targeted hemolysis treatment with single protein therapeutics (e.g. Hp or Hpx treatment). This current study seeks to provide a practical and effective method to purify a protein cocktail containing a comprehensive set of scavenger proteins for potential treatment of various states of hemolysis. This protein cocktail was purified based on a newly published procedure to purify Hp via tangential flow filtration (TFF) of human Cohn Fraction IV (FIV). After Hp purification, the residual permeate stream from a 100 kDa hollow fiber module was retained on a 50 kDa hollow fiber module and was observed to consist of a protein mixture with approximately 40% human serum albumin (HSA), 35% Tf, 10% Hp, and 5% of Hpx. Ceruloplasmin (Cp) and vitamin-D binding protein (VDB) were the other minor protein components in the mixture at ˜5% each. Using this approach, 60 g of the protein cocktail capable of scavenging Hb, heme and iron at a ˜1:2:2 molar ratio was purified from 500 g of FIV. The HSA in the protein cocktail can aid in the neutralization of free heme and free iron, while Cp regulates iron metabolism, and VDB scavenges toxic cell-free actin that is also released during cell lysis. Moreover, given the significant HSA content, this protein cocktail could serve as a promising next generation plasma expander. Taken together, this study provides a new and effective method to produce large quantities of a protein cocktail that is potentially suitable for treatment of hemolytic states including hemorrhagic shock.

Background

Red blood cells (RBCs) constitute ˜45% of the blood volume, and ˜4% of the total body weight in humans. Hemoglobin (Hb) is the major protein contained inside RBCs. Hb is capable of reversible oxygen (O₂) binding and release, thus facilitating delivery and transport of O₂ from the lungs to the tissues. O₂ binding is mediated via the prosthetic heme groups of Hb. However, heme is a highly hydrophobic (due to the porphyrin macrocycle) and reactive molecule (due to the iron atom). Thus, to facilitate aqueous transport of heme, the porphyrin group is tightly bound inside the hydrophobic heme-binding pocket of apohemoglobin (i.e. the apoglobin of Hb). This binding not only enhances heme solubility, but the heme-globin interaction facilitates cooperative O₂ binding and release. However, even when heme is bound inside the hydrophobic heme-binding pocket of apoglobin, the heme groups are reactive, are prone to auto-oxidation, and can be released from the hydrophobic heme-binding pocket⁶. Thus, to maintain Hb and its heme groups in a constrained and reducing environment, Hb is encapsulated inside RBCs. Inside the RBC, the high Hb concentration maintains Hb in its tetrameric quaternary structure, which restricts heme loss, and redox enzymes prevents accumulation of oxidized Hb (i.e. methemoglobin, metHb).

Lysis of RBCs, a phenomenon referred to as hemolysis, leads to the extravasation of cell-free Hb from the blood vessel lumen into the surrounding tissue space. Outside of the protective confines of the RBC, the heme contained inside Hb is susceptible to oxidation⁶. Furthermore, the small size of cell-free Hb (˜5 nm diameter) and dimerization of tetrameric Hb into αβ dimers at low plasma concentrations allows cell-free Hb to easily translocate into vulnerable anatomic sites such as the kidneys and the vascular wall. Extravasation through the blood vessel wall can lead to Hb scavenging of nitric oxide (NO) produced by the endothelium, eliciting vasoconstriction and systemic hypertension. In the kidneys, accumulation of cell-free Hb can lead to hemoglobinuria and acute kidney damage via intrarenal oxidative reactions. Furthermore, cell-free Hb can mediate oxidative damage to proteins, lipids, nucleic acids and other biomolecules. These factors can lead to acute and chronic vascular disease, inflammation, thrombosis, and renal damage.

Not only can cell-free Hb react with the environment outside the RBC, but its breakdown leads to the release of the highly reactive and toxic free heme. The insolubility of free heme in aqueous solution, causes it to bind to hydrophobic plasma proteins, cell membranes and/or lipids which elicits their oxidation such as in the case of oxidized low density lipoprotein (ox-LDL). Free heme also catalyzes covalent cross-linking and subsequent formation of protein aggregates; as well as, protein degradation into small peptides. Furthermore, free heme can serve as a molecular signal, which can alter homeostasis of various systems during hemolysis. Intracellularly, free heme is further broken down into biliverdin, carbon monoxide (CO), and iron via heme-oxygenase-1 (HO-1). Build-up of free iron also has toxic effects. Iron overload induces the formation of reactive oxygen species (ROS), free radicals, and can cause various levels of cellular oxidative damage. ROS and free radicals oxidize lipids, proteins, nucleic acids and other biomolecules.

These toxic and reactive species (cell-free Hb, heme and iron) are controlled during mild to moderate hemolysis via the action of three plasma proteins: haptoglobin (Hp), hemopexin (Hpx) and transferrin (Tf). These serum proteins bind to and neutralize cell-free Hb, free heme, and free iron, respectively. After binding to their target, the complexed forms are less reactive and can be safely cleared from the circulation. The toxic effects of Hb, heme and iron occur when these scavenging proteins are saturated and depleted in the plasma, allowing excess Hb, heme and iron to freely react with the surroundings. Human serum albumin (HSA) is another serum protein that binds and transports both heme and iron. Although not at specific as the covalent bonds present in heme-Hpx or iron-Tf, HSA becomes the main reservoir for heme and iron once the primary scavengers (Hpx and Tf) have been depleted in the plasma.

Common disease states that cause hemolysis include sickle-cell anemia, beta-thalassemia, and malaria. In sickle-cell anemia and beta-thalassemia, genetic traits lead to RBC rupture and subsequent Hb release. However, in malaria, the parasite (Plasmodium spp) uses RBCs to asexually reproduce until the RBC ruptures spilling Hb into the circulation. Furthermore, hemolytic conditions have been shown to occur upon massive blood transfusion, fever, sepsis, hemolytic anemia, viral hemorrhage, extracorporeal circulation, burn injury, and radiation exposure. Finally, one of the major factors for the short ex vivo shelf-life of stored RBCs is the gradual hemolysis that occurs during storage in the blood bag.

In addition to direct organ and tissue damage; iron, heme and Hb are also used as substrates for the proliferation of pathogenic microbes. Even though some microorganisms can sequester iron from holoTf, Hb-Hp or heme-Hpx, they must compete with the binding affinity of these complexed species for their target ligands on clearance cells. Therefore, restricting the bioavailability of iron during pathological states could prevent the proliferation of infectious organisms. Furthermore, hemolysis itself is a highly inflammatory event. Free heme stimulates toll-like receptor 4 (TLR4) leading to tumor necrosis factor alpha (TNF-α) production and the release of high mobility group box 1 (HMGB1) protein, which has been shown to be elevated in sickle-cell anemia patients. Therefore, scavenging and neutralization of Hb, heme and iron inhibits these inflammatory responses and can prevent the synergistic effects of heme/Hb with lipopolysaccharides (LPS) or HMGB1. Recent studies have also demonstrated that Hp may serve as a HMGB1 scavenger in addition to its primary function as a Hb scavenger. Moreover, both Hp and Hpx can induce HO-1 which has anti-inflammatory effects.

Although hemolysis is a multifaceted disease state, most treatment strategies have focused on single protein supplementation therapies consisting of either Hp, Hpx or Tf. Although there has been great success with these studies, they have not been approved for clinical use in the United States and are expensive and complicated serum proteins to be purified at the scale required for hemolysis treatment. In Example 2, we demonstrated that high purity Hp (95% pure) can be purified via tangential flow filtration (TFF) of human Cohn Fraction IV (FIV), a waste product from the plasma fractioning industry. The large molecular weight (MW) difference between Hp (90-900 kDa) compared to that of most serum proteins (<100 kDa) allows it to be purified to a high degree via a size-based separation approach. Using this approach, we employed a series of hollow fiber filters to bracket the plasma proteins based on their MW with a 100 kDa hollow fiber filter as the last stage for Hp retention. However, the permeate from the 100 kDa filter contained ˜65% of the total soluble protein from FIV and 50% of the initial Hb-binding capacity (i.e. Hp). Moreover, based on the composition of FIV, it is expected that this stream (with MW<100 kDa) should contain Hp, Tf, Hpx and HSA, thus yielding a cocktail of proteins potentially suitable for comprehensive treatment of hemolysis. In this study, we retained the 100 kDa permeate derived from Hp production on a 50 kDa hollow fiber filter, and characterized the Hb, iron, and heme binding capacity of the retentate; thus developing a novel, cost-effective and simple protein cocktail for potential treatment of hemolysis.

Materials and Methods

Materials. Sodium phosphate dibasic, sodium phosphate monobasic, sodium chloride, and fumed silica (S5130) were purchased from Sigma Aldrich (St. Louis, Mo.). 0.2 μm Millex-GP PES syringe filters were purchased from Merck Millipore (Billerica, Mass.). A KrosFlo® Research II tangential flow filtration (TFF) system and hollow fiber (HF) filter modules were obtained from Spectrum Laboratories (Rancho Dominguez, Calif.). Human FIV paste from the modified Cohn process of Kistler and Nitschmann was purchased from Seraplex, Inc (Pasadena, Calif.).

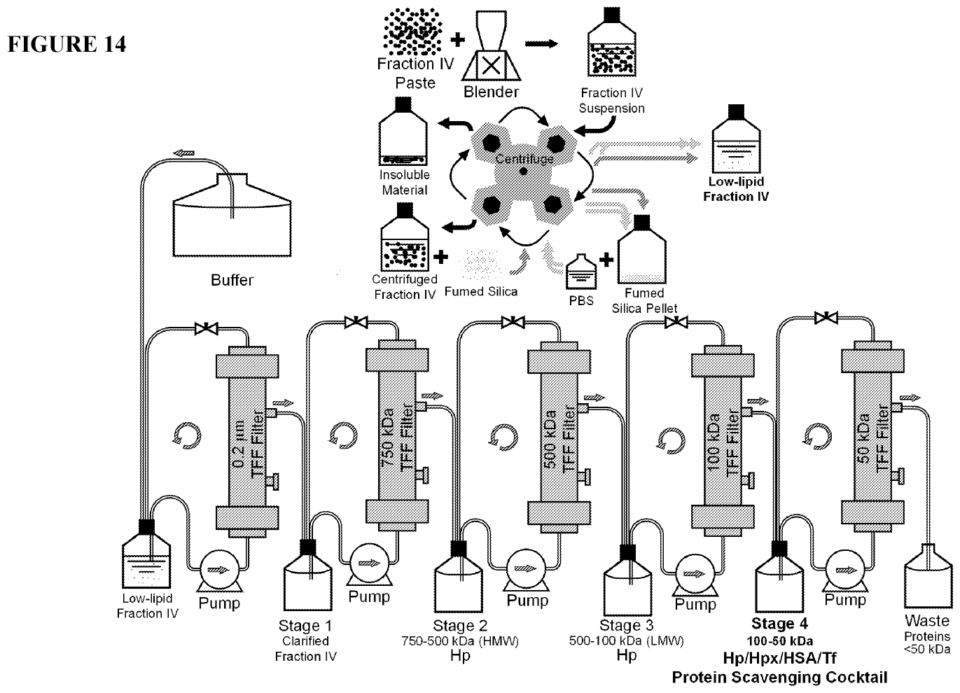

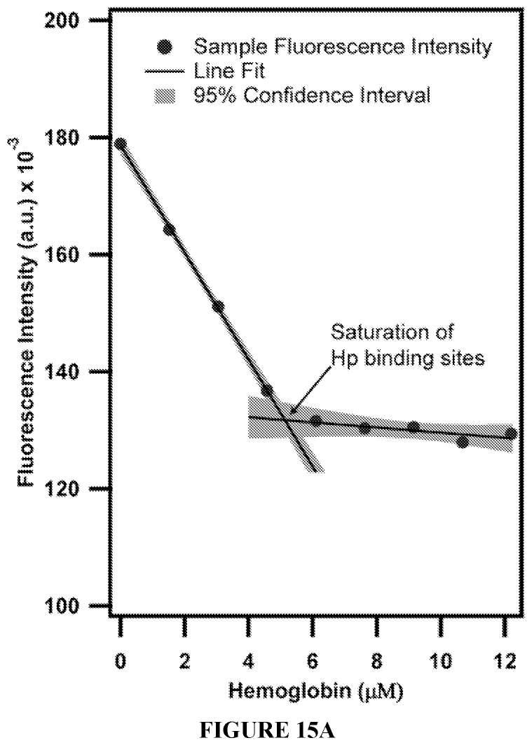

Hb, Heme and Iron Scavenging Protein Cocktail Purification via TFF. Purification of the protein cocktail followed the purification method described in Example 2, with the addition of one additional TFF stage at the end. Briefly, 500 g of FIV was suspended in 5 L of phosphate buffered saline (PBS) and homogenized in a blender. After overnight stirring at 4° C., the ˜5 L solution was centrifuged to remove undissolved lipids. Fumed silica (Sigma Aldrich P #S5130; St. Louis, Mo.) was then added to the supernatant, left stirring overnight at 4° C., and then re-centrifuged with two PBS washes of the fumed silica pellet. The resulting protein solution was then clarified and sterilized on a 0.2 μm hollow fiber (HF) filter and then bracketed using a series of HF modules with decreasing MWCO (750, 500, and 100 kDa). After the last Hp bracket was isolated (i.e. protein retained on the 100 kDa HF filter; Stage 3), a 50 kDa HF module (P/N: 502-E100-05-N) was used to retain the 100 kDa permeate, and remove molecules <50 kDa. After Hp purification was completed, the new bracket (Stage 4) was subject to constant volume diafiltration with 5× volume PBS and finally concentrated to ˜350 mL. A diagram of the full purification process is shown in FIG. 14.

HPLC-Size Exclusion Chromatography (SEC): Samples from the purification process were separated via size exclusion chromatography (SEC) using an Acclaim SEC-1000 (4.6×300 mm) column (Thermo Fisher Scientific, Waltham, Mass.) attached to a Dionex UltiMate 3000 system (Thermo Fisher Scientific, Waltham, Mass.) as described previously.

Hb Concentration. The concentration of Hb in the samples was measured spectrophotometrically via the Winterbourne equations.

Gel Electrophoresis: The purity and composition of the protein cocktail was analyzed via SDS-PAGE using an Invitrogen Mini Gel Tank (Thermo Fisher Scientific, Waltham, Mass.). Samples was prepared according to the manufacturer's guidelines. Gels were loaded with approximately 30 μg of protein per lane and tested under reducing (via addition of 0.1 M dithiothreitol) and non-reducing conditions. To estimate protein purity, gels were slightly overloaded (˜60 μg), imaged on a scanner and densitometric analysis was performed using ImageJ.

Total Protein Assay. Total protein was determined via the Bradford assay.

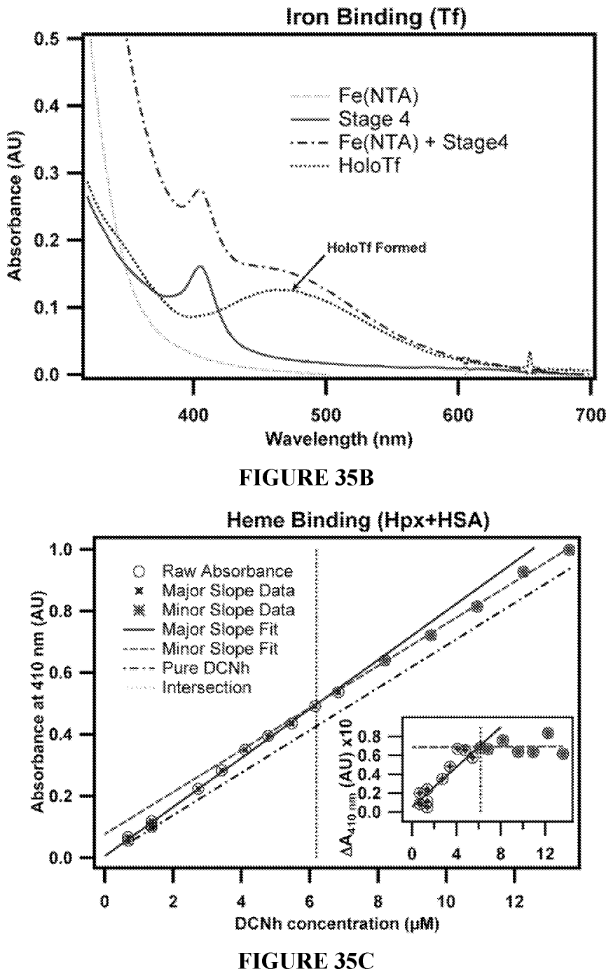

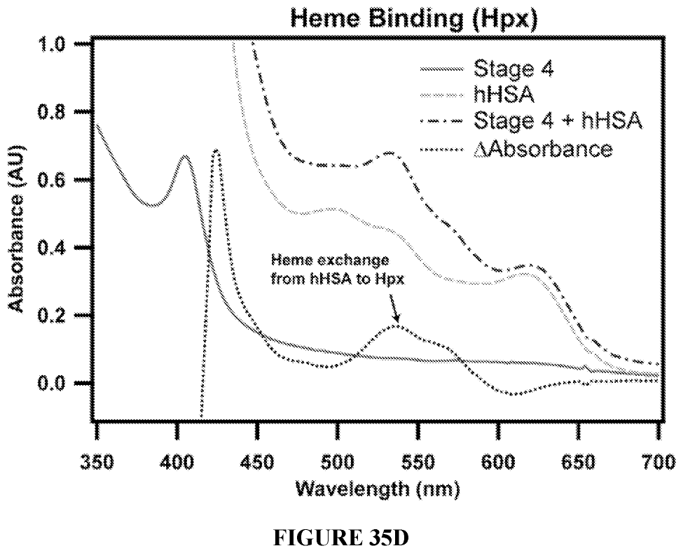

Hb Binding Capacity of Hp: The difference in MW between the Hp-Hb protein complex and pure Hb was used to assess the Hb binding capacity of Hp using HPLC-SEC as previously described. Briefly, samples containing Hp were mixed with excess Hb then separated via HPLC-SEC. The difference in the area under the curve (AUC) between the pure Hb solution, and the mixture of Hb and Hp was used to assess the Hb binding capacity of Hp. A representative HPLC-SEC chromatogram of this assay is shown in FIG. 35A.

Iron Binding Activity: The iron binding capacity of pure Tf and Tf contained in the protein scavenger cocktail was determined via reaction with ferric nitrilotriacetate [Fe(NTA)]. In this solution, iron is monomerically chelated, allowing for a fast reaction with Tf. Briefly, the Tf sample was reacted with excess Fe(NTA), and the equilibrium change in absorbance was measured to obtain the spectra of the formed holo-Tf complex (FIG. 35B). The extinction coefficient of holo-Tf at 465 nm was then used to estimate the concentration of iron bound to Tf. The apo-Tf concentration was determined based on the 465 nm absorbance of the sample prior to addition of Fe(NTA) (contribution of residual heme in the form of metHb was estimated based on the sample absorbance at 404 nm).

Heme Binding Activity: The activity of heme-binding proteins in the purified protein scavenger cocktail (primarily from HSA and Hpx) was determined via the dicyanohemin (DCNh) incorporation assay. Briefly, the sample was mixed with increasing concentrations of DCNh, and the equilibrium absorbance of the Soret peak maxima (410 nm in the case of HSA/Hpx) was measured. The inflection point in the graph of the equilibrium absorbance versus DCNh concentration was used to determine the saturation point of the heme-binding pockets (FIG. 35C). To determine the heme-binding activity of Hpx individually, the protein cocktail was mixed with excess heme-HSA (hHSA) and the change in absorbance was used to determine the concentration of heme-Hpx (FIG. 35D).

Trypsin Digest Mass Spectrometry. Protein identification in the protein cocktail was confirmed using nano-liquid chromatography-nanospray tandem mass spectrometry (LC/MS/MS) on a Thermo Scientific Fusion Orbitrap mass spectrometer equipped with an EASY-Spray™ Sources operated in positive ion mode as described previously.

ELISA. To quantify the concentration of protein components in FIV and inn the protein cocktail, ELISA kits specific for Hp, Tf, HSA, and Hpx were used according to the manufacturer's instructions (R&D Systems Catalog #DHAPGO for Hp, and Eagle BioSciences HTF31-K01 for Tf, HUA39-K01 for HSA, and HPX39-K01 for Hpx).

Results

The purification process outlined in FIG. 14 was performed on three batches for assessment of sample composition, and yield (compared to the raw FIV suspension). These results are summarized in Table 9.

TABLE 9
Summary of the protein scavenging cocktail composition, concentration, and yield.
Percentage composition was assessed via the activity binding assays.

| | Total (g) | ± | Concentration (mg/mL) | ± | Yield (%) | ± |
|---|---|---|---|---|---|---|
| Protein | 61.9 (100%) | 9.4 | 171 | 21 | 50 | 4 |
| Hp ^{a} | 5.97 (10%) | 0.12 | 16.5 (16^{d}) | 0.7 | 49 | 3 |
| Hpx | 2.21 (4%) | 0.42 | 6.1 (8.8^{d}) | 0.9 | (52 ^{d}) | (2) |
| HSA ^{b} | 28.7 (42%) | 4.3 | 79.1 (75 ^{d}) | 10.1 | (88 ^{d}) | (10) |
| apoTf | 18.2 (29%) | 2.7 | 50.1 | 6.03 | — | — |
| Tf ^{c} | 3.05 (34%) | 0.17 | 58.6 (63^{d}) | 5.61 | (81 ^{d}) | (9) |
| Other | 6.12 (10%) | 2.4 | 16.9 | 6.1 | — | — |
| | Concentration (mM) | ± | | | | |
| HbBC | 0.61 | 0.03 | | | | |
| HemeBC | 1.22 | 0.16 | | | | |
| FeBC | 1.25 | 0.15 | | | | |

HbBC = Hb-binding capacity.
HemeBC = heme-binding capacity.
FeBC = iron-binding capacity.
^{a} Hp determined assuming a 1.65 mass binding ratio of Hb:Hp2-2
^{b} HSA determined by the total heme binding capacity excluding Hpx
^{c} Total Tf determined via the non-Hb contribution from the absorbance at 460 nm
^{d} Concentration and yield from ELISA kits

As shown in Table 9, each 500 g batch of FIV yielded more than 60 g of a concentrated protein cocktail composed primarily of HSA, and Tf. The protein cocktail was also comprised of ˜10% and 5% of Hp and Hpx, respectively. The total protein yield and Hp yield of 50% (based on the initial total protein and Hp present in FIV) indicated that ˜75% of the total protein and ˜85% of the Hp was recovered from the 100 kDa permeate (based on a total protein and HbBC loss of 65% and 58% from the Hp purification process, respectively. Thus, only approximately 15% of the total protein and 8% of the Hp were not recovered in one of the stages of the new protein scavenging cocktail purification process. ELISA confirmed the results from the activity binding assays. Interestingly, quantification of Hp via ELISA did not deviate from the results of the Hb-binding assay, which was the case when larger MW Hp fractions were assessed. This was likely due to the lower MW of these Hp species. Moreover, comparing the FIV content of Hpx, HSA and Tf with that retained in the protein cocktail indicated that more than 80% of HSA and Tf was recovered, but only 50% of Hpx was recovered. The loss of protein can be attributed to the filtration of proteins through the 50 kDa filter, unspecific protein binding to the fumed silica or from general processing (i.e. loss of residual fluid in tubing). Finally, analyzing the molar concentration of the Hb, heme and iron binding capacities (HbBC, hemeBC, and FeBC, respectively) of the protein cocktail, it exhibited a ˜1:2:2 molar ratio for binding Hb, heme and iron (on an iron basis).

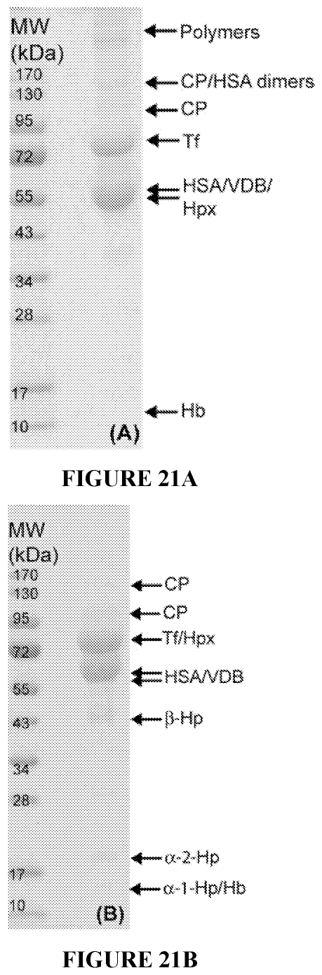

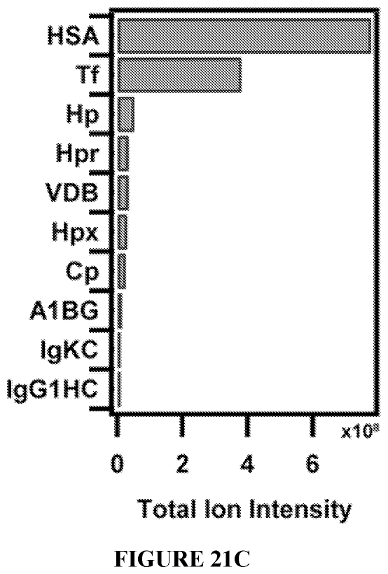

To further analyze the composition of the protein cocktail, samples were analyzed via SDS-PAGE and trypsin digest mass spectrometry (MS). The results of a representative batch are shown in FIG. 21.

All the expected proteins were present in the SDS-PAGE and identified in the mass spectroscopic (MS) analysis. The main proteins in addition to Hp, Hpx, HSA and Tf detected with MS were ceruloplasmin (Cp) and vitamin-D binding protein (VDB). Based on the similar ion intensity of Cp and VDB compared to that of Hpx, it would be expected that these components had similar mass composition compared to Hpx (shown to be ˜5% on the activity binding and ELISA assays). Moreover, the detection of haptoglobin-related protein (Hpr) was likely due to the high sequence identity of Hpr to the Hp1-1 phenotype as shown previously in the literature. To further estimate the protein composition, densitometric analysis was performed on the SDS-PAGE data, and the results are shown in Table 10.

TABLE 10
Composition of the protein scavenging cocktail
based on SDS-PAGE densitometric analysis.

| NON-REDUCED | | | REDUCED | | |
|---|---|---|---|---|---|
| | Compo- | | | Compo- | |
| Species | sition | +/− | Species | sition | +/− |
| HSA/Hpx/VDB | 47.7% | 1.7% | Tf/Hpx | 42.4% | 3.1% |
| Tf | 31.2% | 1.2% | HSA/VDB | 40.3% | 3.4% |
| Polymers (Hp) | 15.2% | 3.0% | β-Hp | 4.8% | 0.1% |
| Cp (135 kDa) | 2.4% | 0.0% | α-2Hp | 3.3% | 0.5% |
| HSA dimer/Cp | 2.4% | 0.1% | α-1Hp/Hb | 3.0% | 1.0% |
| (115 kDa) | | | | | |
| Other | 1.1% | 0.0% | Cp (115 kDa) | 1.9% | 0.0% |
| | | | Other (>140 kDa) | 1.7% | 0.7% |
| | | | Cp (135 kDa) | 1.7% | 0.0% |
| | | | Other (~28 kDa) | 1.6% | 0.4% |
| | | | β-2 glycoprotein | 0.7% | 0.1% |

From densitometric analysis, it was noted that only >90% of the protein cocktail was composed of four major proteins (HSA, Hpx, Hp, and Tf). This result agreed with the activity binding assays, which accounted for 90% of these proteins. Furthermore, the mass percentage composition from the HSA/Hpx, Tf and Hp SDS-PAGE bands were similar to the mass composition of the individual proteins shown in Table 9. Cp was also noticeable on the SDS-PAGE with ˜4% mass composition. Hpx could be estimated on the SDS-PAGE as it alters its apparent MW when reduced. Comparing the percent composition of Tf and the HSA band before and after reduction showed a 7-9% change, indicating a similar composition as the estimation determined via the heme-binding assay (˜5%).

Discussion

A major portion (>90%) of the heme-binding capacity (hemeBC) of the protein scavenging cocktail was attributed to HSA, which can bind to both free heme and free iron. HSA, is a 69 kDa non-glycosylated chain. Circulating at approximately 35-55 mg/mL in plasma, HSA is the most abundant plasma protein with a diverse set of functions. Although HSA has a lower heme-binding affinity (K_{d}≈10 nM) than Hpx (K_{d}<1 pM), heme binding to HSA decreases free heme-mediated oxidative damage. Yet, concentrations of 4× molar excess are required to fully prevent heme-mediated lipid oxidation in vitro. Moreover, unlike Hpx, HSA itself is prone to oxidation due to the bound heme. Similarly, the iron-binding properties of HSA prevents oxidative damage due to the presence of free iron. Furthermore, Hb-mediated lipid peroxidation can be prevented via HSA administration. Thus, HSA can function as an iron and heme transport/carrier until Tf and Hpx can deliver these ligands to their respective clearance receptors. Furthermore, Hpx be recycled upwards of 20× during some states of hemolysis, potentially allowing a small quantity of Hpx to scavenge large quantities of heme. Kinetic studies have also revealed that, upon heme release from Hb, the majority of heme is initially bound to lipoproteins or the RBC membrane. Heme is then transferred first to HSA which transports heme to Hpx, indicating the role has plays in heme transport. Hence, not only can HSA neutralize heme toxicity and aid Hpx-mediated heme clearance, studies have demonstrated a receptor for heme which can explain heme delivery directly via heme-HSA.

Furthermore, in addition to reducing the toxicity of free heme and free iron, HSA is the major antioxidant in plasma. Therefore, HSA administration may aid in preventing oxidative damage during hemolytic states. Interestingly, conditions in which administration of HSA is clinically recommended may benefit from hemolysis treatment proteins. For example, during severe burns, septic shock, organ transplantation, or surgeries, HSA can be administered as a plasma expander. Yet, these conditions have also been shown to have hemolytic traits. Thus, administration of a protein cocktail containing both HSA and hemolysis scavenging proteins could yield better patient outcomes.

The second most abundant protein found in the protein scavenging cocktail was transferrin (Tf). Tf is a ˜80 kDa serum glycoprotein normally present at 2-4 mg/mL in the plasma. Each Tf molecule has two iron binding sites (binds to ferric iron, Fe³⁺), and the Tf-Fe complex maintains the iron in a non-reactive state. During states of hemolysis, Tf saturation with iron increases with a contaminant increase in non-transferrin bound iron (i.e. free iron). Tf may be a synergistic co-therapeutic during hemolysis treatment, since it has been shown that Hp and Hpx both increase iron-dependent neural cell damage when administered in vitro. However, neural damage was reduced when an iron chelating agent was co-administered. Tf is also an antioxidant protein, potentially aiding in the maintenance of redox homeostasis during hemolysis.

The third main component of the protein scavenging cocktail was haptoglobin (Hp). Hp is an α-2 glycoprotein mainly responsible for scavenging cell-free Hb. The MW of Hp varies from approximately 90-900 kDa due to its polymorphism. Although found in most bodily fluids of mammals, it is present in plasma at concentrations normally ranging from 0.5-3 mg/mL. After binding to cell-free Hb, the Hb-Hp complex is scavenged by CD163+ macrophages and monocytes to clear the organism of toxic cell-free Hb. When bound to Hp, the large size of the Hb-Hp complex prevents Hb extravasation into the tissue space, reducing Hb-mediated NO scavenging and vasoconstriction. Furthermore, Hp binding to Hb prevents heme release from Hb, and lowers the ability of Hb to elicit oxidative damage and inflammation. Other interesting properties of Hp are its intrinsic antioxidant potential, chaperone activity and binding to HMGB1 protein. A more detailed description of Hp and its applications can be found in Example 2.

The three other minor components of the protein scavenging cocktail are hemopexin (Hpx), ceruloplasmin (Cp), and vitamin-D binding protein (VDB). Hpx is a ˜60 kDa serum glycoprotein (˜20% carbohydrate) with the highest affinity for free heme (K_{d}<1 pM). Each Hpx molecule can bind one heme molecule and its concentration in plasma ranges from 0.5-1.5 mg/mL. Similar to Hp binding to Hb, Hpx binding to heme prevents the oxidative reactions of heme from occurring. Yet, unlike Hp, after uptake of heme-Hpx and intracellular heme release, Hpx is usually recycled and released intact back into the circulation. Although it has been shown that Hpx may be recycled, serum Hpx levels decrease during hemolytic states indicating that Hpx may be degraded upon receptor mediated uptake. The discrepancy in Hpx uptake and recycling has been attributed to potentially different uptake mechanisms. Hpx also aids during hemolysis by inducing expression of heme-oxygenase-1 and ferritin. These proteins protect the organism from the oxidative and inflammatory stress of heme during hemolysis.

Cp is a ˜120 kDa serum protein responsible for binding and transport of copper. Furthermore, Cp has a major role in iron metabolism as a ferroxidase for oxidation of Fe²⁺ into Fe³⁺ and for stabilization of ferroportin (cellular iron exporter). Oxidation of iron to Fe³⁺ is required for iron binding to Tf (transport) or ferritin (storage). Thus, Cp is vital for proper iron metabolism and genetic Cp deficiencies lead to an accumulation of iron in organs. Moreover, low Cp levels have been associated with severely burnt patients or patients having a high risk for acute organ failure. It has also been shown that Cp activity decreases during trauma and burn injury which contributes to inflammation and hypoferremia. Studies have also demonstrated that copper may be co-endocytosed with heme-Hpx given that copper binding to Hpx intracellularly may aid in heme release. Finally, VDB, also known as GC-globulin, is a ˜55 kDa α-2 globulin which obtained its name based on its role in vitamin-D binding and transport. Yet, VDB has other functions such as an actin scavenger. Serum actin is another toxic species that can be released during hemolysis or tissue damage. Excess actin saturates its natural scavengers, which include gelsolin and VDB. Thus, VDB therapy during hemolysis could prevent actin-mediated toxicity.

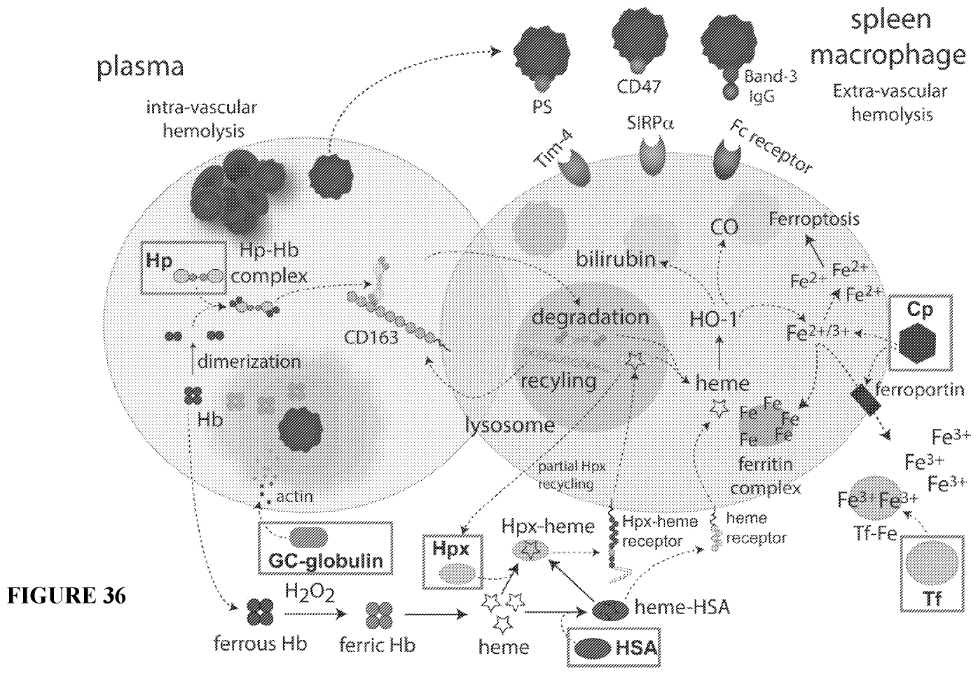

Overall, the protein cocktail components provide a potentially comprehensive treatment strategy for various states of hemolysis. The major roles of the proteins in the cocktail are summarized in FIG. 36.

An important aspect to consider for the protein scavenging cocktail is its advantages over plasma. First, the protein cocktail does not contain immunoglobulins, thus providing a universally transfusable solution. This expands the source of plasma for the cocktail as only 4% of the population is has blood type AB (universal plasma donor). Immunoglobulins have also been attributed to the significant and well-established risk of transfusion-related acute lung injury, which is considered the leading cause of transfusion-related mortality. Unlike plasma, the shelf-life of the cocktail would not be restricted by the loss of coagulation factors. Thus, the cocktail could potentially maintain its efficacy for a much longer period (HSA has a shelf-life of one year stored at 1-25° C.). Moreover, the protein cocktail can be stored at −80° C. and potentially be lyophilized for enhanced protein stability. Furthermore, due to the ethanol precipitation steps used to produce FIV and the extensive nanofiltratrion used to isolate the protein cocktail, the risk of blood-born infectious agents is greatly minimized compared to plasma administration. The source of precursor material for the protein cocktail is also beneficial, since FIV is a waste product stream from the plasma fractioning industry, making the cocktail a cost-effective option for hemolysis treatment. Finally, the concentration of the desired hemolysis scavenging proteins in the protein scavenging cocktail is enhanced compared to plasma. This is shown in Table 11.

TABLE 11
Comparison between plasma, FIV and the protein scavenging
cocktail protein composition by mass.

| | Plasma* | FIV | Protein Cocktail |
|---|---|---|---|
| HSA | 50% | 25% | 40% |
| Tf | 5% | 25% | 35% |
| Hp | 2% | 5% | 10% |
| Hpx | 1% | 5% | 5% |
| Cp** | <1% | — | <5% |
| VDB** | <1% | — | <5% |
| Total Protein (mg/mL) | 60-80 | 25 | 170 |

*Plasma concentration and mass percentage based on experimental data (not shown) and the literature.
**not specifically tested for in this study

As shown in Table 11, the protein scavenging cocktail has more than double the total protein concentration of plasma. Moreover, the HSA composition was slightly reduced, while the composition of Tf, Hp, Hpx, Cp and VDB all had approximately a five-fold or greater increase compared to plasma.

If desired, the HSA content of the cocktail may be further reduced via chromatography techniques described in the literature. Other methods to reduce the HSA content include exposing the protein cocktail to the process conditions used for FIV precipitation or to 40-60% ammonium sulfate. Yet, these processes would likely lead to loss of Tf and Hpx from the cocktail and lower the overall protein yield from processing. One promising technique to remove only HSA from the cocktail would be employ the selective precipitation of HSA with Rivanol. Yet, given the heme and iron binding role of HSA and its antioxidant properties, it may be advantageous simply leaving it as a component in the protein cocktail.

Another benefit of HSA as a component in the protein scavenging cocktail is its extensive ligand binding properties. This allows for a flexible drug delivery vehicle for treatment of the desired condition. In the case of hemolysis treatment, as the major store of NO in vivo, HSA may be used to deliver NO to the vasculature during states of hemolysis, thus preventing hypertension. Nitrite infusions have already been shown to restrict Hb hypertension during hemolysis. NO delivery would require binding of NO to the free Cys34 of HSA to form S-NO HSA (HSA-SNO) prior to administration of the cocktail. HSA-SNO may also be used in wound healing applications while the scavenging proteins would prevent wound infections via iron sequestration.

The high HSA content of the cocktail also make it a promising next generation plasma expander. Similar to plasma or HSA use, the protein cocktail could replenish lost blood volume during hemorrhage, burn, sepsis or other hypovolemic conditions. Furthermore, given the antioxidant, anti-inflammatory, scavenging, and iron metabolism properties of the protein components, the cocktail could aid in reducing the potential toxicity of blood transfusions required post hemorrhage shock and the resultant hemolysis induced by the trauma. Compared to plasma, the major difference may be the lack of coagulation proteins. Yet, there is evidence that suggests that the benefit of plasma resuscitation is associated with its HSA content and not the presence of coagulation factors.

Moreover, there are clinical indications for therapeutic plasma exchange (TPE) in which the replacement fluid is HSA that could be enhanced with the hemolysis treating cocktail. For example, autoimmune hemolytic anemia or ABO incompatible hematopoietic transplants can both lead to intravascular hemolysis. Thus, some TPE treatments may benefit from using the hemolysis scavenger proteins in the cocktail.

Conclusions

The molar binding capacity of the protein scavenger cocktail for Hb, heme, and iron was ˜1:2:2 (Hb:heme:iron). Moreover, the main components of the protein scavenger cocktail consisted of: HSA, Tf, Hp, Hpx, Cp, and VDB. Based on this composition, the cocktail can potentially be used in a variety of applications. These applications include in vivo and in vitro hemolysis neutralization by scavenging free heme, cell-free Hb, and free iron. Furthermore, VDB scavenges free actin released during hemolysis and general cell lysis, and Cp aids in iron metabolism. Another potential promising application for the cocktail is as an enhanced plasma expander given that various indications for plasma expanders are also associated with hemolysis. The composition of the cocktail may be altered either through further processing or supplementation for each indication. Taken together, this study presents a simple and effective method to purify and characterize a protein cocktail capable of scavenging free iron, free heme, and free Hb for possible treatment of states of hemolysis.

The compositions, systems, and methods of the appended claims are not limited in scope by the specific compositions, systems, and methods described herein, which are intended as illustrations of a few aspects of the claims. Any compositions, systems, and methods that are functionally equivalent are intended to fall within the scope of the claims. Various modifications of the compositions, systems, and methods in addition to those shown and described herein are intended to fall within the scope of the appended claims. Further, while only certain representative compositions, systems, and method steps disclosed herein are specifically described, other combinations of the compositions, systems, and method steps also are intended to fall within the scope of the appended claims, even if not specifically recited. Thus, a combination of steps, elements, components, or constituents may be explicitly mentioned herein, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A method of isolating an apoprotein from a protein solution comprising a conjugated protein, wherein the conjugated protein comprises the apoprotein and a hydrophobic ligand associated with the apoprotein, the method comprising:
(i) contacting the conjugated protein with an aqueous solution comprising a water-miscible solvent and a pH modifier, thereby forming a protein solution having a pH of less than 6.5 or greater than 8; and
(ii) filtering the protein solution by ultrafiltration against a filtration membrane having a pore size that separates the apoprotein from the hydrophobic ligand, thereby forming a retentate fraction comprising the apoprotein and a permeate fraction comprising the hydrophobic ligand.

2. The method of claim 1, wherein the hydrophobic ligand is non-covalently associated with the apoprotein.

3. The method of claim 1, wherein the hydrophobic ligand is covalently associated with the apoprotein.

4. The method of any of claims 1-3, where the protein solution has a pH of from 2 to 6, such as from 3 to 6 or from 2 to 5.

5. The method of any of claims 1-3, wherein the protein solution has a pH of from greater than 8 to 11.

6. The method of any of claims 1-5, wherein the filtration membrane is rated for retaining solutes having a molecular weight of from a molecular weight greater than the molecular weight of the hydrophobic ligand to a molecular weight less than the molecular weight of the apoprotein.

7. The method of any of claims 1-6, wherein the ultrafiltration comprises tangential-flow filtration.

8. The method of any of claims 1-7, wherein the ligand comprises a prosthetic group, cofactor, a lipid, a metabolite, or a combination thereof.

9. The method of any of claims 1-8, wherein the conjugated protein comprises a heme protein.

10. The method of claim 9, wherein the conjugated protein comprises hemoglobin, the apoprotein comprises apohemoglobin and the ligand comprises heme.

11. The method of claim 10, wherein the hemoglobin is present in the protein solution at a concentration from 0.1 mg/mL to 10 mg/mL, such as from 0.1 mg/mL to 5 mg/mL or from 0.5 mg/mL to 3 mg/mL.

12. The method of any of claims 1-8, wherein the conjugated protein comprises human serum albumin (HSA).

13. The method of claim 12, wherein the HSA is present in the protein solution at a concentration from 0.5 mg/mL to 25 mg/mL, such as from 10 mg/mL to 20 mg/mL.

14. The method of any of claims 1-13, wherein the water-miscible solvent comprises a polar protic solvent.

15. The method of any of claims 1-14, wherein the water-miscible solvent comprises an alcohol.

16. The method of claim 15, wherein the alcohol comprises ethanol, methanol, or a combination thereof.

17. The method of any of claims 15-16, wherein the aqueous solution comprises from 10% to 90% by volume alcohol.

18. The method of claim 17, wherein the conjugated protein comprises hemoglobin and the aqueous solution comprises from 60% to 90% by volume alcohol.

19. The method of claim 17, wherein the conjugated protein comprises HSA and the aqueous solution comprises from 30% to 60% by volume alcohol.

20. The method of any of claims 1-19, wherein filtering step (ii) comprises buffer exchange.

21. The method of any of claims 1-20, wherein filtering step (ii) comprises continuous diafiltration or dialysis.

22. The method of claim 21, wherein the retentate fraction is spectroscopically monitored during the continuous diafiltration to monitor separation of the hydrophobic ligand from the apoprotein.

23. The method of claim 22, wherein spectroscopically monitoring the retentate fraction comprises monitoring a spectroscopic peak associated with the apoprotein and a spectroscopic peak associated with the conjugated protein.

24. The method of claim 23, wherein filtering step (ii) comprises performing the continuous diafiltration until a relative magnitude of the spectroscopic peak associated with the apoprotein and the spectroscopic peak associated with the conjugated protein suggest that the apoprotein and the conjugated protein are present in the retentate fraction at a molar ratio of at least 9:1.

25. The method of any of claims 1-24, wherein the method further comprises (iii) neutralizing the retentate fraction to isolate the apoprotein.

26. The method of claim 25, wherein the neutralizing step (iii) comprises continuous diafiltration with water or a buffer solution having a pH of from 6.8 to 7.6.

27. The method of any of claims 25-26, wherein the apoprotein isolated in step (iii) comprises less than 1% residual hydrophobic ligand relative to the concentration of apoprotein isolated in step (iii), as measured by UV-Vis spectroscopy.

28. The method of any of claims 25-27, wherein the apoprotein isolated in step (iii) is stable for a period of at least 7 days at 22° C.

29. The method of claim 28, wherein at least 75% of the apoprotein remains soluble in solution after storage at 22° C. for 7 days.

30. The method of any of claims 28-29, wherein at least 75% of the apoprotein remains soluble in solution after storage at 4° C. for 180 days.

31. The method of any of claims 28-30, wherein at least 75% of the apoprotein remains soluble in solution after storage at −80° C. for 180 days.

32. The method of any of claims 25-31, further comprising lyophilizing the apoprotein isolated in step (iii).

33. A method of isolating a ligand from a protein solution comprising a conjugated protein, wherein the conjugated protein comprises the apoprotein and a hydrophobic ligand associated with the apoprotein, the method comprising:
(i) mildly denaturing the conjugated protein to form a protein solution;
(ii) filtering the protein solution by ultrafiltration against a filtration membrane having a pore size that separates the apoprotein from the hydrophobic ligand, thereby forming a retentate fraction comprising the apoprotein and a permeate fraction comprising the hydrophobic ligand.

34. The method of claim 33, wherein mildly denaturing the conjugated protein comprises heating the conjugated protein.

35. The method of claim 34, wherein mildly denaturing the conjugated protein comprises heating the conjugated protein to a temperature of from 40° C. to 60° C.

36. The method of any of claims 33-35, wherein mildly denaturing the conjugated protein comprises contacting the conjugated protein with a pH modifier.

37. The method of any of claims 33-36, wherein mildly denaturing the conjugated protein comprises contacting the conjugated protein with a non-aqueous solvent, such as an alcohol.

38. The method of any of claims 33-37, wherein mildly denaturing the conjugated protein comprises contacting the conjugated protein with a chaotropic agent, such as guanidinium chloride, lithium perchlorate, lithium acetate, magnesium chloride, sodium dodecyl sulfate, thiourea, urea, calcium chloride, or a combination thereof.

39. The method of any of claims 1-38, further comprising covalently modifying the apoprotein to increase stability of the apoprotein in solution.

40. The method of claim 39, wherein covalently modifying the apoprotein comprises pegylating the apoprotein.

41. Apohemoglobin produced by the method of any of claims 1-40.

42. The apohemoglobin of claim 41, wherein the apohemoglobin is characterized by a Soret peak having a maximum absorption ranging from 411-417 nm.

43. A method for isolating a haptoglobin from plasma or a fraction thereof, the method comprising
(i) clarifying the plasma or fraction thereof; and
(ii) filtering the plasma or a fraction thereof by ultrafiltration against a filtration membrane, thereby forming a retentate fraction comprising haptoglobin having a molecular weight of greater than about 100 kDa and a permeate fraction comprising serum proteins and other impurities having a molecular weight of less than about 100 kDa.

44. The method of claim 43, wherein the plasma or fraction thereof comprises plasma fraction IV, plasma fraction V, or a combination thereof.

45. The method of any of claims 43-44, wherein clarifying the plasma or a fraction thereof comprises removing suspended solids from the plasma or fraction thereof.

46. The method of claim 45, wherein removing suspended solids from the plasma or fraction thereof comprises filtering the plasma or a fraction thereof.

47. The method of any of claims 45-46, wherein removing suspended solids from the plasma or fraction thereof comprises contacting the plasma or a fraction thereof with a salting out agent, such as ammonium sulfate.

48. The method of any of claims 45-47, wherein removing suspended solids from the plasma or fraction thereof comprises contacting the plasma or a fraction thereof with a lipid binding agent, such as fumed silica.

49. The method of any of claims 43-48, wherein the ultrafiltration comprises tangential-flow filtration.

50. The method of any of claims 43-49, wherein the method further comprises filtering the permeate fraction comprising serum proteins and other impurities by ultrafiltration against a second filtration membrane, thereby forming a second retentate fraction comprising a blend of proteins having a molecular weight below about 100 kDa and above a cutoff value and a second permeate fraction comprising serum proteins and other impurities having a molecular weight below the cutoff value, wherein the blend of proteins comprises low molecular weight haptoglobin, transferrin, hemopexin, albumin, or a combination thereof.

51. The method of claim 48, wherein the cutoff value is from about 20 kDa to about 70 kDa, such as from about 25 kDa to about 50 kDa.

52. A method for isolating haptoglobin from plasma or a fraction thereof, wherein the method comprises
(i) filtering the plasma or fraction thereof by ultrafiltration against a first filtration membrane, thereby forming a first retentate fraction comprising serum proteins having a molecular weight above a first cutoff value and a first permeate fraction comprising the haptoglobin and serum proteins having a molecular weight below the first cutoff value; and
(ii) filtering the first permeate fraction by ultrafiltration against a second filtration membrane, thereby forming a second retentate fraction comprising Hp2-1 and Hp2-2 having a molecular weight below the first cutoff value and above a second cutoff value; and a second permeate fraction comprising Hp2-1, Hp2-2, and serum proteins having a molecular weight below the second cutoff value.

53. The method of claim 52, wherein the method further comprises:
(iii) filtering the second permeate fraction by tangential-flow filtration against a third filtration membrane, thereby forming a third retentate fraction comprising Hp2-1 and Hp2-2 having a molecular weight below the second cutoff value and above a third cutoff value; and a third permeate fraction comprising low molecular weight haptoglobin, serum proteins and other impurities having a molecular weight below the third cutoff value.

54. The method of claim 53, wherein the method further comprises:
(iv) filtering the third permeate fraction comprising low molecular weight haptoglobin, serum proteins and other impurities by ultrafiltration against a fourth filtration membrane, thereby forming a fourth retentate fraction comprising a blend of proteins having a molecular weight below the third cutoff value and above a fourth cutoff value and a fourth permeate fraction comprising serum proteins and other impurities having a molecular weight below the fourth cutoff value, wherein the blend of proteins comprises low molecular weight haptoglobin, transferrin, hemopexin, albumin, or a combination thereof.

55. The method of any of claims 52-54, wherein the first cutoff value is from about 650 kDa to about 1000 kDa.

56. The method of any of claims 52-55, wherein the second cutoff value is from about 20 kDa to about 700 kDa.

57. The method of any of claims 53-56, wherein the third cutoff value is from about 20 kDa to about 200 kDa.

58. The method of any of claims 54-57, wherein the fourth cutoff value is from about 20 kDa to about 70 kDa.

59. The method of any of claims 53-58, wherein the first cutoff value is about 750 kDa, the second cutoff value is about 500 kDa, and the third cutoff value is about 100 kDa.

60. The method of claim 59, wherein the fourth cutoff value is about 30 kDa.

61. The method of claim 59, wherein the fourth cutoff value is about 50 kDa.

62. The method of any of claims 52-61, wherein the plasma or fraction thereof comprises plasma fraction IV, plasma fraction V, or a combination thereof.

63. The method of any of claims 52-62, wherein the ultrafiltration comprises tangential-flow filtration.

64. The method of any of claims 52-63, wherein the method further comprises clarifying the plasma or fraction thereof prior to step (i).

65. The method of claim 64, wherein removing suspended solids from the plasma or fraction thereof comprises filtering the plasma or a fraction thereof, such as microfiltration of the plasma or a fraction thereof.

66. The method of any of claims 64-65, wherein removing suspended solids from the plasma or fraction thereof comprises contacting the plasma or a fraction thereof with a salting out agent, such as ammonium sulfate.

67. The method of any of claims 64-66, wherein removing suspended solids from the plasma or fraction thereof comprises contacting the plasma or a fraction thereof with a lipid binding agent, such as fumed silica.

68. The method of any of claims 43-67, wherein the method further comprises a solvent/detergent treatment step for virus inactivation at any point during the isolation method.

69. The method of claim 68, wherein the solvent/detergent treatment step removes and/or reduces lipoproteins and/or proteins associated with lipoproteins.

70. The method of any of claims 43-69, wherein the last permeate fraction is collected for re-use as supply buffer for future batches.

71. A haptoglobin fraction prepared by the method of any of claims 43-70.

72. A haptoglobin-containing composition isolated from plasma or a fraction thereof, the composition comprising:
from 10% by weight to 99% by weight haptoglobin, based on the total weight of all proteins in the haptoglobin-containing composition;
from greater than 0% by weight to 30% by weight albumin, alpha-1 antitrypsin, or a combination thereof, based on the total weight of all proteins in the haptoglobin-containing composition; and
from greater than 0% by weight to 20% by weight macroglobulin, based on the total weight of all proteins in the haptoglobin-containing composition

73. The composition of claim 72, wherein the composition comprises from 10% by weight to 80% by weight haptoglobin, based on the total weight of all proteins in the haptoglobin-containing composition.

74. The composition of any of claims 72-73, wherein the albumin comprises polymeric albumin.

75. The composition of any of claims 72-74, wherein the composition comprises from greater than 0% by weight to 30% by weight alpha-1 antitrypsin, based on the total weight of all proteins in the haptoglobin-containing composition.

76. The composition of any of claims 72-75, wherein the composition comprises from greater than 0% by weight to 30% by weight albumin, based on the total weight of all proteins in the haptoglobin-containing composition.

77. The composition of any of claims 72-76, wherein the composition comprises from greater than 0% by weight to 10% by weight macroglobulin, based on the total weight of all proteins in the haptoglobin-containing composition.

78. The composition of any of claims 72-77, wherein the composition comprises from greater than 0% by weight to 15% by weight transferrin, based on the total weight of all proteins in the haptoglobin-containing composition.

79. The composition of any of claims 72-78, wherein the composition further comprises from greater than 0% to 15% by weight of an apolipoprotein, based on the total weight of all proteins in the haptoglobin-containing composition.

80. The composition of any of claims 72-79, wherein the composition further comprises from greater than 0% to 40% by weight of proteins associated with lipoproteins, based on the total weight of all proteins in the haptoglobin-containing composition.

81. The composition of claim 80, wherein the proteins associated with lipoproteins comprise high density lipoprotein.

82. The composition of any of claims 72-81, wherein the composition comprises from 40% by weight to 99% by weight haptoglobin, such as from 60% by weight to 99% by weight haptoglobin, based on the total weight of all proteins in the haptoglobin-containing composition.

83. The composition of any of claims 72-82, wherein the haptoglobin has an average molecular weight of from 80 kDa to 1,000 kDa, such as from 100 kDa to 1,000 kDa.

84. The composition of any of claims 72-83, wherein the composition further comprises vitamin-D binding protein, ceruloplasmin, hemopexin, or a combination thereof.

85. The composition of any of claims 72-84, wherein the composition further comprises an additional protein chosen from transferrin, hemopexin, or a combination thereof.

86. The composition of any of claims 72-85, wherein the haptoglobin is characterized by having residual hemoglobin as characterized by UV-visible spectroscopy of the Soret peak ranging from 402-407 nm.

87. The composition of any of claims 72-86, wherein the haptoglobin is characterized by having residual apohemoglobin as characterized by heme-binding capacity of the sample determined via UV-visible spectroscopy of the Soret peak formed by the heme-binding capacity assay which ranges from 411-417 nm.

88. A haptoglobin-containing composition isolated from plasma or a fraction thereof, the composition comprising:
from 5% by weight to 99% by weight haptoglobin, based on the total weight of all proteins in the haptoglobin-containing composition; and
from 1% by weight to 95% by weight transferrin, based on the total weight of all proteins in the haptoglobin-containing composition;
wherein the composition in substantially free of immunogenic proteins.

89. The composition of claim 88, wherein the immunogenic proteins comprise antibodies.

90. The composition of any of claims 88-89, wherein the composition comprises from 5% by weight to 60% by weight haptoglobin, such as from 5% by weight to 25% by weight haptogloin, based on the total weight of all proteins in the haptoglobin-containing composition.

91. The composition of any of claims 88-90, wherein the composition comprises from 1% by weight to 50% by weight transferrin, such as from 25% by weight to 40% by weight or from 30% by weight to 40% by weight transferrin, based on the total weight of all proteins in the haptoglobin-containing composition.

92. The composition of any of claims 88-91, wherein the composition comprises from 1% by weight to 75% by weight hemopexin, such as from 1% to 40% by weight hemopexin, from 5% to 40% by weight hemopexin, or from 1% by weight to 10% by weight hemopexin, based on the total weight of all proteins in the haptoglobin-containing composition.

93. The composition of any of claims 88-92, wherein the composition comprises from 1% by weight to 70% by weight albumin, such as from 5% to 30% by weight albumin, from 30% by weight to 50% by weight albumin, or from 30% by weight to 60% by weight albumin, based on the total weight of all proteins in the haptoglobin-containing composition.

94. The composition of claim 93, wherein the albumin comprises different molecular weight albumins including polymeric albumin species.

95. The composition of any of claims 88-94, wherein the composition further comprises from 1% by weight to 30% by weight alpha-1 antitrypsin, based on the total weight of all proteins in the haptoglobin-containing composition.

96. The composition of any of claims 88-95, wherein the composition further comprises from greater than 0% to 10% by weight of an apolipoprotein, based on the total weight of all proteins in the haptoglobin-containing composition.

97. The composition of claim 96, wherein the composition further comprises from greater than 0% to 40% by weight of proteins associated with lipoproteins, such as from greater than 0% to 10% by weight of proteins associated with lipoproteins, based on the total weight of all proteins in the haptoglobin-containing composition.

98. The composition of claim 97, wherein the proteins associated with lipoproteins comprise high density lipoprotein.

99. The composition of any of claims 88-98, wherein the haptoglobin has an average molecular weight of from 80 kDa to 1,000 kDa, such as from 100 kDa to 1,000 kDa.

100. The composition of any of claims 88-99, wherein the haptoglobin is characterized by having residual hemoglobin as characterized by UV-visible spectroscopy of the Soret peak ranging from 402-407 nm.

101. The composition of any of claims 88-100, wherein the composition further comprises vitamin-D binding protein, ceruloplasmin, or a combination thereof.

102. The composition of any of claims 88-101, wherein the composition comprises:
from 5% by weight to 15% by weight haptoglobin, based on the total weight of all proteins in the haptoglobin-containing composition;
from 30% by weight to 50% by weight albumin, based on the total weight of all proteins in the haptoglobin-containing composition;
from 1% by weight to 10% by weight hemopexin, based on the total weight of all proteins in the haptoglobin-containing composition; and
from 30% by weight to 40% by weight transferrin, based on the total weight of all proteins in the haptoglobin-containing composition;
wherein the composition in substantially free of immunogenic proteins.

103. The composition of claim 102, wherein the composition further comprises from 1% by weight to 15% by weight vitamin-D binding protein, ceruloplasmin, or a combination thereof, such as from 5% by weight to 15% by weight vitamin-D binding protein, ceruloplasmin, or a combination thereof, based on the total weight of all proteins in the haptoglobin-containing composition.

104. A method of treating hemolysis in a subject comprising administering the composition of any of claims 71-103 to the subject.

105. A method of stabilizing a composition comprising red blood cells, the method comprising adding the composition of any of claims 71-103 to the subject to the composition comprising red blood cells.

106. A method of improving safety of a hemoglobin-based substitute comprising adding the composition of any of claims 71-103 to the hemoglobin-based substitute or co-infusing the composition of any of claims 71-103 with the hemoglobin-based substitute.

107. A method of treating a wound in a subject, the method comprising contacting the wound or a region proximate thereto with the composition of any of claims 71-103.

108. The method of claim 107, wherein contacting the wound or a region proximate thereto comprises topically applying the composition of any of claims 71-103 to the wound or a region proximate thereto.

109. The method of claim 107, wherein contacting the wound or a region proximate thereto comprises injecting the composition of any of claims 71-103 into the wound or a region proximate thereto.

110. A method for treating a microbial infection in a subject, the method comprising administering a therapeutically effective amount of the composition of any of claims 71-103 to the subject.

111. The method of claim 110, wherein the composition is administered locally to a site of the microbial infection.

112. The method of any of claims 110-111, wherein the microbial infection comprises an antibiotic-resistant bacterial strain.

113. The method of any of claims 110-112, wherein the composition of any of claims 71-94 is administered in amount effective to decrease favorability of bacterial growth at a site of the microbial infection.

114. A method of reducing hemolysis in an organ or tissue transplanted or to be transplanted, the method comprising perfusing the organ or tissue with the composition of any of claims 71-103.

115. The method of claim 114, wherein the organ or tissue is perfused ex vivo with red blood cells or a hemoglobin-based blood substitute.

116. A method for isolating a target protein from a solution comprising a plurality of proteins, the method comprising:
(i) filtering the protein solution by ultrafiltration against a first filtration membrane, thereby forming a first retentate fraction comprising impurities having a molecular weight above a first cutoff value and a first permeate fraction comprising the target protein and impurities having a molecular weight below the first cutoff value;
(ii) contacting the first permeate fraction with a binding molecule that selectively associates with the target protein to form a target protein complex having a molecular weight above the first cutoff value; and
(iii) filtering the first permeate fraction by ultrafiltration against a second filtration membrane, thereby forming a second retentate fraction comprising the target protein complex having a molecular weight above the first cutoff value and a second permeate fraction comprising the impurities having a molecular weight below the first cutoff value.

117. The method of claim 116, wherein the method further comprises (iv) contacting the second retentate fraction with a dissociating agent, thereby inducing dissociation of the target protein complex to the target protein and the binding molecule.

118. The method of any of claims 116-117, wherein the method further comprises (v) filtering the second retentate fraction to separate the target protein from the binding molecule and the dissociating agent, thereby isolating the target protein.

119. The method of claim 118, wherein step (v) comprises:
filtering the second retentate fraction by ultrafiltration against a third filtration membrane, thereby forming a third retentate solution comprising the target protein having a molecular weight above a second cutoff value and a second permeate fraction comprising the impurities having a molecular weight below the second cutoff value.

120. The method of any of claims 116-119, wherein the binding molecule comprises an antibody, antibody fragment, antibody mimetic, protein, peptide, oligonucleotide, DNA, RNA, aptamer, organic molecule, intein, split-intein, or combination thereof.

121. The method of any of claims 116-120, wherein the dissociating agent comprises a pH modifier, a salt, a polyelectrolyte, a chaotropic agent, a non-aqueous solvent, or a combination thereof.

122. The method of any of claims 116-121, wherein the ultrafiltration comprises tangential-flow filtration.

123. A method for isolating a plasma protein concentrate from plasma or a fraction thereof, the method comprising
(i) clarifying the plasma or fraction thereof; and
(ii) filtering the plasma or a fraction thereof by ultrafiltration against a filtration membrane, thereby forming a retentate fraction comprising plasma proteins having a molecular weight of greater than a cut-off value and a permeate fraction comprising serum proteins and other impurities having a molecular weight of less than the cut-off value.
(iii) concentrating the proteins retained in step (i) to a therapeutic level.

124. The method of claim 123, wherein the first cut-off value is from 80 kDa to 120 kDa and wherein haptoglobin comprises a majority of the proteins in the plasma protein concentrate.

125. The method of any of claims 123-124, wherein the plasma or fraction thereof comprises plasma fraction IV, plasma fraction V, or similar composition plasma fractions or a combination thereof.

126. The method of any of claims 124-125, wherein the permeate is filtered against a second filtration membrane, thereby forming a retentate fraction comprising of a mixture of haptoglobin, albumin, hemopexin and transferrin having a molecular weight of greater than about 50 kDa value and a permeate fraction comprising serum proteins and other impurities having a molecular weight of less than about 50 kDa.

127. The method of claim 123, wherein the first cut-off value is from 40 kDa to 60 kDa and wherein the plasma protein concentrate comprises a mixture of haptoglobin, albumin, hemopexin and transferrin.

128. The method of claim 127, wherein the plasma or fraction thereof comprises plasma fraction IV, plasma fraction V, or similar composition plasma fractions or a combination thereof.

129. The method of claim 123, wherein the first cut-off value is from 80 kDa to 120 kDa and wherein the plasma protein concentrate comprises immunoglobulins.

130. The method of claim 129, wherein the plasma or fraction thereof comprises plasma fraction II, plasma fraction III, or similar composition plasma fractions or a combination thereof.

131. The method of claim 123, wherein the first cut-off value is from 450 kDa to 550 kDa and wherein the plasma protein concentrate comprises a Von Willebrand factor.

132. The method of claim 131, wherein the plasma or fraction thereof comprises plasma cryoprecipitates or plasma fraction I, or similar composition plasma fractions or a combination thereof.

133. The method of any of claims 131-132, wherein the permeate is filtered against a second filtration membrane, thereby forming a retentate fraction comprising a mixture Factor VIII, fibronectin, fibrinogen and Von Willebrand factor and a permeate fraction comprising serum proteins.

134. The method of claim 123, wherein the first cut-off value is from 170 kDa to 230 kDa and wherein the plasma protein concentrate comprises Factor VIII and Factor I.

135. The method of claim 134, wherein the plasma or fraction thereof comprises plasma fraction I, or similar composition plasma fractions or a combination thereof.

136. The method of claim 123, wherein the first cut-off value is from 40 kDa to 60 kDa and wherein the plasma protein concentrate comprises a mixture of Factor X and prothrombin.

137. The method of claim 136, wherein the permeate is filtered against a second filtration membrane, thereby forming a retentate fraction comprising Factors VIII, Factor I, Factor X and prothrombin.

138. The method of any of claims 136-137, wherein the plasma or fraction thereof comprises plasma fraction I, or similar composition plasma fractions or a combination thereof.

139. The method of claim 123, wherein the first cut-off value is from 50 kDa to 90 kDa and wherein the plasma protein concentrate comprises haptoglobin.

140. The method of claim 139, wherein the plasma or fraction thereof comprises plasma fraction V, or similar composition plasma fractions or a combination thereof.

141. The method of any of claims 139-140, wherein the permeate is filtered against a second filtration membrane, thereby forming a retentate fraction comprising haptoglobin and albumin.

142. The method of claim 123, wherein the first cut-off value is from 80 kDa to 120 kDa and wherein the plasma protein concentrate comprises ceruloplasmin.

143. The method of claim 142, wherein the permeate is filtered against a second filtration membrane, thereby forming a retentate fraction comprising ceruloplasmin and protein C concentrate.

144. The method of any of claims 142-143, wherein the plasma or fraction thereof comprises plasma fraction IV-1, or similar composition plasma fractions or a combination thereof.

145. The method of any of claims 123-144, wherein the plasma or fraction thereof is pre-filtered through a large cut-off filter to remove large impurities and/or lipoproteins.

146. The method of any of claim 1-70 or 116-145, wherein the filtration is performed in stages with filters of equal molecular weight cut off placed in series with permeate from a stage feed to the following stage to yield higher product recovery at a desired purity level.

147. The method of any of claim 1-70 or 116-145, wherein the method further comprises processing the composition to reduce the concentration of albumin in the composition.

148. The method of claim 147, wherein processing the compositions comprises chromatography, precipitation, or a combination thereof.
